(19) United States
(12) Patent Application Publication
(10) Pub. No.: US 2024/0415984 A1
(43) Pub. Date: Dec. 19, 2024

(54) Precise Excisions of Portions of Exons for Treatment of Duchenne Muscular Dystrophy

(71) Applicant: Vertex Pharmaceuticals Incorporated, Boston, MA (US)

(72) Inventors: Tudor Fulga, Boston, MA (US); Yi-Li Min, Boston, MA (US); Foram Ashar, Boston, MA (US); Su Wang, Boston, MA (US); Jianming Liu, Boston, MA (US)

(73) Assignee: Vertex Pharmaceuticals Incorporated, Boston, MA (US)

(21) Appl. No.: 18/826,876

(22) Filed: Sep. 6, 2024

Related U.S. Application Data
(63) Continuation of application No. PCT/US23/63884, filed on Mar. 7, 2023.
(60) Provisional application No. 63/317,819, filed on Mar. 8, 2022.

(51) Int. Cl. A61K 48/00; C12N 9/22; C12N 15/11; C12N 15/90

(52) U.S. Cl. CPC A61K 48/0058; C12N 9/22; C12N 15/11; C12N 15/907; C12N 2310/20

(57) ABSTRACT

Compositions and methods for treating Duchenne Muscular Dystrophy (DMD) and excising small portions of exons of the DMD gene are encompassed.

This application is a bypass continuation of PCT/US2023/63884, filed Mar. 7, 2023, which claims the benefit of priority to U.S. Provisional Application No. 63/317,819, filed Mar. 8, 2022, both of which are incorporated herein by reference in its entirety.

The instant application contains a Sequence Listing which has been submitted electronically in XML format and is hereby incorporated by reference in its entirety. Said XML copy, created on Mar. 6, 2023, is named 01245-0035-00PCT_Sequence_Listing and is 5,435,392 bytes in size.

### INTRODUCTION AND SUMMARY

Muscular dystrophies (MD) are a group of more than 30 genetic diseases characterized by progressive weakness and degeneration of the skeletal muscles that control movement. Duchenne muscular dystrophy (DMD) is one of the most severe forms of MD that affects approximately 1 in 5000 boys and is characterized by progressive muscle weakness and premature death. Cardiomyopathy and heart failure are common, incurable and lethal features of DMD. The disease is caused by mutations in the gene encoding dystrophin (DMD), which result in loss of expression of dystrophin, causing muscle membrane fragility and progressive muscle wasting.

CRISPR-based genome editing can provide sequence-specific cleavage of genomic DNA using a Cas9 and a guide RNA. For example, a nucleic acid encoding the Cas9 enzyme and a nucleic acid encoding for the appropriate guide RNA can be provided on separate vectors or together on a single vector and administered in vivo or in vitro to knockout or correct a genetic mutation, for example. The approximately 20 nucleotides at the 5′ end of the guide RNA serves as the guide or spacer sequence that can be any sequence complementary to one strand of a genomic target location that has an adjacent protospacer adjacent motif (PAM). The PAM sequence is a short sequence adjacent to the Cas9 nuclease cut site that the Cas9 molecule requires for appropriate binding. The nucleotides 3′ of the guide or spacer sequence of the guide RNA serve as a scaffold sequence for interacting with Cas9. When a guide RNA and a Cas9 are expressed, the guide RNA will bind to Cas9 and direct it to the sequence complementary to the guide sequence, where it will then initiate a double-stranded break (DSB). To repair these breaks, cells typically use an error prone mechanism of non-homologous end joining (NHEJ) which can lead to disruption of function in the target gene through insertions or deletion of codons, shifts in the reading frame, or result in a premature stop codon triggering nonsense-mediated decay. See, e.g., Kumar et al. (2018) Front. Mol. Neurosci. Vol. 11, Article 413.

While gene editing strategies using systems (e.g., CRISPR) for treating DMD have been previously explored, these strategies have focused primarily on either: a) cutting at multiple different sites to excise large portions (e.g., one or more exons) of the dystrophin gene (see, e.g., Ousterout et al., 2015, Nat Commun. 6:6244); or b) cutting in a single site to introduce indels that either result in a frame-shifting mutation and/or that destroy a splice acceptor/donor site in the dystrophin gene (see, e.g., Amoassi et al., 2018, Science, 362(6410):86-91). However, there remains a need for additional alternative and effective gene editing strategies for treating diseases like DMD.

Provided herein are compositions and methods for treating DMD utilizing Cas proteins such as Staphylococcus aureus (SaCas9) and Staphylococcus lugdunensis (SluCas9). In some embodiments, pairs of guide RNAs that excise small portions of the DMD gene are provided, where the nucleic acid encoding the pairs of guide RNAs may be on a single nucleic acid molecule.

Accordingly, the following non-limiting embodiments are provided.
Embodiment 1 is a composition comprising one or more guide RNAs or a nucleic acid encoding one or more guide RNAs, wherein the guide RNAs comprise i) a guide sequence of Table 1A or Table 1B; ii) at least 16, 17, 18, 19, or 20 contiguous nucleotides of a guide sequence of Table 1A or Table 1B; iii) a guide sequence that is at least 90% identical to a guide sequence of Table 1A or Table 1B; optionally further comprising a SaCas9 or a nucleic acid encoding a SaCas9 (for SEQ ID NOs: 1000-3081 in Table 1A) or a SluCas9 or a nucleic acid encoding a SluCas9 (for SEQ ID NOs 4000-5226 in Table 1B).
Embodiment 2 is a composition comprising a pair of guide RNAs or a nucleic acid encoding a pair of guide RNAs, wherein the pair of guide RNAs comprise a first and a second guide sequence, wherein the first and second guide sequences are selected from any two guide sequences of Table 1A or any two guide sequences of Table 1B.
Embodiment 3 is a composition comprising: one or more nucleic acid molecules encoding
   a. a SaCas9 or SluCas9; and
   b. a first guide sequence and a second guide sequence, wherein the first and second guide sequence are selected from any two guide sequences of Table 1A or any two guide sequences of Table 1B.
Embodiment 4 is a composition comprising:
   a. a single nucleic acid molecule comprising:
      i. a nucleic acid encoding Staphylococcus aureus Cas9 (SaCas9) or Staphylococcus lugdunensis (SluCas9) and at least one, at least two, or at least three guide RNAs; or
      ii. a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or
      iii. a nucleic acid encoding Staphylococcus aureus Cas9 (SaCas9) or Staphylococcus lugdunensis (SluCas9) and 1, 2, or 3 guide RNAs;
wherein each guide RNA comprises at least one guide sequence of Table 1A or Table 1B; or
   b. two nucleic acid molecules comprising:
      i. a first nucleic acid encoding Staphylococcus aureus Cas9 (SaCas9) or Staphylococcus lugdunensis (SluCas9); and
         1. a second nucleic acid that does not encode a SaCas9 or SluCas9 and encodes any one of the following:
         2. at least one, at least two, at least three, at least four, at least five, or at least six guide RNAs; or
         3. from one to n guide RNAs, wherein n is no more than the maximum number of guide RNAs that can be expressed from said nucleic acid; or
         4. from one to six guide RNAs; or
      ii. a first nucleic acid encoding Staphylococcus aureus Cas9 (SaCas9) or Staphylococcus lugdunensis (SluCas9) and
         1. at least one, at least two, or at least three guide RNAs; or
         2. from one to n guide RNAs, wherein n is no more than the maximum number of guide RNAs that can be expressed from said nucleic acid; or
         3. 1, 2, or 3 guide RNAs; and
      a second nucleic acid that does not encode a SaCas9 or SluCas9, optionally wherein the second nucleic acid comprises any one of the following:
         1. at least one, at least two, at least three, at least four, at least five, or at least six guide RNAs; or
         2. from one to n guide RNAs, wherein n is no more than the maximum number of guide RNAs that can be expressed from said nucleic acid; or
         3. from one to six guide RNAs; or
      iii. a first nucleic acid encoding Staphylococcus aureus Cas9 (SaCas9) or Staphylococcus lugdunensis (SluCas9) and at least one, at least two, or at least three guide RNAs; and
         a second nucleic acid that does not encode a SaCas9 or SluCas9 and encodes from one to six guide RNAs; or
      iv. a first nucleic acid encoding Staphylococcus aureus Cas9 (SaCas9) or Staphylococcus lugdunensis (SluCas9) and at least two guide RNAs, wherein at least one guide RNA binds upstream of a target sequence and at least one guide RNA binds downstream of the target sequence; and
         a second nucleic acid that does not encode a SaCas9 or SluCas9 and encodes at least one additional copy of each of the guide RNAs encoded in the first nucleic acid;
wherein each guide RNA comprises at least one guide sequence of Table 1A (for SaCas9) or Table 1B (for SluCas9).

Embodiment 5 is the composition of any one of embodiments 1-4, comprising a pair of guide RNAs, wherein the pair of guide RNAs are capable of excising a DNA fragment from the DMD gene; wherein the DNA fragment is between 5-250 nucleotides in length.
Embodiment 6 is the composition of embodiment 5, wherein the excised DNA fragment does not comprise an entire exon of the DMD gene.
Embodiment 7 is a composition comprising a single nucleic acid molecule encoding a pair of guide RNAs and a Cas9, wherein the single nucleic acid molecule comprises:
   a. a first nucleic acid encoding a pair of guide RNAs comprising a first and second spacer sequence, wherein the first and second spacer sequences are selected from any two spacer sequences of SEQ ID NOs: 1000-3081; and a second nucleic acid encoding a Staphylococcus aureus Cas9 (SaCas9); or
   b. a first nucleic acid encoding a pair of guide RNAs comprising a first and second spacer sequence, wherein the first and second spacer sequences are selected from any two spacer sequences of SEQ ID NOs: 4000-5226; and a second nucleic acid encoding a Staphylococcus lugdunensis (SluCas9).
Embodiment 8 is a composition comprising one or more nucleic acid molecules encoding a Staphylococcus aureus Cas9 (SaCas9) and a first and a second guide RNA, wherein the first and second guide RNA target different sequences in a DMD gene, wherein the first and a second guide RNA each comprise a sequence that is at least 90% identical to a first and second spacer sequence, wherein the first and second spacer sequences are selected from any two space sequences of Table 1A.
Embodiment 9 is a composition comprising one or more nucleic acid molecules encoding a Staphylococcus lugdunensis (SluCas9) and a first and a second guide RNA, wherein the first and second guide RNA target different sequences in a DMD gene, wherein the first and a second guide RNA each comprise a sequence that is at least 90% identical to a first and second spacer sequence, wherein the first and second spacer sequences are selected from any two spacer sequences of Table 1B.
Embodiment 10 is the composition of any one of the preceding embodiments, comprising at least two guide RNAs, wherein once expressed in vitro or in vivo in combination with an RNA-guided endonuclease, the guide RNAs excise a portion of the exon, wherein the size of the excised portion of the exon is between 5 and 250 nucleotides in length.
Embodiment 11 is the composition of any one of the preceding embodiments, comprising at least two guide RNAs, wherein once expressed in vitro or in vivo in combination with an RNA-guided endonuclease, the guide RNAs excise a portion of the exon, wherein the size of the excised portion of the exon is between 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, and 50 and 75 nucleotides.
Embodiment 12 is the composition of any one of the preceding embodiments, comprising at least two guide RNAs, wherein once expressed in vitro or in vivo in combination with an RNA-guided endonuclease, the guide RNAs excise a portion of the exon, wherein the size of the excised portion of the exon is between 8 and 167 nucleotides.
Embodiment 13 is the composition of any one of the preceding embodiments, wherein the guide RNA is an sgRNA.
Embodiment 14 is the composition of any one of the preceding embodiments, wherein the guide RNA is modified.
Embodiment 15 is the composition of any one of the preceding embodiments, wherein the one or more guide RNAs or nucleic acids are in a vector.
Embodiment 16 is the composition of embodiment 15, wherein the vector is a viral vector.
Embodiment 17 is the composition of embodiment 16, wherein the viral vector is an AAV vector.
Embodiment 18 is the composition of embodiment 17, wherein the AAV vector is an AAV9 vector.
Embodiment 19 is the composition of any one of the preceding embodiments, wherein the promoter for the one or more guide RNAs is hU6c.
Embodiment 20 is the composition of any one of the preceding embodiments, wherein if the one or more guide RNAs is a guide RNA for SaCas9, the one or more guide RNAs comprise a scaffold comprising the sequence of SEQ ID NO: 504.
Embodiment 21 is the composition of any one of the preceding embodiments, wherein if the one or more guide RNAs is a guide RNA for SluCas9, the one or more guide RNAs comprise a scaffold comprising the sequence of SEQ ID NO: 901.
Embodiment 22 is the composition of any one of the preceding embodiments, wherein the one or more guide RNAs are in an AAV vector, wherein the vector comprises from 5′ to 3′ with respect to the plus strand: the reverse complement of a first guide RNA scaffold sequence; the reverse complement of a nucleic acid encoding a first guide RNA guide sequence; the reverse complement of a promoter for expression of the nucleic acid encoding the first guide RNA; a promoter (e.g., CK8e) for expression of a nucleic acid encoding a SaCas9, SluCas9, or a sRGN; a nucleic acid encoding a SaCas9, SluCas9, or a sRGN, a polyadenylation sequence; a promoter for expression of a second sgRNA in the same direction as the promoter for SaCas9, SluCas9, or the sRGN; a second sgRNA guide sequence; and a second sgRNA scaffold sequence.
Embodiment 23 is the composition of any one of the preceding embodiments, wherein the composition comprises a pair of guide RNAs, wherein the first and second guide RNAs in the pair target the same exon in the dystrophin gene.
Embodiment 24 is the composition of embodiment 23, wherein the first guide RNA and the second guide RNA are not the same.
Embodiment 25 is the composition of embodiment 23, wherein the first guide RNA targets a different site in the same exon targeted by the second guide RNA.
Embodiment 26 is the composition of embodiment 23, wherein the exon is selected from the group consisting of exons: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 46, 47, 48, 49, 52, 54, 55, 56, 57, 58, 59, 60, 61, 62, 63, 64, 65, 66, 67, 68, 69, 70, 71, 72, 73, 74, 75, 76, 77, 78, or 79 of the dystrophin gene.
Embodiment 27 is a composition of any one of claims 1-26, wherein the one or more guide RNA sequences, or the first and/or second guide RNA sequence of the pair of guide RNAs, is no more than 16, no more than 17, no more than 18, no more than 19, no more than 20, no more than 21, or no more than 22 nucleotides in length.
Embodiment 28 is a method of treating Duchenne Muscular Dystrophy (DMD), the method comprising delivering to a cell a composition of any one of embodiments 1-27.
Embodiment 29 is a method of excising a portion of the DMD gene, the method comprising delivering to a cell a composition of any one of embodiments 1-27, wherein the size of the excised portion is less than about 250 nucleotides.

### DESCRIPTION OF FIGURES

FIG. 1 is a schematic showing four representative vector designs (Designs 1-4). White arrows indicate directionality of expression of the sgRNA(s), while the black arrows indicate directionality of the Cas9 protein. In a particular embodiment, the Cas9 promoter may be CK8e. “Pol III” refers to a representative promoter for the expression of sgRNAs, “g1” and “g2” each refer to a guide sequence, “scaffold” refers to the scaffold of a guide RNA, and “pa” refers to a polyadenylation sequence.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA” and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. For clarity, the terms “spacer sequence”, “spacer,” “protospacer,” “guide sequence,” or “targeting sequence” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. A guide sequence can be 24, 23, 22, 21, 20 or fewer base pairs in length, e.g., in the case of Staphylococcus lugdunensis (i.e., SluCas9) or Staphylococcus aureus (i.e., SaCas9) and related Cas9 homologs/orthologs. In preferred embodiments, a guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). Shorter or longer sequences can also be used as guides, e.g., 15-, 16-, 17-, 18-, 19-, 20-, 21-, 22-, 23-, 24-, or 25-nucleotides in length. For example, in some embodiments, the guide sequence comprises at least 16, 17, 18, 19, 20, 21, 22, 23, 24, or 25 contiguous nucleotides of a sequence selected from SEQ ID NOs: 1000-3081 (for SaCas9, including SaCas9KKH), and 4000-5226 (for SluCas9). In some embodiments, the guide sequence comprises a sequence selected from SEQ ID NOs: 1000-3081 or 4000-5226. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 16, 17, 18, 19, 20, 21, 22, 23, 24, or 25 contiguous nucleotides of a sequence selected from SEQ ID NOs: 1000-3081 or 4000-5226. In some embodiments, the guide sequence comprises a sequence with about 75%, 80%, 85%, 90%, 95%, 96%, 97%, 98%, 99%, or 100% identity to a sequence selected from SEQ ID NOs: 1000-3081 or 4000-5226. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 16, 17, 18, 19, 20 or more base pairs. In some embodiments, the total length of the target sequence/guide is not more than 16, no more than 17, no more than 18, no more than 19, no more than 20, no more than 22, no more than 23, or no more than 24 nucleotides in length. In some embodiments, the guide sequence and the target region may contain 1-4 mismatches where the guide sequence comprises at least 16, 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

In some embodiments, the guide sequence comprises a sequence selected from SEQ ID NOs: 1000-3081 or 4000-5226, wherein if the 5′ terminal nucleotide is not guanine, one or more guanine (g) is added to the sequence at its 5′ end. In some embodiments, the 5′ g or gg is included in some instances for transcription, for example, for expression by the RNA polymerase III-dependent U6 promoter or the T7 promoter. In some embodiments, a 5′ guanine is added to any one of the guide sequences or pairs of guide sequences disclosed herein.

Target sequences for Cas9s include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “ribonucleoprotein” (RNP) or “RNP complex” refers to a guide RNA together with a Cas9. In some embodiments, the guide RNA guides the Cas9, such as a SluCas9 or a SaCas9, to a target sequence, and the guide RNA hybridizes with and the agent binds to the target sequence, which can be followed by cleaving or nicking (in the context of a modified “nickase” Cas9).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Guide sequences useful in the guide RNA compositions and methods described herein are shown, for example, in Tables 1A-1B, and throughout the specification.

As used herein, a “target sequence” refers to a sequence of nucleic acid in a target gene that has complementarity to at least a portion of the guide sequence of the guide RNA. The interaction of the target sequence and the guide sequence directs a Cas9 to bind, and potentially nick or cleave (depending on the activity of the agent), within or near the target sequence.

As used herein, “treatment” refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. For example, treatment of DMD may comprise alleviating symptoms of DMD.

As used herein, “ameliorating” refers to any beneficial effect on a phenotype or symptom, such as reducing its severity, slowing or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

A “pharmaceutically acceptable excipient” refers to an agent that is included in a pharmaceutical formulation that is not the active ingredient. Pharmaceutically acceptable excipients may e.g., aid in drug delivery or support or enhance stability or bioavailability.

The term “about” or “approximately” means an acceptable error for a particular value as determined by one of ordinary skill in the art, which depends in part on how the value is measured or determined.

As used herein, “Staphylococcus aureus Cas9” may also be referred to as SaCas9, and includes wild type SaCas9 (e.g., SEQ ID NO: 711) and variants thereof. A variant of SaCas9 comprises one or more amino acid changes as compared to SEQ ID NO: 711, including insertion, deletion, or substitution of one or more amino acids, or a chemical modification to one or more amino acids. For clarity, SaCas9KKH is a SaCas9 variant.

As used herein, “Staphylococcus lugdunensis Cas9” may also be referred to as SluCas9, and includes wild type SluCas9 (e.g., SEQ ID NO: 712) and variants thereof. A variant of SluCas9 comprises one or more amino acid changes as compared to SEQ ID NO: 712, including insertion, deletion, or substitution of one or more amino acids, or a chemical modification to one or more amino acids.

### II. Compositions

Provided herein are compositions comprising guide RNAs and pairs of guide RNAs useful for treating Duchenne Muscular Dystrophy (DMD). The provided pairs of guide RNAs, when used with the correct endonuclease, function to precisely delete a small portion (e.g., less than about 250 nucleotides) of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 46, 47, 48, 49, 52, 54, 55, 56, 57, 58, 59, 60, 61, 62, 63, 64, 65, 66, 67, 68, 69, 70, 71, 72, 73, 74, 75, 76, 77, 78, or 79 of the DMD gene. Tables 1A-B provides a listing of guide sequences of guide RNAs, including detailed information regarding these sequences.

### Guides and Guide Pairs

TABLE 1A
Sa guide sequences (human-hg38.12)

| | Guide ID | | | | | |
|---|---|---|---|---|---|---|
| Seq ID No. | or Guide RNA Name | exon_id | enzyme | strand | Guide Sequence | pam |
| 1000 | E1Sa1 | exon1 | SaCas9 | + | CGAAGGTAATTGCCTCCCAGAT | CTGAGT |
| 1001 | E1Sa2 | exon1 | SaCas9 | + | ATCTGAGTCCTGTAGGGGGAAA | GTGAGT |
| 1002 | E1Sa5 | exon1 | SaCas9 | − | AGGCAATTACCTTCGGAGAAAA | ACGAAT |
| 1003 | E2Sa1 | exon2 | SaCas9 | + | AAATTGTGCATTTACCCATTTT | GTGAAT |
| 1004 | E2Sa2 | exon2 | SaCas9 | − | TAAATGCACAATTTTCTAAGGT | AAGAAT |
| 1005 | E2Sa3 | exon2 | SaCas9 | − | TTCAAAAGAAAACATTCACAAA | ATGGGT |
| 1006 | E3Sa1 | exon3 | SaCas9 | − | TGAGAACCTCTTCAGTGACCTA | CAGGAT |
| 1007 | E4Sa1 | exon4 | SaCas9 | + | ATTGTTCAGGGCATGAACTCTT | GTGGAT |
| 1008 | E4Sa4 | exon4 | SaCas9 | − | TGAACAATGTCAACAAGGCACT | GCGGGT |
| 1009 | E4Sa5 | exon4 | SaCas9 | − | AGCCAAAAGAAAAAGGATCCAC | AAGAGT |
| 1010 | E4Sa6 | exon4 | SaCas9 | − | TTTTGTTCTCAGCCAAAAGAAA | AAGGAT |
| 1011 | E5Sa1 | exon5 | SaCas9 | + | TCAGGATTCTTACCTGCCAGTG | GAGGAT |
| 1012 | E5Sa2 | exon5 | SaCas9 | + | GGATTATATTCCAAATCAAACC | AAGAGT |
| 1013 | E5Sa5 | exon5 | SaCas9 | − | ATATAATCCTCCACTGGCAGGT | AAGAAT |
| 1014 | E5Sa6 | exon5 | SaCas9 | − | TAAACTGACTCTTGGTTTGATT | TGGAAT |
| 1015 | E5Sa7 | exon5 | SaCas9 | − | CTTTCTTTAACAGGTTGATTTA | GTGAAT |
| 1016 | E6Sa1 | exon6 | SaCas9 | + | AGTAATCTTCTTACCTATGACT | ATGGAT |
| 1017 | E6Sa2 | exon6 | SaCas9 | + | GTGAAGTTGATTACATTAACCT | GTGGAT |
| 1018 | E6Sa3 | exon6 | SaCas9 | + | TTACATTAACCTGTGGATAATT | ACGAGT |
| 1019 | E6Sa4 | exon6 | SaCas9 | + | TTGATTGTCGGACCCAGCTCAG | GAGAAT |
| 1020 | E6Sa5 | exon6 | SaCas9 | − | CAGCTGGTCTGATGGCCTGGCT | TTGAAT |
| 1021 | E6Sa6 | exon6 | SaCas9 | − | ACAGTGAAAAGATTCTCCTGAG | CTGGGT |
| 1022 | E6Sa7 | exon6 | SaCas9 | − | AATGTAATGAAAAATATCATGG | CTGGAT |
| 1023 | E7Sa1 | exon7 | SaCas9 | + | AGTCCAGAAATTTACCAACCTT | CAGGAT |
| 1024 | E7Sa2 | exon7 | SaCas9 | + | AGAAATTTACCAACCTTCAGGA | TCGAGT |
| 1025 | E7Sa3 | exon7 | SaCas9 | + | GCCTAATTGATATCTGGCGATG | TTGAAT |
| 1026 | E7Sa4 | exon7 | SaCas9 | − | TTTTAGGCCAGACCTATTTGAC | TGGAAT |
| 1027 | E8Sa1 | exon8 | SaCas9 | + | TCCTTTACACACTTTACCTGTT | GAGAAT |
| 1028 | E8Sa2 | exon8 | SaCas9 | + | GCCTTGGCAACATTTCCACTTC | CTGGAT |
| 1029 | E8Sa3 | exon8 | SaCas9 | + | CACTTGTTGAGGCAAAACTTGG | AAGAGT |
| 1030 | E8Sa4 | exon8 | SaCas9 | + | TACATTAAGATGGACTTCTTAT | CTGGAT |
| 1031 | E9Sa1 | exon9 | SaCas9 | + | TGTGAAGGAAATGGGCTCCGTG | TAGGGT |
| 1032 | E9Sa2 | exon9 | SaCas9 | + | AGCCTGTGTGTAGGCATAGCTC | TTGAAT |
| 1033 | E9Sa6 | exon9 | SaCas9 | − | CAGATCACGGTCAGTCTAGCAC | AGGGAT |
| 1034 | E10Sa1 | exon10 | SaCas9 | + | AATTAACGTTTTAGTTTACCTC | ATGAGT |
| 1035 | E10Sa3 | exon10 | SaCas9 | − | GTCATTTGGCAGTTCATTGATG | GAGAGT |
| 1036 | E11Sa1 | exon11 | SaCas9 | + | GCTACCCTGAGGCATTCCCATC | TTGAAT |
| 1037 | E11Sa2 | exon11 | SaCas9 | + | CAATCAGCTTACTTCCCAATTG | TAGAAT |
| 1038 | E11Sa3 | exon11 | SaCas9 | − | TAAATTCAAGATGGGAATGCCT | CAGGGT |
| 1039 | E11Sa4 | exon11 | SaCas9 | − | ATGAATCTCCTAAATTCAAGAT | GGGAAT |
| 1040 | E11Sa5 | exon11 | SaCas9 | − | AGAAACTGAAGTACAAGAGCAG | ATGAAT |
| 1041 | E11Sa6 | exon11 | SaCas9 | − | TGGATTTGACAGCCCATCAGGG | CCGGGT |
| 1042 | E11Sa7 | exon11 | SaCas9 | − | CCTTCTTTGTCAGGGGTACATG | ATGGAT |
| 1043 | E12Sa1 | exon12 | SaCas9 | − | CCAGAATCAGAAACTGAAAGAG | TTGAAT |
| 1044 | E12Sa2 | exon12 | SaCas9 | − | GATCTCCAGAATCAGAAACTGA | AAGAGT |
| 1045 | E12Sa3 | exon12 | SaCas9 | − | ACATAGAGTTTTAATGGATCTC | CAGAAT |
| 1046 | E12Sa4 | exon12 | SaCas9 | − | TTTCAGTTTACATAGAGTTTTA | ATGGAT |
| 1047 | E12Sa5 | exon12 | SaCas9 | − | TCTTTCAAATTTTCAGTTTACA | TAGAGT |
| 1048 | E13Sa1 | exon13 | SaCas9 | + | ATTCATCAACTACCACCACCAT | GTGAGT |
| 1049 | E13Sa2 | exon13 | SaCas9 | + | ACTACCACCACCATGTGAGTGA | GAGAAT |
| 1050 | E13Sa3 | exon13 | SaCas9 | − | ACTCACATGGTGGTGGTAGTTG | ATGAAT |
| 1051 | E13Sa4 | exon13 | SaCas9 | − | AAGATCTAGAACAAGAACAAGT | CAGGGT |
| 1052 | E14Sa1 | exon14 | SaCas9 | + | CAGTAAGACGTTGCCATTTGAG | AAGGAT |
| 1053 | E14Sa2 | exon14 | SaCas9 | − | TCTGTAGATGGACAGAAGACCG | CTGGGT |
| 1054 | E15Sa1 | exon15 | SaCas9 | + | TTTGATCTTTAAAGCCAGTTGT | GTGAAT |
| 1055 | E16Sa1 | exon16 | SaCas9 | + | TATCCAGCCATGCTTCCGTCTT | CTGGGT |
| 1056 | E16Sa2 | exon16 | SaCas9 | + | CAGTGTTGAAAGAAGATCTTGT | TTGAGT |
| 1057 | E16Sa3 | exon16 | SaCas9 | + | GTTGAAAGAAGATCTTGTTTGA | GTGAAT |
| 1058 | E16Sa4 | exon16 | SaCas9 | + | TTTGAGTGAATACAGTTTGCCC | ATGGAT |
| 1059 | E16Sa6 | exon16 | SaCas9 | − | TAATTTAGTCCAAAAACTTGAA | AAGAGT |
| 1060 | E16Sa7 | exon16 | SaCas9 | − | GCTGGATAACTTTGCCCGGTGT | TGGGAT |
| 1061 | E16Sa8 | exon16 | SaCas9 | − | GACCCAGAAGACGGAAGCATGG | CTGGAT |
| 1062 | E16Sa9 | exon16 | SaCas9 | − | ACAAGATCTTCTTTCAACACTG | AAGAAT |
| 1063 | E16Sa10 | exon16 | SaCas9 | − | CTTGTTTTAACAGGTTTTAAAA | GCGGAT |
| 1064 | E17Sa1 | exon17 | SaCas9 | + | TGCTCTCACCTTTTCCTAATTT | CAGAAT |
| 1065 | E17Sa2 | exon17 | SaCas9 | + | GTTCCTCTTGAGCATGCTTTAC | CAGGAT |
| 1066 | E17Sa3 | exon17 | SaCas9 | + | CAGTTGTCTGTGTTAGTGATGG | CTGAGT |
| 1067 | E17Sa4 | exon17 | SaCas9 | − | CCAAAAGAAGAGGCAGATTACT | GTGGAT |
| 1068 | E18Sa1 | exon18 | SaCas9 | + | GACTCTGCAACACAGCTTCTGA | GCGAGT |
| 1069 | E18Sa2 | exon18 | SaCas9 | − | TCAGAAGCTGTGTTGCAGAGTC | CTGAAT |
| 1070 | E18Sa3 | exon18 | SaCas9 | − | TACTCGCTCAGAAGCTGTGTTG | CAGAGT |
| 1071 | E18Sa4 | exon18 | SaCas9 | − | TTGATATAACTGAACTTCACAG | CTGGAT |
| 1072 | E19Sa3 | exon19 | SaCas9 | − | TCAGGCCCTGGTGGAACAGATG | GTGAAT |
| 1073 | E20Sa1 | exon20 | SaCas9 | + | TTTTAATTGCTGTTGGCTCTGA | TGGGGT |
| 1074 | E20Sa2 | exon20 | SaCas9 | + | TGCTGTTGGCTCTGATGGGGTG | GTGGGT |
| 1075 | E20Sa3 | exon20 | SaCas9 | + | TTGGCTCTGATGGGGTGGTGGG | TTGGAT |
| 1076 | E20Sa4 | exon20 | SaCas9 | + | AAGTCTCTCACTTAGCAACTGG | CAGAAT |
| 1077 | E20Sa5 | exon20 | SaCas9 | − | GTCAGTTAAAAATTTGTAAGGT | AAGAAT |
| 1078 | E20Sa6 | exon20 | SaCas9 | − | CTAAGTGAGAGACTTAACTGGC | TGGAGT |
| 1079 | E20Sa7 | exon20 | SaCas9 | − | GAACAACTGAACAGCCGGTGGA | TCGAAT |
| 1080 | E20Sa8 | exon20 | SaCas9 | − | CCTCAGAACAACTGAACAGCCG | GTGGAT |
| 1081 | E21Sa1 | exon21 | SaCas9 | + | CACAAAGTCTGCATCCAGGAAC | ATGGGT |
| 1082 | E21Sa2 | exon21 | SaCas9 | + | CTTTCTCTTTCAGGGCTATGCT | TTGAAT |
| 1083 | E21Sa4 | exon21 | SaCas9 | − | GAAAGGACAAGGACCCATGTTC | CTGGAT |
| 1084 | E22Sa1 | exon22 | SaCas9 | + | ATATTCACAGACCTGCAATTCC | CCGAGT |
| 1085 | E22Sa2 | exon22 | SaCas9 | + | GGTGACACTAAGTTGAGGTATG | GAGAGT |
| 1086 | E22Sa7 | exon22 | SaCas9 | − | GAAATCATGGAGCAGAGACTCG | GGGAAT |
| 1087 | E22Sa8 | exon22 | SaCas9 | − | AGACCATGAGTGCCATCAGGAC | ATGGGT |
| 1088 | E22Sa9 | exon22 | SaCas9 | − | ACCAATGCGCTATCAGGAGACC | ATGAGT |
| 1089 | E23Sa3 | exon23 | SaCas9 | + | ATCTTGAATTACCTGAATTTTT | CGGAGT |
| 1090 | E23Sa4 | exon23 | SaCas9 | − | TTGTCAAAAGCTAGAGGAGCAA | ATGAAT |
| 1091 | E23Sa5 | exon23 | SaCas9 | − | GAAATTAGCCGGAAATATCAAT | CAGAAT |
| 1092 | E24Sa1 | exon24 | SaCas9 | + | TGCACTGTTTCAGCTGCTTTTT | TAGAAT |
| 1093 | E24Sa2 | exon24 | SaCas9 | + | TTCAGCTGCTTTTTTAGAATTT | CTGAAT |
| 1094 | E24Sa3 | exon24 | SaCas9 | + | CAACTTCAGCCATCCATTTCTT | CAGGGT |
| 1095 | E24Sa4 | exon24 | SaCas9 | − | TAAAAAAGCAGCTGAAACAGTG | CAGAGT |
| 1096 | E24Sa5 | exon24 | SaCas9 | − | GAAGGAGGAATGGCCTGCCCTT | GGGGAT |
| 1097 | E24Sa6 | exon24 | SaCas9 | − | GAAGTTGATGTTTTTCTGAAGG | AGGAAT |
| 1098 | E24Sa7 | exon24 | SaCas9 | − | ATCACATACAAACCCTGAAGAA | ATGGAT |
| 1099 | E25Sa1 | exon25 | SaCas9 | + | CCTGTTGGCACATGTGATCCCA | CTGAGT |
| 1100 | E25Sa2 | exon25 | SaCas9 | + | ATCCCACTGAGTGTTAAGTTCT | TTGAGT |
| 1101 | E25Sa3 | exon25 | SaCas9 | + | CATTGACACTGTTTAGACTGGG | CTGAAT |
| 1102 | E25Sa4 | exon25 | SaCas9 | + | TGTTTAGACTGGGCTGAATTGT | CTGAAT |
| 1103 | E25Sa5 | exon25 | SaCas9 | − | ACTCAAAGAACTTAACACTCAG | TGGGAT |
| 1104 | E25Sa6 | exon25 | SaCas9 | − | AAGATAAAGAATGAAGCAGAGC | CAGAGT |
| 1105 | E25Sa7 | exon25 | SaCas9 | − | CAATGAAGGTGGGCAGAAGATA | AAGAAT |
| 1106 | E26Sa1 | exon26 | SaCas9 | + | CAACTGCTTTCTGTAATTCATC | TGGAGT |
| 1107 | E26Sa2 | exon26 | SaCas9 | − | GATTTTGAATATAAAACTCCAG | ATGAAT |
| 1108 | E26Sa3 | exon26 | SaCas9 | − | GAAGAGTATCTTGAGAGAGATT | TTGAAT |
| 1109 | E26Sa4 | exon26 | SaCas9 | − | GAATGGATGACACAAGCTGAAG | AAGAGT |
| 1110 | E26Sa5 | exon26 | SaCas9 | − | AAGATCTATCAGAGATGCACGA | ATGGAT |
| 1111 | E26Sa6 | exon26 | SaCas9 | − | CAGAAAGATCTATCAGAGATGC | ACGAAT |
| 1112 | E27Sa1 | exon27 | SaCas9 | + | AGAGCCACTGGTAGTTGGTGGT | TAGAGT |
| 1113 | E27Sa2 | exon27 | SaCas9 | + | GACACTATTTACAGACTCAGTA | AGGAGT |
| 1114 | E27Sa4 | exon27 | SaCas9 | − | CTACCAGTGGCTCTGCACTAGG | CTGAAT |
| 1115 | E27Sa5 | exon27 | SaCas9 | − | GAAGCGAAAGTGAAACTCCTTA | CTGAGT |
| 1116 | E28Sa1 | exon28 | SaCas9 | + | CAGAGATTTCCTCAGCTCCGCC | AGGAAT |
| 1117 | E28Sa2 | exon28 | SaCas9 | − | GCAAACAAGTGGCTAAATGAAG | TAGAAT |
| 1118 | E28Sa3 | exon28 | SaCas9 | − | TAGGAAGTTTGGGCATGTTGGC | ATGAGT |
| 1119 | E29Sa1 | exon29 | SaCas9 | + | TCTTCATGTAGTTCCCTCCAAC | GAGAAT |
| 1120 | E29Sa2 | exon29 | SaCas9 | + | CATCCATGACTCCGCCATCTGT | TAGGGT |
| 1121 | E29Sa3 | exon29 | SaCas9 | + | CTGTTAGGGTCTGTGCCAATAT | GCGAAT |
| 1122 | E29Sa4 | exon29 | SaCas9 | + | TGTGCCAATATGCGAATCTGAT | TTGGGT |
| 1123 | E29Sa5 | exon29 | SaCas9 | + | CGAATCTGATTTGGGTTATCCT | CTGAAT |
| 1124 | E29Sa6 | exon29 | SaCas9 | − | GACCCTAACAGATGGCGGAGTC | ATGGAT |
| 1125 | E29Sa7 | exon29 | SaCas9 | − | TGGCACAGACCCTAACAGATGG | CGGAGT |
| 1126 | E29Sa8 | exon29 | SaCas9 | − | TGAAAATTTGATGCGACATTCA | GAGGAT |
| 1127 | E30Sa1 | exon30 | SaCas9 | + | ATAAGCTGCCAACTGCTTGTCA | ATGAAT |
| 1128 | E30Sa2 | exon30 | SaCas9 | + | TGTCAATGAATGTGAGGGACTC | CTGGAT |
| 1129 | E30Sa3 | exon30 | SaCas9 | + | GAGGGACTCCTGGATTAAGTGT | AAGGAT |
| 1130 | E30Sa4 | exon30 | SaCas9 | + | ATTTTTCAGTCTCCTGGGCAGA | CTGGAT |
| 1131 | E30Sa8 | exon30 | SaCas9 | − | GAAAAATCCTTACACTTAATCC | AGGAGT |
| 1132 | E31Sa1 | exon31 | SaCas9 | + | TCTCATGACTTGTCAAATCAGA | TTGGAT |
| 1133 | E31Sa3 | exon31 | SaCas9 | − | ATCAGGGGAAGGAGGCTGCCCA | AAGAGT |
| 1134 | E32Sa1 | exon32 | SaCas9 | + | CTGCTCAAAATTGGCTGGTTTC | TGGAAT |
| 1135 | E32Sa2 | exon32 | SaCas9 | − | GCACTTGCCTGCATTGGAAACA | AAGAGT |
| 1136 | E33Sa1 | exon33 | SaCas9 | + | GTTACTCTTTCATCAAGTTCTT | TGGGAT |
| 1137 | E33Sa2 | exon33 | SaCas9 | − | AAAATCCCAAAGAACTTGATGA | AAGAGT |
| 1138 | E33Sa3 | exon33 | SaCas9 | − | AACTTAGAACTTGTATAAAAGT | CTGAGT |
| 1139 | E34Sa1 | exon34 | SaCas9 | + | TTTACCTTTCCCCAGGCAACTT | CAGAAT |
| 1140 | E34Sa3 | exon34 | SaCas9 | − | AGTTGAAGGAATGCCTAGTAAT | TTGGAT |
| 1141 | E34Sa4 | exon34 | SaCas9 | − | TGACAAAGAGATCAGCAGTTGA | AGGAAT |
| 1142 | E34Sa5 | exon34 | SaCas9 | − | GAATGGCTGGCAGCTACAGATA | TGGAAT |
| 1143 | E34Sa6 | exon34 | SaCas9 | − | CGAAAGGAAATGAATGTCTTGA | CAGAAT |
| 1144 | E34Sa7 | exon34 | SaCas9 | − | GTCCCGTAAGATGCGAAAGGAA | ATGAAT |
| 1145 | E35Sa1 | exon35 | SaCas9 | + | TATCCAGTTACTATTCAGAAGA | CTGAGT |
| 1146 | E35Sa2 | exon35 | SaCas9 | + | CCTTCTGTTTCTCAATCTCTTT | TTGAGT |
| 1147 | E35Sa3 | exon35 | SaCas9 | − | ATAGCTGTCACCTCCCGAGCAG | AAGAGT |
| 1148 | E35Sa4 | exon35 | SaCas9 | − | AACTCAGTCTTCTGAATAGTAA | CTGGAT |
| 1149 | E35Sa5 | exon35 | SaCas9 | − | GGTGGAAGATAAACTCAGTCTT | CTGAAT |
| 1150 | E35Sa6 | exon35 | SaCas9 | − | TGAGAAACAGAAGGTGCACCTG | AAGAGT |
| 1151 | E36Sa1 | exon36 | SaCas9 | + | CTTAAGCACGTCTTCTTTTTGC | TGGGGT |
| 1152 | E36Sa2 | exon36 | SaCas9 | + | ATTCATCCAAAAGTGTGTCAGC | CTGAAT |
| 1153 | E36Sa3 | exon36 | SaCas9 | − | ATTCAGGCTGACACACTTTTGG | ATGAAT |
| 1154 | E36Sa4 | exon36 | SaCas9 | − | GATCATTCAGGCTGACACACTT | TTGGAT |
| 1155 | E36Sa5 | exon36 | SaCas9 | − | AGAATGTGGACCACATCACAAA | GTGGAT |
| 1156 | E36Sa6 | exon36 | SaCas9 | − | GAAACACATGGAAACTTTTGAC | CAGAAT |
| 1157 | E37Sa1 | exon37 | SaCas9 | + | AAGTTTGCTGCTTGGTCACGTG | TAGAGT |
| 1158 | E37Sa2 | exon37 | SaCas9 | − | ATCGATTTGCAGCCATTTCACA | CAGAAT |
| 1159 | E37Sa3 | exon37 | SaCas9 | − | AATATAGCGTTTAAAGGCAGAA | CTGAAT |
| 1160 | E38Sa1 | exon38 | SaCas9 | + | CCTCTTTCAGATTCACCCCCTG | CTGAAT |
| 1161 | E38Sa2 | exon38 | SaCas9 | + | GGTTCAAGCAATTTTTGTATAT | CTGAGT |
| 1162 | E38Sa3 | exon38 | SaCas9 | + | TAAACTGCTCCAATTCCTTCAA | AGGAAT |
| 1163 | E38Sa5 | exon38 | SaCas9 | − | GGAGGCTGAAATTCAGCAGGGG | GTGAAT |
| 1164 | E38Sa6 | exon38 | SaCas9 | − | CACTGGAGGCTGAAATTCAGCA | GGGGGT |
| 1165 | E38Sa7 | exon38 | SaCas9 | − | TTTTAGGCCTCCATTCCTTTGA | AGGAAT |
| 1166 | E39Sa1 | exon39 | SaCas9 | − | AAAGAGGAGACAACTTACAACA | AAGAAT |
| 1167 | E39Sa2 | exon39 | SaCas9 | − | GAAGACAATGAGGGTACTGTAA | AAGAAT |
| 1168 | E39Sa3 | exon39 | SaCas9 | − | TTTTGATCAGAATGAAGACAAT | GAGGGT |
| 1169 | E40Sa2 | exon40 | SaCas9 | − | GGCTGATGATCTCCTGAAATGC | TTGGAT |
| 1170 | E41Sa1 | exon41 | SaCas9 | + | ACAACTCACAATTTGTGCAAAG | TTGAGT |
| 1171 | E41Sa2 | exon41 | SaCas9 | + | TTTCCCGCCAGCGCTTGCTGAG | CTGGAT |
| 1172 | E41Sa3 | exon41 | SaCas9 | + | GCCAGCGCTTGCTGAGCTGGAT | CTGAGT |
| 1173 | E41Sa4 | exon41 | SaCas9 | − | AAGACTCAACTTTGCACAAATT | GTGAGT |
| 1174 | E41Sa5 | exon41 | SaCas9 | − | TAGGCAAGCTGAGGGCTTGTCT | GAGGAT |
| 1175 | E41Sa6 | exon41 | SaCas9 | − | ATTGCAGAAGAAGAAAGAGGAG | CTGAAT |
| 1176 | E41Sa7 | exon41 | SaCas9 | − | TTTGCTCAATAGGAAATTGATC | GGGAAT |
| 1177 | E42Sa1 | exon42 | SaCas9 | + | CTTCTAATAGGGCTTGTGAGAC | ATGAGT |
| 1178 | E42Sa2 | exon42 | SaCas9 | − | TTTGAAGATCTCTTTAAGCAAG | AGGAGT |
| 1179 | E43Sa1 | exon43 | SaCas9 | + | GCAATGCTGCTGTCTTCTTGCT | ATGAAT |
| 1180 | E43Sa2 | exon43 | SaCas9 | − | AACAAAATGTACAAGGACCGAC | AAGGGT |
| 1181 | E43Sa3 | exon43 | SaCas9 | − | TGCAAAGTGCAACGCCTGTGGA | AAGGGT |
| 1182 | E43Sa4 | exon43 | SaCas9 | − | ATAGTCTACAACAAAGCTCAGG | TCGGAT |
| 1183 | E46Sa1 | exon46 | SaCas9 | + | GCTGCTCTTTTCCAGGTTCAAG | TGGGAT |
| 1184 | E46Sa3 | exon46 | SaCas9 | − | TCAGAATTTCAAAGAGATTTAA | ATGAAT |
| 1185 | E46Sa4 | exon46 | SaCas9 | − | GAAGAACAAAAGAATATCTTGT | CAGAAT |
| 1186 | E46Sa5 | exon46 | SaCas9 | − | TTTCTCCAGGCTAGAAGAACAA | AAGAAT |
| 1187 | E47Sa1 | exon47 | SaCas9 | + | GCTTATGGGAGCACTTACAAGC | ACGGGT |
| 1188 | E47Sa2 | exon47 | SaCas9 | + | CTCCAGTTTCATTTAATTGTTT | GAGAAT |
| 1189 | E47Sa3 | exon47 | SaCas9 | − | AGCTCAAGCAGACAAATCTCCA | GTGGAT |
| 1190 | E47Sa4 | exon47 | SaCas9 | − | TGGAAGAGTTGCCCCTGCGCCA | GGGAAT |
| 1191 | E47Sa5 | exon47 | SaCas9 | − | TGTCTGTTTCAGTTACTGGTGG | AAGAGT |
| 1192 | E48Sa1 | exon48 | SaCas9 | − | GCTGCTGTGGTTATCTCCTATT | AGGAAT |
| 1193 | E49Sa1 | exon49 | SaCas9 | + | GTTGCTTCATTACCTTCACTGG | CTGAGT |
| 1194 | E49Sa2 | exon49 | SaCas9 | − | AATAGCAGTTCAAGCTAAACAA | CCGGAT |
| 1195 | E52Sa1 | exon52 | SaCas9 | + | TTTCAAATTTTGGGCAGCGGTA | ATGAGT |
| 1196 | E52Sa3 | exon52 | SaCas9 | − | TCAAGAGGCTAGAACAATCATT | ACGGAT |
| 1197 | E52Sa4 | exon52 | SaCas9 | − | TTTGTTCTTACAGGCAACAATG | CAGGAT |
| 1198 | E54Sa1 | exon54 | SaCas9 | + | AATAATGTAATTCATACCTTTT | ATGAAT |
| 1199 | E54Sa2 | exon54 | SaCas9 | + | TGGACTTTTCTGGTATCATCTG | CAGAAT |
| 1200 | E54Sa5 | exon54 | SaCas9 | − | TGGAGAAGCATTCATAAAAGGT | ATGAAT |
| 1201 | E54Sa6 | exon54 | SaCas9 | − | CAGAAAAGTCCACATGATAACA | GAGAAT |
| 1202 | E54Sa7 | exon54 | SaCas9 | − | TGACTTGGCCCTGAAACTTCTC | CGGGAT |
| 1203 | E55Sa1 | exon55 | SaCas9 | + | TTCATCAGCTCTTTTACTCCCT | TGGAGT |
| 1204 | E55Sa2 | exon55 | SaCas9 | + | AGTCTTCTAGGAGCCTTTCCTT | ACGGGT |
| 1205 | E55Sa3 | exon55 | SaCas9 | + | GGGGGAACTGTTGCAGTAATCT | ATGAGT |
| 1206 | E55Sa4 | exon55 | SaCas9 | − | AAAGGCTCCTAGAAGACTCCAA | GGGAGT |
| 1207 | E55Sa5 | exon55 | SaCas9 | − | TGAAACAACTGCCAATGTCCTA | CAGGAT |
| 1208 | E56Sa1 | exon56 | SaCas9 | + | GACTGCATCATCGGAACCTTCC | AGGGAT |
| 1209 | E56Sa2 | exon56 | SaCas9 | + | CATCGGAACCTTCCAGGGATCT | CAGGAT |
| 1210 | E56Sa4 | exon56 | SaCas9 | − | TTTGGATAACATGAACTTCAAG | TGGAGT |
| 1211 | E56Sa5 | exon56 | SaCas9 | − | TGCAGTCCTGTTACAAAGACGT | TTGGAT |
| 1212 | E56Sa6 | exon56 | SaCas9 | − | TCACACAGATGTTTATCACAAC | CTGGAT |
| 1213 | E57Sa1 | exon57 | SaCas9 | − | TTCAGAAGCAGAACGATGTACA | TAGGGT |
| 1214 | E57Sa2 | exon57 | SaCas9 | − | GTGTGGCTACAGCTGAAAGATG | ATGAAT |
| 1215 | E58Sa1 | exon58 | SaCas9 | + | TCAATTACCTCTGGGCTCCTGG | TAGAGT |
| 1216 | E58Sa2 | exon58 | SaCas9 | + | TCAGAAATATTCGTACAGTCTC | AAGAGT |
| 1217 | E58Sa4 | exon58 | SaCas9 | − | TCATGAGTACTCTTGAGACTGT | ACGAAT |
| 1218 | E58Sa5 | exon58 | SaCas9 | − | GAAAACTAAAGAACCTGTAATC | ATGAGT |
| 1219 | E58Sa6 | exon58 | SaCas9 | − | CTCATTTCACAGGCCTTCAAGA | GGGAAT |
| 1220 | E59Sa1 | exon59 | SaCas9 | + | ACTTTCTCGAGGTGATCTTGGA | GAGAGT |
| 1221 | E59Sa2 | exon59 | SaCas9 | + | GAGGAGATCGCCCACGGGCTGC | CAGGAT |
| 1222 | E59Sa3 | exon59 | SaCas9 | + | ATCCGTGGCCTCTTGAAGTTCC | CGGAGT |
| 1223 | E59Sa4 | exon59 | SaCas9 | + | CTTGAAGTTCCCGGAGTCTTTC | AAGGGT |
| 1224 | E59Sa5 | exon59 | SaCas9 | + | TCTATTTTTCTCTGCCAGTCAG | CGGAGT |
| 1225 | E59Sa6 | exon59 | SaCas9 | + | CCTCAGCCTGCTTTCGTAGAAG | CCGAGT |
| 1226 | E59Sa7 | exon59 | SaCas9 | − | CTGCGCCAAGCTGAGGTGATCA | AGGGAT |
| 1227 | E59Sa8 | exon59 | SaCas9 | − | ACTCCGGGAACTTCAAGAGGCC | ACGGAT |
| 1228 | E59Sa9 | exon59 | SaCas9 | − | AAGCAGGCTGAGGAGGTCAATA | CTGAGT |
| 1229 | E59Sa10 | exon59 | SaCas9 | − | GCTGCCTCCTGAGGAGAGAGCC | CAGAAT |
| 1230 | E60Sa1 | exon60 | SaCas9 | + | TCCATCTGGTGTTCAGGTCTTC | CAGAGT |
| 1231 | E60Sa2 | exon60 | SaCas9 | + | TGCTGAGGTTATACGGTGAGAG | CTGAAT |
| 1232 | E61Sa1 | exon61 | SaCas9 | − | CCTCCCAGGTGGCCGTCGAGGA | CCGAGT |
| 1233 | E62Sa1 | exon62 | SaCas9 | − | TCCCTTCTTTTCAGCGTCTGTC | CAGGGT |
| 1234 | E63Sa1 | exon63 | SaCas9 | + | GACTGGTAGAGCTCTGTCATTT | TGGGAT |
| 1235 | E63Sa2 | exon63 | SaCas9 | + | TGGGATGGTCCCAGCAAGTTGT | TTGAGT |
| 1236 | E64Sa1 | exon64 | SaCas9 | + | GCAAAGGGCCTTCTGCAGTCTT | CGGAGT |
| 1237 | E64Sa2 | exon64 | SaCas9 | + | TTTCATGGCAGTCCTATAAGCT | GAGAAT |
| 1238 | E64Sa3 | exon64 | SaCas9 | − | GTTTTCCCTCTTTTCAGCTGAC | CTGAAT |
| 1239 | E65Sa1 | exon65 | SaCas9 | + | TGGTCAAACAATTAATAATCTG | CAGGAT |
| 1240 | E65Sa2 | exon65 | SaCas9 | − | GGATATGTGTCTGAACTGGCTG | CTGAAT |
| 1241 | E65Sa3 | exon65 | SaCas9 | − | TTTGGTCAACGTCCCTCTCTGC | GTGGAT |
| 1242 | E65Sa4 | exon65 | SaCas9 | − | CCTCAAGCAAAATGACCAGCCC | ATGGAT |
| 1243 | E66Sa1 | exon66 | SaCas9 | + | TGCCAGTTTTAAAAGACAGGAC | ACGGAT |
| 1244 | E66Sa3 | exon66 | SaCas9 | − | TTCATAATAGGGGACGAACAGG | GAGGAT |
| 1245 | E67Sa1 | exon67 | SaCas9 | + | TGGCTCAATGTTACTGCCCCCA | AAGGAT |
| 1246 | E67Sa2 | exon67 | SaCas9 | + | ATGCAACTTCACCCAACTGTCT | TGGAAT |
| 1247 | E67Sa3 | exon67 | SaCas9 | + | CTTCACCCAACTGTCTTGGAAT | TTGGAT |
| 1248 | E67Sa4 | exon67 | SaCas9 | + | CCCAACTGTCTTGGAATTTGGA | TAGAAT |
| 1249 | E67Sa5 | exon67 | SaCas9 | − | TTCTATCCAAATTCCAAGACAG | TTGGGT |
| 1250 | E67Sa6 | exon67 | SaCas9 | − | TTCAAGCAAGTGGCAAGTTCAA | CAGGAT |
| 1251 | E68Sa2 | exon68 | SaCas9 | + | GACGGGCAGCCACACCATGGAC | TGGGGT |
| 1252 | E68Sa3 | exon68 | SaCas9 | − | ATCTGCAAAGAGTGTCCAATCA | TTGGAT |
| 1253 | E68Sa4 | exon68 | SaCas9 | − | CAGGCCAAATGTAACATCTGCA | AAGAGT |
| 1254 | E68Sa5 | exon68 | SaCas9 | − | TGGTGTGGCTGCCCGTCCTGCA | CAGAGT |
| 1255 | E68Sa6 | exon68 | SaCas9 | − | TCGAAGCGGCCCTCTTCCTAGA | CTGGAT |
| 1256 | E69Sa2 | exon69 | SaCas9 | + | ACCGGAGTGCAATATTCCACCA | TGGGAT |
| 1257 | E69Sa4 | exon69 | SaCas9 | − | CATAAAATGCACTATCCCATGG | TGGAAT |
| 1258 | E69Sa5 | exon69 | SaCas9 | − | GCCAAAGCTGCTTTTTTTCTGG | TCGAGT |
| 1259 | E69Sa6 | exon69 | SaCas9 | − | GTTTTTGCTCTTTATCAGGTAC | AGGAGT |
| 1260 | E70Sa1 | exon70 | SaCas9 | + | TGCACTGGCAGGTAGCCCATTC | GGGGAT |
| 1261 | E70Sa2 | exon70 | SaCas9 | − | TAGAGGGGGACAACATGGAAAC | GTGAGT |
| 1262 | E70Sa3 | exon70 | SaCas9 | − | AAAGGTATTTTGCGAAGCATCC | CCGAAT |
| 1263 | E71Sa2 | exon71 | SaCas9 | + | AATCTACTGGCCAGAAGTTGAT | CAGAGT |
| 1264 | E71Sa4 | exon71 | SaCas9 | − | ACTTCTGGCCAGTAGATTCTGC | GTGAGT |
| 1265 | E72Sa1 | exon72 | SaCas9 | + | CTGCTAGCATAATGTTCAATGC | GTGAAT |
| 1266 | E72Sa2 | exon72 | SaCas9 | + | TAGCATAATGTTCAATGCGTGA | ATGAGT |
| 1267 | E73Sa1 | exon73 | SaCas9 | − | CTAGCAGAAATGGAAAACAGCA | ATGGAT |
| 1268 | E74Sa1 | exon74 | SaCas9 | + | TGTTTTCTTCCTCAAGATCTGC | TAGGAT |
| 1269 | E74Sa2 | exon74 | SaCas9 | + | GGACTACGAGGCTGGCTCAGGG | GGGAGT |
| 1270 | E74Sa3 | exon74 | SaCas9 | + | GGTTCAAACTTTGGCAGTAATG | CTGGAT |
| 1271 | E74Sa4 | exon74 | SaCas9 | − | CAGATCTTGAGGAAGAAAACAG | GTGAGT |
| 1272 | E74Sa5 | exon74 | SaCas9 | − | GTGAGGAAAGAGGGGAGCTAGA | GAGAAT |
| 1273 | E74Sa6 | exon74 | SaCas9 | − | TGCCCAGATCTTGATTTCCTTA | GAGAGT |
| 1274 | E75Sa1 | exon75 | SaCas9 | + | CCAGCTGTTTATTGTGGTCTTC | CAGGAT |
| 1275 | E75Sa3 | exon75 | SaCas9 | − | CTGGAAGACCACAATAAACAGC | TGGAGT |
| 1276 | E75Sa4 | exon75 | SaCas9 | − | AACACAAAGGCCGCCTGGAAGC | CAGGAT |
| 1277 | E75Sa5 | exon75 | SaCas9 | − | GCCCACCTCTCCCCAGAGTCCC | CGGGAT |
| 1278 | E75Sa6 | exon75 | SaCas9 | − | TGAAATGATGCCCACCTCTCCC | CAGAGT |
| 1279 | E75Sa7 | exon75 | SaCas9 | − | GATGCCAATAGGAATCTGCAAG | CAGAAT |
| 1280 | E76Sa1 | exon76 | SaCas9 | + | GAGTAGCTAGGACACTTACCCA | TGGAGT |
| 1281 | E76Sa3 | exon76 | SaCas9 | − | TGGCAGTCAAACTTCGGACTCC | ATGGGT |
| 1282 | E76Sa4 | exon76 | SaCas9 | − | ACAGCAGTCAGCCTATGCTGCT | CCGAGT |
| 1283 | E76Sa5 | exon76 | SaCas9 | − | TTAGCCCCAGGCAGAGGCCAAA | GTGAAT |
| 1284 | E77Sa1 | exon77 | SaCas9 | + | AGCTTACCTCTTGAACTAGGGA | AGGAGT |
| 1285 | E77Sa2 | exon77 | SaCas9 | + | TCTTGAACTAGGGAAGGAGTTG | TTGAGT |
| 1286 | E77Sa3 | exon77 | SaCas9 | − | AGTCCTCCCCAGGACACAAGCA | CAGGGT |
| 1287 | E78Sa1 | exon78 | SaCas9 | + | TAACCTCTCTCATTGGCTTTCC | AGGGGT |
| 1288 | E79Sa1 | exon79 | SaCas9 | + | GCACTCTATTTACCTCTGATTT | TAGAAT |
| 1289 | E79Sa2 | exon79 | SaCas9 | + | CTTACTTACTTAAACTTCTTAG | TAGGAT |
| 1290 | E79Sa3 | exon79 | SaCas9 | + | AAAGTAACCCCTTGTTTTAAAT | CTGAGT |
| 1291 | E79Sa4 | exon79 | SaCas9 | + | TAAATCTGAGTTTTAAAAATCC | TTGGGT |
| 1292 | E79Sa5 | exon79 | SaCas9 | + | GGTAATTTGTTACCTTAGAGCT | TTGGGT |
| 1293 | E79Sa6 | exon79 | SaCas9 | + | TGAAAATTATGAAGGAAAAAGA | AAGAAT |
| 1294 | E79Sa7 | exon79 | SaCas9 | + | ATAGGTCACACGGTGGTATCTA | TTGAAT |
| 1295 | E79Sa8 | exon79 | SaCas9 | + | GTCACACGGTGGTATCTATTGA | ATGAAT |
| 1296 | E79Sa9 | exon79 | SaCas9 | + | AAAAAGAAATAAAATGGCATGA | AAGAGT |
| 1297 | E79Sa10 | exon79 | SaCas9 | + | TTAACCTGTCTAATCCACCAAG | AAGGGT |
| 1298 | E79Sa11 | exon79 | SaCas9 | + | AAGAAGGGTTTTTTTGTAACAT | TTGAAT |
| 1299 | E79Sa12 | exon79 | SaCas9 | + | GAACTACTCGCAGAGAAATGCA | AAGGAT |
| 1300 | E79Sa13 | exon79 | SaCas9 | + | TGGAAACACAGTTCATGGGCTT | CTGGGT |
| 1301 | E79Sa14 | exon79 | SaCas9 | + | CAATCATCCAATCCTTCACTTA | AAGAGT |
| 1302 | E79Sa15 | exon79 | SaCas9 | + | AAAGAGTGGCCTACTCCTTCAC | AGGGAT |
| 1303 | E79Sa16 | exon79 | SaCas9 | + | TACTCCTTCACAGGGATGGGCT | GGGAAT |
| 1304 | E79Sa17 | exon79 | SaCas9 | + | TATCATCGTCCTGCTACTGCTT | GGGAGT |
| 1305 | E79Sa18 | exon79 | SaCas9 | + | TACCTTCTGATGTTCACAAGGT | CAGAAT |
| 1306 | E79Sa19 | exon79 | SaCas9 | + | TGGCATTGCTAGCAGCAGGAAG | CTGAAT |
| 1307 | E79Sa20 | exon79 | SaCas9 | + | ATTCTAGACCAAGCAGGTAAGC | CTGGAT |
| 1308 | E79Sa21 | exon79 | SaCas9 | + | ACTAGAAGTAATTTCTTTCTAT | TAGGAT |
| 1309 | E79Sa22 | exon79 | SaCas9 | + | ATATGCAAAAAAAGAAAAAGCC | ATGAAT |
| 1310 | E79Sa23 | exon79 | SaCas9 | + | AAGAAAAAGCCATGAATTTCAT | ATGGAT |
| 1311 | E79Sa24 | exon79 | SaCas9 | + | TTTCATATGGATATCACGCCAA | AAGGAT |
| 1312 | E79Sa25 | exon79 | SaCas9 | + | TGTGTGTGTTTGTTTTGTTTTT | AGGGGT |
| 1313 | E79Sa26 | exon79 | SaCas9 | + | ACTGTCTAATCCTCTTTGTTGT | ATGAAT |
| 1314 | E79Sa27 | exon79 | SaCas9 | + | ATGAATATTATAAAAACCATGC | GGGAAT |
| 1315 | E79Sa28 | exon79 | SaCas9 | + | TTATAAAAACCATGCGGGAATC | AGGAGT |
| 1316 | E79Sa29 | exon79 | SaCas9 | − | TAACACCAACACTGTAACATTT | ACGAAT |
| 1317 | E79Sa30 | exon79 | SaCas9 | − | TTTTCAGGTACTGAGTTCTTAC | TTGAGT |
| 1318 | E79Sa31 | exon79 | SaCas9 | − | GTTTTGTCATTGTTTTCAGGTA | CTGAGT |
| 1319 | E79Sa32 | exon79 | SaCas9 | − | TTTTAGACACATTAGCTCTGGA | GTGAGT |
| 1320 | E79Sa33 | exon79 | SaCas9 | − | TTTCTTTTAGACACATTAGCTC | TGGAGT |
| 1321 | E79Sa34 | exon79 | SaCas9 | − | TCATTCTAAAATCAGAGGTAAA | TAGAGT |
| 1322 | E79Sa35 | exon79 | SaCas9 | − | TTTAAAACTCAGATTTAAAACA | AGGGGT |
| 1323 | E79Sa36 | exon79 | SaCas9 | − | ACGCTGGACCTTTTCTTTACCC | AAGGAT |
| 1324 | E79Sa37 | exon79 | SaCas9 | − | TGTTCCTGATAATTGTGCACAC | CTGAGT |
| 1325 | E79Sa38 | exon79 | SaCas9 | − | TATTTCTACCTCACTTTGGTTT | TGGGGT |
| 1326 | E79Sa39 | exon79 | SaCas9 | − | AAGTGTAATTAGCTTTTGGAGA | GTGGGT |
| 1327 | E79Sa40 | exon79 | SaCas9 | − | CATCAAGTGTAATTAGCTTTTG | GAGAGT |
| 1328 | E79Sa41 | exon79 | SaCas9 | − | CATCAAGTGTAATTAGCTTTTG | GAGAGT |
| 1329 | E79Sa42 | exon79 | SaCas9 | − | TGTTACAAAAAAACCCTTCTTG | GTGGAT |
| 1330 | E79Sa43 | exon79 | SaCas9 | − | TTTCCATCCTTTGCATTTCTCT | GCGAGT |
| 1331 | E79Sa44 | exon79 | SaCas9 | − | TAGATTTATTGTCCCATGTGGG | ATGAGT |
| 1332 | E79Sa45 | exon79 | SaCas9 | − | TTTTTAGATTTATTGTCCCATG | TGGGAT |
| 1333 | E79Sa46 | exon79 | SaCas9 | − | CTCTTCTCACAGTCAAAAGGAA | CTGGGT |
| 1334 | E79Sa47 | exon79 | SaCas9 | − | AGGCCACTCTTTAAGTGAAGGA | TTGGAT |
| 1335 | E79Sa48 | exon79 | SaCas9 | − | GGAGTAGGCCACTCTTTAAGTG | AAGGAT |
| 1336 | E79Sa49 | exon79 | SaCas9 | − | GATTCCCAGCCCATCCCTGTGA | AGGAGT |
| 1337 | E79Sa50 | exon79 | SaCas9 | − | ACGATGATAGGGCTGGAGGGCT | ATGGAT |
| 1338 | E79Sa51 | exon79 | SaCas9 | − | ACACAGGACTTATTATATCAGA | GTGAGT |
| 1339 | E79Sa52 | exon79 | SaCas9 | − | GAATACACAGGACTTATTATAT | CAGAGT |
| 1340 | E79Sa53 | exon79 | SaCas9 | − | TTTTTTTAAAGGGAACATGTGA | ATGAAT |
| 1341 | E79Sa54 | exon79 | SaCas9 | − | TGGATTTTTTTAAAGGGAACAT | GTGAAT |
| 1342 | E79Sa55 | exon79 | SaCas9 | − | ACAACGAAAGTAAAGTAAAGTA | TTGGAT |
| 1343 | E79Sa56 | exon79 | SaCas9 | − | AGGCTTACCTGCTTGGTCTAGA | ATGGAT |
| 1344 | E79Sa57 | exon79 | SaCas9 | − | ATCCAGGCTTACCTGCTTGGTC | TAGAAT |
| 1345 | E79Sa58 | exon79 | SaCas9 | − | ATAGTTATATAGATAAAGAGAT | ACGAAT |
| 1346 | E79Sa59 | exon79 | SaCas9 | − | CTGTCAGCTGAGTGGGGCAGGC | TTGAGT |
| 1347 | E79Sa60 | exon79 | SaCas9 | − | CTGCTCATTTGAGAACTGTCAG | CTGAGT |
| 1348 | E79Sa61 | exon79 | SaCas9 | − | ATTTCTTTATATGGAACGCATT | TTGGGT |
| 1349 | E79Sa62 | exon79 | SaCas9 | − | GAAATAAATCTATATTTTTGTG | AAGGGT |
| 1350 | E79Sa63 | exon79 | SaCas9 | − | ACAACAAAGAGGATTAGACAGT | AAGAGT |
| 1351 | E79Sa64 | exon79 | SaCas9 | − | TTTATAATATTCATACAACAAA | GAGGAT |
| 1352 | E79Sa65 | exon79 | SaCas9 | − | GTCATGACAGATGAAGAAGGAG | CAGAAT |
| 1353 | E79Sa66 | exon79 | SaCas9 | − | GCAGATGATTTGGGCAGAGCGA | TGGAGT |
| 1354 | E1SaCas9KKH2 | exon1 | SaCas9KKH | + | TTCTTCCCACCAAAGCATTTTG | AAAAGT |
| 1355 | E1SaCas9KKH3 | exon1 | SaCas9KKH | + | TTTGAAAAGTGTATATCAAGGC | AGCGAT |
| 1356 | E1SaCas9KKH4 | exon1 | SaCas9KKH | + | ATCAAGGCAGCGATAAAAAAAA | CCTGGT |
| 1357 | E1SaCas9KKH5 | exon1 | SaCas9KKH | + | GCAGCGATAAAAAAAACCTGGT | AAAAGT |
| 1358 | E1SaCas9KKH6 | exon1 | SaCas9KKH | + | AAAGTTCTTCAAACTTTATTGC | TCCAGT |
| 1359 | E1SaCas9KKH7 | exon1 | SaCas9KKH | + | TTATTGCTCCAGTAGGCTTAAA | AACAAT |
| 1360 | E1SaCas9KKH8 | exon1 | SaCas9KKH | + | AACAAACTTCAGCAGCTTTAAA | AAAAGT |
| 1361 | E1SaCas9KKH9 | exon1 | SaCas9KKH | + | GCAGCTTTAAAAAAAGTAACAC | TTCAGT |
| 1362 | E1SaCas9KKH10 | exon1 | SaCas9KKH | + | TTTTTCCTATTCGTTTTTCTCC | GAAGGT |
| 1363 | E1SaCas9KKH11 | exon1 | SaCas9KKH | + | TTCCTATTCGTTTTTCTCCGAA | GGTAAT |
| 1364 | E1SaCas9KKH12 | exon1 | SaCas9KKH | + | TTTCTCCGAAGGTAATTGCCTC | CCAGAT |
| 1365 | E1SaCas9KKH13 | exon1 | SaCas9KKH | + | CGAAGGTAATTGCCTCCCAGAT | CTGAGT |
| 1366 | E1SaCas9KKH14 | exon1 | SaCas9KKH | + | CCAGATCTGAGTCCTGTAGGGG | GAAAGT |
| 1367 | E1SaCas9KKH15 | exon1 | SaCas9KKH | + | ATCTGAGTCCTGTAGGGGGAAA | GTGAGT |
| 1368 | E1SaCas9KKH16 | exon1 | SaCas9KKH | + | TGAGTCCTGTAGGGGGAAAGTG | AGTGAT |
| 1369 | E1SaCas9KKH23 | exon1 | SaCas9KKH | − | TGGGAAGAAGTAGAGGACTGTT | GTAAGT |
| 1370 | E1SaCas9KKH24 | exon1 | SaCas9KKH | − | TTTTCAAAATGCTTTGGTGGGA | AGAAGT |
| 1371 | E1SaCas9KKH25 | exon1 | SaCas9KKH | − | TGATATACACTTTTCAAAATGC | TTTGGT |
| 1372 | E1SaCas9KKH26 | exon1 | SaCas9KKH | − | CGCTGCCTTGATATACACTTTT | CAAAAT |
| 1373 | E1SaCas9KKH27 | exon1 | SaCas9KKH | − | ACCAGGTTTTTTTTATCGCTGC | CTTGAT |
| 1374 | E1SaCas9KKH28 | exon1 | SaCas9KKH | − | AATAAAGTTTGAAGAACTTTTA | CCAGGT |
| 1375 | E1SaCas9KKH29 | exon1 | SaCas9KKH | − | GTTTTTAAGCCTACTGGAGCAA | TAAAGT |
| 1376 | E1SaCas9KKH30 | exon1 | SaCas9KKH | − | TCATTGTTTTTAAGCCTACTGG | AGCAAT |
| 1377 | E1SaCas9KKH31 | exon1 | SaCas9KKH | − | TTTTTTAAAGCTGCTGAAGTTT | GTTGGT |
| 1378 | E1SaCas9KKH32 | exon1 | SaCas9KKH | − | GTGTTACTTTTTTTAAAGCTGC | TGAAGT |
| 1379 | E1SaCas9KKH33 | exon1 | SaCas9KKH | − | GAGAAAAACGAATAGGAAAAAC | TGAAGT |
| 1380 | E1SaCas9KKH34 | exon1 | SaCas9KKH | − | AGGCAATTACCTTCGGAGAAAA | ACGAAT |
| 1381 | E1SaCas9KKH35 | exon1 | SaCas9KKH | − | CCTACAGGACTCAGATCTGGGA | GGCAAT |
| 1382 | E1SaCas9KKH36 | exon1 | SaCas9KKH | − | ACTCACTTTCCCCCTACAGGAC | TCAGAT |
| 1383 | E2SaCas9KKH1 | exon2 | SaCas9KKH | + | GTAACAAACCATTCTTACCTTA | GAAAAT |
| 1384 | E2SaCas9KKH2 | exon2 | SaCas9KKH | + | AAATTGTGCATTTACCCATTTT | GTGAAT |
| 1385 | E2SaCas9KKH3 | exon2 | SaCas9KKH | + | TTTGAACATCTTCTCTTTCATC | TAAAAT |
| 1386 | E2SaCas9KKH9 | exon2 | SaCas9KKH | − | TAAATGCACAATTTTCTAAGGT | AAGAAT |
| 1387 | E2SaCas9KKH10 | exon2 | SaCas9KKH | − | AATGGGTAAATGCACAATTTTC | TAAGGT |
| 1388 | E2SaCas9KKH11 | exon2 | SaCas9KKH | − | ACATTCACAAAATGGGTAAATG | CACAAT |
| 1389 | E2SaCas9KKH12 | exon2 | SaCas9KKH | − | AAAGAAAACATTCACAAAATGG | GTAAAT |
| 1390 | E2SaCas9KKH13 | exon2 | SaCas9KKH | | TTCAAAAGAAAACATTCACAAA | ATGGGT |
| 1391 | E2SaCas9KKH14 | exon2 | SaCas9KKH | − | GATGTTCAAAAGAAAACATTCA | CAAAAT |
| 1392 | E2SaCas9KKH15 | exon2 | SaCas9KKH | − | ATTTTGCATTTTAGATGAAAGA | GAAGAT |
| 1393 | E3SaCas9KKH2 | exon3 | SaCas9KKH | + | TTTTGCCCTGTCAGGCCTTCGA | GGAGGT |
| 1394 | E3SaCas9KKH3 | exon3 | SaCas9KKH | + | TCTAGGAGGCGCCTCCCATCCT | GTAGGT |
| 1395 | E3SaCas9KKH4 | exon3 | SaCas9KKH | + | CTCCCATCCTGTAGGTCACTGA | AGAGGT |
| 1396 | E3SaCas9KKH5 | exon3 | SaCas9KKH | + | CCTGTAGGTCACTGAAGAGGTT | CTCAAT |
| 1397 | E3SaCas9KKH6 | exon3 | SaCas9KKH | + | CTCAATATGCTGCTTCCCAAAC | TGAAAT |
| 1398 | E3SaCas9KKH10 | exon3 | SaCas9KKH | − | TCGAAGGCCTGACAGGGCAAAA | ACTGGT |
| 1399 | E3SaCas9KKH11 | exon3 | SaCas9KKH | − | TGAGAACCTCTTCAGTGACCTA | CAGGAT |
| 1400 | E3SaCas9KKH12 | exon3 | SaCas9KKH | − | GAAGCAGCATATTGAGAACCTC | TTCAGT |
| 1401 | E4SaCas9KKH1 | exon4 | SaCas9KKH | + | TACATTATTGTTCTGCAAAACC | CGCAGT |
| 1402 | E4SaCas9KKH2 | exon4 | SaCas9KKH | + | ATTGTTCAGGGCATGAACTCTT | GTGGAT |
| 1403 | E4SaCas9KKH6 | exon4 | SaCas9KKH | − | GGTTTTGCAGAACAATAATGTA | AGTAGT |
| 1404 | E4SaCas9KKH7 | exon4 | SaCas9KKH | − | GCGGGTTTTGCAGAACAATAAT | GTAAGT |
| 1405 | E4SaCas9KKH8 | exon4 | SaCas9KKH | − | GGCACTGCGGGTTTTGCAGAAC | AATAAT |
| 1406 | E4SaCas9KKH9 | exon4 | SaCas9KKH | − | CAAGGCACTGCGGGTTTTGCAG | AACAAT |
| 1407 | E4SaCas9KKH10 | exon4 | SaCas9KKH | − | TGAACAATGTCAACAAGGCACT | GCGGGT |
| 1408 | E4SaCas9KKH11 | exon4 | SaCas9KKH | − | ATCCACAAGAGTTCATGCCCTG | AACAAT |
| 1409 | E4SaCas9KKH12 | exon4 | SaCas9KKH | − | AGCCAAAAGAAAAAGGATCCAC | AAGAGT |
| 1410 | E4SaCas9KKH13 | exon4 | SaCas9KKH | − | TTTTGTTCTCAGCCAAAAGAAA | AAGGAT |
| 1411 | E5SaCas9KKH2 | exon5 | SaCas9KKH | + | TCAGGATTCTTACCTGCCAGTG | GAGGAT |
| 1412 | E5SaCas9KKH3 | exon5 | SaCas9KKH | + | CCTGCCAGTGGAGGATTATATT | CCAAAT |
| 1413 | E5SaCas9KKH4 | exon5 | SaCas9KKH | + | GGATTATATTCCAAATCAAACC | AAGAGT |
| 1414 | E5SaCas9KKH5 | exon5 | SaCas9KKH | + | TATATTCCAAATCAAACCAAGA | GTCAGT |
| 1415 | E5SaCas9KKH6 | exon5 | SaCas9KKH | + | CAAATCAAACCAAGAGTCAGTT | TATGAT |
| 1416 | E5SaCas9KKH7 | exon5 | SaCas9KKH | + | GAGTCAGTTTATGATTTCCATC | TACGAT |
| 1417 | E5SaCas9KKH8 | exon5 | SaCas9KKH | + | GTTTATGATTTCCATCTACGAT | GTCAGT |
| 1418 | E5SaCas9KKH9 | exon5 | SaCas9KKH | + | TTCCATCTACGATGTCAGTACT | TCCAAT |
| 1419 | E5SaCas9KKH10 | exon5 | SaCas9KKH | + | ATGTCAGTACTTCCAATATTCA | CTAAAT |
| 1420 | E5SaCas9KKH16 | exon5 | SaCas9KKH | − | ATATAATCCTCCACTGGCAGGT | AAGAAT |
| 1421 | E5SaCas9KKH17 | exon5 | SaCas9KKH | − | TTTGGAATATAATCCTCCACTG | GCAGGT |
| 1422 | E5SaCas9KKH18 | exon5 | SaCas9KKH | − | TGACTCTTGGTTTGATTTGGAA | TATAAT |
| 1423 | E5SaCas9KKH19 | exon5 | SaCas9KKH | − | TAAACTGACTCTTGGTTTGATT | TGGAAT |
| 1424 | E5SaCas9KKH20 | exon5 | SaCas9KKH | − | GAAATCATAAACTGACTCTTGG | TTTGAT |
| 1425 | E5SaCas9KKH21 | exon5 | SaCas9KKH | − | AGATGGAAATCATAAACTGACT | CTTGGT |
| 1426 | E5SaCas9KKH22 | exon5 | SaCas9KKH | − | TGGAAGTACTGACATCGTAGAT | GGAAAT |
| 1427 | E5SaCas9KKH23 | exon5 | SaCas9KKH | − | GAATATTGGAAGTACTGACATC | GTAGAT |
| 1428 | E5SaCas9KKH24 | exon5 | SaCas9KKH | − | ACAGGTTGATTTAGTGAATATT | GGAAGT |
| 1429 | E5SaCas9KKH25 | exon5 | SaCas9KKH | − | CTTTCTTTAACAGGTTGATTTA | GTGAAT |
| 1430 | E5SaCas9KKH26 | exon5 | SaCas9KKH | − | ACCCCTTTCTTTAACAGGTTGA | TTTAGT |
| 1431 | E6SaCas9KKH1 | exon6 | SaCas9KKH | + | AGTAATCTTCTTACCTATGACT | ATGGAT |
| 1432 | E6SaCas9KKH2 | exon6 | SaCas9KKH | + | TCAAAGCCAGGCCATCAGACCA | GCTGGT |
| 1433 | E6SaCas9KKH3 | exon6 | SaCas9KKH | + | AAGCCAGGCCATCAGACCAGCT | GGTGGT |
| 1434 | E6SaCas9KKH4 | exon6 | SaCas9KKH | + | AGGCCATCAGACCAGCTGGTGG | TGAAGT |
| 1435 | E6SaCas9KKH5 | exon6 | SaCas9KKH | + | CATCAGACCAGCTGGTGGTGAA | GTTGAT |
| 1436 | E6SaCas9KKH6 | exon6 | SaCas9KKH | + | GTGAAGTTGATTACATTAACCT | GTGGAT |
| 1437 | E6SaCas9KKH7 | exon6 | SaCas9KKH | + | AAGTTGATTACATTAACCTGTG | GATAAT |
| 1438 | E6SaCas9KKH8 | exon6 | SaCas9KKH | + | TTACATTAACCTGTGGATAATT | ACGAGT |
| 1439 | E6SaCas9KKH9 | exon6 | SaCas9KKH | + | ATTAACCTGTGGATAATTACGA | GTTGAT |
| 1440 | E6SaCas9KKH10 | exon6 | SaCas9KKH | + | TTGATTGTCGGACCCAGCTCAG | GAGAAT |
| 1441 | E6SaCas9KKH11 | exon6 | SaCas9KKH | + | AGCTCAGGAGAATCTTTTCACT | GTTGGT |
| 1442 | E6SaCas9KKH12 | exon6 | SaCas9KKH | + | AATCTTTTCACTGTTGGTTTGT | TGCAAT |
| 1443 | E6SaCas9KKH13 | exon6 | SaCas9KKH | + | TGTTGGTTTGTTGCAATCCAGC | CATGAT |
| 1444 | E6SaCas9KKH19 | exon6 | SaCas9KKH | − | TTGAATGCTCTCATCCATAGTC | ATAGGT |
| 1445 | E6SaCas9KKH20 | exon6 | SaCas9KKH | − | CCTGGCTTTGAATGCTCTCATC | CATAGT |
| 1446 | E6SaCas9KKH21 | exon6 | SaCas9KKH | − | CAGCTGGTCTGATGGCCTGGCT | TTGAAT |
| 1447 | E6SaCas9KKH22 | exon6 | SaCas9KKH | − | AATCAACTTCACCACCAGCTGG | TCTGAT |
| 1448 | E6SaCas9KKH23 | exon6 | SaCas9KKH | − | AATGTAATCAACTTCACCACCA | GCTGGT |
| 1449 | E6SaCas9KKH24 | exon6 | SaCas9KKH | − | CTCGTAATTATCCACAGGTTAA | TGTAAT |
| 1450 | E6SaCas9KKH25 | exon6 | SaCas9KKH | − | ATCAACTCGTAATTATCCACAG | GTTAAT |
| 1451 | E6SaCas9KKH26 | exon6 | SaCas9KKH | − | GACAATCAACTCGTAATTATCC | ACAGGT |
| 1452 | E6SaCas9KKH27 | exon6 | SaCas9KKH | − | GAGCTGGGTCCGACAATCAACT | CGTAAT |
| 1453 | E6SaCas9KKH28 | exon6 | SaCas9KKH | − | AAGATTCTCCTGAGCTGGGTCC | GACAAT |
| 1454 | E6SaCas9KKH29 | exon6 | SaCas9KKH | − | ACAGTGAAAAGATTCTCCTGAG | CTGGGT |
| 1455 | E6SaCas9KKH30 | exon6 | SaCas9KKH | − | GATTGCAACAAACCAACAGTGA | AAAGAT |
| 1456 | E6SaCas9KKH31 | exon6 | SaCas9KKH | − | CATGGCTGGATTGCAACAAACC | AACAGT |
| 1457 | E6SaCas9KKH32 | exon6 | SaCas9KKH | − | AATGTAATGAAAAATATCATGG | CTGGAT |
| 1458 | E6SaCas9KKH33 | exon6 | SaCas9KKH | − | CATGTAGGTCAAAAATGTAATG | AAAAAT |
| 1459 | E6SaCas9KKH34 | exon6 | SaCas9KKH | − | ATTTCCACATGTAGGTCAAAAA | TGTAAT |
| 1460 | E7SaCas9KKH1 | exon7 | SaCas9KKH | + | AGTCCAGAAATTTACCAACCTT | CAGGAT |
| 1461 | E7SaCas9KKH2 | exon7 | SaCas9KKH | + | AGAAATTTACCAACCTTCAGGA | TCGAGT |
| 1462 | E7SaCas9KKH3 | exon7 | SaCas9KKH | + | AATTTACCAACCTTCAGGATCG | AGTAGT |
| 1463 | E7SaCas9KKH4 | exon7 | SaCas9KKH | + | AGGATCGAGTAGTTTCTCTATG | CCTAAT |
| 1464 | E7SaCas9KKH5 | exon7 | SaCas9KKH | + | TCGAGTAGTTTCTCTATGCCTA | ATTGAT |
| 1465 | E7SaCas9KKH6 | exon7 | SaCas9KKH | + | TCTCTATGCCTAATTGATATCT | GGCGAT |
| 1466 | E7SaCas9KKH7 | exon7 | SaCas9KKH | + | GCCTAATTGATATCTGGCGATG | TTGAAT |
| 1467 | E7SaCas9KKH8 | exon7 | SaCas9KKH | + | TCTGGCGATGTTGAATGCATGT | TCCAGT |
| 1468 | E7SaCas9KKH9 | exon7 | SaCas9KKH | + | GACTGCTGGCAAACCACACTAT | TCCAGT |
| 1469 | E7SaCas9KKH10 | exon7 | SaCas9KKH | + | CTGGCAAACCACACTATTCCAG | TCAAAT |
| 1470 | E7SaCas9KKH11 | exon7 | SaCas9KKH | + | CAAACCACACTATTCCAGTCAA | ATAGGT |
| 1471 | E7SaCas9KKH13 | exon7 | SaCas9KKH | − | GAGAAACTACTCGATCCTGAAG | GTTGGT |
| 1472 | E7SaCas9KKH14 | exon7 | SaCas9KKH | − | CATAGAGAAACTACTCGATCCT | GAAGGT |
| 1473 | E7SaCas9KKH15 | exon7 | SaCas9KKH | − | TCAATTAGGCATAGAGAAACTA | CTCGAT |
| 1474 | E7SaCas9KKH16 | exon7 | SaCas9KKH | − | CATGCATTCAACATCGCCAGAT | ATCAAT |
| 1475 | E7SaCas9KKH17 | exon7 | SaCas9KKH | − | CTGGAACATGCATTCAACATCG | CCAGAT |
| 1476 | E7SaCas9KKH18 | exon7 | SaCas9KKH | − | GACTGGAATAGTGTGGTTTGCC | AGCAGT |
| 1477 | E7SaCas9KKH19 | exon7 | SaCas9KKH | − | CAGACCTATTTGACTGGAATAG | TGTGGT |
| 1478 | E7SaCas9KKH20 | exon7 | SaCas9KKH | − | TAGGCCAGACCTATTTGACTGG | AATAGT |
| 1479 | E7SaCas9KKH21 | exon7 | SaCas9KKH | − | TTTTAGGCCAGACCTATTTGAC | TGGAAT |
| 1480 | E8SaCas9KKH1 | exon8 | SaCas9KKH | + | TCCTTTACACACTTTACCTGTT | GAGAAT |
| 1481 | E8SaCas9KKH2 | exon8 | SaCas9KKH | + | TTTACACACTTTACCTGTTGAG | AATAGT |
| 1482 | E8SaCas9KKH3 | exon8 | SaCas9KKH | + | TTTACCTGTTGAGAATAGTGCA | TTTGAT |
| 1483 | E8SaCas9KKH4 | exon8 | SaCas9KKH | + | ACCTGTTGAGAATAGTGCATTT | GATGAT |
| 1484 | E8SaCas9KKH5 | exon8 | SaCas9KKH | + | TAGTGCATTTGATGATGTAACT | GAAAAT |
| 1485 | E8SaCas9KKH6 | exon8 | SaCas9KKH | + | GATGTAACTGAAAATGTTCTTC | TTTAGT |
| 1486 | E8SaCas9KKH7 | exon8 | SaCas9KKH | + | AAAATGTTCTTCTTTAGTCACT | TTAGGT |
| 1487 | E8SaCas9KKH8 | exon8 | SaCas9KKH | + | GCCTTGGCAACATTTCCACTTC | CTGGAT |
| 1488 | E8SaCas9KKH9 | exon8 | SaCas9KKH | + | ACATTTCCACTTCCTGGATGGC | TTCAAT |
| 1489 | E8SaCas9KKH10 | exon8 | SaCas9KKH | + | CACTTGTTGAGGCAAAACTTGG | AAGAGT |
| 1490 | E8SaCas9KKH11 | exon8 | SaCas9KKH | + | TTGTTGAGGCAAAACTTGGAAG | AGTGAT |
| 1491 | E8SaCas9KKH12 | exon8 | SaCas9KKH | + | GAGGCAAAACTTGGAAGAGTGA | TGTGAT |
| 1492 | E8SaCas9KKH13 | exon8 | SaCas9KKH | + | GGAAGAGTGATGTGATGTACAT | TAAGAT |
| 1493 | E8SaCas9KKH14 | exon8 | SaCas9KKH | + | TACATTAAGATGGACTTCTTAT | CTGGAT |
| 1494 | E8SaCas9KKH15 | exon8 | SaCas9KKH | + | TTAAGATGGACTTCTTATCTGG | ATAGGT |
| 1495 | E8SaCas9KKH16 | exon8 | SaCas9KKH | + | AGATGGACTTCTTATCTGGATA | GGTGGT |
| 1496 | E8SaCas9KKH18 | exon8 | SaCas9KKH | − | CAAATGCACTATTCTCAACAGG | TAAAGT |
| 1497 | E8SaCas9KKH19 | exon8 | SaCas9KKH | − | ATCATCAAATGCACTATTCTCA | ACAGGT |
| 1498 | E8SaCas9KKH20 | exon8 | SaCas9KKH | − | AAGAACATTTTCAGTTACATCA | TCAAAT |
| 1499 | E8SaCas9KKH21 | exon8 | SaCas9KKH | − | AAAGTGACTAAAGAAGAACATT | TTCAGT |
| 1500 | E8SaCas9KKH22 | exon8 | SaCas9KKH | − | TGGAAATGTTGCCAAGGCCACC | TAAAGT |
| 1501 | E8SaCas9KKH23 | exon8 | SaCas9KKH | − | GCATTGAAGCCATCCAGGAAGT | GGAAAT |
| 1502 | E8SaCas9KKH24 | exon8 | SaCas9KKH | − | AAGTGAGCATTGAAGCCATCCA | GGAAGT |
| 1503 | E8SaCas9KKH25 | exon8 | SaCas9KKH | − | CACTCTTCCAAGTTTTGCCTCA | ACAAGT |
| 1504 | E8SaCas9KKH26 | exon8 | SaCas9KKH | − | TAATGTACATCACATCACTCTT | CCAAGT |
| 1505 | E8SaCas9KKH27 | exon8 | SaCas9KKH | − | CCTATCCAGATAAGAAGTCCAT | CTTAAT |
| 1506 | E8SaCas9KKH28 | exon8 | SaCas9KKH | − | GTTGATACCACCTATCCAGATA | AGAAGT |
| 1507 | E8SaCas9KKH29 | exon8 | SaCas9KKH | − | TACAGATGTTGATACCACCTAT | CCAGAT |
| 1508 | E9SaCas9KKH1 | exon9 | SaCas9KKH | + | TAAATGTTGACAGACCTGTGAA | GGAAAT |
| 1509 | E9SaCas9KKH2 | exon9 | SaCas9KKH | + | TGTGAAGGAAATGGGCTCCGTG | TAGGGT |
| 1510 | E9SaCas9KKH3 | exon9 | SaCas9KKH | + | GAAATGGGCTCCGTGTAGGGTC | AGAGGT |
| 1511 | E9SaCas9KKH4 | exon9 | SaCas9KKH | + | ATGGGCTCCGTGTAGGGTCAGA | GGTGGT |
| 1512 | E9SaCas9KKH5 | exon9 | SaCas9KKH | + | AGCCTGTGTGTAGGCATAGCTC | TTGAAT |
| 1513 | E9SaCas9KKH6 | exon9 | SaCas9KKH | + | TCTTGAATCGAGGCTTAGGGGA | AGAAGT |
| 1514 | E9SaCas9KKH7 | exon9 | SaCas9KKH | + | CATATCCCTGTGCTAGACTGAC | CGTGAT |
| 1515 | E9SaCas9KKH11 | exon9 | SaCas9KKH | − | CTACACGGAGCCCATTTCCTTC | ACAGGT |
| 1516 | E9SaCas9KKH12 | exon9 | SaCas9KKH | − | GAGAGAACTTCTTCCCCTAAGC | CTCGAT |
| 1517 | E9SaCas9KKH13 | exon9 | SaCas9KKH | − | CAGATCACGGTCAGTCTAGCAC | AGGGAT |
| 1518 | E9SaCas9KKH14 | exon9 | SaCas9KKH | − | AAACCCTTCTCTGCAGATCACG | GTCAGT |
| 1519 | E10SaCas9KKH2 | exon10 | SaCas9KKH | + | AATTAACGTTTTAGTTTACCTC | ATGAGT |
| 1520 | E10SaCas9KKH3 | exon10 | SaCas9KKH | + | TAGTTTACCTCATGAGTATGAA | ACTGGT |
| 1521 | E10SaCas9KKH4 | exon10 | SaCas9KKH | + | CTTTCACCACTTCCACATCATT | AGAAAT |
| 1522 | E10SaCas9KKH5 | exon10 | SaCas9KKH | + | ATTAGAAATCTCTCCTTGTGCT | TGCAAT |
| 1523 | E10SaCas9KKH6 | exon10 | SaCas9KKH | + | TGTGTCCTCAGCAGAAAGAAGC | CACGAT |
| 1524 | E10SaCas9KKH7 | exon10 | SaCas9KKH | + | GTCCTCAGCAGAAAGAAGCCAC | GATAAT |
| 1525 | E10SaCas9KKH8 | exon10 | SaCas9KKH | + | GATAATACTTCTTCTAAAGCTG | TTTGAT |
| 1526 | E10SaCas9KKH9 | exon10 | SaCas9KKH | + | ACTTCTTCTAAAGCTGTTTGAT | AACGGT |
| 1527 | E10SaCas9KKH10 | exon10 | SaCas9KKH | + | TCTAAAGCTGTTTGATAACGGT | CCAGGT |
| 1528 | E10SaCas9KKH11 | exon10 | SaCas9KKH | + | GTCCAGGTTTACTTCACTCTCC | ATCAAT |
| 1529 | E10SaCas9KKH12 | exon10 | SaCas9KKH | + | TTCACTCTCCATCAATGAACTG | CCAAAT |
| 1530 | E10SaCas9KKH13 | exon10 | SaCas9KKH | + | AATGACTTGTCTTCAGGAGCTT | CCAAAT |
| 1531 | E10SaCas9KKH19 | exon10 | SaCas9KKH | − | TGAAAGACCAGTTTCATACTCA | TGAGGT |
| 1532 | E10SaCas9KKH20 | exon10 | SaCas9KKH | − | AATGATGTGGAAGTGGTGAAAG | ACCAGT |
| 1533 | E10SaCas9KKH21 | exon10 | SaCas9KKH | − | GAGAGATTTCTAATGATGTGGA | AGTGGT |
| 1534 | E10SaCas9KKH22 | exon10 | SaCas9KKH | − | AAGGAGAGATTTCTAATGATGT | GGAAGT |
| 1535 | E10SaCas9KKH23 | exon10 | SaCas9KKH | − | GCAAGCACAAGGAGAGATTTCT | AATGAT |
| 1536 | E10SaCas9KKH24 | exon10 | SaCas9KKH | − | ATTGCAAGCACAAGGAGAGATT | TCTAAT |
| 1537 | E10SaCas9KKH25 | exon10 | SaCas9KKH | − | AGGACACATTGCAAGCACAAGG | AGAGAT |
| 1538 | E10SaCas9KKH26 | exon10 | SaCas9KKH | − | ACCGTTATCAAACAGCTTTAGA | AGAAGT |
| 1539 | E10SaCas9KKH27 | exon10 | SaCas9KKH | − | TTGGCAGTTCATTGATGGAGAG | TGAAGT |
| 1540 | E10SaCas9KKH28 | exon10 | SaCas9KKH | − | GTCATTTGGCAGTTCATTGATG | GAGAGT |
| 1541 | E10SaCas9KKH29 | exon10 | SaCas9KKH | − | AAGACAAGTCATTTGGCAGTTC | ATTGAT |
| 1542 | E10SaCas9KKH30 | exon10 | SaCas9KKH | − | AGCTCCTGAAGACAAGTCATTT | GGCAGT |
| 1543 | E10SaCas9KKH31 | exon10 | SaCas9KKH | − | CAGCATTTGGAAGCTCCTGAAG | ACAAGT |
| 1544 | E11SaCas9KKH1 | exon11 | SaCas9KKH | + | GCTACCCTGAGGCATTCCCATC | TTGAAT |
| 1545 | E11SaCas9KKH2 | exon11 | SaCas9KKH | + | AGGCATTCCCATCTTGAATTTA | GGAGAT |
| 1546 | E11SaCas9KKH3 | exon11 | SaCas9KKH | + | GGAGATTCATCTGCTCTTGTAC | TTCAGT |
| 1547 | E11SaCas9KKH4 | exon11 | SaCas9KKH | + | TTGTACTTCAGTTTCTTCATCT | TCTGAT |
| 1548 | E11SaCas9KKH5 | exon11 | SaCas9KKH | + | TACTTCAGTTTCTTCATCTTCT | GATAAT |
| 1549 | E11SaCas9KKH6 | exon11 | SaCas9KKH | + | CATCTTCTGATAATTTTCCTGT | TCCAAT |
| 1550 | E11SaCas9KKH7 | exon11 | SaCas9KKH | + | TCCTGTTCCAATCAGCTTACTT | CCCAAT |
| 1551 | E11SaCas9KKH8 | exon11 | SaCas9KKH | + | CAATCAGCTTACTTCCCAATTG | TAGAAT |
| 1552 | E11SaCas9KKH9 | exon11 | SaCas9KKH | + | TGTAGAATATTACCAACCCGGC | CCTGAT |
| 1553 | E11SaCas9KKH10 | exon11 | SaCas9KKH | + | CCAACCCGGCCCTGATGGGCTG | TCAAAT |
| 1554 | E11SaCas9KKH15 | exon11 | SaCas9KKH | − | CTAGCATGGAAAAACAAAGCAA | GTAAGT |
| 1555 | E11SaCas9KKH16 | exon11 | SaCas9KKH | − | GTAGCTAGCATGGAAAAACAAA | GCAAGT |
| 1556 | E11SaCas9KKH17 | exon11 | SaCas9KKH | − | TAAATTCAAGATGGGAATGCCT | CAGGGT |
| 1557 | E11SaCas9KKH18 | exon11 | SaCas9KKH | − | ATGAATCTCCTAAATTCAAGAT | GGGAAT |
| 1558 | E11SaCas9KKH19 | exon11 | SaCas9KKH | − | GAGCAGATGAATCTCCTAAATT | CAAGAT |
| 1559 | E11SaCas9KKH20 | exon11 | SaCas9KKH | − | AGTACAAGAGCAGATGAATCTC | CTAAAT |
| 1560 | E11SaCas9KKH21 | exon11 | SaCas9KKH | − | AGAAACTGAAGTACAAGAGCAG | ATGAAT |
| 1561 | E11SaCas9KKH22 | exon11 | SaCas9KKH | − | ATGAAGAAACTGAAGTACAAGA | GCAGAT |
| 1562 | E11SaCas9KKH23 | exon11 | SaCas9KKH | − | AATTATCAGAAGATGAAGAAAC | TGAAGT |
| 1563 | E11SaCas9KKH24 | exon11 | SaCas9KKH | − | GATTGGAACAGGAAAATTATCA | GAAGAT |
| 1564 | E11SaCas9KKH25 | exon11 | SaCas9KKH | − | GGAAGTAAGCTGATTGGAACAG | GAAAAT |
| 1565 | E11SaCas9KKH26 | exon11 | SaCas9KKH | − | ATATTCTACAATTGGGAAGTAA | GCTGAT |
| 1566 | E11SaCas9KKH27 | exon11 | SaCas9KKH | − | GGTTGGTAATATTCTACAATTG | GGAAGT |
| 1567 | E11SaCas9KKH28 | exon11 | SaCas9KKH | − | CAGGGCCGGGTTGGTAATATTC | TACAAT |
| 1568 | E11SaCas9KKH29 | exon11 | SaCas9KKH | − | GACAGCCCATCAGGGCCGGGTT | GGTAAT |
| 1569 | E11SaCas9KKH30 | exon11 | SaCas9KKH | − | TTTGACAGCCCATCAGGGCCGG | GTTGGT |
| 1570 | E11SaCas9KKH31 | exon11 | SaCas9KKH | − | TGGATTTGACAGCCCATCAGGG | CCGGGT |
| 1571 | E11SaCas9KKH32 | exon11 | SaCas9KKH | − | CCTTCTTTGTCAGGGGTACATG | ATGGAT |
| 1572 | E11SaCas9KKH33 | exon11 | SaCas9KKH | − | ACTTCCTTCTTTGTCAGGGGTA | CATGAT |
| 1573 | E12SaCas9KKH1 | exon12 | SaCas9KKH | + | TTATGTTGTTGTACTTGGCGTT | TTAGGT |
| 1574 | E12SaCas9KKH2 | exon12 | SaCas9KKH | + | TGTACTTGGCGTTTTAGGTCTT | CAAGAT |
| 1575 | E12SaCas9KKH3 | exon12 | SaCas9KKH | + | TTGGCGTTTTAGGTCTTCAAGA | TCAGGT |
| 1576 | E12SaCas9KKH4 | exon12 | SaCas9KKH | + | GTTCTTTCTTCTGTTTTTGTTA | GCCAGT |
| 1577 | E12SaCas9KKH5 | exon12 | SaCas9KKH | + | TGTTAGCCAGTCATTCAACTCT | TTCAGT |
| 1578 | E12SaCas9KKH6 | exon12 | SaCas9KKH | + | CAGTCATTCAACTCTTTCAGTT | TCTGAT |
| 1579 | E12SaCas9KKH7 | exon12 | SaCas9KKH | + | AACTCTTTCAGTTTCTGATTCT | GGAGAT |
| 1580 | E12SaCas9KKH8 | exon12 | SaCas9KKH | + | CCATTAAAACTCTATGTAAACT | GAAAAT |
| 1581 | E12SaCas9KKH9 | exon12 | SaCas9KKH | − | ACGCCAAGTACAACAACATAAG | GTAGGT |
| 1582 | E12SaCas9KKH10 | exon12 | SaCas9KKH | − | TAAAACGCCAAGTACAACAACA | TAAGGT |
| 1583 | E12SaCas9KKH11 | exon12 | SaCas9KKH | − | CTGATCTTGAAGACCTAAAACG | CCAAGT |
| 1584 | E12SaCas9KKH12 | exon12 | SaCas9KKH | − | AATGGAGGAAGAGCCTCTTGGA | CCTGAT |
| 1585 | E12SaCas9KKH13 | exon12 | SaCas9KKH | − | CAAAAACAGAAGAAAGAACAAG | GAAAAT |
| 1586 | E12SaCas9KKH14 | exon12 | SaCas9KKH | − | CCAGAATCAGAAACTGAAAGAG | TTGAAT |
| 1587 | E12SaCas9KKH15 | exon12 | SaCas9KKH | − | GATCTCCAGAATCAGAAACTGA | AAGAGT |
| 1588 | E12SaCas9KKH16 | exon12 | SaCas9KKH | − | ACATAGAGTTTTAATGGATCTC | CAGAAT |
| 1589 | E12SaCas9KKH17 | exon12 | SaCas9KKH | − | TTTCAGTTTACATAGAGTTTTA | ATGGAT |
| 1590 | E12SaCas9KKH18 | exon12 | SaCas9KKH | − | AAATTTTCAGTTTACATAGAGT | TTTAAT |
| 1591 | E12SaCas9KKH19 | exon12 | SaCas9KKH | − | TCTTTCAAATTTTCAGTTTACA | TAGAGT |
| 1592 | E13SaCas9KKH2 | exon13 | SaCas9KKH | + | CCTTAAGTTGTTCTTCCAAAGC | AGCAGT |
| 1593 | E13SaCas9KKH3 | exon13 | SaCas9KKH | + | TGTTCTTCCAAAGCAGCAGTTG | CGTGAT |
| 1594 | E13SaCas9KKH4 | exon13 | SaCas9KKH | + | AGCAGCAGTTGCGTGATCTCCA | CTAGAT |
| 1595 | E13SaCas9KKH5 | exon13 | SaCas9KKH | + | ATTCATCAACTACCACCACCAT | GTGAGT |
| 1596 | E13SaCas9KKH6 | exon13 | SaCas9KKH | + | ACTACCACCACCATGTGAGTGA | GAGAAT |
| 1597 | E13SaCas9KKH7 | exon13 | SaCas9KKH | + | TTGACCCTGACTTGTTCTTGTT | CTAGAT |
| 1598 | E13SaCas9KKH13 | exon13 | SaCas9KKH | − | GCTTTGGAAGAACAACTTAAGG | TCAGAT |
| 1599 | E13SaCas9KKH14 | exon13 | SaCas9KKH | − | CTGCTGCTTTGGAAGAACAACT | TAAGGT |
| 1600 | E13SaCas9KKH15 | exon13 | SaCas9KKH | − | GGTGGTAGTTGATGAATCTAGT | GGAGAT |
| 1601 | E13SaCas9KKH16 | exon13 | SaCas9KKH | − | CATGGTGGTGGTAGTTGATGAA | TCTAGT |
| 1602 | E13SaCas9KKH17 | exon13 | SaCas9KKH | − | ACTCACATGGTGGTGGTAGTTG | ATGAAT |
| 1603 | E13SaCas9KKH18 | exon13 | SaCas9KKH | − | TCTCACTCACATGGTGGTGGTA | GTTGAT |
| 1604 | E13SaCas9KKH19 | exon13 | SaCas9KKH | − | ATTCTCTCACTCACATGGTGGT | GGTAGT |
| 1605 | E13SaCas9KKH20 | exon13 | SaCas9KKH | − | TCAATTCTCTCACTCACATGGT | GGTGGT |
| 1606 | E13SaCas9KKH21 | exon13 | SaCas9KKH | − | GGGTCAATTCTCTCACTCACAT | GGTGGT |
| 1607 | E13SaCas9KKH22 | exon13 | SaCas9KKH | − | TCAGGGTCAATTCTCTCACTCA | CATGGT |
| 1608 | E13SaCas9KKH23 | exon13 | SaCas9KKH | − | TCTAGAACAAGAACAAGTCAGG | GTCAAT |
| 1609 | E13SaCas9KKH24 | exon13 | SaCas9KKH | − | AAGATCTAGAACAAGAACAAGT | CAGGGT |
| 1610 | E13SaCas9KKH25 | exon13 | SaCas9KKH | − | TTCAAGAAGATCTAGAACAAGA | ACAAGT |
| 1611 | E13SaCas9KKH26 | exon13 | SaCas9KKH | − | TTTTATCTTTCAGGTGCTTCAA | GAAGAT |
| 1612 | E14SaCas9KKH1 | exon14 | SaCas9KKH | + | CTCACACATGACACACCTGTTC | TTCAGT |
| 1613 | E14SaCas9KKH2 | exon14 | SaCas9KKH | + | CAGTAAGACGTTGCCATTTGAG | AAGGAT |
| 1614 | E14SaCas9KKH3 | exon14 | SaCas9KKH | + | AGGATGTCTTGTAAAAGAACCC | AGCGGT |
| 1615 | E14SaCas9KKH4 | exon14 | SaCas9KKH | + | CCCAGCGGTCTTCTGTCCATCT | ACAGAT |
| 1616 | E14SaCas9KKH5 | exon14 | SaCas9KKH | + | GTCCATCTACAGATGTTTGCCC | ATCGAT |
| 1617 | E14SaCas9KKH6 | exon14 | SaCas9KKH | + | ACAGATGTTTGCCCATCGATCT | CCCAAT |
| 1618 | E14SaCas9KKH8 | exon14 | SaCas9KKH | − | AATGGCAACGTCTTACTGAAGA | ACAGGT |
| 1619 | E14SaCas9KKH9 | exon14 | SaCas9KKH | − | GTTCTTTTACAAGACATCCTTC | TCAAAT |
| 1620 | E14SaCas9KKH10 | exon14 | SaCas9KKH | − | TCTGTAGATGGACAGAAGACCG | CTGGGT |
| 1621 | E14SaCas9KKH11 | exon14 | SaCas9KKH | − | GGAGATCGATGGGCAAACATCT | GTAGAT |
| 1622 | E14SaCas9KKH12 | exon14 | SaCas9KKH | − | TCTCTTCTCCAGGTATTGGGAG | ATCGAT |
| 1623 | E14SaCas9KKH13 | exon14 | SaCas9KKH | − | ATTGTCTCTTCTCCAGGTATTG | GGAGAT |
| 1624 | E15SaCas9KKH2 | exon15 | SaCas9KKH | + | ACATACGGCCAGTTTTTGAAGA | CTTGAT |
| 1625 | E15SaCas9KKH3 | exon15 | SaCas9KKH | + | TGAAGACTTGATAACATTTCAT | TTTGAT |
| 1626 | E15SaCas9KKH4 | exon15 | SaCas9KKH | + | ACATTTCATTTTGATCTTTAAA | GCCAGT |
| 1627 | E15SaCas9KKH5 | exon15 | SaCas9KKH | + | TTTGATCTTTAAAGCCAGTTGT | GTGAAT |
| 1628 | E15SaCas9KKH10 | exon15 | SaCas9KKH | − | TAAAGATCAAAATGAAATGTTA | TCAAGT |
| 1629 | E15SaCas9KKH11 | exon15 | SaCas9KKH | − | CAACTGGCTTTAAAGATCAAAA | TGAAAT |
| 1630 | E15SaCas9KKH12 | exon15 | SaCas9KKH | − | TCACACAACTGGCTTTAAAGAT | CAAAAT |
| 1631 | E15SaCas9KKH13 | exon15 | SaCas9KKH | − | CAAGATTCACACAACTGGCTTT | AAAGAT |
| 1632 | E15SaCas9KKH14 | exon15 | SaCas9KKH | − | CAGAAAAAGAAGATGCAGTGAA | CAAGAT |
| 1633 | E15SaCas9KKH15 | exon15 | SaCas9KKH | − | CATGGCTTTCAGAAAAAGAAGA | TGCAGT |
| 1634 | E15SaCas9KKH16 | exon15 | SaCas9KKH | − | TAGTGCATGGCTTTCAGAAAAA | GAAGAT |
| 1635 | E15SaCas9KKH17 | exon15 | SaCas9KKH | − | CTTTAATGTCTTGCAGTGCCTT | TTTAGT |
| 1636 | E16SaCas9KKH1 | exon16 | SaCas9KKH | + | ACTAACCTGTGCTGTACTCTTT | TCAAGT |
| 1637 | E16SaCas9KKH2 | exon16 | SaCas9KKH | + | GTACTCTTTTCAAGTTTTTGGA | CTAAAT |
| 1638 | E16SaCas9KKH3 | exon16 | SaCas9KKH | + | ACTAAATTATCCCAACACCGGG | CAAAGT |
| 1639 | E16SaCas9KKH4 | exon16 | SaCas9KKH | + | TATCCAGCCATGCTTCCGTCTT | CTGGGT |
| 1640 | E16SaCas9KKH5 | exon16 | SaCas9KKH | + | CTTCTGGGTCACTGACTTATTC | TTCAGT |
| 1641 | E16SaCas9KKH6 | exon16 | SaCas9KKH | + | GACTTATTCTTCAGTGTTGAAA | GAAGAT |
| 1642 | E16SaCas9KKH7 | exon16 | SaCas9KKH | + | CAGTGTTGAAAGAAGATCTTGT | TTGAGT |
| 1643 | E16SaCas9KKH8 | exon16 | SaCas9KKH | + | GTTGAAAGAAGATCTTGTTTGA | GTGAAT |
| 1644 | E16SaCas9KKH9 | exon16 | SaCas9KKH | + | AAGAAGATCTTGTTTGAGTGAA | TACAGT |
| 1645 | E16SaCas9KKH10 | exon16 | SaCas9KKH | + | TTTGAGTGAATACAGTTTGCCC | ATGGAT |
| 1646 | E16SaCas9KKH11 | exon16 | SaCas9KKH | + | CCCATGGATTGCTTTTTCTTTT | CTAGAT |
| 1647 | E16SaCas9KKH15 | exon16 | SaCas9KKH | − | TGAAAAGAGTACAGCACAGGTT | AGTGAT |
| 1648 | E16SaCas9KKH16 | exon16 | SaCas9KKH | − | ACTTGAAAAGAGTACAGCACAG | GTTAGT |
| 1649 | E16SaCas9KKH17 | exon16 | SaCas9KKH | − | AAAAACTTGAAAAGAGTACAGC | ACAGGT |
| 1650 | E16SaCas9KKH18 | exon16 | SaCas9KKH | − | TAATTTAGTCCAAAAACTTGAA | AAGAGT |
| 1651 | E16SaCas9KKH19 | exon16 | SaCas9KKH | − | ACTTTGCCCGGTGTTGGGATAA | TTTAGT |
| 1652 | E16SaCas9KKH20 | exon16 | SaCas9KKH | − | GGATAACTTTGCCCGGTGTTGG | GATAAT |
| 1653 | E16SaCas9KKH21 | exon16 | SaCas9KKH | − | GCTGGATAACTTTGCCCGGTGT | TGGGAT |
| 1654 | E16SaCas9KKH22 | exon16 | SaCas9KKH | − | GAAGCATGGCTGGATAACTTTG | CCCGGT |
| 1655 | E16SaCas9KKH23 | exon16 | SaCas9KKH | − | GACCCAGAAGACGGAAGCATGG | CTGGAT |
| 1656 | E16SaCas9KKH24 | exon16 | SaCas9KKH | − | TTCTTTCAACACTGAAGAATAA | GTCAGT |
| 1657 | E16SaCas9KKH25 | exon16 | SaCas9KKH | − | GATCTTCTTTCAACACTGAAGA | ATAAGT |
| 1658 | E16SaCas9KKH26 | exon16 | SaCas9KKH | − | ACAAGATCTTCTTTCAACACTG | AAGAAT |
| 1659 | E16SaCas9KKH27 | exon16 | SaCas9KKH | − | GGGCAAACTGTATTCACTCAAA | CAAGAT |
| 1660 | E16SaCas9KKH28 | exon16 | SaCas9KKH | − | AAAGCGGATCTAGAAAAGAAAA | AGCAAT |
| 1661 | E16SaCas9KKH29 | exon16 | SaCas9KKH | − | CTTGTTTTAACAGGTTTTAAAA | GCGGAT |
| 1662 | E17SaCas9KKH1 | exon17 | SaCas9KKH | + | GCTTAAGATGCTCTCACCTTTT | CCTAAT |
| 1663 | E17SaCas9KKH2 | exon17 | SaCas9KKH | + | TGCTCTCACCTTTTCCTAATTT | CAGAAT |
| 1664 | E17SaCas9KKH3 | exon17 | SaCas9KKH | + | ACCTTTTCCTAATTTCAGAATC | CACAGT |
| 1665 | E17SaCas9KKH4 | exon17 | SaCas9KKH | + | TTTTCCTAATTTCAGAATCCAC | AGTAAT |
| 1666 | E17SaCas9KKH5 | exon17 | SaCas9KKH | + | AGTAATCTGCCTCTTCTTTTGG | GGAGGT |
| 1667 | E17SaCas9KKH6 | exon17 | SaCas9KKH | + | AATCTGCCTCTTCTTTTGGGGA | GGTGGT |
| 1668 | E17SaCas9KKH7 | exon17 | SaCas9KKH | + | CTGCCTCTTCTTTTGGGGAGGT | GGTGGT |
| 1669 | E17SaCas9KKH8 | exon17 | SaCas9KKH | + | CTTCTTTTGGGGAGGTGGTGGT | GGAAGT |
| 1670 | E17SaCas9KKH9 | exon17 | SaCas9KKH | + | GTTCCTCTTGAGCATGCTTTAC | CAGGAT |
| 1671 | E17SaCas9KKH10 | exon17 | SaCas9KKH | + | GCTTTACCAGGATCTGTTCCCT | TGTGGT |
| 1672 | E17SaCas9KKH11 | exon17 | SaCas9KKH | + | GGATCTGTTCCCTTGTGGTCAC | CGTAGT |
| 1673 | E17SaCas9KKH12 | exon17 | SaCas9KKH | + | TCACCGTAGTTACTGTTTCCAT | TACAGT |
| 1674 | E17SaCas9KKH13 | exon17 | SaCas9KKH | + | TGTTTCCATTACAGTTGTCTGT | GTTAGT |
| 1675 | E17SaCas9KKH14 | exon17 | SaCas9KKH | + | TTCCATTACAGTTGTCTGTGTT | AGTGAT |
| 1676 | E17SaCas9KKH15 | exon17 | SaCas9KKH | + | CAGTTGTCTGTGTTAGTGATGG | CTGAGT |
| 1677 | E17SaCas9KKH16 | exon17 | SaCas9KKH | + | TTGTCTGTGTTAGTGATGGCTG | AGTGGT |
| 1678 | E17SaCas9KKH17 | exon17 | SaCas9KKH | + | TCTGTGTTAGTGATGGCTGAGT | GGTGGT |
| 1679 | E17SaCas9KKH18 | exon17 | SaCas9KKH | + | GCTGAGTGGTGGTGACAGCCTG | TGAAAT |
| 1680 | E17SaCas9KKH22 | exon17 | SaCas9KKH | − | ACTGTGGATTCTGAAATTAGGA | AAAGGT |
| 1681 | E17SaCas9KKH23 | exon17 | SaCas9KKH | − | AGAGGCAGATTACTGTGGATTC | TGAAAT |
| 1682 | E17SaCas9KKH24 | exon17 | SaCas9KKH | − | CCAAAAGAAGAGGCAGATTACT | GTGGAT |
| 1683 | E17SaCas9KKH25 | exon17 | SaCas9KKH | − | CACCACCTCCCCAAAAGAAGAG | GCAGAT |
| 1684 | E17SaCas9KKH26 | exon17 | SaCas9KKH | − | CGGTGACCACAAGGGAACAGAT | CCTGGT |
| 1685 | E17SaCas9KKH27 | exon17 | SaCas9KKH | − | TAACTACGGTGACCACAAGGGA | ACAGAT |
| 1686 | E17SaCas9KKH28 | exon17 | SaCas9KKH | − | CAACTGTAATGGAAACAGTAAC | TACGGT |
| 1687 | E17SaCas9KKH29 | exon17 | SaCas9KKH | − | TAACACAGACAACTGTAATGGA | AACAGT |
| 1688 | E17SaCas9KKH30 | exon17 | SaCas9KKH | − | AGCCATCACTAACACAGACAAC | TGTAAT |
| 1689 | E18SaCas9KKH1 | exon18 | SaCas9KKH | + | TAACCTACATTGACTTTTTCTT | TTAAGT |
| 1690 | E18SaCas9KKH2 | exon18 | SaCas9KKH | + | TTGACTTTTTCTTTTAAGTCTG | AGAAGT |
| 1691 | E18SaCas9KKH3 | exon18 | SaCas9KKH | + | CTGAGAAGTTGCCTTCCTTCCG | AAAGAT |
| 1692 | E18SaCas9KKH4 | exon18 | SaCas9KKH | + | GTTGCCTTCCTTCCGAAAGATT | GCAAAT |
| 1693 | E18SaCas9KKH5 | exon18 | SaCas9KKH | + | GACTCTGCAACACAGCTTCTGA | GCGAGT |
| 1694 | E18SaCas9KKH6 | exon18 | SaCas9KKH | + | TCTGCAACACAGCTTCTGAGCG | AGTAAT |
| 1695 | E18SaCas9KKH7 | exon18 | SaCas9KKH | + | TTCTGAGCGAGTAATCCAGCTG | TGAAGT |
| 1696 | E18SaCas9KKH8 | exon18 | SaCas9KKH | + | AGCGAGTAATCCAGCTGTGAAG | TTCAGT |
| 1697 | E18SaCas9KKH12 | exon18 | SaCas9KKH | − | AGACTTAAAAGAAAAAGTCAAT | GTAGGT |
| 1698 | E18SaCas9KKH13 | exon18 | SaCas9KKH | − | CTTCTCAGACTTAAAAGAAAAA | GTCAAT |
| 1699 | E18SaCas9KKH14 | exon18 | SaCas9KKH | − | GCAACTTCTCAGACTTAAAAGA | AAAAGT |
| 1700 | E18SaCas9KKH15 | exon18 | SaCas9KKH | − | CTGTGTTGCAGAGTCCTGAATT | TGCAAT |
| 1701 | E18SaCas9KKH16 | exon18 | SaCas9KKH | − | TCAGAAGCTGTGTTGCAGAGTC | CTGAAT |
| 1702 | E18SaCas9KKH17 | exon18 | SaCas9KKH | − | TACTCGCTCAGAAGCTGTGTTG | CAGAGT |
| 1703 | E18SaCas9KKH18 | exon18 | SaCas9KKH | − | TTGATATAACTGAACTTCACAG | CTGGAT |
| 1704 | E18SaCas9KKH19 | exon18 | SaCas9KKH | − | TTTTTGCTGTCTTAGGTTGGAT | GTTGAT |
| 1705 | E19SaCas9KKH2 | exon19 | SaCas9KKH | + | CATCTGTTCCACCAGGGCCTGA | GCTGAT |
| 1706 | E19SaCas9KKH3 | exon19 | SaCas9KKH | + | CTGAGCTGATCTGCTGGCATCT | TGCAGT |
| 1707 | E19SaCas9KKH11 | exon19 | SaCas9KKH | − | CCTGGTGGAACAGATGGTGAAT | GGTAAT |
| 1708 | E19SaCas9KKH12 | exon19 | SaCas9KKH | − | GGCCCTGGTGGAACAGATGGTG | AATGGT |
| 1709 | E19SaCas9KKH13 | exon19 | SaCas9KKH | − | TCAGGCCCTGGTGGAACAGATG | GTGAAT |
| 1710 | E19SaCas9KKH14 | exon19 | SaCas9KKH | − | CAGCTCAGGCCCTGGTGGAACA | GATGGT |
| 1711 | E19SaCas9KKH15 | exon19 | SaCas9KKH | − | GATCAGCTCAGGCCCTGGTGGA | ACAGAT |
| 1712 | E19SaCas9KKH16 | exon19 | SaCas9KKH | − | ATGCCAGCAGATCAGCTCAGGC | CCTGGT |
| 1713 | E19SaCas9KKH17 | exon19 | SaCas9KKH | − | TTCAGAAAACTGCAAGATGCCA | GCAGAT |
| 1714 | E19SaCas9KKH18 | exon19 | SaCas9KKH | − | AGCTGAGAAGTTCAGAAAACTG | CAAGAT |
| 1715 | E19SaCas9KKH19 | exon19 | SaCas9KKH | − | GCCATAGAGCGAGAAAAAGCTG | AGAAGT |
| 1716 | E20SaCas9KKH1 | exon20 | SaCas9KKH | + | AAGGAGAAGAGATTCTTACCTT | ACAAAT |
| 1717 | E20SaCas9KKH2 | exon20 | SaCas9KKH | + | CCTTACAAATTTTTAACTGACT | TTTAAT |
| 1718 | E20SaCas9KKH3 | exon20 | SaCas9KKH | + | CTGACTTTTAATTGCTGTTGGC | TCTGAT |
| 1719 | E20SaCas9KKH4 | exon20 | SaCas9KKH | + | TTTTAATTGCTGTTGGCTCTGA | TGGGGT |
| 1720 | E20SaCas9KKH5 | exon20 | SaCas9KKH | + | TAATTGCTGTTGGCTCTGATGG | GGTGGT |
| 1721 | E20SaCas9KKH6 | exon20 | SaCas9KKH | + | TGCTGTTGGCTCTGATGGGGTG | GTGGGT |
| 1722 | E20SaCas9KKH7 | exon20 | SaCas9KKH | + | TTGGCTCTGATGGGGTGGTGGG | TTGGAT |
| 1723 | E20SaCas9KKH8 | exon20 | SaCas9KKH | + | GGGGTGGTGGGTTGGATTTTCA | ACCAGT |
| 1724 | E20SaCas9KKH9 | exon20 | SaCas9KKH | + | GTTGGATTTTCAACCAGTTTTC | AGCAGT |
| 1725 | E20SaCas9KKH10 | exon20 | SaCas9KKH | + | GGATTTTCAACCAGTTTTCAGC | AGTAGT |
| 1726 | E20SaCas9KKH11 | exon20 | SaCas9KKH | + | TTCAGCAGTAGTTGTCATCTGC | TCCAAT |
| 1727 | E20SaCas9KKH12 | exon20 | SaCas9KKH | + | GTCATCTGCTCCAATTGTTGTA | GCTGAT |
| 1728 | E20SaCas9KKH13 | exon20 | SaCas9KKH | + | ATTGTTGTAGCTGATTATAGAA | AGCGAT |
| 1729 | E20SaCas9KKH14 | exon20 | SaCas9KKH | + | GTTGTAGCTGATTATAGAAAGC | GATGAT |
| 1730 | E20SaCas9KKH15 | exon20 | SaCas9KKH | + | TTATAGAAAGCGATGATGTTGT | TCTGAT |
| 1731 | E20SaCas9KKH16 | exon20 | SaCas9KKH | + | ATGATGTTGTTCTGATACTCCA | GCCAGT |
| 1732 | E20SaCas9KKH17 | exon20 | SaCas9KKH | + | GTTGTTCTGATACTCCAGCCAG | TTAAGT |
| 1733 | E20SaCas9KKH18 | exon20 | SaCas9KKH | + | AAGTCTCTCACTTAGCAACTGG | CAGAAT |
| 1734 | E20SaCas9KKH19 | exon20 | SaCas9KKH | + | TCTCACTTAGCAACTGGCAGAA | TTCGAT |
| 1735 | E20SaCas9KKH20 | exon20 | SaCas9KKH | + | GCAGAATTCGATCCACCGGCTG | TTCAGT |
| 1736 | E20SaCas9KKH21 | exon20 | SaCas9KKH | + | TGTTCAGTTGTTCTGAGGCTTG | TTTGAT |
| 1737 | E20SaCas9KKH25 | exon20 | SaCas9KKH | − | GTCAGTTAAAAATTTGTAAGGT | AAGAAT |
| 1738 | E20SaCas9KKH26 | exon20 | SaCas9KKH | − | TTAAAAGTCAGTTAAAAATTTG | TAAGGT |
| 1739 | E20SaCas9KKH27 | exon20 | SaCas9KKH | − | CAACAGCAATTAAAAGTCAGTT | AAAAAT |
| 1740 | E20SaCas9KKH28 | exon20 | SaCas9KKH | − | TCAGAGCCAACAGCAATTAAAA | GTCAGT |
| 1741 | E20SaCas9KKH29 | exon20 | SaCas9KKH | − | CCCATCAGAGCCAACAGCAATT | AAAAGT |
| 1742 | E20SaCas9KKH30 | exon20 | SaCas9KKH | − | CCACCACCCCATCAGAGCCAAC | AGCAAT |
| 1743 | E20SaCas9KKH31 | exon20 | SaCas9KKH | − | CAACTACTGCTGAAAACTGGTT | GAAAAT |
| 1744 | E20SaCas9KKH32 | exon20 | SaCas9KKH | − | CAGATGACAACTACTGCTGAAA | ACTGGT |
| 1745 | E20SaCas9KKH33 | exon20 | SaCas9KKH | − | ATAATCAGCTACAACAATTGGA | GCAGAT |
| 1746 | E20SaCas9KKH34 | exon20 | SaCas9KKH | − | ATCGCTTTCTATAATCAGCTAC | AACAAT |
| 1747 | E20SaCas9KKH35 | exon20 | SaCas9KKH | − | TCAGAACAACATCATCGCTTTC | TATAAT |
| 1748 | E20SaCas9KKH36 | exon20 | SaCas9KKH | − | CTAAGTGAGAGACTTAACTGGC | TGGAGT |
| 1749 | E20SaCas9KKH37 | exon20 | SaCas9KKH | − | GTGGATCGAATTCTGCCAGTTG | CTAAGT |
| 1750 | E20SaCas9KKH38 | exon20 | SaCas9KKH | − | AACAGCCGGTGGATCGAATTCT | GCCAGT |
| 1751 | E20SaCas9KKH39 | exon20 | SaCas9KKH | − | GAACAACTGAACAGCCGGTGGA | TCGAAT |
| 1752 | E20SaCas9KKH40 | exon20 | SaCas9KKH | − | CCTCAGAACAACTGAACAGCCG | GTGGAT |
| 1753 | E20SaCas9KKH41 | exon20 | SaCas9KKH | − | CAAGCCTCAGAACAACTGAACA | GCCGGT |
| 1754 | E20SaCas9KKH42 | exon20 | SaCas9KKH | − | TTTCTTTCTAGAGGGTGTTAAT | GCAGAT |
| 1755 | E20SaCas9KKH43 | exon20 | SaCas9KKH | − | TATTTTTTTCTTTCTAGAGGGT | GTTAAT |
| 1756 | E21SaCas9KKH1 | exon21 | SaCas9KKH | + | TGCACATCAGAAAAGACTTGCT | TAAAAT |
| 1757 | E21SaCas9KKH2 | exon21 | SaCas9KKH | + | ACATCAGAAAAGACTTGCTTAA | AATGAT |
| 1758 | E21SaCas9KKH3 | exon21 | SaCas9KKH | + | TTAAAATGATTTGTAAAGGCCA | CAAAGT |
| 1759 | E21SaCas9KKH4 | exon21 | SaCas9KKH | + | CACAAAGTCTGCATCCAGGAAC | ATGGGT |
| 1760 | E21SaCas9KKH5 | exon21 | SaCas9KKH | + | CTTTCTCTTTCAGGGCTATGCT | TTGAAT |
| 1761 | E21SaCas9KKH6 | exon21 | SaCas9KKH | + | TTTCAGGGCTATGCTTTGAATT | TTTAAT |
| 1762 | E21SaCas9KKH7 | exon21 | SaCas9KKH | + | CTATGCTTTGAATTTTTAATCG | TTCAAT |
| 1763 | E21SaCas9KKH8 | exon21 | SaCas9KKH | + | TTGAATTTTTAATCGTTCAATT | TGAGGT |
| 1764 | E21SaCas9KKH9 | exon21 | SaCas9KKH | + | TTTAATCGTTCAATTTGAGGTT | GAAGAT |
| 1765 | E21SaCas9KKH10 | exon21 | SaCas9KKH | + | TCGTTCAATTTGAGGTTGAAGA | TCTGAT |
| 1766 | E21SaCas9KKH11 | exon21 | SaCas9KKH | + | ATTTGAGGTTGAAGATCTGATA | GCCGGT |
| 1767 | E21SaCas9KKH16 | exon21 | SaCas9KKH | − | AGAGAGAAAGAGCTACAGACAA | GTAAGT |
| 1768 | E21SaCas9KKH17 | exon21 | SaCas9KKH | − | GGCCAGAGAGAAAGAGCTACAG | ACAAGT |
| 1769 | E21SaCas9KKH18 | exon21 | SaCas9KKH | − | AAATCATTTTAAGCAAGTCTTT | TCTGAT |
| 1770 | E21SaCas9KKH19 | exon21 | SaCas9KKH | − | TGGCCTTTACAAATCATTTTAA | GCAAGT |
| 1771 | E21SaCas9KKH20 | exon21 | SaCas9KKH | − | GGATGCAGACTTTGTGGCCTTT | ACAAAT |
| 1772 | E21SaCas9KKH21 | exon21 | SaCas9KKH | − | GAAAGGACAAGGACCCATGTTC | CTGGAT |
| 1773 | E21SaCas9KKH22 | exon21 | SaCas9KKH | − | TTCAACCTCAAATTGAACGATT | AAAAAT |
| 1774 | E21SaCas9KKH23 | exon21 | SaCas9KKH | − | TCAGATCTTCAACCTCAAATTG | AACGAT |
| 1775 | E21SaCas9KKH24 | exon21 | SaCas9KKH | − | ACCGGCTATCAGATCTTCAACC | TCAAAT |
| 1776 | E21SaCas9KKH25 | exon21 | SaCas9KKH | − | ACAGGATGAAGTCAACCGGCTA | TCAGAT |
| 1777 | E22SaCas9KKH2 | exon22 | SaCas9KKH | + | ATATTCACAGACCTGCAATTCC | CCGAGT |
| 1778 | E22SaCas9KKH3 | exon22 | SaCas9KKH | + | GCAATTCCCCGAGTCTCTGCTC | CATGAT |
| 1779 | E22SaCas9KKH4 | exon22 | SaCas9KKH | + | CCGAGTCTCTGCTCCATGATTT | CATAGT |
| 1780 | E22SaCas9KKH5 | exon22 | SaCas9KKH | + | GTCTCTGCTCCATGATTTCATA | GTCGGT |
| 1781 | E22SaCas9KKH6 | exon22 | SaCas9KKH | 1 | CATGATTTCATAGTCGGTGACA | CTAAGT |
| 1782 | E22SaCas9KKH7 | exon22 | SaCas9KKH | + | TTCATAGTCGGTGACACTAAGT | TGAGGT |
| 1783 | E22SaCas9KKH8 | exon22 | SaCas9KKH | 1 | GGTGACACTAAGTTGAGGTATG | GAGAGT |
| 1784 | E22SaCas9KKH9 | exon22 | SaCas9KKH | + | CACTAAGTTGAGGTATGGAGAG | TTTGGT |
| 1785 | E22SaCas9KKH10 | exon22 | SaCas9KKH | + | TTTCTGACTGCTGGACCCATGT | CCTGAT |
| 1786 | E22SaCas9KKH11 | exon22 | SaCas9KKH | + | GGACCCATGTCCTGATGGCACT | CATGGT |
| 1787 | E22SaCas9KKH12 | exon22 | SaCas9KKH | + | GTCCTGATGGCACTCATGGTCT | CCTGAT |
| 1788 | E22SaCas9KKH13 | exon22 | SaCas9KKH | + | ACTCATGGTCTCCTGATAGCGC | ATTGGT |
| 1789 | E22SaCas9KKH14 | exon22 | SaCas9KKH | + | TCTCCTGATAGCGCATTGGTGG | CAAAGT |
| 1790 | E22SaCas9KKH20 | exon22 | SaCas9KKH | − | TGGAGCAGAGACTCGGGGAATT | GCAGGT |
| 1791 | E22SaCas9KKH21 | exon22 | SaCas9KKH | − | GAAATCATGGAGCAGAGACTCG | GGGAAT |
| 1792 | E22SaCas9KKH22 | exon22 | SaCas9KKH | − | CTCAACTTAGTGTCACCGACTA | TGAAAT |
| 1793 | E22SaCas9KKH23 | exon22 | SaCas9KKH | − | AACCAAACTCTCCATACCTCAA | CTTAGT |
| 1794 | E22SaCas9KKH24 | exon22 | SaCas9KKH | − | AGTGCCATCAGGACATGGGTCC | AGCAGT |
| 1795 | E22SaCas9KKH25 | exon22 | SaCas9KKH | − | AGACCATGAGTGCCATCAGGAC | ATGGGT |
| 1796 | E22SaCas9KKH26 | exon22 | SaCas9KKH | − | ACCAATGCGCTATCAGGAGACC | ATGAGT |
| 1797 | E22SaCas9KKH27 | exon22 | SaCas9KKH | − | ATAAAGTTTTTGACACTTTGCC | ACCAAT |
| 1798 | E23SaCas9KKH3 | exon23 | SaCas9KKH | + | ATCTTGAATTACCTGAATTTTT | CGGAGT |
| 1799 | E23SaCas9KKH4 | exon23 | SaCas9KKH | + | TTCATTTGCTCCTCTAGCTTTT | GACAAT |
| 1800 | E23SaCas9KKH5 | exon23 | SaCas9KKH | + | AGGAGAGCTTCTTCCAGCGTCC | CTCAAT |
| 1801 | E23SaCas9KKH6 | exon23 | SaCas9KKH | + | CTTCCAGCGTCCCTCAATTTCT | TCAAAT |
| 1802 | E23SaCas9KKH7 | exon23 | SaCas9KKH | + | GCGTCCCTCAATTTCTTCAAAT | TCTGAT |
| 1803 | E23SaCas9KKH8 | exon23 | SaCas9KKH | + | CCCTCAATTTCTTCAAATTCTG | ATTGAT |
| 1804 | E23SaCas9KKH9 | exon23 | SaCas9KKH | + | CAAATTCTGATTGATATTTCCG | GCTAAT |
| 1805 | E23SaCas9KKH10 | exon23 | SaCas9KKH | + | GCGCTTTCTTCGACATCTCTTT | CACAGT |
| 1806 | E23SaCas9KKH11 | exon23 | SaCas9KKH | + | CTTTCTTCGACATCTCTTTCAC | AGTGGT |
| 1807 | E23SaCas9KKH12 | exon23 | SaCas9KKH | + | GACATCTCTTTCACAGTGGTGC | TGAGAT |
| 1808 | E23SaCas9KKH13 | exon23 | SaCas9KKH | + | ATCTCTTTCACAGTGGTGCTGA | GATAGT |
| 1809 | E23SaCas9KKH17 | exon23 | SaCas9KKH | − | TGAATAAACTCCGAAAAATTCA | GGTAAT |
| 1810 | E23SaCas9KKH18 | exon23 | SaCas9KKH | − | AAATGAATAAACTCCGAAAAAT | TCAGGT |
| 1811 | E23SaCas9KKH19 | exon23 | SaCas9KKH | − | AGGAGCAAATGAATAAACTCCG | AAAAAT |
| 1812 | E23SaCas9KKH20 | exon23 | SaCas9KKH | − | TTGTCAAAAGCTAGAGGAGCAA | ATGAAT |
| 1813 | E23SaCas9KKH21 | exon23 | SaCas9KKH | − | AGCATTGTCAAAAGCTAGAGGA | GCAAAT |
| 1814 | E23SaCas9KKH22 | exon23 | SaCas9KKH | − | GCTGGAAGAAGCTCTCCTCCCA | GCTGGT |
| 1815 | E23SaCas9KKH23 | exon23 | SaCas9KKH | − | GGAAATATCAATCAGAATTTGA | AGAAAT |
| 1816 | E23SaCas9KKH24 | exon23 | SaCas9KKH | − | GAAATTAGCCGGAAATATCAAT | CAGAAT |
| 1817 | E23SaCas9KKH25 | exon23 | SaCas9KKH | − | CCCTCTGAAATTAGCCGGAAAT | ATCAAT |
| 1818 | E23SaCas9KKH26 | exon23 | SaCas9KKH | − | AAAGCGCCCTCTGAAATTAGCC | GGAAAT |
| 1819 | E23SaCas9KKH27 | exon23 | SaCas9KKH | − | AGATGTCGAAGAAAGCGCCCTC | TGAAAT |
| 1820 | E23SaCas9KKH28 | exon23 | SaCas9KKH | − | ACTATCTCAGCACCACTGTGAA | AGAGAT |
| 1821 | E23SaCas9KKH29 | exon23 | SaCas9KKH | − | ACAAAGTTCTCTGCAAGAGCAA | CAAAGT |
| 1822 | E23SaCas9KKH30 | exon23 | SaCas9KKH | − | AAAAATTGTTTTTTAGGCTTTA | CAAAGT |
| 1823 | E24SaCas9KKH1 | exon24 | SaCas9KKH | + | TGCACTGTTTCAGCTGCTTTTT | TAGAAT |
| 1824 | E24SaCas9KKH2 | exon24 | SaCas9KKH | + | TTCAGCTGCTTTTTTAGAATTT | CTGAAT |
| 1825 | E24SaCas9KKH3 | exon24 | SaCas9KKH | + | CAACTTCAGCCATCCATTTCTT | CAGGGT |
| 1826 | E24SaCas9KKH4 | exon24 | SaCas9KKH | + | ATCCATTTCTTCAGGGTTTGTA | TGTGAT |
| 1827 | E24SaCas9KKH7 | exon24 | SaCas9KKH | − | AAGCAGCTGAAACAGTGCAGAG | TAAGAT |
| 1828 | E24SaCas9KKH8 | exon24 | SaCas9KKH | − | TAAAAAAGCAGCTGAAACAGTG | CAGAGT |
| 1829 | E24SaCas9KKH9 | exon24 | SaCas9KKH | − | GAAATTCTAAAAAAGCAGCTGA | AACAGT |
| 1830 | E24SaCas9KKH10 | exon24 | SaCas9KKH | − | AATGGCCTGCCCTTGGGGATTC | AGAAAT |
| 1831 | E24SaCas9KKH11 | exon24 | SaCas9KKH | − | GAAGGAGGAATGGCCTGCCCTT | GGGGAT |
| 1832 | E24SaCas9KKH12 | exon24 | SaCas9KKH | − | GAAGTTGATGTTTTTCTGAAGG | AGGAAT |
| 1833 | E24SaCas9KKH13 | exon24 | SaCas9KKH | − | CCTGAAGAAATGGATGGCTGAA | GTTGAT |
| 1834 | E24SaCas9KKH14 | exon24 | SaCas9KKH | − | AAACCCTGAAGAAATGGATGGC | TGAAGT |
| 1835 | E24SaCas9KKH15 | exon24 | SaCas9KKH | − | ATCACATACAAACCCTGAAGAA | ATGGAT |
| 1836 | E24SaCas9KKH16 | exon24 | SaCas9KKH | − | CAGAATCACATACAAACCCTGA | AGAAAT |
| 1837 | E25SaCas9KKH1 | exon25 | SaCas9KKH | + | ATTGTCTATACCTGTTGGCACA | TGTGAT |
| 1838 | E25SaCas9KKH2 | exon25 | SaCas9KKH | + | CCTGTTGGCACATGTGATCCCA | CTGAGT |
| 1839 | E25SaCas9KKH3 | exon25 | SaCas9KKH | + | GCACATGTGATCCCACTGAGTG | TTAAGT |
| 1840 | E25SaCas9KKH4 | exon25 | SaCas9KKH | + | ATCCCACTGAGTGTTAAGTTCT | TTGAGT |
| 1841 | E25SaCas9KKH5 | exon25 | SaCas9KKH | + | GTTAAGTTCTTTGAGTTCTGTC | TCAAGT |
| 1842 | E25SaCas9KKH6 | exon25 | SaCas9KKH | + | CATTGACACTGTTTAGACTGGG | CTGAAT |
| 1843 | E25SaCas9KKH7 | exon25 | SaCas9KKH | + | TGTTTAGACTGGGCTGAATTGT | CTGAAT |
| 1844 | E25SaCas9KKH12 | exon25 | SaCas9KKH | − | CTCAGTGGGATCACATGTGCCA | ACAGGT |
| 1845 | E25SaCas9KKH13 | exon25 | SaCas9KKH | − | ACTCAAAGAACTTAACACTCAG | TGGGAT |
| 1846 | E25SaCas9KKH14 | exon25 | SaCas9KKH | − | ACAGAACTCAAAGAACTTAACA | CTCAGT |
| 1847 | E25SaCas9KKH15 | exon25 | SaCas9KKH | − | AAGATAAAGAATGAAGCAGAGC | CAGAGT |
| 1848 | E25SaCas9KKH16 | exon25 | SaCas9KKH | − | CAATGAAGGTGGGCAGAAGATA | AAGAAT |
| 1849 | E25SaCas9KKH17 | exon25 | SaCas9KKH | − | ACAGTGTCAATGAAGGTGGGCA | GAAGAT |
| 1850 | E25SaCas9KKH18 | exon25 | SaCas9KKH | − | GCCCAGTCTAAACAGTGTCAAT | GAAGGT |
| 1851 | E25SaCas9KKH19 | exon25 | SaCas9KKH | − | AATTCAGCCCAGTCTAAACAGT | GTCAAT |
| 1852 | E25SaCas9KKH20 | exon25 | SaCas9KKH | − | TCAGACAATTCAGCCCAGTCTA | AACAGT |
| 1853 | E25SaCas9KKH21 | exon25 | SaCas9KKH | − | CAGTGATATTCAGACAATTCAG | CCCAGT |
| 1854 | E25SaCas9KKH22 | exon25 | SaCas9KKH | − | AGCTTTTAGTCAGTGATATTCA | GACAAT |
| 1855 | E25SaCas9KKH23 | exon25 | SaCas9KKH | − | TTTATTTTCTTAGCTTTTAGTC | AGTGAT |
| 1856 | E25SaCas9KKH24 | exon25 | SaCas9KKH | − | TGATTTATTTTCTTAGCTTTTA | GTCAGT |
| 1857 | E26SaCas9KKH1 | exon26 | SaCas9KKH | + | CTTCATCTCTTCAACTGCTTTC | TGTAAT |
| 1858 | E26SaCas9KKH2 | exon26 | SaCas9KKH | + | CAACTGCTTTCTGTAATTCATC | TGGAGT |
| 1859 | E26SaCas9KKH3 | exon26 | SaCas9KKH | + | AATTCATCTGGAGTTTTATATT | CAAAAT |
| 1860 | E26SaCas9KKH4 | exon26 | SaCas9KKH | + | GTTTTATATTCAAAATCTCTCT | CAAGAT |
| 1861 | E26SaCas9KKH5 | exon26 | SaCas9KKH | + | TTGTGTCATCCATTCGTGCATC | TCTGAT |
| 1862 | E26SaCas9KKH6 | exon26 | SaCas9KKH | + | GTCATCCATTCGTGCATCTCTG | ATAGAT |
| 1863 | E26SaCas9KKH7 | exon26 | SaCas9KKH | + | CTGATAGATCTTTCTGGAGGCT | TACAGT |
| 1864 | E26SaCas9KKH10 | exon26 | SaCas9KKH | − | TACAGAAAGCAGTTGAAGAGAT | GAAGGT |
| 1865 | E26SaCas9KKH11 | exon26 | SaCas9KKH | − | ATGAATTACAGAAAGCAGTTGA | AGAGAT |
| 1866 | E26SaCas9KKH12 | exon26 | SaCas9KKH | − | AAACTCCAGATGAATTACAGAA | AGCAGT |
| 1867 | E26SaCas9KKH13 | exon26 | SaCas9KKH | − | GATTTTGAATATAAAACTCCAG | ATGAAT |
| 1868 | E26SaCas9KKH14 | exon26 | SaCas9KKH | − | GAGAGATTTTGAATATAAAACT | CCAGAT |
| 1869 | E26SaCas9KKH15 | exon26 | SaCas9KKH | − | GAAGAGTATCTTGAGAGAGATT | TTGAAT |
| 1870 | E26SaCas9KKH16 | exon26 | SaCas9KKH | − | AGCTGAAGAAGAGTATCTTGAG | AGAGAT |
| 1871 | E26SaCas9KKH17 | exon26 | SaCas9KKH | − | GAATGGATGACACAAGCTGAAG | AAGAGT |
| 1872 | E26SaCas9KKH18 | exon26 | SaCas9KKH | − | AAGATCTATCAGAGATGCACGA | ATGGAT |
| 1873 | E26SaCas9KKH19 | exon26 | SaCas9KKH | − | CAGAAAGATCTATCAGAGATGC | ACGAAT |
| 1874 | E26SaCas9KKH20 | exon26 | SaCas9KKH | − | TAAGCCTCCAGAAAGATCTATC | AGAGAT |
| 1875 | E26SaCas9KKH21 | exon26 | SaCas9KKH | − | GGAGAAAACTGTAAGCCTCCAG | AAAGAT |
| 1876 | E26SaCas9KKH22 | exon26 | SaCas9KKH | − | TGCCAGAAAGGAGGCCTTGAAG | GGAGGT |
| 1877 | E27SaCas9KKH1 | exon27 | SaCas9KKH | + | ACCAAGAAAAGCAACTGACTTC | CAAAGT |
| 1878 | E27SaCas9KKH2 | exon27 | SaCas9KKH | + | AAGTCTTGCATTTCCCATTCAG | CCTAGT |
| 1879 | E27SaCas9KKH3 | exon27 | SaCas9KKH | + | CCATTCAGCCTAGTGCAGAGCC | ACTGGT |
| 1880 | E27SaCas9KKH4 | exon27 | SaCas9KKH | + | TTCAGCCTAGTGCAGAGCCACT | GGTAGT |
| 1881 | E27SaCas9KKH5 | exon27 | SaCas9KKH | + | GCCTAGTGCAGAGCCACTGGTA | GTTGGT |
| 1882 | E27SaCas9KKH6 | exon27 | SaCas9KKH | + | TAGTGCAGAGCCACTGGTAGTT | GGTGGT |
| 1883 | E27SaCas9KKH7 | exon27 | SaCas9KKH | + | AGAGCCACTGGTAGTTGGTGGT | TAGAGT |
| 1884 | E27SaCas9KKH8 | exon27 | SaCas9KKH | + | CTGGTAGTTGGTGGTTAGAGTT | TCAAGT |
| 1885 | E27SaCas9KKH9 | exon27 | SaCas9KKH | + | CTTTTTTAAGGCCTCTTGTGCT | ACAGGT |
| 1886 | E27SaCas9KKH10 | exon27 | SaCas9KKH | + | GAGCTATGACACTATTTACAGA | CTCAGT |
| 1887 | E27SaCas9KKH11 | exon27 | SaCas9KKH | + | GACACTATTTACAGACTCAGTA | AGGAGT |
| 1888 | E27SaCas9KKH18 | exon27 | SaCas9KKH | − | TGGGAAATGCAAGACTTTGGAA | GTCAGT |
| 1889 | E27SaCas9KKH19 | exon27 | SaCas9KKH | − | TGAATGGGAAATGCAAGACTTT | GGAAGT |
| 1890 | E27SaCas9KKH20 | exon27 | SaCas9KKH | − | TGGCTCTGCACTAGGCTGAATG | GGAAAT |
| 1891 | E27SaCas9KKH21 | exon27 | SaCas9KKH | − | CTACCAGTGGCTCTGCACTAGG | CTGAAT |
| 1892 | E27SaCas9KKH22 | exon27 | SaCas9KKH | − | CTTGAAACTCTAACCACCAACT | ACCAGT |
| 1893 | E27SaCas9KKH23 | exon27 | SaCas9KKH | − | GAAACTCCTTACTGAGTCTGTA | AATAGT |
| 1894 | E27SaCas9KKH24 | exon27 | SaCas9KKH | − | AGTGAAACTCCTTACTGAGTCT | GTAAAT |
| 1895 | E27SaCas9KKH25 | exon27 | SaCas9KKH | − | GAAGCGAAAGTGAAACTCCTTA | CTGAGT |
| 1896 | E27SaCas9KKH26 | exon27 | SaCas9KKH | + | AAGAGGCCCAACAAAAAGAAGC | GAAAGT |
| 1897 | E28SaCas9KKH1 | exon28 | SaCas9KKH | + | TACAACTTACATCTAGCACCTC | AGAGAT |
| 1898 | E28SaCas9KKH2 | exon28 | SaCas9KKH | + | CAGAGATTTCCTCAGCTCCGCC | AGGAAT |
| 1899 | E28SaCas9KKH3 | exon28 | SaCas9KKH | + | CCTCAGCTCCGCCAGGAATGTT | TTCAGT |
| 1900 | E28SaCas9KKH4 | exon28 | SaCas9KKH | + | CAGCTCCGCCAGGAATGTTTTC | AGTGGT |
| 1901 | E28SaCas9KKH5 | exon28 | SaCas9KKH | + | GCCAGGAATGTTTTCAGTGGTT | TTAAGT |
| 1902 | E28SaCas9KKH6 | exon28 | SaCas9KKH | + | AATGTTTTCAGTGGTTTTAAGT | TTAAAT |
| 1903 | E28SaCas9KKH7 | exon28 | SaCas9KKH | + | TTTAGCCACTTGTTTGCTTTCT | CCAAGT |
| 1904 | E28SaCas9KKH8 | exon28 | SaCas9KKH | + | CTTGTTTGCTTTCTCCAAGTAT | GACAAT |
| 1905 | E28SaCas9KKH17 | exon28 | SaCas9KKH | − | GGAAATCTCTGAGGTGCTAGAT | GTAAGT |
| 1906 | E28SaCas9KKH18 | exon28 | SaCas9KKH | − | AGCTGAGGAAATCTCTGAGGTG | CTAGAT |
| 1907 | E28SaCas9KKH19 | exon28 | SaCas9KKH | − | CTGGCGGAGCTGAGGAAATCTC | TGAGGT |
| 1908 | E28SaCas9KKH20 | exon28 | SaCas9KKH | − | AAAACATTCCTGGCGGAGCTGA | GGAAAT |
| 1909 | E28SaCas9KKH21 | exon28 | SaCas9KKH | − | GCAAACAAGTGGCTAAATGAAG | TAGAAT |
| 1910 | E28SaCas9KKH22 | exon28 | SaCas9KKH | − | AGAAAGCAAACAAGTGGCTAAA | TGAAGT |
| 1911 | E28SaCas9KKH23 | exon28 | SaCas9KKH | − | CTTGGAGAAAGCAAACAAGTGG | CTAAAT |
| 1912 | E28SaCas9KKH24 | exon28 | SaCas9KKH | − | TTGTCATACTTGGAGAAAGCAA | ACAAGT |
| 1913 | E28SaCas9KKH25 | exon28 | SaCas9KKH | − | TAGGAAGTTTGGGCATGTTGGC | ATGAGT |
| 1914 | E29SaCas9KKH1 | exon29 | SaCas9KKH | + | CACTTATCTTCATACCTCTTCA | TGTAGT |
| 1915 | E29SaCas9KKH2 | exon29 | SaCas9KKH | + | TCTTCATGTAGTTCCCTCCAAC | GAGAAT |
| 1916 | E29SaCas9KKH3 | exon29 | SaCas9KKH | + | ATGTAGTTCCCTCCAACGAGAA | TTAAAT |
| 1917 | E29SaCas9KKH4 | exon29 | SaCas9KKH | + | CCTCCAACGAGAATTAAATGTC | TCAAGT |
| 1918 | E29SaCas9KKH5 | exon29 | SaCas9KKH | + | AATTAAATGTCTCAAGTTCCTC | ATTGAT |
| 1919 | E29SaCas9KKH6 | exon29 | SaCas9KKH | + | CATCCATGACTCCGCCATCTGT | TAGGGT |
| 1920 | E29SaCas9KKH7 | exon29 | SaCas9KKH | + | TCCGCCATCTGTTAGGGTCTGT | GCCAAT |
| 1921 | E29SaCas9KKH8 | exon29 | SaCas9KKH | + | CTGTTAGGGTCTGTGCCAATAT | GCGAAT |
| 1922 | E29SaCas9KKH9 | exon29 | SaCas9KKH | + | AGGGTCTGTGCCAATATGCGAA | TCTGAT |
| 1923 | E29SaCas9KKH10 | exon29 | SaCas9KKH | + | TGTGCCAATATGCGAATCTGAT | TTGGGT |
| 1924 | E29SaCas9KKH11 | exon29 | SaCas9KKH | + | CGAATCTGATTTGGGTTATCCT | CTGAAT |
| 1925 | E29SaCas9KKH12 | exon29 | SaCas9KKH | + | GGGTTATCCTCTGAATGTCGCA | TCAAAT |
| 1926 | E29SaCas9KKH13 | exon29 | SaCas9KKH | + | CTCTGAATGTCGCATCAAATTT | TCAAGT |
| 1927 | E29SaCas9KKH20 | exon29 | SaCas9KKH | − | CTCGTTGGAGGGAACTACATGA | AGAGGT |
| 1928 | E29SaCas9KKH21 | exon29 | SaCas9KKH | − | AATCAATGAGGAACTTGAGACA | TTTAAT |
| 1929 | E29SaCas9KKH22 | exon29 | SaCas9KKH | − | TGGCGGAGTCATGGATGAGCTA | ATCAAT |
| 1930 | E29SaCas9KKH23 | exon29 | SaCas9KKH | − | CAGATGGCGGAGTCATGGATGA | GCTAAT |
| 1931 | E29SaCas9KKH24 | exon29 | SaCas9KKH | − | GACCCTAACAGATGGCGGAGTC | ATGGAT |
| 1932 | E29SaCas9KKH25 | exon29 | SaCas9KKH | − | TGGCACAGACCCTAACAGATGG | CGGAGT |
| 1933 | E29SaCas9KKH26 | exon29 | SaCas9KKH | − | TCGCATATTGGCACAGACCCTA | ACAGAT |
| 1934 | E29SaCas9KKH27 | exon29 | SaCas9KKH | − | GACATTCAGAGGATAACCCAAA | TCAGAT |
| 1935 | E29SaCas9KKH28 | exon29 | SaCas9KKH | − | GATGCGACATTCAGAGGATAAC | CCAAAT |
| 1936 | E29SaCas9KKH29 | exon29 | SaCas9KKH | − | TGAAAATTTGATGCGACATTCA | GAGGAT |
| 1937 | E29SaCas9KKH30 | exon29 | SaCas9KKH | − | ATGTGTTTCAGTCACTTGAAAA | TTTGAT |
| 1938 | E29SaCas9KKH31 | exon29 | SaCas9KKH | − | TGCAAATGTGTTTCAGTCACTT | GAAAAT |
| 1939 | E30SaCas9KKH1 | exon30 | SaCas9KKH | + | TTTGAGCTGCGTCCACCTTGTC | TGCAAT |
| 1940 | E30SaCas9KKH2 | exon30 | SaCas9KKH | + | CAATATAAGCTGCCAACTGCTT | GTCAAT |
| 1941 | E30SaCas9KKH3 | exon30 | SaCas9KKH | + | ATAAGCTGCCAACTGCTTGTCA | ATGAAT |
| 1942 | E30SaCas9KKH4 | exon30 | SaCas9KKH | + | TGTCAATGAATGTGAGGGACTC | CTGGAT |
| 1943 | E30SaCas9KKH5 | exon30 | SaCas9KKH | + | ATGAATGTGAGGGACTCCTGGA | TTAAGT |
| 1944 | E30SaCas9KKH6 | exon30 | SaCas9KKH | + | GAGGGACTCCTGGATTAAGTGT | AAGGAT |
| 1945 | E30SaCas9KKH7 | exon30 | SaCas9KKH | + | CCTGGATTAAGTGTAAGGATTT | TTCAGT |
| 1946 | E30SaCas9KKH8 | exon30 | SaCas9KKH | + | ATTTTTCAGTCTCCTGGGCAGA | CTGGAT |
| 1947 | E30SaCas9KKH13 | exon30 | SaCas9KKH | − | TCAAATGCCTCAGGAAGCCCAG | GCAAGT |
| 1948 | E30SaCas9KKH14 | exon30 | SaCas9KKH | − | TTGCAGACAAGGTGGACGCAGC | TCAAAT |
| 1949 | E30SaCas9KKH15 | exon30 | SaCas9KKH | − | AGTTGGCAGCTTATATTGCAGA | CAAGGT |
| 1950 | E30SaCas9KKH16 | exon30 | SaCas9KKH | − | GAGTCCCTCACATTCATTGACA | AGCAGT |
| 1951 | E30SaCas9KKH17 | exon30 | SaCas9KKH | − | GAAAAATCCTTACACTTAATCC | AGGAGT |
| 1952 | E30SaCas9KKH18 | exon30 | SaCas9KKH | − | AGGAGACTGAAAAATCCTTACA | CTTAAT |
| 1953 | E30SaCas9KKH19 | exon30 | SaCas9KKH | − | ATCCAGTCTGCCCAGGAGACTG | AAAAAT |
| 1954 | E30SaCas9KKH20 | exon30 | SaCas9KKH | − | CAAAAGTTGCTTGAACAGAGCA | TCCAGT |
| 1955 | E30SaCas9KKH21 | exon30 | SaCas9KKH | − | TCTTTTTAGGCTGTAAGGAGGC | AAAAGT |
| 1956 | E31SaCas9KKH1 | exon31 | SaCas9KKH | + | AAATAACATATACCTGTGCAAC | ATCAAT |
| 1957 | E31SaCas9KKH2 | exon31 | SaCas9KKH | + | ACTCTTTGGGCAGCCTCCTTCC | CCTGAT |
| 1958 | E31SaCas9KKH3 | exon31 | SaCas9KKH | + | TATGTTTCTTCATTTCTTCTAA | ACTGAT |
| 1959 | E31SaCas9KKH4 | exon31 | SaCas9KKH | + | TCTAAACTGATCTCATGACTTG | TCAAAT |
| 1960 | E31SaCas9KKH5 | exon31 | SaCas9KKH | + | ACTGATCTCATGACTTGTCAAA | TCAGAT |
| 1961 | E31SaCas9KKH6 | exon31 | SaCas9KKH | + | TCTCATGACTTGTCAAATCAGA | TTGGAT |
| 1962 | E31SaCas9KKH9 | exon31 | SaCas9KKH | − | TCCTGTCTCAGATTGATGTTGC | ACAGGT |
| 1963 | E31SaCas9KKH10 | exon31 | SaCas9KKH | − | TGCCCAAAGAGTCCTGTCTCAG | ATTGAT |
| 1964 | E31SaCas9KKH11 | exon31 | SaCas9KKH | − | AGGCTGCCCAAAGAGTCCTGTC | TCAGAT |
| 1965 | E31SaCas9KKH12 | exon31 | SaCas9KKH | − | ATCAGGGGAAGGAGGCTGCCCA | AAGAGT |
| 1966 | E31SaCas9KKH13 | exon31 | SaCas9KKH | − | CAGTTTAGAAGAAATGAAGAAA | CATAAT |
| 1967 | E31SaCas9KKH14 | exon31 | SaCas9KKH | − | CAAGTCATGAGATCAGTTTAGA | AGAAAT |
| 1968 | E31SaCas9KKH15 | exon31 | SaCas9KKH | − | ATCTGATTTGACAAGTCATGAG | ATCAGT |
| 1969 | E31SaCas9KKH16 | exon31 | SaCas9KKH | − | TCCAATCTGATTTGACAAGTCA | TGAGAT |
| 1970 | E31SaCas9KKH17 | exon31 | SaCas9KKH | − | ACAGAAAATCCAATCTGATTTG | ACAAGT |
| 1971 | E31SaCas9KKH18 | exon31 | SaCas9KKH | − | ATCCTCCCAACAGAAAATCCAA | TCTGAT |
| 1972 | E31SaCas9KKH19 | exon31 | SaCas9KKH | − | ATGCTATCCTCCCAACAGAAAA | TCCAAT |
| 1973 | E32SaCas9KKH2 | exon32 | SaCas9KKH | + | CCACCAGAAATACATACCACAC | AATGAT |
| 1974 | E32SaCas9KKH3 | exon32 | SaCas9KKH | + | TTCCTGTTCCACACTCTTTGTT | TCCAAT |
| 1975 | E32SaCas9KKH4 | exon32 | SaCas9KKH | + | ACACTCTTTGTTTCCAATGCAG | GCAAGT |
| 1976 | E32SaCas9KKH5 | exon32 | SaCas9KKH | + | GCAAGTGCATCTTCACTTCATC | TAAAAT |
| 1977 | E32SaCas9KKH6 | exon32 | SaCas9KKH | + | TTACTTTCTTGTAGACGCTGCT | CAAAAT |
| 1978 | E32SaCas9KKH7 | exon32 | SaCas9KKH | + | TTGTAGACGCTGCTCAAAATTG | GCTGGT |
| 1979 | E32SaCas9KKH8 | exon32 | SaCas9KKH | + | CTGCTCAAAATTGGCTGGTTTC | TGGAAT |
| 1980 | E32SaCas9KKH9 | exon32 | SaCas9KKH | + | CTCAAAATTGGCTGGTTTCTGG | AATAAT |
| 1981 | E32SaCas9KKH10 | exon32 | SaCas9KKH | + | TCGAAACTTCATGGAGACATCT | TGTAAT |
| 1982 | E32SaCas9KKH16 | exon32 | SaCas9KKH | − | TACAGTCACAGCTAAATCATTG | TGTGGT |
| 1983 | E32SaCas9KKH17 | exon32 | SaCas9KKH | − | ACAGGAAGTAGTACAGTCACAG | CTAAAT |
| 1984 | E32SaCas9KKH18 | exon32 | SaCas9KKH | − | AAGAGTGTGGAACAGGAAGTAG | TACAGT |
| 1985 | E32SaCas9KKH19 | exon32 | SaCas9KKH | − | AAACAAAGAGTGTGGAACAGGA | AGTAGT |
| 1986 | E32SaCas9KKH20 | exon32 | SaCas9KKH | − | TGGAAACAAAGAGTGTGGAACA | GGAAGT |
| 1987 | E32SaCas9KKH21 | exon32 | SaCas9KKH | − | GCACTTGCCTGCATTGGAAACA | AAGAGT |
| 1988 | E32SaCas9KKH22 | exon32 | SaCas9KKH | − | GTAAGATGATTTTAGATGAAGT | GAAGAT |
| 1989 | E32SaCas9KKH23 | exon32 | SaCas9KKH | − | AAGAAAGTAAGATGATTTTAGA | TGAAGT |
| 1990 | E32SaCas9KKH24 | exon32 | SaCas9KKH | − | TCTACAAGAAAGTAAGATGATT | TTAGAT |
| 1991 | E32SaCas9KKH25 | exon32 | SaCas9KKH | − | AGCAGCGTCTACAAGAAAGTAA | GATGAT |
| 1992 | E32SaCas9KKH26 | exon32 | SaCas9KKH | − | TTGAGCAGCGTCTACAAGAAAG | TAAGAT |
| 1993 | E32SaCas9KKH27 | exon32 | SaCas9KKH | − | CAATTTTGAGCAGCGTCTACAA | GAAAGT |
| 1994 | E32SaCas9KKH28 | exon32 | SaCas9KKH | − | GTTTCGATTATTCCAGAAACCA | GCCAAT |
| 1995 | E32SaCas9KKH29 | exon32 | SaCas9KKH | − | TTACAAGATGTCTCCATGAAGT | TTCGAT |
| 1996 | E32SaCas9KKH30 | exon32 | SaCas9KKH | − | AAAAAATTACAAGATGTCTCCA | TGAAGT |
| 1997 | E32SaCas9KKH31 | exon32 | SaCas9KKH | − | ACTGTCCTTACAGAAAAAATTA | CAAGAT |
| 1998 | E33SaCas9KKH1 | exon33 | SaCas9KKH | + | CACACCTTTGCTCCCAGCTCAT | TATAAT |
| 1999 | E33SaCas9KKH2 | exon33 | SaCas9KKH | + | CTTTGCTCCCAGCTCATTATAA | TGCAAT |
| 2000 | E33SaCas9KKH3 | exon33 | SaCas9KKH | + | TTTCAAAGCTGTTACTCTTTCA | TCAAGT |
| 2001 | E33SaCas9KKH4 | exon33 | SaCas9KKH | + | GTTACTCTTTCATCAAGTTCTT | TGGGAT |
| 2002 | E33SaCas9KKH5 | exon33 | SaCas9KKH | + | GATTTTCCGTCTGCTTTTTCTG | TACAAT |
| 2003 | E33SaCas9KKH6 | exon33 | SaCas9KKH | + | GCTTTTTCTGTACAATCTGACG | TCCAGT |
| 2004 | E33SaCas9KKH7 | exon33 | SaCas9KKH | + | TTCACTTCACTCAGACTTTTAT | ACAAGT |
| 2005 | E33SaCas9KKH8 | exon33 | SaCas9KKH | + | CACTCAGACTTTTATACAAGTT | CTAAGT |
| 2006 | E33SaCas9KKH11 | exon33 | SaCas9KKH | − | TGCATTATAATGAGCTGGGAGC | AAAGGT |
| 2007 | E33SaCas9KKH12 | exon33 | SaCas9KKH | − | AGTAACAGCTTTGAAATTGCAT | TATAAT |
| 2008 | E33SaCas9KKH13 | exon33 | SaCas9KKH | − | CTTGATGAAAGAGTAACAGCTT | TGAAAT |
| 2009 | E33SaCas9KKH14 | exon33 | SaCas9KKH | − | AAAATCCCAAAGAACTTGATGA | AAGAGT |
| 2010 | E33SaCas9KKH15 | exon33 | SaCas9KKH | − | GCAGACGGAAAATCCCAAAGAA | CTTGAT |
| 2011 | E33SaCas9KKH16 | exon33 | SaCas9KKH | − | GATTGTACAGAAAAAGCAGACG | GAAAAT |
| 2012 | E33SaCas9KKH17 | exon33 | SaCas9KKH | − | AAATGGTGATAAAGACTGGACG | TCAGAT |
| 2013 | E33SaCas9KKH18 | exon33 | SaCas9KKH | − | AAGTGAAGTCTGAAGTGGAAAT | GGTGAT |
| 2014 | E33SaCas9KKH19 | exon33 | SaCas9KKH | − | GTGAAGTGAAGTCTGAAGTGGA | AATGGT |
| 2015 | E33SaCas9KKH20 | exon33 | SaCas9KKH | − | TGAGTGAAGTGAAGTCTGAAGT | GGAAAT |
| 2016 | E33SaCas9KKH21 | exon33 | SaCas9KKH | − | AAAGTCTGAGTGAAGTGAAGTC | TGAAGT |
| 2017 | E33SaCas9KKH22 | exon33 | SaCas9KKH | − | TTGTATAAAAGTCTGAGTGAAG | TGAAGT |
| 2018 | E33SaCas9KKH23 | exon33 | SaCas9KKH | − | AGAACTTGTATAAAAGTCTGAG | TGAAGT |
| 2019 | E33SaCas9KKH24 | exon33 | SaCas9KKH | − | AACTTAGAACTTGTATAAAAGT | CTGAGT |
| 2020 | E33SaCas9KKH25 | exon33 | SaCas9KKH | − | TGTTTAAACTTAGAACTTGTAT | AAAAGT |
| 2021 | E34SaCas9KKH1 | exon34 | SaCas9KKH | + | TTTACCTTTCCCCAGGCAACTT | CAGAAT |
| 2022 | E34SaCas9KKH2 | exon34 | SaCas9KKH | + | TTTCCCCAGGCAACTTCAGAAT | CCAAAT |
| 2023 | E34SaCas9KKH3 | exon34 | SaCas9KKH | + | ATTACTAGGCATTCCTTCAACT | GCTGAT |
| 2024 | E34SaCas9KKH4 | exon34 | SaCas9KKH | + | TCCTTCAACTGCTGATCTCTTT | GTCAAT |
| 2025 | E34SaCas9KKH5 | exon34 | SaCas9KKH | + | CATTTCCTTTCGCATCTTACGG | GACAAT |
| 2026 | E34SaCas9KKH12 | exon34 | SaCas9KKH | − | TGGATTCTGAAGTTGCCTGGGG | AAAGGT |
| 2027 | E34SaCas9KKH13 | exon34 | SaCas9KKH | − | GAATGCCTAGTAATTTGGATTC | TGAAGT |
| 2028 | E34SaCas9KKH14 | exon34 | SaCas9KKH | − | AGTTGAAGGAATGCCTAGTAAT | TTGGAT |
| 2029 | E34SaCas9KKH15 | exon34 | SaCas9KKH | − | ATCAGCAGTTGAAGGAATGCCT | AGTAAT |
| 2030 | E34SaCas9KKH16 | exon34 | SaCas9KKH | − | GAGATCAGCAGTTGAAGGAATG | CCTAGT |
| 2031 | E34SaCas9KKH17 | exon34 | SaCas9KKH | − | TGACAAAGAGATCAGCAGTTGA | AGGAAT |
| 2032 | E34SaCas9KKH18 | exon34 | SaCas9KKH | − | ATATGGAATTGACAAAGAGATC | AGCAGT |
| 2033 | E34SaCas9KKH19 | exon34 | SaCas9KKH | − | GCTACAGATATGGAATTGACAA | AGAGAT |
| 2034 | E34SaCas9KKH20 | exon34 | SaCas9KKH | − | GAATGGCTGGCAGCTACAGATA | TGGAAT |
| 2035 | E34SaCas9KKH21 | exon34 | SaCas9KKH | − | CTTGACAGAATGGCTGGCAGCT | ACAGAT |
| 2036 | E34SaCas9KKH22 | exon34 | SaCas9KKH | − | CGAAAGGAAATGAATGTCTTGA | CAGAAT |
| 2037 | E34SaCas9KKH23 | exon34 | SaCas9KKH | − | GTCCCGTAAGATGCGAAAGGAA | ATGAAT |
| 2038 | E34SaCas9KKH24 | exon34 | SaCas9KKH | − | AATTGTCCCGTAAGATGCGAAA | GGAAAT |
| 2039 | E34SaCas9KKH25 | exon34 | SaCas9KKH | − | AGAAATGCTTGAAATTGTCCCG | TAAGAT |
| 2040 | E34SaCas9KKH26 | exon34 | SaCas9KKH | − | AAGCAACAGTTGGAGAAATGCT | TGAAAT |
| 2041 | E34SaCas9KKH27 | exon34 | SaCas9KKH | − | ACAGAAAGAAAGCAACAGTTGG | AGAAAT |
| 2042 | E34SaCas9KKH28 | exon34 | SaCas9KKH | − | TTTCAGGTAACAGAAAGAAAGC | AACAGT |
| 2043 | E35SaCas9KKH1 | exon35 | SaCas9KKH | + | CTAGCCTTTTCTCTTACCAACA | AAAGAT |
| 2044 | E35SaCas9KKH2 | exon35 | SaCas9KKH | + | GATTTAACCACTCTTCTGCTCG | GGAGGT |
| 2045 | E35SaCas9KKH3 | exon35 | SaCas9KKH | + | TCTGCTCGGGAGGTGACAGCTA | TCCAGT |
| 2046 | E35SaCas9KKH4 | exon35 | SaCas9KKH | + | TATCCAGTTACTATTCAGAAGA | CTGAGT |
| 2047 | E35SaCas9KKH5 | exon35 | SaCas9KKH | + | TTTTCAAGGCCTCTCCTACCTC | TGTGAT |
| 2048 | E35SaCas9KKH6 | exon35 | SaCas9KKH | + | TCTCCTACCTCTGTGATACTCT | TCAGGT |
| 2049 | E35SaCas9KKH7 | exon35 | SaCas9KKH | + | TCTTCAGGTGCACCTTCTGTTT | CTCAAT |
| 2050 | E35SaCas9KKH8 | exon35 | SaCas9KKH | + | CCTTCTGTTTCTCAATCTCTTT | TTGAGT |
| 2051 | E35SaCas9KKH10 | exon35 | SaCas9KKH | − | CAGAAGAGTGGTTAAATCTTTT | GTTGGT |
| 2052 | E35SaCas9KKH11 | exon35 | SaCas9KKH | − | CACCTCCCGAGCAGAAGAGTGG | TTAAAT |
| 2053 | E35SaCas9KKH12 | exon35 | SaCas9KKH | − | GCTGTCACCTCCCGAGCAGAAG | AGTGGT |
| 2054 | E35SaCas9KKH13 | exon35 | SaCas9KKH | − | ATAGCTGTCACCTCCCGAGCAG | AAGAGT |
| 2055 | E35SaCas9KKH14 | exon35 | SaCas9KKH | − | AACTCAGTCTTCTGAATAGTAA | CTGGAT |
| 2056 | E35SaCas9KKH15 | exon35 | SaCas9KKH | − | GGAAGATAAACTCAGTCTTCTG | AATAGT |
| 2057 | E35SaCas9KKH16 | exon35 | SaCas9KKH | − | GGTGGAAGATAAACTCAGTCTT | CTGAAT |
| 2058 | E35SaCas9KKH17 | exon35 | SaCas9KKH | − | GGAGACGTTGGTGGAAGATAAA | CTCAGT |
| 2059 | E35SaCas9KKH18 | exon35 | SaCas9KKH | − | GGGCAAGAAGGAGACGTTGGTG | GAAGAT |
| 2060 | E35SaCas9KKH19 | exon35 | SaCas9KKH | − | CAGTTTTGGGCAAGAAGGAGAC | GTTGGT |
| 2061 | E35SaCas9KKH20 | exon35 | SaCas9KKH | − | CAGAGGTAGGAGAGGCCTTGAA | AACAGT |
| 2062 | E35SaCas9KKH21 | exon35 | SaCas9KKH | − | AGGTGCACCTGAAGAGTATCAC | AGAGGT |
| 2063 | E35SaCas9KKH22 | exon35 | SaCas9KKH | − | TGAGAAACAGAAGGTGCACCTG | AAGAGT |
| 2064 | E35SaCas9KKH23 | exon35 | SaCas9KKH | − | CTCAAAAAGAGATTGAGAAACA | GAAGGT |
| 2065 | E35SaCas9KKH24 | exon35 | SaCas9KKH | − | CCTTTTCACAGGCTACTCAAAA | AGAGAT |
| 2066 | E36SaCas9KKH1 | exon36 | SaCas9KKH | + | CTTAAGCACGTCTTCTTTTTGC | TGGGGT |
| 2067 | E36SaCas9KKH2 | exon36 | SaCas9KKH | + | TTTTTGCTGGGGTTTCTTTTTC | TCTGAT |
| 2068 | E36SaCas9KKH3 | exon36 | SaCas9KKH | + | TTTCTTTTTCTCTGATTCATCC | AAAAGT |
| 2069 | E36SaCas9KKH4 | exon36 | SaCas9KKH | + | ATTCATCCAAAAGTGTGTCAGC | CTGAAT |
| 2070 | E36SaCas9KKH5 | exon36 | SaCas9KKH | + | CATCCAAAAGTGTGTCAGCCTG | AATGAT |
| 2071 | E36SaCas9KKH6 | exon36 | SaCas9KKH | + | TGTCAGCCTGAATGATCCACTT | TGTGAT |
| 2072 | E36SaCas9KKH7 | exon36 | SaCas9KKH | + | GCCTGAATGATCCACTTTGTGA | TGTGGT |
| 2073 | E36SaCas9KKH8 | exon36 | SaCas9KKH | + | CACTTTGTGATGTGGTCCACAT | TCTGGT |
| 2074 | E36SaCas9KKH9 | exon36 | SaCas9KKH | + | TGATGTGGTCCACATTCTGGTC | AAAAGT |
| 2075 | E36SaCas9KKH10 | exon36 | SaCas9KKH | + | TGGTCAAAAGTTTCCATGTGTT | TCTGGT |
| 2076 | E36SaCas9KKH11 | exon36 | SaCas9KKH | + | TTTCCATGTGTTTCTGGTATTC | CTTAAT |
| 2077 | E36SaCas9KKH15 | exon36 | SaCas9KKH | − | AAAAGAAGACGTGCTTAAGGTA | GCAAAT |
| 2078 | E36SaCas9KKH16 | exon36 | SaCas9KKH | − | CCCAGCAAAAAGAAGACGTGCT | TAAGGT |
| 2079 | E36SaCas9KKH17 | exon36 | SaCas9KKH | − | ATTCAGGCTGACACACTTTTGG | ATGAAT |
| 2080 | E36SaCas9KKH18 | exon36 | SaCas9KKH | − | GATCATTCAGGCTGACACACTT | TTGGAT |
| 2081 | E36SaCas9KKH19 | exon36 | SaCas9KKH | − | AGAATGTGGACCACATCACAAA | GTGGAT |
| 2082 | E36SaCas9KKH20 | exon36 | SaCas9KKH | − | GACCAGAATGTGGACCACATCA | CAAAGT |
| 2083 | E36SaCas9KKH21 | exon36 | SaCas9KKH | − | GAAACACATGGAAACTTTTGAC | CAGAAT |
| 2084 | E37SaCas9KKH1 | exon37 | SaCas9KKH | + | TTGGAGTAGATCTTCCTACCTT | TCCAGT |
| 2085 | E37SaCas9KKH2 | exon37 | SaCas9KKH | + | TAGATCTTCCTACCTTTCCAGT | CTTAAT |
| 2086 | E37SaCas9KKH3 | exon37 | SaCas9KKH | + | CCTTTCCAGTCTTAATTCTGTG | TGAAAT |
| 2087 | E37SaCas9KKH4 | exon37 | SaCas9KKH | + | CTTAATTCTGTGTGAAATGGCT | GCAAAT |
| 2088 | E37SaCas9KKH5 | exon37 | SaCas9KKH | + | ATTCTGTGTGAAATGGCTGCAA | ATCGAT |
| 2089 | E37SaCas9KKH6 | exon37 | SaCas9KKH | + | CTGTGTGAAATGGCTGCAAATC | GATGGT |
| 2090 | E37SaCas9KKH7 | exon37 | SaCas9KKH | + | CTGCAAATCGATGGTTGAGCTC | TGAGAT |
| 2091 | E37SaCas9KKH8 | exon37 | SaCas9KKH | + | GAGCTCTGAGATTTGGGGCTCT | ACTAAT |
| 2092 | E37SaCas9KKH9 | exon37 | SaCas9KKH | + | ATTTGGGGCTCTACTAATTTCC | TGCAGT |
| 2093 | E37SaCas9KKH10 | exon37 | SaCas9KKH | + | TGGGGCTCTACTAATTTCCTGC | AGTGGT |
| 2094 | E37SaCas9KKH11 | exon37 | SaCas9KKH | + | ACTAATTTCCTGCAGTGGTCAC | CGCGGT |
| 2095 | E37SaCas9KKH12 | exon37 | SaCas9KKH | + | CAGTGGTCACCGCGGTTTGCCA | TCAAGT |
| 2096 | E37SaCas9KKH13 | exon37 | SaCas9KKH | + | CGGTTTGCCATCAAGTTTGCTG | CTTGGT |
| 2097 | E37SaCas9KKH14 | exon37 | SaCas9KKH | + | AAGTTTGCTGCTTGGTCACGTG | TAGAGT |
| 2098 | E37SaCas9KKH15 | exon37 | SaCas9KKH | + | GTCCACCTTTGGGCGTATGTCA | TTCAGT |
| 2099 | E37SaCas9KKH19 | exon37 | SaCas9KKH | − | TTTCACACAGAATTAAGACTGG | AAAGGT |
| 2100 | E37SaCas9KKH20 | exon37 | SaCas9KKH | − | ATCGATTTGCAGCCATTTCACA | CAGAAT |
| 2101 | E37SaCas9KKH21 | exon37 | SaCas9KKH | − | CCCCAAATCTCAGAGCTCAACC | ATCGAT |
| 2102 | E37SaCas9KKH22 | exon37 | SaCas9KKH | − | ACTGCAGGAAATTAGTAGAGCC | CCAAAT |
| 2103 | E37SaCas9KKH23 | exon37 | SaCas9KKH | − | ACCGCGGTGACCACTGCAGGAA | ATTAGT |
| 2104 | E37SaCas9KKH24 | exon37 | SaCas9KKH | − | GCAAACCGCGGTGACCACTGCA | GGAAAT |
| 2105 | E37SaCas9KKH25 | exon37 | SaCas9KKH | − | AGCAGCAAACTTGATGGCAAAC | CGCGGT |
| 2106 | E37SaCas9KKH26 | exon37 | SaCas9KKH | − | CTACACGTGACCAAGCAGCAAA | CTTGAT |
| 2107 | E37SaCas9KKH27 | exon37 | SaCas9KKH | − | CAGAACTGAATGACATACGCCC | AAAGGT |
| 2108 | E37SaCas9KKH28 | exon37 | SaCas9KKH | − | AATATAGCGTTTAAAGGCAGAA | CTGAAT |
| 2109 | E38SaCas9KKH2 | exon38 | SaCas9KKH | + | AACCAATTTACCATATCTTTAT | TGAAGT |
| 2110 | E38SaCas9KKH3 | exon38 | SaCas9KKH | + | TCTTTATTGAAGTCTTCCTCTT | TCAGAT |
| 2111 | E38SaCas9KKH4 | exon38 | SaCas9KKH | + | CCTCTTTCAGATTCACCCCCTG | CTGAAT |
| 2112 | E38SaCas9KKH5 | exon38 | SaCas9KKH | + | CACCCCCTGCTGAATTTCAGCC | TCCAGT |
| 2113 | E38SaCas9KKH6 | exon38 | SaCas9KKH | + | CCCCTGCTGAATTTCAGCCTCC | AGTGGT |
| 2114 | E38SaCas9KKH7 | exon38 | SaCas9KKH | + | AATTTCAGCCTCCAGTGGTTCA | AGCAAT |
| 2115 | E38SaCas9KKH8 | exon38 | SaCas9KKH | + | GGTTCAAGCAATTTTTGTATAT | CTGAGT |
| 2116 | E38SaCas9KKH9 | exon38 | SaCas9KKH | + | TTGTATATCTGAGTTAAACTGC | TCCAAT |
| 2117 | E38SaCas9KKH10 | exon38 | SaCas9KKH | + | TAAACTGCTCCAATTCCTTCAA | AGGAAT |
| 2118 | E38SaCas9KKH18 | exon38 | SaCas9KKH | − | GGAAGACTTCAATAAAGATATG | GTAAAT |
| 2119 | E38SaCas9KKH19 | exon38 | SaCas9KKH | − | AAGAGGAAGACTTCAATAAAGA | TATGGT |
| 2120 | E38SaCas9KKH20 | exon38 | SaCas9KKH | − | TCTGAAAGAGGAAGACTTCAAT | AAAGAT |
| 2121 | E38SaCas9KKH21 | exon38 | SaCas9KKH | − | GGTGAATCTGAAAGAGGAAGAC | TTCAAT |
| 2122 | E38SaCas9KKH22 | exon38 | SaCas9KKH | − | GGAGGCTGAAATTCAGCAGGGG | GTGAAT |
| 2123 | E38SaCas9KKH23 | exon38 | SaCas9KKH | − | CACTGGAGGCTGAAATTCAGCA | GGGGGT |
| 2124 | E38SaCas9KKH24 | exon38 | SaCas9KKH | − | AATTGCTTGAACCACTGGAGGC | TGAAAT |
| 2125 | E38SaCas9KKH25 | exon38 | SaCas9KKH | − | GAGCAGTTTAACTCAGATATAC | AAAAAT |
| 2126 | E38SaCas9KKH26 | exon38 | SaCas9KKH | − | GAAGGAATTGGAGCAGTTTAAC | TCAGAT |
| 2127 | E38SaCas9KKH27 | exon38 | SaCas9KKH | − | TCCATTCCTTTGAAGGAATTGG | AGCAGT |
| 2128 | E38SaCas9KKH28 | exon38 | SaCas9KKH | − | TTTTAGGCCTCCATTCCTTTGA | AGGAAT |
| 2129 | E39SaCas9KKH2 | exon39 | SaCas9KKH | + | TTTCCTCTCGCTTTCTCTCATC | TGTGAT |
| 2130 | E39SaCas9KKH3 | exon39 | SaCas9KKH | + | CTCTCATCTGTGATTCTTTGTT | GTAAGT |
| 2131 | E39SaCas9KKH4 | exon39 | SaCas9KKH | + | TTGTAAGTTGTCTCCTCTTTGC | AACAAT |
| 2132 | E39SaCas9KKH5 | exon39 | SaCas9KKH | + | CTCCTCTTTGCAACAATTCTTT | TACAGT |
| 2133 | E39SaCas9KKH6 | exon39 | SaCas9KKH | + | ACAGTACCCTCATTGTCTTCAT | TCTGAT |
| 2134 | E39SaCas9KKH11 | exon39 | SaCas9KKH | − | TACAGACAAAACATAATGCTCT | CAAGGT |
| 2135 | E39SaCas9KKH12 | exon39 | SaCas9KKH | − | ACAGCAGCTGTTACAGACAAAA | CATAAT |
| 2136 | E39SaCas9KKH13 | exon39 | SaCas9KKH | − | ATGAGAGAAAGCGAGAGGAAAT | AAAGAT |
| 2137 | E39SaCas9KKH14 | exon39 | SaCas9KKH | − | TCACAGATGAGAGAAAGCGAGA | GGAAAT |
| 2138 | E39SaCas9KKH15 | exon39 | SaCas9KKH | − | AGACAACTTACAACAAAGAATC | ACAGAT |
| 2139 | E39SaCas9KKH16 | exon39 | SaCas9KKH | − | AAAGAGGAGACAACTTACAACA | AAGAAT |
| 2140 | E39SaCas9KKH17 | exon39 | SaCas9KKH | − | GAAGACAATGAGGGTACTGTAA | AAGAAT |
| 2141 | E39SaCas9KKH18 | exon39 | SaCas9KKH | − | TTTTGATCAGAATGAAGACAAT | GAGGGT |
| 2142 | E39SaCas9KKH19 | exon39 | SaCas9KKH | − | TGTTGTTTTTGATCAGAATGAA | GACAAT |
| 2143 | E40SaCas9KKH1 | exon40 | SaCas9KKH | + | TATTTTCCTTTCATCTCTGGGC | TCAGGT |
| 2144 | E40SaCas9KKH2 | exon40 | SaCas9KKH | + | ATCTCTGGGCTCAGGTAGGCTG | GCTAAT |
| 2145 | E40SaCas9KKH3 | exon40 | SaCas9KKH | + | CAGGTAGGCTGGCTAATTTTTT | TTCAAT |
| 2146 | E40SaCas9KKH4 | exon40 | SaCas9KKH | + | TCAATGTCATCCAAGCATTTCA | GGAGAT |
| 2147 | E40SaCas9KKH5 | exon40 | SaCas9KKH | + | AGATCATCAGCCTGCCTCTTGT | ACTGAT |
| 2148 | E40SaCas9KKH6 | exon40 | SaCas9KKH | + | GCCTGCCTCTTGTACTGATACC | ACTGAT |
| 2149 | E40SaCas9KKH7 | exon40 | SaCas9KKH | + | TCTTGTACTGATACCACTGATG | AGAAAT |
| 2150 | E40SaCas9KKH8 | exon40 | SaCas9KKH | + | GCCTTTTTTCTTCTTTGAGACC | TCAAAT |
| 2151 | E40SaCas9KKH14 | exon40 | SaCas9KKH | − | CCAGAGATGAAAGGAAAATAAA | GGTAAT |
| 2152 | E40SaCas9KKH15 | exon40 | SaCas9KKH | − | AGCCCAGAGATGAAAGGAAAAT | AAAGGT |
| 2153 | E40SaCas9KKH16 | exon40 | SaCas9KKH | − | TACCTGAGCCCAGAGATGAAAG | GAAAAT |
| 2154 | E40SaCas9KKH17 | exon40 | SaCas9KKH | − | ATTAGCCAGCCTACCTGAGCCC | AGAGAT |
| 2155 | E40SaCas9KKH18 | exon40 | SaCas9KKH | − | AAATGCTTGGATGACATTGAAA | AAAAAT |
| 2156 | E40SaCas9KKH19 | exon40 | SaCas9KKH | − | GGCTGATGATCTCCTGAAATGC | TTGGAT |
| 2157 | E40SaCas9KKH20 | exon40 | SaCas9KKH | − | AAGAGGCAGGCTGATGATCTCC | TGAAAT |
| 2158 | E40SaCas9KKH21 | exon40 | SaCas9KKH | − | GTATCAGTACAAGAGGCAGGCT | GATGAT |
| 2159 | E40SaCas9KKH22 | exon40 | SaCas9KKH | − | GTGGTATCAGTACAAGAGGCAG | GCTGAT |
| 2160 | E40SaCas9KKH23 | exon40 | SaCas9KKH | − | CTAGAAATTTCTCATCAGTGGT | ATCAGT |
| 2161 | E40SaCas9KKH24 | exon40 | SaCas9KKH | − | AAGGCTCTAGAAATTTCTCATC | AGTGGT |
| 2162 | E40SaCas9KKH25 | exon40 | SaCas9KKH | − | AAAAAGGCTCTAGAAATTTCTC | ATCAGT |
| 2163 | E40SaCas9KKH26 | exon40 | SaCas9KKH | − | CTCAAAGAAGAAAAAAGGCTCT | AGAAAT |
| 2164 | E40SaCas9KKH27 | exon40 | SaCas9KKH | − | TGTCTGCACCATGAACAGGATT | TGAGGT |
| 2165 | E41SaCas9KKH2 | exon41 | SaCas9KKH | + | CAGTAACAACTCACAATTTGTG | CAAAGT |
| 2166 | E41SaCas9KKH3 | exon41 | SaCas9KKH | + | ACAACTCACAATTTGTGCAAAG | TTGAGT |
| 2167 | E41SaCas9KKH4 | exon41 | SaCas9KKH | + | AAAGTTGAGTCTTCGAAACTGA | GCAAAT |
| 2168 | E41SaCas9KKH5 | exon41 | SaCas9KKH | + | TTCGAAACTGAGCAAATTTGCT | CTCAAT |
| 2169 | E41SaCas9KKH6 | exon41 | SaCas9KKH | + | TTTCCCGCCAGCGCTTGCTGAG | CTGGAT |
| 2170 | E41SaCas9KKH7 | exon41 | SaCas9KKH | + | GCCAGCGCTTGCTGAGCTGGAT | CTGAGT |
| 2171 | E41SaCas9KKH8 | exon41 | SaCas9KKH | + | ATTCAGCTCCTCTTTCTTCTTC | TGCAAT |
| 2172 | E41SaCas9KKH9 | exon41 | SaCas9KKH | + | TCCTCTTTCTTCTTCTGCAATT | CCCGAT |
| 2173 | E41SaCas9KKH10 | exon41 | SaCas9KKH | + | CTTTCTTCTTCTGCAATTCCCG | ATCAAT |
| 2174 | E41SaCas9KKH12 | exon41 | SaCas9KKH | − | AAGACTCAACTTTGCACAAATT | GTGAGT |
| 2175 | E41SaCas9KKH13 | exon41 | SaCas9KKH | − | AGTTTCGAAGACTCAACTTTGC | ACAAAT |
| 2176 | E41SaCas9KKH14 | exon41 | SaCas9KKH | − | CGGGAAATTGAGAGCAAATTTG | CTCAGT |
| 2177 | E41SaCas9KKH15 | exon41 | SaCas9KKH | − | AAGCGCTGGCGGGAAATTGAGA | GCAAAT |
| 2178 | E41SaCas9KKH16 | exon41 | SaCas9KKH | − | TCCAGCTCAGCAAGCGCTGGCG | GGAAAT |
| 2179 | E41SaCas9KKH17 | exon41 | SaCas9KKH | − | CCGCAATGGCAGTGGAGCCAAC | TCAGAT |
| 2180 | E41SaCas9KKH18 | exon41 | SaCas9KKH | − | TGTCTGAGGATGGGGCCGCAAT | GGCAGT |
| 2181 | E41SaCas9KKH19 | exon41 | SaCas9KKH | − | AGGGCTTGTCTGAGGATGGGGC | CGCAAT |
| 2182 | E41SaCas9KKH20 | exon41 | SaCas9KKH | − | TAGGCAAGCTGAGGGCTTGTCT | GAGGAT |
| 2183 | E41SaCas9KKH21 | exon41 | SaCas9KKH | − | AGAAGAAGAAAGAGGAGCTGAA | TGCAGT |
| 2184 | E41SaCas9KKH22 | exon41 | SaCas9KKH | − | ATTGCAGAAGAAGAAAGAGGAG | CTGAAT |
| 2185 | E41SaCas9KKH23 | exon41 | SaCas9KKH | − | TTTGCTCAATAGGAAATTGATC | GGGAAT |
| 2186 | E42SaCas9KKH1 | exon42 | SaCas9KKH | + | TTCAGAGACTCCTCTTGCTTAA | AGAGAT |
| 2187 | E42SaCas9KKH2 | exon42 | SaCas9KKH | + | TCCTCTTGCTTAAAGAGATCTT | CAAAGT |
| 2188 | E42SaCas9KKH3 | exon42 | SaCas9KKH | + | AGATCTTCAAAGTCCTTAGCAC | AGAGGT |
| 2189 | E42SaCas9KKH4 | exon42 | SaCas9KKH | + | AGCACAGAGGTCAGGAGCATTG | AGAAGT |
| 2190 | E42SaCas9KKH5 | exon42 | SaCas9KKH | + | AGCATTGAGAAGTTGTTCCACT | TCTAAT |
| 2191 | E42SaCas9KKH6 | exon42 | SaCas9KKH | + | CTTCTAATAGGGCTTGTGAGAC | ATGAGT |
| 2192 | E42SaCas9KKH7 | exon42 | SaCas9KKH | + | CTAATAGGGCTTGTGAGACATG | AGTGAT |
| 2193 | E42SaCas9KKH8 | exon42 | SaCas9KKH | + | GGGCTTGTGAGACATGAGTGAT | TTCAGT |
| 2194 | E42SaCas9KKH9 | exon42 | SaCas9KKH | + | TGTGAGACATGAGTGATTTCAG | TCAAAT |
| 2195 | E42SaCas9KKH10 | exon42 | SaCas9KKH | + | AGACATGAGTGATTTCAGTCAA | ATAAGT |
| 2196 | E42SaCas9KKH11 | exon42 | SaCas9KKH | + | TCAAATAAGTAGAAGGCACATA | AGAAAT |
| 2197 | E42SaCas9KKH12 | exon42 | SaCas9KKH | + | AAGAAATTTCCAAAGGCATGTC | TTCAGT |
| 2198 | E42SaCas9KKH13 | exon42 | SaCas9KKH | + | TCACCATCATCGTTTCTTCACG | GACAGT |
| 2199 | E42SaCas9KKH14 | exon42 | SaCas9KKH | + | ATCGTTTCTTCACGGACAGTGT | GCTGGT |
| 2200 | E42SaCas9KKH17 | exon42 | SaCas9KKH | − | TCTTTAAGCAAGAGGAGTCTCT | GAAGGT |
| 2201 | E42SaCas9KKH18 | exon42 | SaCas9KKH | − | TTTGAAGATCTCTTTAAGCAAG | AGGAGT |
| 2202 | E42SaCas9KKH19 | exon42 | SaCas9KKH | − | TGACCTCTGTGCTAAGGACTTT | GAAGAT |
| 2203 | E42SaCas9KKH20 | exon42 | SaCas9KKH | − | CCTATTAGAAGTGGAACAACTT | CTCAAT |
| 2204 | E42SaCas9KKH21 | exon42 | SaCas9KKH | − | CTCATGTCTCACAAGCCCTATT | AGAAGT |
| 2205 | E42SaCas9KKH22 | exon42 | SaCas9KKH | − | ATGTGCCTTCTACTTATTTGAC | TGAAAT |
| 2206 | E42SaCas9KKH23 | exon42 | SaCas9KKH | − | TGATGACTGAAGACATGCCTTT | GGAAAT |
| 2207 | E42SaCas9KKH24 | exon42 | SaCas9KKH | − | CTGTCCGTGAAGAAACGATGAT | GGTGAT |
| 2208 | E42SaCas9KKH25 | exon42 | SaCas9KKH | − | ACACTGTCCGTGAAGAAACGAT | GATGGT |
| 2209 | E42SaCas9KKH26 | exon42 | SaCas9KKH | − | AGCACACTGTCCGTGAAGAAAC | GATGAT |
| 2210 | E42SaCas9KKH27 | exon42 | SaCas9KKH | − | ACCAGCACACTGTCCGTGAAGA | AACGAT |
| 2211 | E43SaCas9KKH1 | exon43 | SaCas9KKH | + | AATATATGTGTTACCTACCCTT | GTCGGT |
| 2212 | E43SaCas9KKH2 | exon43 | SaCas9KKH | + | ATTTTGTTAACTTTTTCCCATT | GGAAAT |
| 2213 | E43SaCas9KKH3 | exon43 | SaCas9KKH | + | CCTTTCCACAGGCGTTGCACTT | TGCAAT |
| 2214 | E43SaCas9KKH4 | exon43 | SaCas9KKH | + | GCAATGCTGCTGTCTTCTTGCT | ATGAAT |
| 2215 | E43SaCas9KKH5 | exon43 | SaCas9KKH | + | ATGCTGCTGTCTTCTTGCTATG | AATAAT |
| 2216 | E43SaCas9KKH6 | exon43 | SaCas9KKH | + | CTGTCTTCTTGCTATGAATAAT | GTCAAT |
| 2217 | E43SaCas9KKH7 | exon43 | SaCas9KKH | + | TTGTAGACTATCTTTTATATTC | TGTAAT |
| 2218 | E43SaCas9KKH11 | exon43 | SaCas9KKH | − | AAATGTACAAGGACCGACAAGG | GTAGGT |
| 2219 | E43SaCas9KKH12 | exon43 | SaCas9KKH | − | AACAAAATGTACAAGGACCGAC | AAGGGT |
| 2220 | E43SaCas9KKH13 | exon43 | SaCas9KKH | − | ATTTCCAATGGGAAAAAGTTAA | CAAAAT |
| 2221 | E43SaCas9KKH14 | exon43 | SaCas9KKH | − | CCCAGCTTGATTTCCAATGGGA | AAAAGT |
| 2222 | E43SaCas9KKH15 | exon43 | SaCas9KKH | − | GAAGCTCTCTCCCAGCTTGATT | TCCAAT |
| 2223 | E43SaCas9KKH16 | exon43 | SaCas9KKH | − | GCTACAGGAAGCTCTCTCCCAG | CTTGAT |
| 2224 | E43SaCas9KKH17 | exon43 | SaCas9KKH | − | TGCAAAGTGCAACGCCTGTGGA | AAGGGT |
| 2225 | E43SaCas9KKH18 | exon43 | SaCas9KKH | − | TAGCAAGAAGACAGCAGCATTG | CAAAGT |
| 2226 | E43SaCas9KKH19 | exon43 | SaCas9KKH | − | ATAGTCTACAACAAAGCTCAGG | TCGGAT |
| 2227 | E43SaCas9KKH20 | exon43 | SaCas9KKH | − | AAAAGATAGTCTACAACAAAGC | TCAGGT |
| 2228 | E43SaCas9KKH21 | exon43 | SaCas9KKH | − | TTTTATATTACAGAATATAAAA | GATAGT |
| 2229 | E43SaCas9KKH22 | exon43 | SaCas9KKH | − | AATTTTTATATTACAGAATATA | AAAGAT |
| 2230 | E46SaCas9KKH2 | exon46 | SaCas9KKH | + | CTTGACTTGCTCAAGCTTTTCT | TTTAGT |
| 2231 | E46SaCas9KKH3 | exon46 | SaCas9KKH | + | TTCTTTTAGTTGCTGCTCTTTT | CCAGGT |
| 2232 | E46SaCas9KKH4 | exon46 | SaCas9KKH | + | TAGTTGCTGCTCTTTTCCAGGT | TCAAGT |
| 2233 | E46SaCas9KKH5 | exon46 | SaCas9KKH | + | GCTGCTCTTTTCCAGGTTCAAG | TGGGAT |
| 2234 | E46SaCas9KKH6 | exon46 | SaCas9KKH | + | TTCCAGGTTCAAGTGGGATACT | AGCAAT |
| 2235 | E46SaCas9KKH7 | exon46 | SaCas9KKH | + | ATCTGCTTCCTCCAACCATAAA | ACAAAT |
| 2236 | E46SaCas9KKH8 | exon46 | SaCas9KKH | + | TCCAACCATAAAACAAATTCAT | TTAAAT |
| 2237 | E46SaCas9KKH9 | exon46 | SaCas9KKH | + | AACAAATTCATTTAAATCTCTT | TGAAAT |
| 2238 | E46SaCas9KKH10 | exon46 | SaCas9KKH | + | TTAAATCTCTTTGAAATTCTGA | CAAGAT |
| 2239 | E46SaCas9KKH14 | exon46 | SaCas9KKH | − | AAGAAAAGCTTGAGCAAGTCAA | GGTAAT |
| 2240 | E46SaCas9KKH15 | exon46 | SaCas9KKH | − | TAAAAGAAAAGCTTGAGCAAGT | CAAGGT |
| 2241 | E46SaCas9KKH16 | exon46 | SaCas9KKH | − | AGCAACTAAAAGAAAAGCTTGA | GCAAGT |
| 2242 | E46SaCas9KKH17 | exon46 | SaCas9KKH | − | GTTGGAGGAAGCAGATAACATT | GCTAGT |
| 2243 | E46SaCas9KKH18 | exon46 | SaCas9KKH | − | ATTTGTTTTATGGTTGGAGGAA | GCAGAT |
| 2244 | E46SaCas9KKH19 | exon46 | SaCas9KKH | − | AGAGATTTAAATGAATTTGTTT | TATGGT |
| 2245 | E46SaCas9KKH20 | exon46 | SaCas9KKH | − | TCAGAATTTCAAAGAGATTTAA | ATGAAT |
| 2246 | E46SaCas9KKH21 | exon46 | SaCas9KKH | − | CTTGTCAGAATTTCAAAGAGAT | TTAAAT |
| 2247 | E46SaCas9KKH22 | exon46 | SaCas9KKH | − | GAATATCTTGTCAGAATTTCAA | AGAGAT |
| 2248 | E46SaCas9KKH23 | exon46 | SaCas9KKH | − | GAAGAACAAAAGAATATCTTGT | CAGAAT |
| 2249 | E46SaCas9KKH24 | exon46 | SaCas9KKH | − | TTTCTCCAGGCTAGAAGAACAA | AAGAAT |
| 2250 | E47SaCas9KKH1 | exon47 | SaCas9KKH | + | TAATGTCTAACCTTTATCCACT | GGAGAT |
| 2251 | E47SaCas9KKH2 | exon47 | SaCas9KKH | + | ATTTGTCTGCTTGAGCTTATTT | TCAAGT |
| 2252 | E47SaCas9KKH3 | exon47 | SaCas9KKH | + | GCTTATGGGAGCACTTACAAGC | ACGGGT |
| 2253 | E47SaCas9KKH4 | exon47 | SaCas9KKH | + | GAGCACTTACAAGCACGGGTCC | TCCAGT |
| 2254 | E47SaCas9KKH5 | exon47 | SaCas9KKH | + | AAGCACGGGTCCTCCAGTTTCA | TTTAAT |
| 2255 | E47SaCas9KKH6 | exon47 | SaCas9KKH | + | CTCCAGTTTCATTTAATTGTTT | GAGAAT |
| 2256 | E47SaCas9KKH7 | exon47 | SaCas9KKH | + | CTGGCGCAGGGGCAACTCTTCC | ACCAGT |
| 2257 | E47SaCas9KKH10 | exon47 | SaCas9KKH | − | AGCAGACAAATCTCCAGTGGAT | AAAGGT |
| 2258 | E47SaCas9KKH11 | exon47 | SaCas9KKH | − | AGCTCAAGCAGACAAATCTCCA | GTGGAT |
| 2259 | E47SaCas9KKH12 | exon47 | SaCas9KKH | − | AATAAGCTCAAGCAGACAAATC | TCCAGT |
| 2260 | E47SaCas9KKH13 | exon47 | SaCas9KKH | − | ACTTGAAAATAAGCTCAAGCAG | ACAAAT |
| 2261 | E47SaCas9KKH14 | exon47 | SaCas9KKH | − | CCCAGAAGAGCAAGATAAACTT | GAAAAT |
| 2262 | E47SaCas9KKH15 | exon47 | SaCas9KKH | − | TGCTCCCATAAGCCCAGAAGAG | CAAGAT |
| 2263 | E47SaCas9KKH16 | exon47 | SaCas9KKH | − | TGAAACTGGAGGACCCGTGCTT | GTAAGT |
| 2264 | E47SaCas9KKH17 | exon47 | SaCas9KKH | − | GCGCCAGGGAATTCTCAAACAA | TTAAAT |
| 2265 | E47SaCas9KKH18 | exon47 | SaCas9KKH | − | CCCCTGCGCCAGGGAATTCTCA | AACAAT |
| 2266 | E47SaCas9KKH19 | exon47 | SaCas9KKH | − | TGGAAGAGTTGCCCCTGCGCCA | GGGAAT |
| 2267 | E47SaCas9KKH20 | exon47 | SaCas9KKH | − | TGTCTGTTTCAGTTACTGGTGG | AAGAGT |
| 2268 | E48SaCas9KKH1 | exon48 | SaCas9KKH | + | CAAAAAGTTCCCTACCTTAACG | TCAAAT |
| 2269 | E48SaCas9KKH2 | exon48 | SaCas9KKH | + | AAAGTTCCCTACCTTAACGTCA | AATGGT |
| 2270 | E48SaCas9KKH3 | exon48 | SaCas9KKH | + | ACCTTAACGTCAAATGGTCCTT | CTTGGT |
| 2271 | E48SaCas9KKH4 | exon48 | SaCas9KKH | + | AACGTCAAATGGTCCTTCTTGG | TTTGGT |
| 2272 | E48SaCas9KKH5 | exon48 | SaCas9KKH | + | TCAAATGGTCCTTCTTGGTTTG | GTTGGT |
| 2273 | E48SaCas9KKH6 | exon48 | SaCas9KKH | + | GTCCTTCTTGGTTTGGTTGGTT | ATAAAT |
| 2274 | E48SaCas9KKH7 | exon48 | SaCas9KKH | + | TTTGGTTGGTTATAAATTTCCA | ACTGAT |
| 2275 | E48SaCas9KKH8 | exon48 | SaCas9KKH | + | GGTTATAAATTTCCAACTGATT | CCTAAT |
| 2276 | E48SaCas9KKH9 | exon48 | SaCas9KKH | + | AATTTCCAACTGATTCCTAATA | GGAGAT |
| 2277 | E48SaCas9KKH10 | exon48 | SaCas9KKH | + | CTAATAGGAGATAACCACAGCA | GCAGAT |
| 2278 | E48SaCas9KKH11 | exon48 | SaCas9KKH | + | ATAGGAGATAACCACAGCAGCA | GATGAT |
| 2279 | E48SaCas9KKH12 | exon48 | SaCas9KKH | + | AGCAGATGATTTAACTGCTCTT | CAAGGT |
| 2280 | E48SaCas9KKH13 | exon48 | SaCas9KKH | + | TCAAGCTTTTTTTCAAGCTGCC | CAAGGT |
| 2281 | E48SaCas9KKH14 | exon48 | SaCas9KKH | + | GCCCAAGGTCTTTTATTTGAGC | TTCAAT |
| 2282 | E48SaCas9KKH15 | exon48 | SaCas9KKH | + | AGCTTCAATTTCTCCTTGTTTC | TCAGGT |
| 2283 | E48SaCas9KKH20 | exon48 | SaCas9KKH | − | ACCAAGAAGGACCATTTGACGT | TAAGGT |
| 2284 | E48SaCas9KKH21 | exon48 | SaCas9KKH | − | TATCTCCTATTAGGAATCAGTT | GGAAAT |
| 2285 | E48SaCas9KKH22 | exon48 | SaCas9KKH | − | CTGTGGTTATCTCCTATTAGGA | ATCAGT |
| 2286 | E48SaCas9KKH23 | exon48 | SaCas9KKH | − | GCTGCTGTGGTTATCTCCTATT | AGGAAT |
| 2287 | E48SaCas9KKH24 | exon48 | SaCas9KKH | − | GAGCAGTTAAATCATCTGCTGC | TGTGGT |
| 2288 | E48SaCas9KKH25 | exon48 | SaCas9KKH | − | GCTTGAAGACCTTGAAGAGCAG | TTAAAT |
| 2289 | E48SaCas9KKH26 | exon48 | SaCas9KKH | − | AAAAAGCTTGAAGACCTTGAAG | AGCAGT |
| 2290 | E48SaCas9KKH27 | exon48 | SaCas9KKH | − | AGAAACAAGGAGAAATTGAAGC | TCAAAT |
| 2291 | E48SaCas9KKH28 | exon48 | SaCas9KKH | − | GAGCTTTACCTGAGAAACAAGG | AGAAAT |
| 2292 | E49SaCas9KKH1 | exon49 | SaCas9KKH | + | GTTGCTTCATTACCTTCACTGG | CTGAGT |
| 2293 | E49SaCas9KKH2 | exon49 | SaCas9KKH | + | CATTACCTTCACTGGCTGAGTG | GCTGGT |
| 2294 | E49SaCas9KKH3 | exon49 | SaCas9KKH | + | TGAGTGGCTGGTTTTTCCTTGT | ACAAAT |
| 2295 | E49SaCas9KKH4 | exon49 | SaCas9KKH | + | TGTACAAATGCTGCCCTTTAGA | CAAAAT |
| 2296 | E49SaCas9KKH5 | exon49 | SaCas9KKH | + | TTTAGACAAAATCTCTTCCACA | TCCGGT |
| 2297 | E49SaCas9KKH6 | exon49 | SaCas9KKH | + | GTTGTTTAGCTTGAACTGCTAT | TTCAGT |
| 2298 | E49SaCas9KKH10 | exon49 | SaCas9KKH | − | AACCAGCCACTCAGCCAGTGAA | GGTAAT |
| 2299 | E49SaCas9KKH11 | exon49 | SaCas9KKH | − | AAAAACCAGCCACTCAGCCAGT | GAAGGT |
| 2300 | E49SaCas9KKH12 | exon49 | SaCas9KKH | − | ACAAGGAAAAACCAGCCACTCA | GCCAGT |
| 2301 | E49SaCas9KKH13 | exon49 | SaCas9KKH | − | AAGCTAAACAACCGGATGTGGA | AGAGAT |
| 2302 | E49SaCas9KKH14 | exon49 | SaCas9KKH | − | AATAGCAGTTCAAGCTAAACAA | CCGGAT |
| 2303 | E49SaCas9KKH15 | exon49 | SaCas9KKH | − | CTTTTCCCCAGGAAACTGAAAT | AGCAGT |
| 2304 | E49SaCas9KKH16 | exon49 | SaCas9KKH | − | TGGGTTCTTTTCCCCAGGAAAC | TGAAAT |
| 2305 | E52SaCas9KKH2 | exon52 | SaCas9KKH | + | TGTTAAAAAACTTACTTCGATC | CGTAAT |
| 2306 | E52SaCas9KKH3 | exon52 | SaCas9KKH | + | TAAAAAACTTACTTCGATCCGT | AATGAT |
| 2307 | E52SaCas9KKH4 | exon52 | SaCas9KKH | + | TCCGTAATGATTGTTCTAGCCT | CTTGAT |
| 2308 | E52SaCas9KKH5 | exon52 | SaCas9KKH | + | TGATTGTTCTAGCCTCTTGATT | GCTGGT |
| 2309 | E52SaCas9KKH6 | exon52 | SaCas9KKH | + | TCTTGATTGCTGGTCTTGTTTT | TCAAAT |
| 2310 | E52SaCas9KKH7 | exon52 | SaCas9KKH | + | TCTTGTTTTTCAAATTTTGGGC | AGCGGT |
| 2311 | E52SaCas9KKH8 | exon52 | SaCas9KKH | + | TGTTTTTCAAATTTTGGGCAGC | GGTAAT |
| 2312 | E52SaCas9KKH9 | exon52 | SaCas9KKH | + | TTTCAAATTTTGGGCAGCGGTA | ATGAGT |
| 2313 | E52SaCas9KKH10 | exon52 | SaCas9KKH | + | TCCAACTGGGGACGCCTCTGTT | CCAAAT |
| 2314 | E52SaCas9KKH14 | exon52 | SaCas9KKH | − | AGAACAATCATTACGGATCGAA | GTAAGT |
| 2315 | E52SaCas9KKH15 | exon52 | SaCas9KKH | − | GGCTAGAACAATCATTACGGAT | CGAAGT |
| 2316 | E52SaCas9KKH16 | exon52 | SaCas9KKH | − | TCAAGAGGCTAGAACAATCATT | ACGGAT |
| 2317 | E52SaCas9KKH17 | exon52 | SaCas9KKH | − | AGACCAGCAATCAAGAGGCTAG | AACAAT |
| 2318 | E52SaCas9KKH18 | exon52 | SaCas9KKH | − | CCAAAATTTGAAAAACAAGACC | AGCAAT |
| 2319 | E52SaCas9KKH19 | exon52 | SaCas9KKH | − | GGAAGAACTCATTACCGCTGCC | CAAAAT |
| 2320 | E52SaCas9KKH20 | exon52 | SaCas9KKH | − | CAGGATTTGGAACAGAGGCGTC | CCCAGT |
| 2321 | E52SaCas9KKH21 | exon52 | SaCas9KKH | − | TTTGTTCTTACAGGCAACAATG | CAGGAT |
| 2322 | E54SaCas9KKH1 | exon54 | SaCas9KKH | + | AATAATGTAATTCATACCTTTT | ATGAAT |
| 2323 | E54SaCas9KKH2 | exon54 | SaCas9KKH | + | TATGAATGCTTCTCCAAGAGGC | ATTGAT |
| 2324 | E54SaCas9KKH3 | exon54 | SaCas9KKH | + | TCTCTGTTATCATGTGGACTTT | TCTGGT |
| 2325 | E54SaCas9KKH4 | exon54 | SaCas9KKH | + | TGGACTTTTCTGGTATCATCTG | CAGAAT |
| 2326 | E54SaCas9KKH5 | exon54 | SaCas9KKH | + | ACTTTTCTGGTATCATCTGCAG | AATAAT |
| 2327 | E54SaCas9KKH6 | exon54 | SaCas9KKH | + | ATCATCTGCAGAATAATCCCGG | AGAAGT |
| 2328 | E54SaCas9KKH7 | exon54 | SaCas9KKH | + | TAATCCCGGAGAAGTTTCAGGG | CCAAGT |
| 2329 | E54SaCas9KKH8 | exon54 | SaCas9KKH | + | TCTACATTTGTCTGCCACTGGC | GGAGGT |
| 2330 | E54SaCas9KKH9 | exon54 | SaCas9KKH | + | CGGAGGTCTTTGGCCAACTGCT | ATAGAT |
| 2331 | E54SaCas9KKH12 | exon54 | SaCas9KKH | − | TGGAGAAGCATTCATAAAAGGT | ATGAAT |
| 2332 | E54SaCas9KKH13 | exon54 | SaCas9KKH | − | GCCTCTTGGAGAAGCATTCATA | AAAGGT |
| 2333 | E54SaCas9KKH14 | exon54 | SaCas9KKH | − | AGTCCACATGATAACAGAGAAT | ATCAAT |
| 2334 | E54SaCas9KKH15 | exon54 | SaCas9KKH | − | CAGAAAAGTCCACATGATAACA | GAGAAT |
| 2335 | E54SaCas9KKH16 | exon54 | SaCas9KKH | − | CAGATGATACCAGAAAAGTCCA | CATGAT |
| 2336 | E54SaCas9KKH17 | exon54 | SaCas9KKH | − | ATTATTCTGCAGATGATACCAG | AAAAGT |
| 2337 | E54SaCas9KKH18 | exon54 | SaCas9KKH | − | ACTTCTCCGGGATTATTCTGCA | GATGAT |
| 2338 | E54SaCas9KKH19 | exon54 | SaCas9KKH | − | GAAACTTCTCCGGGATTATTCT | GCAGAT |
| 2339 | E54SaCas9KKH20 | exon54 | SaCas9KKH | − | TGACTTGGCCCTGAAACTTCTC | CGGGAT |
| 2340 | E54SaCas9KKH21 | exon54 | SaCas9KKH | − | GTGGCAGACAAATGTAGATGTG | GCAAAT |
| 2341 | E54SaCas9KKH22 | exon54 | SaCas9KKH | − | CCTCCGCCAGTGGCAGACAAAT | GTAGAT |
| 2342 | E54SaCas9KKH23 | exon54 | SaCas9KKH | − | CAAAGACCTCCGCCAGTGGCAG | ACAAAT |
| 2343 | E54SaCas9KKH24 | exon54 | SaCas9KKH | − | TAGCAGTTGGCCAAAGACCTCC | GCCAGT |
| 2344 | E55SaCas9KKH1 | exon55 | SaCas9KKH | + | TTCATCAGCTCTTTTACTCCCT | TGGAGT |
| 2345 | E55SaCas9KKH2 | exon55 | SaCas9KKH | + | AGTCTTCTAGGAGCCTTTCCTT | ACGGGT |
| 2346 | E55SaCas9KKH3 | exon55 | SaCas9KKH | + | GGGTAGCATCCTGTAGGACATT | GGCAGT |
| 2347 | E55SaCas9KKH4 | exon55 | SaCas9KKH | + | GTAAGCCAGGCAAGAAACTTTT | CCAGGT |
| 2348 | E55SaCas9KKH5 | exon55 | SaCas9KKH | + | TTCCAGGTCCAGGGGGAACTGT | TGCAGT |
| 2349 | E55SaCas9KKH6 | exon55 | SaCas9KKH | + | CAGGTCCAGGGGGAACTGTTGC | AGTAAT |
| 2350 | E55SaCas9KKH7 | exon55 | SaCas9KKH | + | GGGGGAACTGTTGCAGTAATCT | ATGAGT |
| 2351 | E55SaCas9KKH11 | exon55 | SaCas9KKH | − | AGAGCTGATGAAACAATGGCAA | GTAAGT |
| 2352 | E55SaCas9KKH12 | exon55 | SaCas9KKH | − | TAAAAGAGCTGATGAAACAATG | GCAAGT |
| 2353 | E55SaCas9KKH13 | exon55 | SaCas9KKH | − | AAGGGAGTAAAAGAGCTGATGA | AACAAT |
| 2354 | E55SaCas9KKH14 | exon55 | SaCas9KKH | − | AAGACTCCAAGGGAGTAAAAGA | GCTGAT |
| 2355 | E55SaCas9KKH15 | exon55 | SaCas9KKH | − | AAAGGCTCCTAGAAGACTCCAA | GGGAGT |
| 2356 | E55SaCas9KKH16 | exon55 | SaCas9KKH | − | TGAAACAACTGCCAATGTCCTA | CAGGAT |
| 2357 | E55SaCas9KKH17 | exon55 | SaCas9KKH | − | GCTTACAGAAGCTGAAACAACT | GCCAAT |
| 2358 | E55SaCas9KKH18 | exon55 | SaCas9KKH | − | CAACAGTTCCCCCTGGACCTGG | AAAAGT |
| 2359 | E55SaCas9KKH19 | exon55 | SaCas9KKH | − | GAAGAAACTCATAGATTACTGC | AACAGT |
| 2360 | E55SaCas9KKH20 | exon55 | SaCas9KKH | − | GAGGCTGCTTTGGAAGAAACTC | ATAGAT |
| 2361 | E56SaCas9KKH2 | exon56 | SaCas9KKH | + | CCTAATGTTGAGAGACTTTTTC | CGAAGT |
| 2362 | E56SaCas9KKH3 | exon56 | SaCas9KKH | + | TTTTTCCGAAGTTCACTCCACT | TGAAGT |
| 2363 | E56SaCas9KKH4 | exon56 | SaCas9KKH | + | GACTGCATCATCGGAACCTTCC | AGGGAT |
| 2364 | E56SaCas9KKH5 | exon56 | SaCas9KKH | + | CATCGGAACCTTCCAGGGATCT | CAGGAT |
| 2365 | E56SaCas9KKH6 | exon56 | SaCas9KKH | + | AGGATTTTTTGGCTGTTTTCAT | CCAGGT |
| 2366 | E56SaCas9KKH7 | exon56 | SaCas9KKH | + | TTTTGGCTGTTTTCATCCAGGT | TGTGAT |
| 2367 | E56SaCas9KKH8 | exon56 | SaCas9KKH | + | TGTGATAAACATCTGTGTGAGC | TTCAAT |
| 2368 | E56SaCas9KKH9 | exon56 | SaCas9KKH | + | GTGTGAGCTTCAATTTCACCTT | GGAGGT |
| 2369 | E56SaCas9KKH12 | exon56 | SaCas9KKH | − | CTTCGGAAAAAGTCTCTCAACA | TTAGGT |
| 2370 | E56SaCas9KKH13 | exon56 | SaCas9KKH | − | TTCAAGTGGAGTGAACTTCGGA | AAAAGT |
| 2371 | E56SaCas9KKH14 | exon56 | SaCas9KKH | − | TTTGGATAACATGAACTTCAAG | TGGAGT |
| 2372 | E56SaCas9KKH15 | exon56 | SaCas9KKH | − | AGACGTTTGGATAACATGAACT | TCAAGT |
| 2373 | E56SaCas9KKH16 | exon56 | SaCas9KKH | − | TGCAGTCCTGTTACAAAGACGT | TTGGAT |
| 2374 | E56SaCas9KKH17 | exon56 | SaCas9KKH | − | GATCCCTGGAAGGTTCCGATGA | TGCAGT |
| 2375 | E56SaCas9KKH18 | exon56 | SaCas9KKH | − | CCTGAGATCCCTGGAAGGTTCC | GATGAT |
| 2376 | E56SaCas9KKH19 | exon56 | SaCas9KKH | − | AATCCTGAGATCCCTGGAAGGT | TCCGAT |
| 2377 | E56SaCas9KKH20 | exon56 | SaCas9KKH | − | CCAAAAAATCCTGAGATCCCTG | GAAGGT |
| 2378 | E56SaCas9KKH21 | exon56 | SaCas9KKH | − | GATGAAAACAGCCAAAAAATCC | TGAGAT |
| 2379 | E56SaCas9KKH22 | exon56 | SaCas9KKH | − | ACAACCTGGATGAAAACAGCCA | AAAAAT |
| 2380 | E56SaCas9KKH23 | exon56 | SaCas9KKH | − | TCACACAGATGTTTATCACAAC | CTGGAT |
| 2381 | E56SaCas9KKH24 | exon56 | SaCas9KKH | − | CCAAGGTGAAATTGAAGCTCAC | ACAGAT |
| 2382 | E56SaCas9KKH25 | exon56 | SaCas9KKH | − | CTGTCCTGTAGGACCTCCAAGG | TGAAAT |
| 2383 | E56SaCas9KKH26 | exon56 | SaCas9KKH | − | TCCTGCTGTCCTGTAGGACCTC | CAAGGT |
| 2384 | E57SaCas9KKH1 | exon57 | SaCas9KKH | + | TCGTTCTGCTTCTGAACTGCTG | GAAAGT |
| 2385 | E57SaCas9KKH2 | exon57 | SaCas9KKH | + | TCTGAACTGCTGGAAAGTCGCC | TCCAAT |
| 2386 | E57SaCas9KKH3 | exon57 | SaCas9KKH | + | AACTGCTGGAAAGTCGCCTCCA | ATAGGT |
| 2387 | E57SaCas9KKH4 | exon57 | SaCas9KKH | + | GCCTCCAATAGGTGCCTGCCGG | CTTAAT |
| 2388 | E57SaCas9KKH5 | exon57 | SaCas9KKH | + | ATCTTTCAGCTGTAGCCACACC | AGAAGT |
| 2389 | E57SaCas9KKH6 | exon57 | SaCas9KKH | + | CACACCAGAAGTTCCTGCAGAG | AAAGGT |
| 2390 | E57SaCas9KKH7 | exon57 | SaCas9KKH | + | AGAGAAAGGTGCAGACGCTTCC | ACTGGT |
| 2391 | E57SaCas9KKH8 | exon57 | SaCas9KKH | + | TTCCACTGGTCAGAACTGGCTT | CCAAAT |
| 2392 | E57SaCas9KKH9 | exon57 | SaCas9KKH | − | TTCAGAAGCAGAACGATGTACA | TAGGGT |
| 2393 | E57SaCas9KKH10 | exon57 | SaCas9KKH | − | CTTTCCAGCAGTTCAGAAGCAG | AACGAT |
| 2394 | E57SaCas9KKH11 | exon57 | SaCas9KKH | − | CACCTATTGGAGGCGACTTTCC | AGCAGT |
| 2395 | E57SaCas9KKH12 | exon57 | SaCas9KKH | − | GTGTGGCTACAGCTGAAAGATG | ATGAAT |
| 2396 | E57SaCas9KKH13 | exon57 | SaCas9KKH | − | TCTGGTGTGGCTACAGCTGAAA | GATGAT |
| 2397 | E57SaCas9KKH14 | exon57 | SaCas9KKH | − | ACTTCTGGTGTGGCTACAGCTG | AAAGAT |
| 2398 | E57SaCas9KKH15 | exon57 | SaCas9KKH | − | TGCACCTTTCTCTGCAGGAACT | TCTGGT |
| 2399 | E57SaCas9KKH16 | exon57 | SaCas9KKH | − | TCCCATTTGGAAGCCAGTTCTG | ACCAGT |
| 2400 | E57SaCas9KKH17 | exon57 | SaCas9KKH | − | CTTTTTCAGGTCCCATTTGGAA | GCCAGT |
| 2401 | E58SaCas9KKH2 | exon58 | SaCas9KKH | + | CACATTCAATTACCTCTGGGCT | CCTGGT |
| 2402 | E58SaCas9KKH3 | exon58 | SaCas9KKH | + | TCAATTACCTCTGGGCTCCTGG | TAGAGT |
| 2403 | E58SaCas9KKH4 | exon58 | SaCas9KKH | + | TCTGGGCTCCTGGTAGAGTTTC | TCTAGT |
| 2404 | E58SaCas9KKH5 | exon58 | SaCas9KKH | + | TCCTTCCAAAGGCTGCTCTGTC | AGAAAT |
| 2405 | E58SaCas9KKH6 | exon58 | SaCas9KKH | + | GCTGCTCTGTCAGAAATATTCG | TACAGT |
| 2406 | E58SaCas9KKH7 | exon58 | SaCas9KKH | + | TCAGAAATATTCGTACAGTCTC | AAGAGT |
| 2407 | E58SaCas9KKH8 | exon58 | SaCas9KKH | + | TTCGTACAGTCTCAAGAGTACT | CATGAT |
| 2408 | E58SaCas9KKH9 | exon58 | SaCas9KKH | + | AGTCTCAAGAGTACTCATGATT | ACAGGT |
| 2409 | E58SaCas9KKH10 | exon58 | SaCas9KKH | + | GAGTACTCATGATTACAGGTTC | TTTAGT |
| 2410 | E58SaCas9KKH11 | exon58 | SaCas9KKH | + | CATGATTACAGGTTCTTTAGTT | TTCAAT |
| 2411 | E58SaCas9KKH17 | exon58 | SaCas9KKH | − | GAAACTCTACCAGGAGCCCAGA | GGTAAT |
| 2412 | E58SaCas9KKH18 | exon58 | SaCas9KKH | − | AGAGAAACTCTACCAGGAGCCC | AGAGGT |
| 2413 | E58SaCas9KKH19 | exon58 | SaCas9KKH | − | TCATGAGTACTCTTGAGACTGT | ACGAAT |
| 2414 | E58SaCas9KKH20 | exon58 | SaCas9KKH | − | GAAAACTAAAGAACCTGTAATC | ATGAGT |
| 2415 | E58SaCas9KKH21 | exon58 | SaCas9KKH | − | GGGAATTGAAAACTAAAGAACC | TGTAAT |
| 2416 | E58SaCas9KKH22 | exon58 | SaCas9KKH | − | CTCATTTCACAGGCCTTCAAGA | GGGAAT |
| 2417 | E59SaCas9KKH2 | exon59 | SaCas9KKH | + | GTAGACGGTACCTTGACTTTCT | CGAGGT |
| 2418 | E59SaCas9KKH3 | exon59 | SaCas9KKH | + | GACGGTACCTTGACTTTCTCGA | GGTGAT |
| 2419 | E59SaCas9KKH4 | exon59 | SaCas9KKH | + | ACTTTCTCGAGGTGATCTTGGA | GAGAGT |
| 2420 | E59SaCas9KKH5 | exon59 | SaCas9KKH | + | TCTCGAGGTGATCTTGGAGAGA | GTCAAT |
| 2421 | E59SaCas9KKH6 | exon59 | SaCas9KKH | + | TGATCTTGGAGAGAGTCAATGA | GGAGAT |
| 2422 | E59SaCas9KKH7 | exon59 | SaCas9KKH | + | GAGGAGATCGCCCACGGGCTGC | CAGGAT |
| 2423 | E59SaCas9KKH8 | exon59 | SaCas9KKH | + | CGCCCACGGGCTGCCAGGATCC | CTTGAT |
| 2424 | E59SaCas9KKH9 | exon59 | SaCas9KKH | + | ATCACCTCAGCTTGGCGCAGCT | TGAGGT |
| 2425 | E59SaCas9KKH10 | exon59 | SaCas9KKH | + | GTCCAGCTCATCCGTGGCCTCT | TGAAGT |
| 2426 | E59SaCas9KKH11 | exon59 | SaCas9KKH | + | ATCCGTGGCCTCTTGAAGTTCC | CGGAGT |
| 2427 | E59SaCas9KKH12 | exon59 | SaCas9KKH | + | CTTGAAGTTCCCGGAGTCTTTC | AAGGGT |
| 2428 | E59SaCas9KKH13 | exon59 | SaCas9KKH | + | AGGGTCTCATCTATTTTTCTCT | GCCAGT |
| 2429 | E59SaCas9KKH14 | exon59 | SaCas9KKH | + | TCTATTTTTCTCTGCCAGTCAG | CGGAGT |
| 2430 | E59SaCas9KKH15 | exon59 | SaCas9KKH | + | TTTCTCTGCCAGTCAGCGGAGT | GCAGGT |
| 2431 | E59SaCas9KKH16 | exon59 | SaCas9KKH | + | CTGCCAGTCAGCGGAGTGCAGG | TTCAAT |
| 2432 | E59SaCas9KKH17 | exon59 | SaCas9KKH | + | AGTGCAGGTTCAATTTTTCCCA | CTCAGT |
| 2433 | E59SaCas9KKH18 | exon59 | SaCas9KKH | + | CCTCAGCCTGCTTTCGTAGAAG | CCGAGT |
| 2434 | E59SaCas9KKH19 | exon59 | SaCas9KKH | + | GCTCTCTCCTCAGGAGGCAGCT | CTAAAT |
| 2435 | E59SaCas9KKH22 | exon59 | SaCas9KKH | − | TCCAAGATCACCTCGAGAAAGT | CAAGGT |
| 2436 | E59SaCas9KKH23 | exon59 | SaCas9KKH | − | ACTCTCTCCAAGATCACCTCGA | GAAAGT |
| 2437 | E59SaCas9KKH24 | exon59 | SaCas9KKH | − | CGATCTCCTCATTGACTCTCTC | CAAGAT |
| 2438 | E59SaCas9KKH25 | exon59 | SaCas9KKH | − | CAAGGGATCCTGGCAGCCCGTG | GGCGAT |
| 2439 | E59SaCas9KKH26 | exon59 | SaCas9KKH | − | CTGCGCCAAGCTGAGGTGATCA | AGGGAT |
| 2440 | E59SaCas9KKH27 | exon59 | SaCas9KKH | − | ACCTCAAGCTGCGCCAAGCTGA | GGTGAT |
| 2441 | E59SaCas9KKH28 | exon59 | SaCas9KKH | − | TGGACCTCAAGCTGCGCCAAGC | TGAGGT |
| 2442 | E59SaCas9KKH29 | exon59 | SaCas9KKH | − | ACTCCGGGAACTTCAAGAGGCC | ACGGAT |
| 2443 | E59SaCas9KKH30 | exon59 | SaCas9KKH | − | CTCCGCTGACTGGCAGAGAAAA | ATAGAT |
| 2444 | E59SaCas9KKH31 | exon59 | SaCas9KKH | − | TGCACTCCGCTGACTGGCAGAG | AAAAAT |
| 2445 | E59SaCas9KKH32 | exon59 | SaCas9KKH | − | GAGGAGGTCAATACTGAGTGGG | AAAAAT |
| 2446 | E59SaCas9KKH33 | exon59 | SaCas9KKH | − | AAGCAGGCTGAGGAGGTCAATA | CTGAGT |
| 2447 | E59SaCas9KKH34 | exon59 | SaCas9KKH | − | TCTACGAAAGCAGGCTGAGGAG | GTCAAT |
| 2448 | E59SaCas9KKH35 | exon59 | SaCas9KKH | − | GGCTTCTACGAAAGCAGGCTGA | GGAGGT |
| 2449 | E59SaCas9KKH36 | exon59 | SaCas9KKH | − | GCTGCCTCCTGAGGAGAGAGCC | CAGAAT |
| 2450 | E60SaCas9KKH1 | exon60 | SaCas9KKH | + | TGCTTACCTGCAGAAGCTTCCA | TCTGGT |
| 2451 | E60SaCas9KKH2 | exon60 | SaCas9KKH | + | TGCAGAAGCTTCCATCTGGTGT | TCAGGT |
| 2452 | E60SaCas9KKH3 | exon60 | SaCas9KKH | + | TCCATCTGGTGTTCAGGTCTTC | CAGAGT |
| 2453 | E60SaCas9KKH4 | exon60 | SaCas9KKH | + | GTGTTCAGGTCTTCCAGAGTGC | TGAGGT |
| 2454 | E60SaCas9KKH5 | exon60 | SaCas9KKH | + | GTCTTCCAGAGTGCTGAGGTTA | TACGGT |
| 2455 | E60SaCas9KKH6 | exon60 | SaCas9KKH | + | TGCTGAGGTTATACGGTGAGAG | CTGAAT |
| 2456 | E60SaCas9KKH7 | exon60 | SaCas9KKH | + | TATACGGTGAGAGCTGAATGCC | CAAAGT |
| 2457 | E60SaCas9KKH8 | exon60 | SaCas9KKH | + | ACGGTGAGAGCTGAATGCCCAA | AGTGGT |
| 2458 | E60SaCas9KKH9 | exon60 | SaCas9KKH | + | CCCAAAGTGGTAAGCTGGCGAG | CAAGGT |
| 2459 | E60SaCas9KKH10 | exon60 | SaCas9KKH | + | GGCTCACGTTCTCTTTCAGAGG | CGCAAT |
| 2460 | E60SaCas9KKH11 | exon60 | SaCas9KKH | + | TTTCAGAGGCGCAATTTCTCCT | CGAAGT |
| 2461 | E60SaCas9KKH15 | exon60 | SaCas9KKH | − | TGAACACCAGATGGAAGCTTCT | GCAGGT |
| 2462 | E60SaCas9KKH16 | exon60 | SaCas9KKH | − | AGCACTCTGGAAGACCTGAACA | CCAGAT |
| 2463 | E60SaCas9KKH17 | exon60 | SaCas9KKH | − | TCTGAAAGAGAACGTGAGCCAC | GTCAAT |
| 2464 | E60SaCas9KKH18 | exon60 | SaCas9KKH | − | ATTGCACACAGGCACTTCGAGG | AGAAAT |
| 2465 | E61SaCas9KKH1 | exon61 | SaCas9KKH | + | GCTGAAAATGACTTACTGGAAA | GAAAGT |
| 2466 | E61SaCas9KKH2 | exon61 | SaCas9KKH | + | TGACTTACTGGAAAGAAAGTGC | TGAGAT |
| 2467 | E61SaCas9KKH3 | exon61 | SaCas9KKH | + | AGAAAGTGCTGAGATGCTGGAC | CAAAGT |
| 2468 | E61SaCas9KKH4 | exon61 | SaCas9KKH | + | TGGGCTTCATGCAGCTGCCTGA | CTCGGT |
| 2469 | E61SaCas9KKH7 | exon61 | SaCas9KKH | − | GCATCTCAGCACTTTCTTTCCA | GTAAGT |
| 2470 | E61SaCas9KKH8 | exon61 | SaCas9KKH | − | TCCAGCATCTCAGCACTTTCTT | TCCAGT |
| 2471 | E61SaCas9KKH9 | exon61 | SaCas9KKH | − | GCTGCATGAAGCCCACAGGGAC | TTTGGT |
| 2472 | E61SaCas9KKH10 | exon61 | SaCas9KKH | − | CCTCCCAGGTGGCCGTCGAGGA | CCGAGT |
| 2473 | E62SaCas9KKH2 | exon62 | SaCas9KKH | + | GTGATACTTCCAACTTACTTGA | TATAGT |
| 2474 | E62SaCas9KKH3 | exon62 | SaCas9KKH | + | TATAGTAGGGCACTTTGTTTGG | CGAGAT |
| 2475 | E62SaCas9KKH5 | exon62 | SaCas9KKH | − | ACAAAGTGCCCTACTATATCAA | GTAAGT |
| 2476 | E62SaCas9KKH6 | exon62 | SaCas9KKH | − | CCAAACAAAGTGCCCTACTATA | TCAAGT |
| 2477 | E62SaCas9KKH7 | exon62 | SaCas9KKH | − | GGGAGAGAGCCATCTCGCCAAA | CAAAGT |
| 2478 | E62SaCas9KKH8 | exon62 | SaCas9KKH | − | TCCCTTCTTTTCAGCGTCTGTC | CAGGGT |
| 2479 | E63SaCas9KKH1 | exon63 | SaCas9KKH | + | ATGGCCATGTCCTTACCTAAAG | ACTGGT |
| 2480 | E63SaCas9KKH2 | exon63 | SaCas9KKH | + | GACTGGTAGAGCTCTGTCATTT | TGGGAT |
| 2481 | E63SaCas9KKH3 | exon63 | SaCas9KKH | + | TGGTAGAGCTCTGTCATTTTGG | GATGGT |
| 2482 | E63SaCas9KKH4 | exon63 | SaCas9KKH | + | CTGTCATTTTGGGATGGTCCCA | GCAAGT |
| 2483 | E63SaCas9KKH5 | exon63 | SaCas9KKH | + | TGGGATGGTCCCAGCAAGTTGT | TTGAGT |
| 2484 | E63SaCas9KKH7 | exon63 | SaCas9KKH | − | AATGACAGAGCTCTACCAGTCT | TTAGGT |
| 2485 | E63SaCas9KKH8 | exon63 | SaCas9KKH | − | CATCCCAAAATGACAGAGCTCT | ACCAGT |
| 2486 | E63SaCas9KKH9 | exon63 | SaCas9KKH | − | AAACAACTTGCTGGGACCATCC | CAAAAT |
| 2487 | E64SaCas9KKH1 | exon64 | SaCas9KKH | + | ATACTTACAGCAAAGGGCCTTC | TGCAGT |
| 2488 | E64SaCas9KKH2 | exon64 | SaCas9KKH | + | GCAAAGGGCCTTCTGCAGTCTT | CGGAGT |
| 2489 | E64SaCas9KKH3 | exon64 | SaCas9KKH | + | TCTGCAGTCTTCGGAGTTTCAT | GGCAGT |
| 2490 | E64SaCas9KKH4 | exon64 | SaCas9KKH | + | TTTCATGGCAGTCCTATAAGCT | GAGAAT |
| 2491 | E64SaCas9KKH5 | exon64 | SaCas9KKH | + | TAAGCTGAGAATCTGACATTAT | TCAGGT |
| 2492 | E64SaCas9KKH9 | exon64 | SaCas9KKH | − | AGACTGCAGAAGGCCCTTTGCT | GTAAGT |
| 2493 | E64SaCas9KKH10 | exon64 | SaCas9KKH | − | TTTTCAGCTGACCTGAATAATG | TCAGAT |
| 2494 | E64SaCas9KKH11 | exon64 | SaCas9KKH | − | TTCCCTCTTTTCAGCTGACCTG | AATAAT |
| 2495 | E64SaCas9KKH12 | exon64 | SaCas9KKH | − | GTTTTCCCTCTTTTCAGCTGAC | CTGAAT |
| 2496 | E65SaCas9KKH1 | exon65 | SaCas9KKH | + | TACGTATCATAAACATTCAGCA | GCCAGT |
| 2497 | E65SaCas9KKH2 | exon65 | SaCas9KKH | + | TCCACGCAGAGAGGGACGTTGA | CCAAAT |
| 2498 | E65SaCas9KKH3 | exon65 | SaCas9KKH | + | AAATTGTTGTGCTCTTGCTCCA | GGCGGT |
| 2499 | E65SaCas9KKH4 | exon65 | SaCas9KKH | + | TGTGCTCTTGCTCCAGGCGGTC | ATAAAT |
| 2500 | E65SaCas9KKH5 | exon65 | SaCas9KKH | + | GCTCTTGCTCCAGGCGGTCATA | AATAGT |
| 2501 | E65SaCas9KKH6 | exon65 | SaCas9KKH | + | CTTGCTCCAGGCGGTCATAAAT | AGTGGT |
| 2502 | E65SaCas9KKH7 | exon65 | SaCas9KKH | + | AGGCGGTCATAAATAGTGGTCA | AACAAT |
| 2503 | E65SaCas9KKH8 | exon65 | SaCas9KKH | + | GGTCATAAATAGTGGTCAAACA | ATTAAT |
| 2504 | E65SaCas9KKH9 | exon65 | SaCas9KKH | + | CATAAATAGTGGTCAAACAATT | AATAAT |
| 2505 | E65SaCas9KKH10 | exon65 | SaCas9KKH | + | TGGTCAAACAATTAATAATCTG | CAGGAT |
| 2506 | E65SaCas9KKH11 | exon65 | SaCas9KKH | + | ATAATCTGCAGGATATCCATGG | GCTGGT |
| 2507 | E65SaCas9KKH12 | exon65 | SaCas9KKH | + | TCCATGGGCTGGTCATTTTGCT | TGAGGT |
| 2508 | E65SaCas9KKH13 | exon65 | SaCas9KKH | + | TGGTCATTTTGCTTGAGGTTGT | GCTGGT |
| 2509 | E65SaCas9KKH14 | exon65 | SaCas9KKH | + | TCACATGCAGCTGACAGGCTCA | AGAGAT |
| 2510 | E65SaCas9KKH18 | exon65 | SaCas9KKH | − | TCTGAACTGGCTGCTGAATGTT | TATGAT |
| 2511 | E65SaCas9KKH19 | exon65 | SaCas9KKH | − | GGATATGTGTCTGAACTGGCTG | CTGAAT |
| 2512 | E65SaCas9KKH20 | exon65 | SaCas9KKH | − | TTTGGTCAACGTCCCTCTCTGC | GTGGAT |
| 2513 | E65SaCas9KKH21 | exon65 | SaCas9KKH | − | GCCTGGAGCAAGAGCACAACAA | TTTGGT |
| 2514 | E65SaCas9KKH22 | exon65 | SaCas9KKH | − | TGACCGCCTGGAGCAAGAGCAC | AACAAT |
| 2515 | E65SaCas9KKH23 | exon65 | SaCas9KKH | − | GCCCATGGATATCCTGCAGATT | ATTAAT |
| 2516 | E65SaCas9KKH24 | exon65 | SaCas9KKH | − | ATGACCAGCCCATGGATATCCT | GCAGAT |
| 2517 | E65SaCas9KKH25 | exon65 | SaCas9KKH | − | CCTCAAGCAAAATGACCAGCCC | ATGGAT |
| 2518 | E65SaCas9KKH26 | exon65 | SaCas9KKH | − | CTTGGACCAGCACAACCTCAAG | CAAAAT |
| 2519 | E65SaCas9KKH27 | exon65 | SaCas9KKH | − | TCTCTTGAGCCTGTCAGCTGCA | TGTGAT |
| 2520 | E66SaCas9KKH1 | exon66 | SaCas9KKH | + | GACTTACATCTGTACTTGTCTT | CCAAAT |
| 2521 | E66SaCas9KKH2 | exon66 | SaCas9KKH | + | CTTCCAAATGTGCTTTACACAG | GGAAAT |
| 2522 | E66SaCas9KKH3 | exon66 | SaCas9KKH | + | CCAAATGTGCTTTACACAGGGA | AATGAT |
| 2523 | E66SaCas9KKH4 | exon66 | SaCas9KKH | + | GTGCTTTACACAGGGAAATGAT | GCCAGT |
| 2524 | E66SaCas9KKH5 | exon66 | SaCas9KKH | + | TGCCAGTTTTAAAAGACAGGAC | ACGGAT |
| 2525 | E66SaCas9KKH9 | exon66 | SaCas9KKH | − | CATTTGGAAGACAAGTACAGAT | GTAAGT |
| 2526 | E66SaCas9KKH10 | exon66 | SaCas9KKH | − | AAAGCACATTTGGAAGACAAGT | ACAGAT |
| 2527 | E66SaCas9KKH11 | exon66 | SaCas9KKH | − | CTGTGTAAAGCACATTTGGAAG | ACAAGT |
| 2528 | E66SaCas9KKH12 | exon66 | SaCas9KKH | − | TTCATAATAGGGGACGAACAGG | GAGGAT |
| 2529 | E67SaCas9KKH2 | exon67 | SaCas9KKH | + | GGAAGCAGCTCCGGACACTTGG | CTCAAT |
| 2530 | E67SaCas9KKH3 | exon67 | SaCas9KKH | + | TGGCTCAATGTTACTGCCCCCA | AAGGAT |
| 2531 | E67SaCas9KKH4 | exon67 | SaCas9KKH | + | ATGCAACTTCACCCAACTGTCT | TGGAAT |
| 2532 | E67SaCas9KKH5 | exon67 | SaCas9KKH | + | CTTCACCCAACTGTCTTGGAAT | TTGGAT |
| 2533 | E67SaCas9KKH6 | exon67 | SaCas9KKH | + | CCCAACTGTCTTGGAATTTGGA | TAGAAT |
| 2534 | E67SaCas9KKH7 | exon67 | SaCas9KKH | + | TGCAGAAGGAGGCCCAGCCTGC | GCTGGT |
| 2535 | E67SaCas9KKH8 | exon67 | SaCas9KKH | + | GAGGCCCAGCCTGCGCTGGTCA | CAAAAT |
| 2536 | E67SaCas9KKH9 | exon67 | SaCas9KKH | + | GTTGAACTTGCCACTTGCTTGA | AAAGGT |
| 2537 | E67SaCas9KKH13 | exon67 | SaCas9KKH | − | TGTCCGGAGCTGCTTCCAATTT | GTAAGT |
| 2538 | E67SaCas9KKH14 | exon67 | SaCas9KKH | − | GAGCCAAGTGTCCGGAGCTGCT | TCCAAT |
| 2539 | E67SaCas9KKH15 | exon67 | SaCas9KKH | − | CTTTGGGGGCAGTAACATTGAG | CCAAGT |
| 2540 | E67SaCas9KKH16 | exon67 | SaCas9KKH | − | GGGTGAAGTTGCATCCTTTGGG | GGCAGT |
| 2541 | E67SaCas9KKH17 | exon67 | SaCas9KKH | − | TCCAAATTCCAAGACAGTTGGG | TGAAGT |
| 2542 | E67SaCas9KKH18 | exon67 | SaCas9KKH | − | TTCTATCCAAATTCCAAGACAG | TTGGGT |
| 2543 | E67SaCas9KKH19 | exon67 | SaCas9KKH | − | CATGATTCTATCCAAATTCCAA | GACAGT |
| 2544 | E67SaCas9KKH20 | exon67 | SaCas9KKH | − | GCCTCCTTCTGCATGATTCTAT | CCAAAT |
| 2545 | E67SaCas9KKH21 | exon67 | SaCas9KKH | − | GCGCAGGCTGGGCCTCCTTCTG | CATGAT |
| 2546 | E67SaCas9KKH22 | exon67 | SaCas9KKH | − | TTCAAGCAAGTGGCAAGTTCAA | CAGGAT |
| 2547 | E67SaCas9KKH23 | exon67 | SaCas9KKH | − | TGTAGACCTTTTCAAGCAAGTG | GCAAGT |
| 2548 | E67SaCas9KKH24 | exon67 | SaCas9KKH | − | CTTCCTTTGTAGACCTTTTCAA | GCAAGT |
| 2549 | E68SaCas9KKH3 | exon68 | SaCas9KKH | + | TTTTTGGTTCCTAATACCTGAA | TCCAAT |
| 2550 | E68SaCas9KKH4 | exon68 | SaCas9KKH | + | TTGGTTCCTAATACCTGAATCC | AATGAT |
| 2551 | E68SaCas9KKH5 | exon68 | SaCas9KKH | + | ATCCAATGATTGGACACTCTTT | GCAGAT |
| 2552 | E68SaCas9KKH6 | exon68 | SaCas9KKH | + | TCTTTGCAGATGTTACATTTGG | CCTGAT |
| 2553 | E68SaCas9KKH7 | exon68 | SaCas9KKH | + | TGTTACATTTGGCCTGATGCTT | GGCAGT |
| 2554 | E68SaCas9KKH8 | exon68 | SaCas9KKH | + | GACGGGCAGCCACACCATGGAC | TGGGGT |
| 2555 | E68SaCas9KKH9 | exon68 | SaCas9KKH | + | CAGCCACACCATGGACTGGGGT | TCCAGT |
| 2556 | E68SaCas9KKH10 | exon68 | SaCas9KKH | + | ATGGACTGGGGTTCCAGTCTCA | TCCAGT |
| 2557 | E68SaCas9KKH11 | exon68 | SaCas9KKH | + | TCCAGTCTAGGAAGAGGGCCGC | TTCGAT |
| 2558 | E68SaCas9KKH13 | exon68 | SaCas9KKH | − | AAAGAGTGTCCAATCATTGGAT | TCAGGT |
| 2559 | E68SaCas9KKH14 | exon68 | SaCas9KKH | − | ATCTGCAAAGAGTGTCCAATCA | TTGGAT |
| 2560 | E68SaCas9KKH15 | exon68 | SaCas9KKH | − | AATGTAACATCTGCAAAGAGTG | TCCAAT |
| 2561 | E68SaCas9KKH16 | exon68 | SaCas9KKH | − | CAGGCCAAATGTAACATCTGCA | AAGAGT |
| 2562 | E68SaCas9KKH17 | exon68 | SaCas9KKH | − | GCAGAAACTGCCAAGCATCAGG | CCAAAT |
| 2563 | E68SaCas9KKH18 | exon68 | SaCas9KKH | − | TGGTGTGGCTGCCCGTCCTGCA | CAGAGT |
| 2564 | E68SaCas9KKH19 | exon68 | SaCas9KKH | − | GGATGAGACTGGAACCCCAGTC | CATGGT |
| 2565 | E68SaCas9KKH20 | exon68 | SaCas9KKH | − | CTAGACTGGATGAGACTGGAAC | CCCAGT |
| 2566 | E68SaCas9KKH21 | exon68 | SaCas9KKH | − | TCGAAGCGGCCCTCTTCCTAGA | CTGGAT |
| 2567 | E68SaCas9KKH22 | exon68 | SaCas9KKH | − | CTTTGCAGGCTAATAATAAGCC | AGAGAT |
| 2568 | E69SaCas9KKH2 | exon69 | SaCas9KKH | + | GCTGGCGTCAAACTTACCGGAG | TGCAAT |
| 2569 | E69SaCas9KKH3 | exon69 | SaCas9KKH | + | ACCGGAGTGCAATATTCCACCA | TGGGAT |
| 2570 | E69SaCas9KKH4 | exon69 | SaCas9KKH | + | GGAGTGCAATATTCCACCATGG | GATAGT |
| 2571 | E69SaCas9KKH5 | exon69 | SaCas9KKH | + | GACCAGAAAAAAAGCAGCTTTG | GCAGAT |
| 2572 | E69SaCas9KKH6 | exon69 | SaCas9KKH | + | AAAAAGCAGCTTTGGCAGATGT | CATAAT |
| 2573 | E69SaCas9KKH7 | exon69 | SaCas9KKH | + | CAGCTTTGGCAGATGTCATAAT | TAAAGT |
| 2574 | E69SaCas9KKH8 | exon69 | SaCas9KKH | + | TAAAGTGCTTTAGACTCCTGTA | CCTGAT |
| 2575 | E69SaCas9KKH10 | exon69 | SaCas9KKH | − | CATGGTGGAATATTGCACTCCG | GTAAGT |
| 2576 | E69SaCas9KKH11 | exon69 | SaCas9KKH | − | ATCCCATGGTGGAATATTGCAC | TCCGGT |
| 2577 | E69SaCas9KKH12 | exon69 | SaCas9KKH | − | CATAAAATGCACTATCCCATGG | TGGAAT |
| 2578 | E69SaCas9KKH13 | exon69 | SaCas9KKH | − | AAGGCCATAAAATGCACTATCC | CATGGT |
| 2579 | E69SaCas9KKH14 | exon69 | SaCas9KKH | − | CTGGTCGAGTTGCAAAAGGCCA | TAAAAT |
| 2580 | E69SaCas9KKH15 | exon69 | SaCas9KKH | − | GCCAAAGCTGCTTTTTTTCTGG | TCGAGT |
| 2581 | E69SaCas9KKH16 | exon69 | SaCas9KKH | | CATCTGCCAAAGCTGCTTTTTT | TCTGGT |
| 2582 | E69SaCas9KKH17 | exon69 | SaCas9KKH | − | CAGGTACAGGAGTCTAAAGCAC | TTTAAT |
| 2583 | E69SaCas9KKH18 | exon69 | SaCas9KKH | − | GTTTTTGCTCTTTATCAGGTAC | AGGAGT |
| 2584 | E70SaCas9KKH1 | exon70 | SaCas9KKH | + | TTTCCATGTTGTCCCCCTCTAA | GACAGT |
| 2585 | E70SaCas9KKH2 | exon70 | SaCas9KKH | + | CCCTCTAAGACAGTCTGCACTG | GCAGGT |
| 2586 | E70SaCas9KKH3 | exon70 | SaCas9KKH | + | TGCACTGGCAGGTAGCCCATTC | GGGGAT |
| 2587 | E70SaCas9KKH4 | exon70 | SaCas9KKH | + | TAGCCCATTCGGGGATGCTTCG | CAAAAT |
| 2588 | E70SaCas9KKH5 | exon70 | SaCas9KKH | + | GGGGATGCTTCGCAAAATACCT | TTTGGT |
| 2589 | E70SaCas9KKH6 | exon70 | SaCas9KKH | + | CTTCGCAAAATACCTTTTGGTT | CGAAAT |
| 2590 | E70SaCas9KKH7 | exon70 | SaCas9KKH | + | CCTTTTGGTTCGAAATTTGTTT | TTTAGT |
| 2591 | E70SaCas9KKH8 | exon70 | SaCas9KKH | + | AATTTGTTTTTTAGTACCTTGG | CAAAGT |
| 2592 | E70SaCas9KKH9 | exon70 | SaCas9KKH | + | GGCAAAGTCTCGAACATCTTCT | CCTGAT |
| 2593 | E70SaCas9KKH10 | exon70 | SaCas9KKH | + | AGTCTCGAACATCTTCTCCTGA | TGTAGT |
| 2594 | E70SaCas9KKH15 | exon70 | SaCas9KKH | − | AGGGGGACAACATGGAAACGTG | AGTAGT |
| 2595 | E70SaCas9KKH16 | exon70 | SaCas9KKH | − | TAGAGGGGGACAACATGGAAAC | GTGAGT |
| 2596 | E70SaCas9KKH17 | exon70 | SaCas9KKH | − | AGCATCCCCGAATGGGCTACCT | GCCAGT |
| 2597 | E70SaCas9KKH18 | exon70 | SaCas9KKH | − | AAAGGTATTTTGCGAAGCATCC | CCGAAT |
| 2598 | E70SaCas9KKH19 | exon70 | SaCas9KKH | − | CTAAAAAACAAATTTCGAACCA | AAAGGT |
| 2599 | E70SaCas9KKH20 | exon70 | SaCas9KKH | − | GACTTTGCCAAGGTACTAAAAA | ACAAAT |
| 2600 | E70SaCas9KKH21 | exon70 | SaCas9KKH | − | GAGAAGATGTTCGAGACTTTGC | CAAGGT |
| 2601 | E70SaCas9KKH22 | exon70 | SaCas9KKH | − | TCCCTTTTAGACTACATCAGGA | GAAGAT |
| 2602 | E71SaCas9KKH2 | exon71 | SaCas9KKH | + | ACTCACGCAGAATCTACTGGCC | AGAAGT |
| 2603 | E71SaCas9KKH3 | exon71 | SaCas9KKH | + | ACGCAGAATCTACTGGCCAGAA | GTTGAT |
| 2604 | E71SaCas9KKH4 | exon71 | SaCas9KKH | + | AATCTACTGGCCAGAAGTTGAT | CAGAGT |
| 2605 | E71SaCas9KKH9 | exon71 | SaCas9KKH | − | ACTTCTGGCCAGTAGATTCTGC | GTGAGT |
| 2606 | E71SaCas9KKH10 | exon71 | SaCas9KKH | − | TACTCTGATCAACTTCTGGCCA | GTAGAT |
| 2607 | E71SaCas9KKH11 | exon71 | SaCas9KKH | − | CCGTTACTCTGATCAACTTCTG | GCCAGT |
| 2608 | E71SaCas9KKH12 | exon71 | SaCas9KKH | − | TTTTGTTTTGCAGTCCCGTTAC | TCTGAT |
| 2609 | E72SaCas9KKH1 | exon72 | SaCas9KKH | + | CAACTAGTCTCATACCTGCTAG | CATAAT |
| 2610 | E72SaCas9KKH2 | exon72 | SaCas9KKH | + | TCTCATACCTGCTAGCATAATG | TTCAAT |
| 2611 | E72SaCas9KKH3 | exon72 | SaCas9KKH | + | CTGCTAGCATAATGTTCAATGC | GTGAAT |
| 2612 | E72SaCas9KKH4 | exon72 | SaCas9KKH | + | TAGCATAATGTTCAATGCGTGA | ATGAGT |
| 2613 | E72SaCas9KKH11 | exon72 | SaCas9KKH | − | TCACGCATTGAACATTATGCTA | GCAGGT |
| 2614 | E72SaCas9KKH12 | exon72 | SaCas9KKH | − | CTCGTCCCCTCAGCTTTCACAC | GATGAT |
| 2615 | E72SaCas9KKH13 | exon72 | SaCas9KKH | − | TGCCTCGTCCCCTCAGCTTTCA | CACGAT |
| 2616 | E73SaCas9KKH1 | exon73 | SaCas9KKH | + | ATACTTACATGCTCTCATTAGG | AGAGAT |
| 2617 | E73SaCas9KKH2 | exon73 | SaCas9KKH | + | TCATTAGGAGAGATGCTATCAT | TTAGAT |
| 2618 | E73SaCas9KKH3 | exon73 | SaCas9KKH | + | AGGAGAGATGCTATCATTTAGA | TAAGAT |
| 2619 | E73SaCas9KKH4 | exon73 | SaCas9KKH | + | TGCTGTTTTCCATTTCTGCTAG | CCTGAT |
| 2620 | E73SaCas9KKH6 | exon73 | SaCas9KKH | − | GCATCTCTCCTAATGAGAGCAT | GTAAGT |
| 2621 | E73SaCas9KKH7 | exon73 | SaCas9KKH | − | TTATCTAAATGATAGCATCTCT | CCTAAT |
| 2622 | E73SaCas9KKH8 | exon73 | SaCas9KKH | − | AAACAGCAATGGATCTTATCTA | AATGAT |
| 2623 | E73SaCas9KKH9 | exon73 | SaCas9KKH | − | GGAAAACAGCAATGGATCTTAT | CTAAAT |
| 2624 | E73SaCas9KKH10 | exon73 | SaCas9KKH | − | CTAGCAGAAATGGAAAACAGCA | ATGGAT |
| 2625 | E73SaCas9KKH11 | exon73 | SaCas9KKH | − | CAGGCTAGCAGAAATGGAAAAC | AGCAAT |
| 2626 | E73SaCas9KKH12 | exon73 | SaCas9KKH | − | TTACGTTTTTTATCAGGCTAGC | AGAAAT |
| 2627 | E74SaCas9KKH1 | exon74 | SaCas9KKH | + | AAAACTCACCTGTTTTCTTCCT | CAAGAT |
| 2628 | E74SaCas9KKH2 | exon74 | SaCas9KKH | + | TGTTTTCTTCCTCAAGATCTGC | TAGGAT |
| 2629 | E74SaCas9KKH3 | exon74 | SaCas9KKH | + | CCCCTCTTTCCTCACTCTCTAA | GGAAAT |
| 2630 | E74SaCas9KKH4 | exon74 | SaCas9KKH | + | TTTCCTCACTCTCTAAGGAAAT | CAAGAT |
| 2631 | E74SaCas9KKH5 | exon74 | SaCas9KKH | + | GGACTACGAGGCTGGCTCAGGG | GGGAGT |
| 2632 | E74SaCas9KKH6 | exon74 | SaCas9KKH | + | CGAGGCTGGCTCAGGGGGGAGT | CCTGGT |
| 2633 | E74SaCas9KKH7 | exon74 | SaCas9KKH | + | GGGGAGTCCTGGTTCAAACTTT | GGCAGT |
| 2634 | E74SaCas9KKH8 | exon74 | SaCas9KKH | + | GAGTCCTGGTTCAAACTTTGGC | AGTAAT |
| 2635 | E74SaCas9KKH9 | exon74 | SaCas9KKH | + | GGTTCAAACTTTGGCAGTAATG | CTGGAT |
| 2636 | E74SaCas9KKH10 | exon74 | SaCas9KKH | + | CTTTGGCAGTAATGCTGGATTA | ACAAAT |
| 2637 | E74SaCas9KKH16 | exon74 | SaCas9KKH | − | CAGATCTTGAGGAAGAAAACAG | GTGAGT |
| 2638 | E74SaCas9KKH17 | exon74 | SaCas9KKH | − | CTAGCAGATCTTGAGGAAGAAA | ACAGGT |
| 2639 | E74SaCas9KKH18 | exon74 | SaCas9KKH | − | AGGGGAGCTAGAGAGAATCCTA | GCAGAT |
| 2640 | E74SaCas9KKH19 | exon74 | SaCas9KKH | − | GTGAGGAAAGAGGGGAGCTAGA | GAGAAT |
| 2641 | E74SaCas9KKH20 | exon74 | SaCas9KKH | − | TGCCCAGATCTTGATTTCCTTA | GAGAGT |
| 2642 | E74SaCas9KKH21 | exon74 | SaCas9KKH | − | AGCCTCGTAGTCCTGCCCAGAT | CTTGAT |
| 2643 | E74SaCas9KKH22 | exon74 | SaCas9KKH | − | TGAGCCAGCCTCGTAGTCCTGC | CCAGAT |
| 2644 | E74SaCas9KKH23 | exon74 | SaCas9KKH | − | GGACTCCCCCCTGAGCCAGCCT | CGTAGT |
| 2645 | E74SaCas9KKH24 | exon74 | SaCas9KKH | − | TTTGTTAATCCAGCATTACTGC | CAAAGT |
| 2646 | E74SaCas9KKH25 | exon74 | SaCas9KKH | − | TTTCCAGAGATGATGAACATTT | GTTAAT |
| 2647 | E75SaCas9KKH1 | exon75 | SaCas9KKH | + | AACTGTGACTCCAGCTGTTTAT | TGTGGT |
| 2648 | E75SaCas9KKH2 | exon75 | SaCas9KKH | + | CCAGCTGTTTATTGTGGTCTTC | CAGGAT |
| 2649 | E75SaCas9KKH3 | exon75 | SaCas9KKH | + | TTCCAGGCGGCCTTTGTGTTGA | CGCAGT |
| 2650 | E75SaCas9KKH4 | exon75 | SaCas9KKH | + | GTTGACGCAGTAGCTTGGCCTC | AGCAAT |
| 2651 | E75SaCas9KKH5 | exon75 | SaCas9KKH | + | CAGCATCCCGGGGACTCTGGGG | AGAGGT |
| 2652 | E75SaCas9KKH6 | exon75 | SaCas9KKH | + | GGGCATCATTTCAGGAGGGGAC | GGCAGT |
| 2653 | E75SaCas9KKH7 | exon75 | SaCas9KKH | + | TTATGTTCGTGCTGCTGCTTTA | GACGGT |
| 2654 | E75SaCas9KKH8 | exon75 | SaCas9KKH | + | TTTAGACGGTCATATTCTGCTT | GCAGAT |
| 2655 | E75SaCas9KKH12 | exon75 | SaCas9KKH | − | ACAGGCTAAGGCAGCTGCTGGA | GCAAGT |
| 2656 | E75SaCas9KKH13 | exon75 | SaCas9KKH | − | GACCACAATAAACAGCTGGAGT | CACAGT |
| 2657 | E75SaCas9KKH14 | exon75 | SaCas9KKH | − | CTGGAAGACCACAATAAACAGC | TGGAGT |
| 2658 | E75SaCas9KKH15 | exon75 | SaCas9KKH | − | CAGGATGCAAATCCTGGAAGAC | CACAAT |
| 2659 | E75SaCas9KKH16 | exon75 | SaCas9KKH | − | AAGGCCGCCTGGAAGCCAGGAT | GCAAAT |
| 2660 | E75SaCas9KKH17 | exon75 | SaCas9KKH | − | AACACAAAGGCCGCCTGGAAGC | CAGGAT |
| 2661 | E75SaCas9KKH18 | exon75 | SaCas9KKH | − | GCCCACCTCTCCCCAGAGTCCC | CGGGAT |
| 2662 | E75SaCas9KKH19 | exon75 | SaCas9KKH | − | TGAAATGATGCCCACCTCTCCC | CAGAGT |
| 2663 | E75SaCas9KKH20 | exon75 | SaCas9KKH | − | CCCCACTGCCGTCCCCTCCTGA | AATGAT |
| 2664 | E75SaCas9KKH21 | exon75 | SaCas9KKH | − | TGTCCCCACTGCCGTCCCCTCC | TGAAAT |
| 2665 | E75SaCas9KKH22 | exon75 | SaCas9KKH | − | GATGCCAATAGGAATCTGCAAG | CAGAAT |
| 2666 | E76SaCas9KKH1 | exon76 | SaCas9KKH | + | GAGTAGCTAGGACACTTACCCA | TGGAGT |
| 2667 | E76SaCas9KKH2 | exon76 | SaCas9KKH | + | TAGGACACTTACCCATGGAGTC | CGAAGT |
| 2668 | E76SaCas9KKH3 | exon76 | SaCas9KKH | + | GACTGCTGTCGGACCTCTGTAG | AGAGGT |
| 2669 | E76SaCas9KKH4 | exon76 | SaCas9KKH | + | TCACTTTGGCCTCTGCCTGGGG | CTAAGT |
| 2670 | E76SaCas9KKH7 | exon76 | SaCas9KKH | − | AGTCAAACTTCGGACTCCATGG | GTAAGT |
| 2671 | E76SaCas9KKH8 | exon76 | SaCas9KKH | − | TGGCAGTCAAACTTCGGACTCC | ATGGGT |
| 2672 | E76SaCas9KKH9 | exon76 | SaCas9KKH | − | GCCTATGCTGCTCCGAGTGGTT | GGCAGT |
| 2673 | E76SaCas9KKH10 | exon76 | SaCas9KKH | − | GCAGTCAGCCTATGCTGCTCCG | AGTGGT |
| 2674 | E76SaCas9KKH11 | exon76 | SaCas9KKH | − | ACAGCAGTCAGCCTATGCTGCT | CCGAGT |
| 2675 | E76SaCas9KKH12 | exon76 | SaCas9KKH | − | TACCTCTCTACAGAGGTCCGAC | AGCAGT |
| 2676 | E76SaCas9KKH13 | exon76 | SaCas9KKH | − | TCCTCTCCTTCTACCTCTCTAC | AGAGGT |
| 2677 | E76SaCas9KKH14 | exon76 | SaCas9KKH | − | CAGAGGCCAAAGTGAATGGCAC | AACGGT |
| 2678 | E76SaCas9KKH15 | exon76 | SaCas9KKH | − | TTAGCCCCAGGCAGAGGCCAAA | GTGAAT |
| 2679 | E76SaCas9KKH16 | exon76 | SaCas9KKH | − | TGACTTAGCCCCAGGCAGAGGC | CAAAGT |
| 2680 | E77SaCas9KKH1 | exon77 | SaCas9KKH | + | AGCTTACCTCTTGAACTAGGGA | AGGAGT |
| 2681 | E77SaCas9KKH2 | exon77 | SaCas9KKH | + | TCTTGAACTAGGGAAGGAGTTG | TTGAGT |
| 2682 | E77SaCas9KKH3 | exon77 | SaCas9KKH | + | CTTGTGTCCTGGGGAGGACTGA | GAAGAT |
| 2683 | E77SaCas9KKH4 | exon77 | SaCas9KKH | + | GGACTGAGAAGATCTTCCTCAC | CTTAAT |
| 2684 | E77SaCas9KKH10 | exon77 | SaCas9KKH | − | CAACAACTCCTTCCCTAGTTCA | AGAGGT |
| 2685 | E77SaCas9KKH11 | exon77 | SaCas9KKH | − | GGAGCAACTCAACAACTCCTTC | CCTAGT |
| 2686 | E77SaCas9KKH12 | exon77 | SaCas9KKH | − | ACACAAGCACAGGGTTAGAGGA | GGTGAT |
| 2687 | E77SaCas9KKH13 | exon77 | SaCas9KKH | − | AGGACACAAGCACAGGGTTAGA | GGAGGT |
| 2688 | E77SaCas9KKH14 | exon77 | SaCas9KKH | − | AGTCCTCCCCAGGACACAAGCA | CAGGGT |
| 2689 | E77SaCas9KKH15 | exon77 | SaCas9KKH | − | TTATTAAGGTGAGGAAGATCTT | CTCAGT |
| 2690 | E77SaCas9KKH16 | exon77 | SaCas9KKH | − | TTTTTGCTTTTATTAAGGTGAG | GAAGAT |
| 2691 | E78SaCas9KKH1 | exon78 | SaCas9KKH | + | TAACCTCTCTCATTGGCTTTCC | AGGGGT |
| 2692 | E78SaCas9KKH2 | exon78 | SaCas9KKH | + | GCTTTCCAGGGGTATTTCTTCC | TTTAAT |
| 2693 | E78SaCas9KKH6 | exon78 | SaCas9KKH | − | CCCTGGAAAGCCAATGAGAGAG | GTTAGT |
| 2694 | E78SaCas9KKH7 | exon78 | SaCas9KKH | − | ATACCCCTGGAAAGCCAATGAG | AGAGGT |
| 2695 | E78SaCas9KKH8 | exon78 | SaCas9KKH | − | AAGGAAGAAATACCCCTGGAAA | GCCAAT |
| 2696 | E79SaCas9KKH1 | exon79 | SaCas9KKH | + | GTAACATAACTGCGTGCTTTAT | TGAGAT |
| 2697 | E79SaCas9KKH2 | exon79 | SaCas9KKH | + | AACTGCGTGCTTTATTGAGATA | CACAGT |
| 2698 | E79SaCas9KKH3 | exon79 | SaCas9KKH | + | GCTTTATTGAGATACACAGTAA | AGCAGT |
| 2699 | E79SaCas9KKH4 | exon79 | SaCas9KKH | + | GAGATACACAGTAAAGCAGTAC | TATAAT |
| 2700 | E79SaCas9KKH5 | exon79 | SaCas9KKH | + | ACACAGTAAAGCAGTACTATAA | TACAAT |
| 2701 | E79SaCas9KKH6 | exon79 | SaCas9KKH | + | CAGTAAAGCAGTACTATAATAC | AATAGT |
| 2702 | E79SaCas9KKH7 | exon79 | SaCas9KKH | + | TAATACAATAGTAAGGCATATA | TTTGGT |
| 2703 | E79SaCas9KKH8 | exon79 | SaCas9KKH | + | CAATAGTAAGGCATATATTTGG | TGAAGT |
| 2704 | E79SaCas9KKH9 | exon79 | SaCas9KKH | + | GTAAGGCATATATTTGGTGAAG | TCTGAT |
| 2705 | E79SaCas9KKH10 | exon79 | SaCas9KKH | + | TTGGTGAAGTCTGATATGTTGT | GAAAAT |
| 2706 | E79SaCas9KKH11 | exon79 | SaCas9KKH | + | GAAGTCTGATATGTTGTGAAAA | TGCAGT |
| 2707 | E79SaCas9KKH12 | exon79 | SaCas9KKH | + | TGTTGTGAAAATGCAGTAAAAC | TGAAGT |
| 2708 | E79SaCas9KKH13 | exon79 | SaCas9KKH | + | ATGCAGTAAAACTGAAGTTTAA | AAAAAT |
| 2709 | E79SaCas9KKH14 | exon79 | SaCas9KKH | + | CAGTAAAACTGAAGTTTAAAAA | AATAAT |
| 2710 | E79SaCas9KKH15 | exon79 | SaCas9KKH | + | CTGAAGTTTAAAAAAATAATTC | GTAAAT |
| 2711 | E79SaCas9KKH16 | exon79 | SaCas9KKH | + | TAAAAAAATAATTCGTAAATGT | TACAGT |
| 2712 | E79SaCas9KKH17 | exon79 | SaCas9KKH | + | AATAATTCGTAAATGTTACAGT | GTTGGT |
| 2713 | E79SaCas9KKH18 | exon79 | SaCas9KKH | + | TGTTACAGTGTTGGTGTTAAAA | CACAAT |
| 2714 | E79SaCas9KKH19 | exon79 | SaCas9KKH | + | TTGGTGTTAAAACACAATATAT | TATGAT |
| 2715 | E79SaCas9KKH20 | exon79 | SaCas9KKH | + | AAAACACAATATATTATGATAC | TCAAGT |
| 2716 | E79SaCas9KKH21 | exon79 | SaCas9KKH | + | TATTATGATACTCAAGTAAGAA | CTCAGT |
| 2717 | E79SaCas9KKH22 | exon79 | SaCas9KKH | + | AAGTAAGAACTCAGTACCTGAA | AACAAT |
| 2718 | E79SaCas9KKH23 | exon79 | SaCas9KKH | + | AAAACAATGACAAAACATGCCA | TGTGAT |
| 2719 | E79SaCas9KKH24 | exon79 | SaCas9KKH | + | ACATGCCATGTGATGTTTATGC | TTCAGT |
| 2720 | E79SaCas9KKH25 | exon79 | SaCas9KKH | + | GATGTTTATGCTTCAGTTACAT | TATGAT |
| 2721 | E79SaCas9KKH26 | exon79 | SaCas9KKH | + | ATGCTTCAGTTACATTATGATT | TACAGT |
| 2722 | E79SaCas9KKH27 | exon79 | SaCas9KKH | + | TCAGTTACATTATGATTTACAG | TTTAAT |
| 2723 | E79SaCas9KKH28 | exon79 | SaCas9KKH | + | CATTATGATTTACAGTTTAATA | CTTGGT |
| 2724 | E79SaCas9KKH29 | exon79 | SaCas9KKH | + | TATGATTTACAGTTTAATACTT | GGTGGT |
| 2725 | E79SaCas9KKH30 | exon79 | SaCas9KKH | + | GGTGGTTATAAAGAACACAACA | CGAAAT |
| 2726 | E79SaCas9KKH31 | exon79 | SaCas9KKH | + | GGTTATAAAGAACACAACACGA | AATAAT |
| 2727 | E79SaCas9KKH32 | exon79 | SaCas9KKH | + | AGAACACAACACGAAATAATGT | CCAAAT |
| 2728 | E79SaCas9KKH33 | exon79 | SaCas9KKH | + | CACAACACGAAATAATGTCCAA | ATTAAT |
| 2729 | E79SaCas9KKH34 | exon79 | SaCas9KKH | + | TAATGTCCAAATTAATTATGCT | TAAAAT |
| 2730 | E79SaCas9KKH35 | exon79 | SaCas9KKH | + | AAATTAATTATGCTTAAAATGC | AGCAAT |
| 2731 | E79SaCas9KKH36 | exon79 | SaCas9KKH | + | CTTAAAATGCAGCAATAAAGCT | CTCAAT |
| 2732 | E79SaCas9KKH37 | exon79 | SaCas9KKH | + | CAATAAAGCTCTCAATTTTTGT | TCAAAT |
| 2733 | E79SaCas9KKH38 | exon79 | SaCas9KKH | + | ATTATGACAGACTCACTCCAGA | GCTAAT |
| 2734 | E79SaCas9KKH39 | exon79 | SaCas9KKH | + | AATGTGTCTAAAAGAAAAACAA | AAAGAT |
| 2735 | E79SaCas9KKH40 | exon79 | SaCas9KKH | + | AAGAAAAACAAAAAGATTAAAA | CAAAAT |
| 2736 | E79SaCas9KKH41 | exon79 | SaCas9KKH | + | TTATTTATGCACTCTATTTACC | TCTGAT |
| 2737 | E79SaCas9KKH42 | exon79 | SaCas9KKH | + | GCACTCTATTTACCTCTGATTT | TAGAAT |
| 2738 | E79SaCas9KKH43 | exon79 | SaCas9KKH | + | TGAAACTTACTTACTTAAACTT | CTTAGT |
| 2739 | E79SaCas9KKH44 | exon79 | SaCas9KKH | + | CTTACTTACTTAAACTTCTTAG | TAGGAT |
| 2740 | E79SaCas9KKH45 | exon79 | SaCas9KKH | + | ACTTAAACTTCTTAGTAGGATG | TAAAGT |
| 2741 | E79SaCas9KKH46 | exon79 | SaCas9KKH | + | GGATGTAAAGTAACCCCTTGTT | TTAAAT |
| 2742 | E79SaCas9KKH47 | exon79 | SaCas9KKH | + | AAAGTAACCCCTTGTTTTAAAT | CTGAGT |
| 2743 | E79SaCas9KKH48 | exon79 | SaCas9KKH | + | CCTTGTTTTAAATCTGAGTTTT | AAAAAT |
| 2744 | E79SaCas9KKH49 | exon79 | SaCas9KKH | + | TAAATCTGAGTTTTAAAAATCC | TTGGGT |
| 2745 | E79SaCas9KKH50 | exon79 | SaCas9KKH | + | TTTAAAAATCCTTGGGTAAAGA | AAAGGT |
| 2746 | E79SaCas9KKH51 | exon79 | SaCas9KKH | + | GGTCCAGCGTCACATAAAGGAA | AAAAAT |
| 2747 | E79SaCas9KKH52 | exon79 | SaCas9KKH | + | GGAAAAAAATGCAAGACAAAAA | CCAAAT |
| 2748 | E79SaCas9KKH53 | exon79 | SaCas9KKH | + | CAAGACAAAAACCAAATCTTCA | TGTAAT |
| 2749 | E79SaCas9KKH54 | exon79 | SaCas9KKH | + | CAAAAACCAAATCTTCATGTAA | TTTGGT |
| 2750 | E79SaCas9KKH55 | exon79 | SaCas9KKH | + | AAACCAAATCTTCATGTAATTT | GGTAAT |
| 2751 | E79SaCas9KKH56 | exon79 | SaCas9KKH | + | GGTAATTTGTTACCTTAGAGCT | TTGGGT |
| 2752 | E79SaCas9KKH57 | exon79 | SaCas9KKH | + | TTAGAGCTTTGGGTTTTCTTTT | GAAAAT |
| 2753 | E79SaCas9KKH58 | exon79 | SaCas9KKH | + | TGAAAATTATGAAGGAAAAAGA | AAGAAT |
| 2754 | E79SaCas9KKH59 | exon79 | SaCas9KKH | + | AGAAAGAATTATAAAGGAAAAA | GAAAAT |
| 2755 | E79SaCas9KKH60 | exon79 | SaCas9KKH | + | TTATAAAGGAAAAAGAAAATAA | CGCAAT |
| 2756 | E79SaCas9KKH61 | exon79 | SaCas9KKH | + | GAAAAAGAAAATAACGCAATGG | ACAAGT |
| 2757 | E79SaCas9KKH62 | exon79 | SaCas9KKH | + | AAAGAAAATAACGCAATGGACA | AGTGGT |
| 2758 | E79SaCas9KKH63 | exon79 | SaCas9KKH | + | GACAAGTGGTGAAGCTGTGAAC | TCAGGT |
| 2759 | E79SaCas9KKH64 | exon79 | SaCas9KKH | + | TGAAGCTGTGAACTCAGGTGTG | CACAAT |
| 2760 | E79SaCas9KKH65 | exon79 | SaCas9KKH | + | ATTATCAGGAACACCCCAAAAC | CAAAGT |
| 2761 | E79SaCas9KKH66 | exon79 | SaCas9KKH | + | CAGGAACACCCCAAAACCAAAG | TGAGGT |
| 2762 | E79SaCas9KKH67 | exon79 | SaCas9KKH | + | CACCCCAAAACCAAAGTGAGGT | AGAAAT |
| 2763 | E79SaCas9KKH68 | exon79 | SaCas9KKH | + | AGAAATAGCATGAGAAGCCGTG | TTTGAT |
| 2764 | E79SaCas9KKH69 | exon79 | SaCas9KKH | + | AGCATGAGAAGCCGTGTTTGAT | GTTAAT |
| 2765 | E79SaCas9KKH70 | exon79 | SaCas9KKH | + | TGAGAAGCCGTGTTTGATGTTA | ATTAAT |
| 2766 | E79SaCas9KKH71 | exon79 | SaCas9KKH | + | AAGCCGTGTTTGATGTTAATTA | ATTAAT |
| 2767 | E79SaCas9KKH72 | exon79 | SaCas9KKH | + | GTTTGATGTTAATTAATTAATT | ATTAAT |
| 2768 | E79SaCas9KKH73 | exon79 | SaCas9KKH | + | TGATGTTAATTAATTAATTATT | AATAAT |
| 2769 | E79SaCas9KKH74 | exon79 | SaCas9KKH | + | GGACAAAACCCACTCTCCAAAA | GCTAAT |
| 2770 | E79SaCas9KKH75 | exon79 | SaCas9KKH | + | CACTCTCCAAAAGCTAATTACA | CTTGAT |
| 2771 | E79SaCas9KKH76 | exon79 | SaCas9KKH | + | GATGTCAGCCCACTCTCCAAAA | GCTAAT |
| 2772 | E79SaCas9KKH77 | exon79 | SaCas9KKH | + | CACTCTCCAAAAGCTAATTACA | CTTGAT |
| 2773 | E79SaCas9KKH78 | exon79 | SaCas9KKH | + | AAAGCTAATTACACTTGATGTC | AGAGGT |
| 2774 | E79SaCas9KKH79 | exon79 | SaCas9KKH | + | ATTACACTTGATGTCAGAGGTA | ACAGAT |
| 2775 | E79SaCas9KKH80 | exon79 | SaCas9KKH | + | GATGTCAGAGGTAACAGATTTG | CAAAAT |
| 2776 | E79SaCas9KKH81 | exon79 | SaCas9KKH | + | GAGGTAACAGATTTGCAAAATT | ATAGGT |
| 2777 | E79SaCas9KKH82 | exon79 | SaCas9KKH | + | AGATTTGCAAAATTATAGGTCA | CACGGT |
| 2778 | E79SaCas9KKH83 | exon79 | SaCas9KKH | + | TTTGCAAAATTATAGGTCACAC | GGTGGT |
| 2779 | E79SaCas9KKH84 | exon79 | SaCas9KKH | + | ATAGGTCACACGGTGGTATCTA | TTGAAT |
| 2780 | E79SaCas9KKH85 | exon79 | SaCas9KKH | + | GTCACACGGTGGTATCTATTGA | ATGAAT |
| 2781 | E79SaCas9KKH86 | exon79 | SaCas9KKH | + | ACACGGTGGTATCTATTGAATG | AATGAT |
| 2782 | E79SaCas9KKH87 | exon79 | SaCas9KKH | + | GTATCTATTGAATGAATGATTT | AAAAAT |
| 2783 | E79SaCas9KKH88 | exon79 | SaCas9KKH | + | ATGAATGATTTAAAAATCAAAA | AGAAAT |
| 2784 | E79SaCas9KKH89 | exon79 | SaCas9KKH | + | TGATTTAAAAATCAAAAAGAAA | TAAAAT |
| 2785 | E79SaCas9KKH90 | exon79 | SaCas9KKH | + | AAAAAGAAATAAAATGGCATGA | AAGAGT |
| 2786 | E79SaCas9KKH91 | exon79 | SaCas9KKH | + | TGAAAGAGTAAAGCTTTTTCCT | ACCAGT |
| 2787 | E79SaCas9KKH92 | exon79 | SaCas9KKH | + | TGAGCTTTTCTCCTCTTTTTTG | AGCAAT |
| 2788 | E79SaCas9KKH93 | exon79 | SaCas9KKH | + | GTTTGTTTATATATTTAACCTG | TCTAAT |
| 2789 | E79SaCas9KKH94 | exon79 | SaCas9KKH | + | TTAACCTGTCTAATCCACCAAG | AAGGGT |
| 2790 | E79SaCas9KKH95 | exon79 | SaCas9KKH | + | AAGAAGGGTTTTTTTGTAACAT | TTGAAT |
| 2791 | E79SaCas9KKH96 | exon79 | SaCas9KKH | + | AGGGTTTTTTTGTAACATTTGA | ATCAAT |
| 2792 | E79SaCas9KKH97 | exon79 | SaCas9KKH | + | ACCTGTGTGGAACTACTCGCAG | AGAAAT |
| 2793 | E79SaCas9KKH98 | exon79 | SaCas9KKH | + | GAACTACTCGCAGAGAAATGCA | AAGGAT |
| 2794 | E79SaCas9KKH99 | exon79 | SaCas9KKH | + | AGAGAAATGCAAAGGATGGAAA | CACAGT |
| 2795 | E79SaCas9KKH100 | exon79 | SaCas9KKH | + | TGGAAACACAGTTCATGGGCTT | CTGGGT |
| 2796 | E79SaCas9KKH101 | exon79 | SaCas9KKH | + | AACACAGTTCATGGGCTTCTGG | GTTGAT |
| 2797 | E79SaCas9KKH102 | exon79 | SaCas9KKH | + | ATGGGCTTCTGGGTTGATACCT | GTCAGT |
| 2798 | E79SaCas9KKH103 | exon79 | SaCas9KKH | + | TGGGTTGATACCTGTCAGTATC | ACAAAT |
| 2799 | E79SaCas9KKH104 | exon79 | SaCas9KKH | + | TGATACCTGTCAGTATCACAAA | TGTGAT |
| 2800 | E79SaCas9KKH105 | exon79 | SaCas9KKH | + | GCTACTGTTTTACACCTTTTCC | CAAAGT |
| 2801 | E79SaCas9KKH106 | exon79 | SaCas9KKH | + | CTTTTCCCAAAGTTTATTTATT | TTAAAT |
| 2802 | E79SaCas9KKH107 | exon79 | SaCas9KKH | + | TTTAAAAACTCATCCCACATGG | GACAAT |
| 2803 | E79SaCas9KKH108 | exon79 | SaCas9KKH | + | AAAACTCATCCCACATGGGACA | ATAAAT |
| 2804 | E79SaCas9KKH109 | exon79 | SaCas9KKH | + | AAAAGCAACAAAAACCAAACCA | CCCAGT |
| 2805 | E79SaCas9KKH110 | exon79 | SaCas9KKH | + | CCTTTTGACTGTGAGAAGAGGG | CATAAT |
| 2806 | E79SaCas9KKH111 | exon79 | SaCas9KKH | + | TTTGACTGTGAGAAGAGGGCAT | AATAAT |
| 2807 | E79SaCas9KKH112 | exon79 | SaCas9KKH | + | CTGTGAGAAGAGGGCATAATAA | TTTAGT |
| 2808 | E79SaCas9KKH113 | exon79 | SaCas9KKH | + | GAAGAGGGCATAATAATTTAGT | TGTAAT |
| 2809 | E79SaCas9KKH114 | exon79 | SaCas9KKH | + | ACAGAGAACTTTATGTATTATG | AACAAT |
| 2810 | E79SaCas9KKH115 | exon79 | SaCas9KKH | + | CTTTATGTATTATGAACAATCA | TCCAAT |
| 2811 | E79SaCas9KKH116 | exon79 | SaCas9KKH | + | CAATCATCCAATCCTTCACTTA | AAGAGT |
| 2812 | E79SaCas9KKH117 | exon79 | SaCas9KKH | + | AAAGAGTGGCCTACTCCTTCAC | AGGGAT |
| 2813 | E79SaCas9KKH118 | exon79 | SaCas9KKH | + | TACTCCTTCACAGGGATGGGCT | GGGAAT |
| 2814 | E79SaCas9KKH119 | exon79 | SaCas9KKH | + | TATCATCGTCCTGCTACTGCTT | GGGAGT |
| 2815 | E79SaCas9KKH120 | exon79 | SaCas9KKH | + | TCCTGCTACTGCTTGGGAGTTA | AAAAAT |
| 2816 | E79SaCas9KKH121 | exon79 | SaCas9KKH | + | GCTTGGGAGTTAAAAAATACCT | TCTGAT |
| 2817 | E79SaCas9KKH122 | exon79 | SaCas9KKH | + | AAAAAATACCTTCTGATGTTCA | CAAGGT |
| 2818 | E79SaCas9KKH123 | exon79 | SaCas9KKH | + | TACCTTCTGATGTTCACAAGGT | CAGAAT |
| 2819 | E79SaCas9KKH124 | exon79 | SaCas9KKH | + | TGTTCACAAGGTCAGAATACCT | TCTGAT |
| 2820 | E79SaCas9KKH125 | exon79 | SaCas9KKH | + | CACAAGGTCAGAATACCTTCTG | ATTGAT |
| 2821 | E79SaCas9KKH126 | exon79 | SaCas9KKH | + | ATTGATTTCCACTGAAGCATCA | TTAAAT |
| 2822 | E79SaCas9KKH127 | exon79 | SaCas9KKH | + | TTCCACTGAAGCATCATTAAAT | CTAAAT |
| 2823 | E79SaCas9KKH128 | exon79 | SaCas9KKH | + | TGGCATTGCTAGCAGCAGGAAG | CTGAAT |
| 2824 | E79SaCas9KKH129 | exon79 | SaCas9KKH | + | CTAGCAGCAGGAAGCTGAATGT | ATCAAT |
| 2825 | E79SaCas9KKH130 | exon79 | SaCas9KKH | + | CAGCAGGAAGCTGAATGTATCA | ATCAAT |
| 2826 | E79SaCas9KKH131 | exon79 | SaCas9KKH | + | AGGAAGCTGAATGTATCAATCA | ATCAAT |
| 2827 | E79SaCas9KKH132 | exon79 | SaCas9KKH | + | AGCTGAATGTATCAATCAATCA | ATCAAT |
| 2828 | E79SaCas9KKH133 | exon79 | SaCas9KKH | + | CAATCAATCAATCAACCAACCA | ACCGAT |
| 2829 | E79SaCas9KKH134 | exon79 | SaCas9KKH | + | AACCAACCAACCGATTACTCAC | TCTGAT |
| 2830 | E79SaCas9KKH135 | exon79 | SaCas9KKH | + | ACCAACCGATTACTCACTCTGA | TATAAT |
| 2831 | E79SaCas9KKH136 | exon79 | SaCas9KKH | + | ACCGATTACTCACTCTGATATA | ATAAGT |
| 2832 | E79SaCas9KKH137 | exon79 | SaCas9KKH | + | TTCATTCACATGTTCCCTTTAA | AAAAAT |
| 2833 | E79SaCas9KKH138 | exon79 | SaCas9KKH | + | TCACATGTTCCCTTTAAAAAAA | TCCAAT |
| 2834 | E79SaCas9KKH139 | exon79 | SaCas9KKH | + | AATACTTTACTTTACTTTCGTT | GTCAGT |
| 2835 | E79SaCas9KKH140 | exon79 | SaCas9KKH | + | TACTTTACTTTCGTTGTCAGTG | GAAAGT |
| 2836 | E79SaCas9KKH141 | exon79 | SaCas9KKH | + | TCGTTGTCAGTGGAAAGTTGTT | TAAAAT |
| 2837 | E79SaCas9KKH142 | exon79 | SaCas9KKH | + | ATGAGAAACATCTGGAGCTGTA | GACAAT |
| 2838 | E79SaCas9KKH143 | exon79 | SaCas9KKH | + | CATCTGGAGCTGTAGACAATGT | TTTAGT |
| 2839 | E79SaCas9KKH144 | exon79 | SaCas9KKH | + | GGAGCTGTAGACAATGTTTTAG | TGTGGT |
| 2840 | E79SaCas9KKH145 | exon79 | SaCas9KKH | + | TGTAGACAATGTTTTAGTGTGG | TGTAGT |
| 2841 | E79SaCas9KKH146 | exon79 | SaCas9KKH | + | CCTCCTGGCTTCCGGCTCCGGG | AAAAAT |
| 2842 | E79SaCas9KKH147 | exon79 | SaCas9KKH | + | GGAAAAATCCATTCTAGACCAA | GCAGGT |
| 2843 | E79SaCas9KKH148 | exon79 | SaCas9KKH | + | ATTCTAGACCAAGCAGGTAAGC | CTGGAT |
| 2844 | E79SaCas9KKH149 | exon79 | SaCas9KKH | + | AGGTAAGCCTGGATGACTGACT | AGAAGT |
| 2845 | E79SaCas9KKH150 | exon79 | SaCas9KKH | + | TAAGCCTGGATGACTGACTAGA | AGTAAT |
| 2846 | E79SaCas9KKH151 | exon79 | SaCas9KKH | + | ACTAGAAGTAATTTCTTTCTAT | TAGGAT |
| 2847 | E79SaCas9KKH152 | exon79 | SaCas9KKH | + | TAGGATGTGACATGAACATTTA | AAAAAT |
| 2848 | E79SaCas9KKH153 | exon79 | SaCas9KKH | + | ACATGAACATTTAAAAAATGGA | AAAAGT |
| 2849 | E79SaCas9KKH154 | exon79 | SaCas9KKH | + | GAACATTTAAAAAATGGAAAAA | GTCAGT |
| 2850 | E79SaCas9KKH155 | exon79 | SaCas9KKH | + | AAAATGGAAAAAGTCAGTCTAT | AGAAAT |
| 2851 | E79SaCas9KKH156 | exon79 | SaCas9KKH | + | CGTATCTCTTTATCTATATAAC | TATAGT |
| 2852 | E79SaCas9KKH157 | exon79 | SaCas9KKH | + | GTATTTATATACTTATAGACAT | ATAGAT |
| 2853 | E79SaCas9KKH158 | exon79 | SaCas9KKH | + | TACTTATAGACATATAGATATA | TGAAAT |
| 2854 | E79SaCas9KKH159 | exon79 | SaCas9KKH | + | CTCAAGCCTGCCCCACTCAGCT | GACAGT |
| 2855 | E79SaCas9KKH160 | exon79 | SaCas9KKH | + | TGCCCCACTCAGCTGACAGTTC | TCAAAT |
| 2856 | E79SaCas9KKH161 | exon79 | SaCas9KKH | + | CTCAGCTGACAGTTCTCAAATG | AGCAGT |
| 2857 | E79SaCas9KKH162 | exon79 | SaCas9KKH | + | GACAGTTCTCAAATGAGCAGTG | TGTAGT |
| 2858 | E79SaCas9KKH163 | exon79 | SaCas9KKH | + | AGTTCTCAAATGAGCAGTGTGT | AGTAGT |
| 2859 | E79SaCas9KKH164 | exon79 | SaCas9KKH | + | AATGAGCAGTGTGTAGTAGTCA | TTTGGT |
| 2860 | E79SaCas9KKH165 | exon79 | SaCas9KKH | + | GCAGTGTGTAGTAGTCATTTGG | TGTGGT |
| 2861 | E79SaCas9KKH166 | exon79 | SaCas9KKH | + | GTGTGTAGTAGTCATTTGGTGT | GGTGGT |
| 2862 | E79SaCas9KKH167 | exon79 | SaCas9KKH | + | AGTCATTTGGTGTGGTGGTAGA | GGAAGT |
| 2863 | E79SaCas9KKH168 | exon79 | SaCas9KKH | + | TGGTGGTAGAGGAAGTCTTATC | TTTAAT |
| 2864 | E79SaCas9KKH169 | exon79 | SaCas9KKH | + | ATATGCAAAAAAAGAAAAAGCC | ATGAAT |
| 2865 | E79SaCas9KKH170 | exon79 | SaCas9KKH | + | AAGAAAAAGCCATGAATTTCAT | ATGGAT |
| 2866 | E79SaCas9KKH171 | exon79 | SaCas9KKH | + | TTTCATATGGATATCACGCCAA | AAGGAT |
| 2867 | E79SaCas9KKH172 | exon79 | SaCas9KKH | + | ATATCACGCCAAAAGGATGCAA | AACAAT |
| 2868 | E79SaCas9KKH173 | exon79 | SaCas9KKH | + | GATGCAAAACAATGCGCTGCCT | CAAAGT |
| 2869 | E79SaCas9KKH174 | exon79 | SaCas9KKH | + | TGTGTGTGTTTGTTTTGTTTTT | AGGGGT |
| 2870 | E79SaCas9KKH175 | exon79 | SaCas9KKH | + | AACTTTTTTTTATAAAGCACAC | TTTAGT |
| 2871 | E79SaCas9KKH176 | exon79 | SaCas9KKH | + | TTTTATAAAGCACACTTTAGTT | TACAAT |
| 2872 | E79SaCas9KKH177 | exon79 | SaCas9KKH | + | ACAATCTTTCTTTATAACTGTT | ATAAAT |
| 2873 | E79SaCas9KKH178 | exon79 | SaCas9KKH | + | TGTTATAAATTTTTAAACAACC | CAAAAT |
| 2874 | E79SaCas9KKH179 | exon79 | SaCas9KKH | + | ACCCAAAATGCGTTCCATATAA | AGAAAT |
| 2875 | E79SaCas9KKH180 | exon79 | SaCas9KKH | + | ATGCGTTCCATATAAAGAAATG | GCAAGT |
| 2876 | E79SaCas9KKH181 | exon79 | SaCas9KKH | + | AAATGGCAAGTTATTTAGCTAT | CAAGAT |
| 2877 | E79SaCas9KKH182 | exon79 | SaCas9KKH | + | ATTTAGCTATCAAGATTTTACA | TGTAGT |
| 2878 | E79SaCas9KKH183 | exon79 | SaCas9KKH | + | TAGTTTTCTTATAACTTTTTTG | TACAAT |
| 2879 | E79SaCas9KKH184 | exon79 | SaCas9KKH | + | TAAAACCTGCCATTGTTAACAA | AACAAT |
| 2880 | E79SaCas9KKH185 | exon79 | SaCas9KKH | + | CAATAACAGACTTAGAAACTAC | TGAAAT |
| 2881 | E79SaCas9KKH186 | exon79 | SaCas9KKH | + | AGACTTAGAAACTACTGAAATC | TACAGT |
| 2882 | E79SaCas9KKH187 | exon79 | SaCas9KKH | + | TAGAAACTACTGAAATCTACAG | TATAAT |
| 2883 | E79SaCas9KKH188 | exon79 | SaCas9KKH | + | GTATAATACCACTACCCTTCAC | AAAAAT |
| 2884 | E79SaCas9KKH189 | exon79 | SaCas9KKH | + | TACCACTACCCTTCACAAAAAT | ATAGAT |
| 2885 | E79SaCas9KKH190 | exon79 | SaCas9KKH | + | TATTTCTTGTAAACTCTTACTG | TCTAAT |
| 2886 | E79SaCas9KKH191 | exon79 | SaCas9KKH | + | ACTGTCTAATCCTCTTTGTTGT | ATGAAT |
| 2887 | E79SaCas9KKH192 | exon79 | SaCas9KKH | + | ATGAATATTATAAAAACCATGC | GGGAAT |
| 2888 | E79SaCas9KKH193 | exon79 | SaCas9KKH | + | TTATAAAAACCATGCGGGAATC | AGGAGT |
| 2889 | E79SaCas9KKH194 | exon79 | SaCas9KKH | + | TGCTCCTTCTTCATCTGTCATG | ACTGAT |
| 2890 | E79SaCas9KKH195 | exon79 | SaCas9KKH | + | ACTAAGGACTCCATCGCTCTGC | CCAAAT |
| 2891 | E79SaCas9KKH197 | exon79 | SaCas9KKH | − | AAAGCACGCAGTTATGTTACAA | AAAAGT |
| 2892 | E79SaCas9KKH198 | exon79 | SaCas9KKH | − | TACTGTGTATCTCAATAAAGCA | CGCAGT |
| 2893 | E79SaCas9KKH199 | exon79 | SaCas9KKH | − | ATAGTACTGCTTTACTGTGTAT | CTCAAT |
| 2894 | E79SaCas9KKH200 | exon79 | SaCas9KKH | − | AATATATGCCTTACTATTGTAT | TATAGT |
| 2895 | E79SaCas9KKH201 | exon79 | SaCas9KKH | − | TTTCACAACATATCAGACTTCA | CCAAAT |
| 2896 | E79SaCas9KKH202 | exon79 | SaCas9KKH | − | TTTACGAATTATTTTTTTAAAC | TTCAGT |
| 2897 | E79SaCas9KKH203 | exon79 | SaCas9KKH | − | TAACACCAACACTGTAACATTT | ACGAAT |
| 2898 | E79SaCas9KKH204 | exon79 | SaCas9KKH | − | TACTGAGTTCTTACTTGAGTAT | CATAAT |
| 2899 | E79SaCas9KKH205 | exon79 | SaCas9KKH | − | TTTTCAGGTACTGAGTTCTTAC | TTGAGT |
| 2900 | E79SaCas9KKH206 | exon79 | SaCas9KKH | − | GTTTTGTCATTGTTTTCAGGTA | CTGAGT |
| 2901 | E79SaCas9KKH207 | exon79 | SaCas9KKH | − | ATGGCATGTTTTGTCATTGTTT | TCAGGT |
| 2902 | E79SaCas9KKH208 | exon79 | SaCas9KKH | − | CACCAAGTATTAAACTGTAAAT | CATAAT |
| 2903 | E79SaCas9KKH209 | exon79 | SaCas9KKH | − | TATAACCACCAAGTATTAAACT | GTAAAT |
| 2904 | E79SaCas9KKH210 | exon79 | SaCas9KKH | − | CGTGTTGTGTTCTTTATAACCA | CCAAGT |
| 2905 | E79SaCas9KKH211 | exon79 | SaCas9KKH | − | TTATTGCTGCATTTTAAGCATA | ATTAAT |
| 2906 | E79SaCas9KKH212 | exon79 | SaCas9KKH | − | AGCTTTATTGCTGCATTTTAAG | CATAAT |
| 2907 | E79SaCas9KKH213 | exon79 | SaCas9KKH | − | GAGTCTGTCATAATATTTGAAC | AAAAAT |
| 2908 | E79SaCas9KKH214 | exon79 | SaCas9KKH | − | ATTAGCTCTGGAGTGAGTCTGT | CATAAT |
| 2909 | E79SaCas9KKH215 | exon79 | SaCas9KKH | − | TTTTAGACACATTAGCTCTGGA | GTGAGT |
| 2910 | E79SaCas9KKH216 | exon79 | SaCas9KKH | − | TTTCTTTTAGACACATTAGCTC | TGGAGT |
| 2911 | E79SaCas9KKH217 | exon79 | SaCas9KKH | − | TAGAGTGCATAAATAATTTTGT | TTTAAT |
| 2912 | E79SaCas9KKH218 | exon79 | SaCas9KKH | − | TCAGAGGTAAATAGAGTGCATA | AATAAT |
| 2913 | E79SaCas9KKH219 | exon79 | SaCas9KKH | − | AAATCAGAGGTAAATAGAGTGC | ATAAAT |
| 2914 | E79SaCas9KKH220 | exon79 | SaCas9KKH | − | TCATTCTAAAATCAGAGGTAAA | TAGAGT |
| 2915 | E79SaCas9KKH221 | exon79 | SaCas9KKH | − | AAGTTTCATTCTAAAATCAGAG | GTAAAT |
| 2916 | E79SaCas9KKH222 | exon79 | SaCas9KKH | − | AAGTAAGTTTCATTCTAAAATC | AGAGGT |
| 2917 | E79SaCas9KKH223 | exon79 | SaCas9KKH | − | TTTAAGTAAGTAAGTTTCATTC | TAAAAT |
| 2918 | E79SaCas9KKH224 | exon79 | SaCas9KKH | − | TCCTACTAAGAAGTTTAAGTAA | GTAAGT |
| 2919 | E79SaCas9KKH225 | exon79 | SaCas9KKH | − | TACATCCTACTAAGAAGTTTAA | GTAAGT |
| 2920 | E79SaCas9KKH226 | exon79 | SaCas9KKH | − | ACTTTACATCCTACTAAGAAGT | TTAAGT |
| 2921 | E79SaCas9KKH227 | exon79 | SaCas9KKH | − | GGGGTTACTTTACATCCTACTA | AGAAGT |
| 2922 | E79SaCas9KKH228 | exon79 | SaCas9KKH | − | TTTAAAACTCAGATTTAAAACA | AGGGGT |
| 2923 | E79SaCas9KKH229 | exon79 | SaCas9KKH | − | TTTACCCAAGGATTTTTAAAAC | TCAGAT |
| 2924 | E79SaCas9KKH230 | exon79 | SaCas9KKH | − | ACGCTGGACCTTTTCTTTACCC | AAGGAT |
| 2925 | E79SaCas9KKH231 | exon79 | SaCas9KKH | − | AAATTACCAAATTACATGAAGA | TTTGGT |
| 2926 | E79SaCas9KKH232 | exon79 | SaCas9KKH | − | GTAACAAATTACCAAATTACAT | GAAGAT |
| 2927 | E79SaCas9KKH233 | exon79 | SaCas9KKH | − | AAAGCTCTAAGGTAACAAATTA | CCAAAT |
| 2928 | E79SaCas9KKH234 | exon79 | SaCas9KKH | − | GAAAACCCAAAGCTCTAAGGTA | ACAAAT |
| 2929 | E79SaCas9KKH235 | exon79 | SaCas9KKH | − | TTCAAAAGAAAACCCAAAGCTC | TAAGGT |
| 2930 | E79SaCas9KKH236 | exon79 | SaCas9KKH | − | TTATAATTCTTTCTTTTTCCTT | CATAAT |
| 2931 | E79SaCas9KKH237 | exon79 | SaCas9KKH | − | TTGCGTTATTTTCTTTTTCCTT | TATAAT |
| 2932 | E79SaCas9KKH238 | exon79 | SaCas9KKH | − | TGTTCCTGATAATTGTGCACAC | CTGAGT |
| 2933 | E79SaCas9KKH239 | exon79 | SaCas9KKH | − | ACTTTGGTTTTGGGGTGTTCCT | GATAAT |
| 2934 | E79SaCas9KKH240 | exon79 | SaCas9KKH | − | CTCACTTTGGTTTTGGGGTGTT | CCTGAT |
| 2935 | E79SaCas9KKH241 | exon79 | SaCas9KKH | − | TATTTCTACCTCACTTTGGTTT | TGGGGT |
| 2936 | E79SaCas9KKH242 | exon79 | SaCas9KKH | − | TCTCATGCTATTTCTACCTCAC | TTTGGT |
| 2937 | E79SaCas9KKH243 | exon79 | SaCas9KKH | − | TTTTGTCCATTATTAATAATTA | ATTAAT |
| 2938 | E79SaCas9KKH244 | exon79 | SaCas9KKH | − | TGGGTTTTGTCCATTATTAATA | ATTAAT |
| 2939 | E79SaCas9KKH245 | exon79 | SaCas9KKH | − | AGAGTGGGTTTTGTCCATTATT | AATAAT |
| 2940 | E79SaCas9KKH246 | exon79 | SaCas9KKH | − | TGGAGAGTGGGTTTTGTCCATT | ATTAAT |
| 2941 | E79SaCas9KKH247 | exon79 | SaCas9KKH | − | AAGTGTAATTAGCTTTTGGAGA | GTGGGT |
| 2942 | E79SaCas9KKH248 | exon79 | SaCas9KKH | − | CATCAAGTGTAATTAGCTTTTG | GAGAGT |
| 2943 | E79SaCas9KKH249 | exon79 | SaCas9KKH | − | TGGAGAGTGGGCTGACATCAAG | TGTAAT |
| 2944 | E79SaCas9KKH250 | exon79 | SaCas9KKH | − | GCTTTTGGAGAGTGGGCTGACA | TCAAGT |
| 2945 | E79SaCas9KKH251 | exon79 | SaCas9KKH | − | CATCAAGTGTAATTAGCTTTTG | GAGAGT |
| 2946 | E79SaCas9KKH252 | exon79 | SaCas9KKH | − | ATCTGTTACCTCTGACATCAAG | TGTAAT |
| 2947 | E79SaCas9KKH253 | exon79 | SaCas9KKH | − | TGCAAATCTGTTACCTCTGACA | TCAAGT |
| 2948 | E79SaCas9KKH254 | exon79 | SaCas9KKH | − | CCACCGTGTGACCTATAATTTT | GCAAAT |
| 2949 | E79SaCas9KKH255 | exon79 | SaCas9KKH | − | CAATAGATACCACCGTGTGACC | TATAAT |
| 2950 | E79SaCas9KKH256 | exon79 | SaCas9KKH | − | TGATTTTTAAATCATTCATTCA | ATAGAT |
| 2951 | E79SaCas9KKH257 | exon79 | SaCas9KKH | − | TTTTTGATTTTTAAATCATTCA | TTCAAT |
| 2952 | E79SaCas9KKH258 | exon79 | SaCas9KKH | − | CCATTTTATTTCTTTTTGATTT | TTAAAT |
| 2953 | E79SaCas9KKH259 | exon79 | SaCas9KKH | − | CTTTCATGCCATTTTATTTCTT | TTTGAT |
| 2954 | E79SaCas9KKH260 | exon79 | SaCas9KKH | − | AGCTCAAGAGGAAAAGCTAAGG | ACTGGT |
| 2955 | E79SaCas9KKH261 | exon79 | SaCas9KKH | − | TTAAATATATAAACAAACAAAC | AAAAAT |
| 2956 | E79SaCas9KKH262 | exon79 | SaCas9KKH | − | CCTTCTTGGTGGATTAGACAGG | TTAAAT |
| 2957 | E79SaCas9KKH263 | exon79 | SaCas9KKH | − | AAAACCCTTCTTGGTGGATTAG | ACAGGT |
| 2958 | E79SaCas9KKH264 | exon79 | SaCas9KKH | − | TGTTACAAAAAAACCCTTCTTG | GTGGAT |
| 2959 | E79SaCas9KKH265 | exon79 | SaCas9KKH | − | CAAATGTTACAAAAAAACCCTT | CTTGGT |
| 2960 | E79SaCas9KKH266 | exon79 | SaCas9KKH | − | GTAAGAAAGAAGGCAAATTGAT | TCAAAT |
| 2961 | E79SaCas9KKH267 | exon79 | SaCas9KKH | − | AAGTAAGTAAGAAAGAAGGCAA | ATTGAT |
| 2962 | E79SaCas9KKH268 | exon79 | SaCas9KKH | − | TTGTAAGTAAGTAAGAAAGAAG | GCAAAT |
| 2963 | E79SaCas9KKH269 | exon79 | SaCas9KKH | − | GTAGTTCCACACAGGTTTGTAA | GTAAGT |
| 2964 | E79SaCas9KKH270 | exon79 | SaCas9KKH | − | GCGAGTAGTTCCACACAGGTTT | GTAAGT |
| 2965 | E79SaCas9KKH271 | exon79 | SaCas9KKH | − | ATTTCTCTGCGAGTAGTTCCAC | ACAGGT |
| 2966 | E79SaCas9KKH272 | exon79 | SaCas9KKH | − | CCATCCTTTGCATTTCTCTGCG | AGTAGT |
| 2967 | E79SaCas9KKH273 | exon79 | SaCas9KKH | − | TTTCCATCCTTTGCATTTCTCT | GCGAGT |
| 2968 | E79SaCas9KKH274 | exon79 | SaCas9KKH | − | CCCCATCACATTTGTGATACTG | ACAGGT |
| 2969 | E79SaCas9KKH275 | exon79 | SaCas9KKH | − | AAAACAGTAGCCCCATCACATT | TGTGAT |
| 2970 | E79SaCas9KKH276 | exon79 | SaCas9KKH | − | TAAACTTTGGGAAAAGGTGTAA | AACAGT |
| 2971 | E79SaCas9KKH277 | exon79 | SaCas9KKH | − | TTAAAATAAATAAACTTTGGGA | AAAGGT |
| 2972 | E79SaCas9KKH278 | exon79 | SaCas9KKH | − | ATGCCACAAGACATAATTTAAA | ATAAAT |
| 2973 | E79SaCas9KKH279 | exon79 | SaCas9KKH | − | TTAAATGCCACAAGACATAATT | TAAAAT |
| 2974 | E79SaCas9KKH280 | exon79 | SaCas9KKH | − | TGAGTTTTTAAATGCCACAAGA | CATAAT |
| 2975 | E79SaCas9KKH281 | exon79 | SaCas9KKH | − | TTGTCCCATGTGGGATGAGTTT | TTAAAT |
| 2976 | E79SaCas9KKH282 | exon79 | SaCas9KKH | − | TAGATTTATTGTCCCATGTGGG | ATGAGT |
| 2977 | E79SaCas9KKH283 | exon79 | SaCas9KKH | − | TTTTTAGATTTATTGTCCCATG | TGGGAT |
| 2978 | E79SaCas9KKH284 | exon79 | SaCas9KKH | − | TGGTTTGGTTTTTGTTGCTTTT | TTAGAT |
| 2979 | E79SaCas9KKH285 | exon79 | SaCas9KKH | − | ACAGTCAAAAGGAACTGGGTGG | TTTGGT |
| 2980 | E79SaCas9KKH286 | exon79 | SaCas9KKH | − | TTCTCACAGTCAAAAGGAACTG | GGTGGT |
| 2981 | E79SaCas9KKH287 | exon79 | SaCas9KKH | − | CTCTTCTCACAGTCAAAAGGAA | CTGGGT |
| 2982 | E79SaCas9KKH288 | exon79 | SaCas9KKH | − | CTAAATTATTATGCCCTCTTCT | CACAGT |
| 2983 | E79SaCas9KKH289 | exon79 | SaCas9KKH | − | ATAAAGTTCTCTGTAATTACAA | CTAAAT |
| 2984 | E79SaCas9KKH290 | exon79 | SaCas9KKH | − | GTTCATAATACATAAAGTTCTC | TGTAAT |
| 2985 | E79SaCas9KKH291 | exon79 | SaCas9KKH | − | TTGGATGATTGTTCATAATACA | TAAAGT |
| 2986 | E79SaCas9KKH292 | exon79 | SaCas9KKH | − | AGTGAAGGATTGGATGATTGTT | CATAAT |
| 2987 | E79SaCas9KKH293 | exon79 | SaCas9KKH | − | CCACTCTTTAAGTGAAGGATTG | GATGAT |
| 2988 | E79SaCas9KKH294 | exon79 | SaCas9KKH | − | AGGCCACTCTTTAAGTGAAGGA | TTGGAT |
| 2989 | E79SaCas9KKH295 | exon79 | SaCas9KKH | − | GGAGTAGGCCACTCTTTAAGTG | AAGGAT |
| 2990 | E79SaCas9KKH296 | exon79 | SaCas9KKH | − | CTGTGAAGGAGTAGGCCACTCT | TTAAGT |
| 2991 | E79SaCas9KKH297 | exon79 | SaCas9KKH | − | GATTCCCAGCCCATCCCTGTGA | AGGAGT |
| 2992 | E79SaCas9KKH298 | exon79 | SaCas9KKH | − | ACGATGATAGGGCTGGAGGGCT | ATGGAT |
| 2993 | E79SaCas9KKH299 | exon79 | SaCas9KKH | − | AACTCCCAAGCAGTAGCAGGAC | GATGAT |
| 2994 | E79SaCas9KKH300 | exon79 | SaCas9KKH | − | TTTAACTCCCAAGCAGTAGCAG | GACGAT |
| 2995 | E79SaCas9KKH301 | exon79 | SaCas9KKH | − | AGAAGGTATTTTTTAACTCCCA | AGCAGT |
| 2996 | E79SaCas9KKH302 | exon79 | SaCas9KKH | − | TATTCTGACCTTGTGAACATCA | GAAGGT |
| 2997 | E79SaCas9KKH303 | exon79 | SaCas9KKH | − | ATGCTTCAGTGGAAATCAATCA | GAAGGT |
| 2998 | E79SaCas9KKH304 | exon79 | SaCas9KKH | − | ATTTAATGATGCTTCAGTGGAA | ATCAAT |
| 2999 | E79SaCas9KKH305 | exon79 | SaCas9KKH | − | TTAGATTTAATGATGCTTCAGT | GGAAAT |
| 3000 | E79SaCas9KKH306 | exon79 | SaCas9KKH | − | CACGATTTAGATTTAATGATGC | TTCAGT |
| 3001 | E79SaCas9KKH307 | exon79 | SaCas9KKH | − | AGCAATGCCACGATTTAGATTT | AATGAT |
| 3002 | E79SaCas9KKH308 | exon79 | SaCas9KKH | − | GCTAGCAATGCCACGATTTAGA | TTTAAT |
| 3003 | E79SaCas9KKH309 | exon79 | SaCas9KKH | − | CTGCTGCTAGCAATGCCACGAT | TTAGAT |
| 3004 | E79SaCas9KKH310 | exon79 | SaCas9KKH | − | AGCTTCCTGCTGCTAGCAATGC | CACGAT |
| 3005 | E79SaCas9KKH311 | exon79 | SaCas9KKH | − | ATACATTCAGCTTCCTGCTGCT | AGCAAT |
| 3006 | E79SaCas9KKH312 | exon79 | SaCas9KKH | − | TGGTTGGTTGATTGATTGATTG | ATTGAT |
| 3007 | E79SaCas9KKH313 | exon79 | SaCas9KKH | − | CGGTTGGTTGGTTGATTGATTG | ATTGAT |
| 3008 | E79SaCas9KKH314 | exon79 | SaCas9KKH | − | TAATCGGTTGGTTGGTTGATTG | ATTGAT |
| 3009 | E79SaCas9KKH315 | exon79 | SaCas9KKH | − | TGAGTAATCGGTTGGTTGGTTG | ATTGAT |
| 3010 | E79SaCas9KKH316 | exon79 | SaCas9KKH | − | AGAGTGAGTAATCGGTTGGTTG | GTTGAT |
| 3011 | E79SaCas9KKH317 | exon79 | SaCas9KKH | − | TATCAGAGTGAGTAATCGGTTG | GTTGGT |
| 3012 | E79SaCas9KKH318 | exon79 | SaCas9KKH | − | ATTATATCAGAGTGAGTAATCG | GTTGGT |
| 3013 | E79SaCas9KKH319 | exon79 | SaCas9KKH | − | ACTTATTATATCAGAGTGAGTA | ATCGGT |
| 3014 | E79SaCas9KKH320 | exon79 | SaCas9KKH | − | CAGGACTTATTATATCAGAGTG | AGTAAT |
| 3015 | E79SaCas9KKH321 | exon79 | SaCas9KKH | − | ACACAGGACTTATTATATCAGA | GTGAGT |
| 3016 | E79SaCas9KKH322 | exon79 | SaCas9KKH | − | GAATACACAGGACTTATTATAT | CAGAGT |
| 3017 | E79SaCas9KKH323 | exon79 | SaCas9KKH | − | TTTTTTTAAAGGGAACATGTGA | ATGAAT |
| 3018 | E79SaCas9KKH324 | exon79 | SaCas9KKH | − | TGGATTTTTTTAAAGGGAACAT | GTGAAT |
| 3019 | E79SaCas9KKH325 | exon79 | SaCas9KKH | − | ACAACGAAAGTAAAGTAAAGTA | TTGGAT |
| 3020 | E79SaCas9KKH326 | exon79 | SaCas9KKH | − | TCCACTGACAACGAAAGTAAAG | TAAAGT |
| 3021 | E79SaCas9KKH327 | exon79 | SaCas9KKH | − | AACTTTCCACTGACAACGAAAG | TAAAGT |
| 3022 | E79SaCas9KKH328 | exon79 | SaCas9KKH | − | TAAACAACTTTCCACTGACAAC | GAAAGT |
| 3023 | E79SaCas9KKH329 | exon79 | SaCas9KKH | − | CACTAAAACATTGTCTACAGCT | CCAGAT |
| 3024 | E79SaCas9KKH330 | exon79 | SaCas9KKH | − | AGGCTTACCTGCTTGGTCTAGA | ATGGAT |
| 3025 | E79SaCas9KKH331 | exon79 | SaCas9KKH | − | ATCCAGGCTTACCTGCTTGGTC | TAGAAT |
| 3026 | E79SaCas9KKH332 | exon79 | SaCas9KKH | − | GTCAGTCATCCAGGCTTACCTG | CTTGGT |
| 3027 | E79SaCas9KKH333 | exon79 | SaCas9KKH | − | TAATAGAAAGAAATTACTTCTA | GTCAGT |
| 3028 | E79SaCas9KKH334 | exon79 | SaCas9KKH | − | ATCCTAATAGAAAGAAATTACT | TCTAGT |
| 3029 | E79SaCas9KKH335 | exon79 | SaCas9KKH | − | TTCATGTCACATCCTAATAGAA | AGAAAT |
| 3030 | E79SaCas9KKH336 | exon79 | SaCas9KKH | − | TTTTTAAATGTTCATGTCACAT | CCTAAT |
| 3031 | E79SaCas9KKH337 | exon79 | SaCas9KKH | − | ATAGACTGACTTTTTCCATTTT | TTAAAT |
| 3032 | E79SaCas9KKH338 | exon79 | SaCas9KKH | − | ATAGTTATATAGATAAAGAGAT | ACGAAT |
| 3033 | E79SaCas9KKH339 | exon79 | SaCas9KKH | − | AATACTATAGTTATATAGATAA | AGAGAT |
| 3034 | E79SaCas9KKH340 | exon79 | SaCas9KKH | − | AGTATATAAATACTATAGTTAT | ATAGAT |
| 3035 | E79SaCas9KKH341 | exon79 | SaCas9KKH | − | ATGTCTATAAGTATATAAATAC | TATAGT |
| 3036 | E79SaCas9KKH342 | exon79 | SaCas9KKH | − | ATATCTATATGTCTATAAGTAT | ATAAAT |
| 3037 | E79SaCas9KKH343 | exon79 | SaCas9KKH | − | CATTTCATATATCTATATGTCT | ATAAGT |
| 3038 | E79SaCas9KKH344 | exon79 | SaCas9KKH | − | CTGTCAGCTGAGTGGGGCAGGC | TTGAGT |
| 3039 | E79SaCas9KKH345 | exon79 | SaCas9KKH | − | CTGCTCATTTGAGAACTGTCAG | CTGAGT |
| 3040 | E79SaCas9KKH346 | exon79 | SaCas9KKH | − | TAAGACTTCCTCTACCACCACA | CCAAAT |
| 3041 | E79SaCas9KKH347 | exon79 | SaCas9KKH | − | GCTTTTTCTTTTTTTGCATATT | AAAGAT |
| 3042 | E79SaCas9KKH348 | exon79 | SaCas9KKH | − | TCCTTTTGGCGTGATATCCATA | TGAAAT |
| 3043 | E79SaCas9KKH349 | exon79 | SaCas9KKH | − | GCATTGTTTTGCATCCTTTTGG | CGTGAT |
| 3044 | E79SaCas9KKH350 | exon79 | SaCas9KKH | − | AACTAAAGTGTGCTTTATAAAA | AAAAGT |
| 3045 | E79SaCas9KKH351 | exon79 | SaCas9KKH | − | GTTATAAAGAAAGATTGTAAAC | TAAAGT |
| 3046 | E79SaCas9KKH352 | exon79 | SaCas9KKH | − | AAAATTTATAACAGTTATAAAG | AAAGAT |
| 3047 | E79SaCas9KKH353 | exon79 | SaCas9KKH | − | TTTGGGTTGTTTAAAAATTTAT | AACAGT |
| 3048 | E79SaCas9KKH354 | exon79 | SaCas9KKH | − | TGGAACGCATTTTGGGTTGTTT | AAAAAT |
| 3049 | E79SaCas9KKH355 | exon79 | SaCas9KKH | − | ATTTCTTTATATGGAACGCATT | TTGGGT |
| 3050 | E79SaCas9KKH356 | exon79 | SaCas9KKH | − | ACTACATGTAAAATCTTGATAG | CTAAAT |
| 3051 | E79SaCas9KKH357 | exon79 | SaCas9KKH | − | ATAAGAAAACTACATGTAAAAT | CTTGAT |
| 3052 | E79SaCas9KKH358 | exon79 | SaCas9KKH | − | AAAGTTATAAGAAAACTACATG | TAAAAT |
| 3053 | E79SaCas9KKH359 | exon79 | SaCas9KKH | − | ACACGTCTATGCAATTGTACAA | AAAAGT |
| 3054 | E79SaCas9KKH360 | exon79 | SaCas9KKH | − | AATGGCAGGTTTTACACGTCTA | TGCAAT |
| 3055 | E79SaCas9KKH361 | exon79 | SaCas9KKH | − | TGTTATTGTTTTGTTAACAATG | GCAGGT |
| 3056 | E79SaCas9KKH362 | exon79 | SaCas9KKH | − | CTAAGTCTGTTATTGTTTTGTT | AACAAT |
| 3057 | E79SaCas9KKH363 | exon79 | SaCas9KKH | − | ATACTGTAGATTTCAGTAGTTT | CTAAGT |
| 3058 | E79SaCas9KKH364 | exon79 | SaCas9KKH | − | GTGGTATTATACTGTAGATTTC | AGTAGT |
| 3059 | E79SaCas9KKH365 | exon79 | SaCas9KKH | − | GTAGTGGTATTATACTGTAGAT | TTCAGT |
| 3060 | E79SaCas9KKH366 | exon79 | SaCas9KKH | − | TGAAGGGTAGTGGTATTATACT | GTAGAT |
| 3061 | E79SaCas9KKH367 | exon79 | SaCas9KKH | − | AATCTATATTTTTGTGAAGGGT | AGTGGT |
| 3062 | E79SaCas9KKH368 | exon79 | SaCas9KKH | − | ATAAATCTATATTTTTGTGAAG | GGTAGT |
| 3063 | E79SaCas9KKH369 | exon79 | SaCas9KKH | − | GAAATAAATCTATATTTTTGTG | AAGGGT |
| 3064 | E79SaCas9KKH370 | exon79 | SaCas9KKH | | AGACAGTAAGAGTTTACAAGAA | ATAAAT |
| 3065 | E79SaCas9KKH371 | exon79 | SaCas9KKH | − | GATTAGACAGTAAGAGTTTACA | AGAAAT |
| 3066 | E79SaCas9KKH372 | exon79 | SaCas9KKH | − | ACAACAAAGAGGATTAGACAGT | AAGAGT |
| 3067 | E79SaCas9KKH373 | exon79 | SaCas9KKH | − | ATTCATACAACAAAGAGGATTA | GACAGT |
| 3068 | E79SaCas9KKH374 | exon79 | SaCas9KKH | − | TTTATAATATTCATACAACAAA | GAGGAT |
| 3069 | E79SaCas9KKH375 | exon79 | SaCas9KKH | − | CTCCTGATTCCCGCATGGTTTT | TATAAT |
| 3070 | E79SaCas9KKH376 | exon79 | SaCas9KKH | − | GTTTTACAACTCCTGATTCCCG | CATGGT |
| 3071 | E79SaCas9KKH377 | exon79 | SaCas9KKH | − | GCAGAATAAATGTTTTACAACT | CCTGAT |
| 3072 | E79SaCas9KKH378 | exon79 | SaCas9KKH | − | TGACAGATGAAGAAGGAGCAGA | ATAAAT |
| 3073 | E79SaCas9KKH379 | exon79 | SaCas9KKH | − | GTCATGACAGATGAAGAAGGAG | CAGAAT |
| 3074 | E79SaCas9KKH380 | exon79 | SaCas9KKH | − | GGAGTCCTTAGTATCAGTCATG | ACAGAT |
| 3075 | E79SaCas9KKH381 | exon79 | SaCas9KKH | − | GCAGAGCGATGGAGTCCTTAGT | ATCAGT |
| 3076 | E79SaCas9KKH382 | exon79 | SaCas9KKH | − | ATTTGGGCAGAGCGATGGAGTC | CTTAGT |
| 3077 | E79SaCas9KKH383 | exon79 | SaCas9KKH | − | GCAGATGATTTGGGCAGAGCGA | TGGAGT |
| 3078 | E79SaCas9KKH384 | exon79 | SaCas9KKH | − | ACATGGCAGATGATTTGGGCAG | AGCGAT |
| 3079 | E79SaCas9KKH385 | exon79 | SaCas9KKH | − | AGGAAGTCTTTTCCACATGGCA | GATGAT |
| 3080 | E79SaCas9KKH386 | exon79 | SaCas9KKH | − | TGTAGGAAGTCTTTTCCACATG | GCAGAT |
| 3081 | E79SaCas9KKH387 | exon79 | SaCas9KKH | − | TTGTTTTCCAGGACACAATGTA | GGAAGT |

TABLE 1B
Slu guide sequences (human-hg38.12)

| Seq | Guide ID or | exon_ | | | | |
|---|---|---|---|---|---|---|
| ID No. | Guide RNA Name | id | enzyme | strand | Guide Sequence | pam |
| 4000 | E1Slu1 | exon1 | SluCas9 | + | AGCATTTTGAAAAGTGTATATC | AAGG |
| 4001 | E1Slu2 | exon1 | SluCas9 | + | TCAAGGCAGCGATAAAAAAAAC | CTGG |
| 4002 | E1Slu3 | exon1 | SluCas9 | + | TCTTCAAACTTTATTGCTCCAG | TAGG |
| 4003 | E1Slu4 | exon1 | SluCas9 | + | TTTTCCTATTCGTTTTTCTCCG | AAGG |
| 4004 | E1Slu5 | exon1 | SluCas9 | + | TGCCTCCCAGATCTGAGTCCTG | TAGG |
| 4005 | E1Slu6 | exon1 | SluCas9 | + | GCCTCCCAGATCTGAGTCCTGT | AGGG |
| 4006 | E1Slu7 | exon1 | SluCas9 | + | CCTCCCAGATCTGAGTCCTGTA | GGGG |
| 4007 | E1Slu8 | exon1 | SluCas9 | + | CTCCCAGATCTGAGTCCTGTAG | GGGG |
| 4008 | E1Slu9 | exon1 | SluCas9 | − | AATGCTTTGGTGGGAAGAAGTA | GAGG |
| 4009 | E1Slu10 | exon1 | SluCas9 | − | TACACTTTTCAAAATGCTTTGG | TGGG |
| 4010 | E1Slu11 | exon1 | SluCas9 | − | ATACACTTTTCAAAATGCTTTG | GTGG |
| 4011 | E1Slu12 | exon1 | SluCas9 | − | GATATACACTTTTCAAAATGCT | TTGG |
| 4012 | E1Slu13 | exon1 | SluCas9 | − | ATAAAGTTTGAAGAACTTTTAC | CAGG |
| 4013 | E1Slu14 | exon1 | SluCas9 | − | TTTCTCATTGTTTTTAAGCCTA | CTGG |
| 4014 | E1Slu15 | exon1 | SluCas9 | − | TTTTTAAAGCTGCTGAAGTTTG | TTGG |
| 4015 | E1Slu16 | exon1 | SluCas9 | − | ATTACCTTCGGAGAAAAACGAA | TAGG |
| 4016 | E1Slu17 | exon1 | SluCas9 | − | CAGATCTGGGAGGCAATTACCT | TCGG |
| 4017 | E1Slu18 | exon1 | SluCas9 | − | CCCCTACAGGACTCAGATCTGG | GAGG |
| 4018 | E1Slu19 | exon1 | SluCas9 | − | TTCCCCCTACAGGACTCAGATC | TGGG |
| 4019 | E1Slu20 | exon1 | SluCas9 | − | TTTCCCCCTACAGGACTCAGAT | CTGG |
| 4020 | E1Slu21 | exon1 | SluCas9 | − | GGGATCACTCACTTTCCCCCTA | CAGG |
| 4021 | E2Slu3 | exon2 | SluCas9 | − | ATGGGTAAATGCACAATTTTCT | AAGG |
| 4022 | E2Slu4 | exon2 | SluCas9 | − | TCAAAAGAAAACATTCACAAAA | TGGG |
| 4023 | E2Slu5 | exon2 | SluCas9 | − | TTCAAAAGAAAACATTCACAAA | ATGG |
| 4024 | E3Slu1 | exon3 | SluCas9 | + | CACATACCAGTTTTTGCCCTGT | CAGG |
| 4025 | E3Slu2 | exon3 | SluCas9 | + | GTTTTTGCCCTGTCAGGCCTTC | GAGG |
| 4026 | E3Slu3 | exon3 | SluCas9 | + | TTTGCCCTGTCAGGCCTTCGAG | GAGG |
| 4027 | E3Slu4 | exon3 | SluCas9 | + | CTGTCAGGCCTTCGAGGAGGTC | TAGG |
| 4028 | E3Slu5 | exon3 | SluCas9 | + | TCAGGCCTTCGAGGAGGTCTAG | GAGG |
| 4029 | E3Slu6 | exon3 | SluCas9 | + | CTAGGAGGCGCCTCCCATCCTG | TAGG |
| 4030 | E3Slu7 | exon3 | SluCas9 | + | TCCCATCCTGTAGGTCACTGAA | GAGG |
| 4031 | E3Slu8 | exon3 | SluCas9 | − | CGAAGGCCTGACAGGGCAAAAA | CTGG |
| 4032 | E3Slu9 | exon3 | SluCas9 | − | TAGACCTCCTCGAAGGCCTGAC | AGGG |
| 4033 | E3Slu10 | exon3 | SluCas9 | − | CTAGACCTCCTCGAAGGCCTGA | CAGG |
| 4034 | E3Slu11 | exon3 | SluCas9 | − | AGGCGCCTCCTAGACCTCCTCG | AAGG |
| 4035 | E3Slu12 | exon3 | SluCas9 | − | TCTTCAGTGACCTACAGGATGG | GAGG |
| 4036 | E3Slu13 | exon3 | SluCas9 | − | ACCTCTTCAGTGACCTACAGGA | TGGG |
| 4037 | E3Slu14 | exon3 | SluCas9 | − | AACCTCTTCAGTGACCTACAGG | ATGG |
| 4038 | E3Slu15 | exon3 | SluCas9 | − | TGAGAACCTCTTCAGTGACCTA | CAGG |
| 4039 | E4Slu1 | exon4 | SluCas9 | + | GCAGTGCCTTGTTGACATTGTT | CAGG |
| 4040 | E4Slu2 | exon4 | SluCas9 | + | CAGTGCCTTGTTGACATTGTTC | AGGG |
| 4041 | E4Slu3 | exon4 | SluCas9 | + | ATTGTTCAGGGCATGAACTCTT | GTGG |
| 4042 | E4Slu4 | exon4 | SluCas9 | + | ACTCTTGTGGATCCTTTTTCTT | TTGG |
| 4043 | E4Slu8 | exon4 | SluCas9 | − | GAACAATGTCAACAAGGCACTG | CGGG |
| 4044 | E4Slu9 | exon4 | SluCas9 | − | TGAACAATGTCAACAAGGCACT | GCGG |
| 4045 | E4Slu10 | exon4 | SluCas9 | − | TCATGCCCTGAACAATGTCAAC | AAGG |
| 4046 | E4Slu11 | exon4 | SluCas9 | − | TTTTGTTCTCAGCCAAAAGAAA | AAGG |
| 4047 | E5Slu1 | exon5 | SluCas9 | + | TCATCAGGATTCTTACCTGCCA | GTGG |
| 4048 | E5Slu2 | exon5 | SluCas9 | + | TCAGGATTCTTACCTGCCAGTG | GAGG |
| 4049 | E5Slu10 | exon5 | SluCas9 | − | TTGGAATATAATCCTCCACTGG | CAGG |
| 4050 | E5Slu11 | exon5 | SluCas9 | − | TGATTTGGAATATAATCCTCCA | CTGG |
| 4051 | E5Slu12 | exon5 | SluCas9 | − | ATAAACTGACTCTTGGTTTGAT | TTGG |
| 4052 | E5Slu13 | exon5 | SluCas9 | − | GATGGAAATCATAAACTGACTC | TTGG |
| 4053 | E5Slu14 | exon5 | SluCas9 | − | ATTGGAAGTACTGACATCGTAG | ATGG |
| 4054 | E5Slu15 | exon5 | SluCas9 | − | TAACAGGTTGATTTAGTGAATA | TTGG |
| 4055 | E6Slu1 | exon6 | SluCas9 | + | AGTAATCTTCTTACCTATGACT | ATGG |
| 4056 | E6Slu2 | exon6 | SluCas9 | + | TATGGATGAGAGCATTCAAAGC | CAGG |
| 4057 | E6Slu3 | exon6 | SluCas9 | + | CAAAGCCAGGCCATCAGACCAG | CTGG |
| 4058 | E6Slu4 | exon6 | SluCas9 | + | AGCCAGGCCATCAGACCAGCTG | GTGG |
| 4059 | E6Slu5 | exon6 | SluCas9 | + | GTGAAGTTGATTACATTAACCT | GTGG |
| 4060 | E6Slu6 | exon6 | SluCas9 | + | GTGGATAATTACGAGTTGATTG | TCGG |
| 4061 | E6Slu7 | exon6 | SluCas9 | + | GAGTTGATTGTCGGACCCAGCT | CAGG |
| 4062 | E6Slu8 | exon6 | SluCas9 | + | GCTCAGGAGAATCTTTTCACTG | TTGG |
| 4063 | E6Slu10 | exon6 | SluCas9 | − | TGAATGCTCTCATCCATAGTCA | TAGG |
| 4064 | E6Slu11 | exon6 | SluCas9 | − | CACCACCAGCTGGTCTGATGGC | CTGG |
| 4065 | E6Slu12 | exon6 | SluCas9 | − | AACTTCACCACCAGCTGGTCTG | ATGG |
| 4066 | E6Slu13 | exon6 | SluCas9 | − | ATGTAATCAACTTCACCACCAG | CTGG |
| 4067 | E6Slu14 | exon6 | SluCas9 | − | ACAATCAACTCGTAATTATCCA | CAGG |
| 4068 | E6Slu15 | exon6 | SluCas9 | − | CAGTGAAAAGATTCTCCTGAGC | TGGG |
| 4069 | E6Slu16 | exon6 | SluCas9 | − | ACAGTGAAAAGATTCTCCTGAG | CTGG |
| 4070 | E6Slu17 | exon6 | SluCas9 | − | AATGTAATGAAAAATATCATGG | CTGG |
| 4071 | E6Slu18 | exon6 | SluCas9 | − | CAAAAATGTAATGAAAAATATC | ATGG |
| 4072 | E7Slu1 | exon7 | SluCas9 | + | AGTCCAGAAATTTACCAACCTT | CAGG |
| 4073 | E7Slu2 | exon7 | SluCas9 | + | TTTCTCTATGCCTAATTGATAT | CTGG |
| 4074 | E7Slu3 | exon7 | SluCas9 | + | GAATGCATGTTCCAGTCGTTGT | GTGG |
| 4075 | E7Slu4 | exon7 | SluCas9 | + | CCAGTCGTTGTGTGGCTGACTG | CTGG |
| 4076 | E7Slu5 | exon7 | SluCas9 | + | AAACCACACTATTCCAGTCAAA | TAGG |
| 4077 | E7Slu6 | exon7 | SluCas9 | + | ACACTATTCCAGTCAAATAGGT | CTGG |
| 4078 | E7Slu8 | exon7 | SluCas9 | − | AGAAACTACTCGATCCTGAAGG | TTGG |
| 4079 | E7Slu9 | exon7 | SluCas9 | − | ATAGAGAAACTACTCGATCCTG | AAGG |
| 4080 | E7Slu10 | exon7 | SluCas9 | − | TTCAACATCGCCAGATATCAAT | TAGG |
| 4081 | E7Slu11 | exon7 | SluCas9 | − | CCAGCAGTCAGCCACACAACGA | CTGG |
| 4082 | E7Slu12 | exon7 | SluCas9 | − | AGACCTATTTGACTGGAATAGT | GTGG |
| 4083 | E7Slu13 | exon7 | SluCas9 | − | GTTTTAGGCCAGACCTATTTGA | CTGG |
| 4084 | E8Slu1 | exon8 | SluCas9 | + | AAATGTTCTTCTTTAGTCACTT | TAGG |
| 4085 | E8Slu2 | exon8 | SluCas9 | + | TGTTCTTCTTTAGTCACTTTAG | GTGG |
| 4086 | E8Slu3 | exon8 | SluCas9 | + | TCTTTAGTCACTTTAGGTGGCC | TTGG |
| 4087 | E8Slu4 | exon8 | SluCas9 | + | GCCTTGGCAACATTTCCACTTC | CTGG |
| 4088 | E8Slu5 | exon8 | SluCas9 | + | TGGCAACATTTCCACTTCCTGG | ATGG |
| 4089 | E8Slu6 | exon8 | SluCas9 | + | ATGGCTTCAATGCTCACTTGTT | GAGG |
| 4090 | E8Slu7 | exon8 | SluCas9 | + | TGCTCACTTGTTGAGGCAAAAC | TTGG |
| 4091 | E8Slu8 | exon8 | SluCas9 | 1 | GAGTGATGTGATGTACATTAAG | ATGG |
| 4092 | E8Slu9 | exon8 | SluCas9 | + | TACATTAAGATGGACTTCTTAT | CTGG |
| 4093 | E8Slu10 | exon8 | SluCas9 | + | TAAGATGGACTTCTTATCTGGA | TAGG |
| 4094 | E8Slu11 | exon8 | SluCas9 | + | GATGGACTTCTTATCTGGATAG | GTGG |
| 4095 | E8Slu13 | exon8 | SluCas9 | − | TCATCAAATGCACTATTCTCAA | CAGG |
| 4096 | E8Slu14 | exon8 | SluCas9 | − | TCCAGGAAGTGGAAATGTTGCC | AAGG |
| 4097 | E8Slu15 | exon8 | SluCas9 | − | GAGCATTGAAGCCATCCAGGAA | GTGG |
| 4098 | E8Slu16 | exon8 | SluCas9 | − | ACAAGTGAGCATTGAAGCCATC | CAGG |
| 4099 | E9Slu1 | exon9 | SluCas9 | + | AGTAAATGTTGACAGACCTGTG | AAGG |
| 4100 | E9Slu2 | exon9 | SluCas9 | + | TGTTGACAGACCTGTGAAGGAA | ATGG |
| 4101 | E9Slu3 | exon9 | SluCas9 | + | GTTGACAGACCTGTGAAGGAAA | TGGG |
| 4102 | E9Slu4 | exon9 | SluCas9 | + | TGTGAAGGAAATGGGCTCCGTG | TAGG |
| 4103 | E9Slu5 | exon9 | SluCas9 | + | GTGAAGGAAATGGGCTCCGTGT | AGGG |
| 4104 | E9Slu6 | exon9 | SluCas9 | + | AAATGGGCTCCGTGTAGGGTCA | GAGG |
| 4105 | E9Slu7 | exon9 | SluCas9 | + | TGGGCTCCGTGTAGGGTCAGAG | GTGG |
| 4106 | E9Slu8 | exon9 | SluCas9 | + | GGTGACATAAGCAGCCTGTGTG | TAGG |
| 4107 | E9Slu9 | exon9 | SluCas9 | + | GTGTAGGCATAGCTCTTGAATC | GAGG |
| 4108 | E9Slu10 | exon9 | SluCas9 | + | GCATAGCTCTTGAATCGAGGCT | TAGG |
| 4109 | E9Slu11 | exon9 | SluCas9 | + | CATAGCTCTTGAATCGAGGCTT | AGGG |
| 4110 | E9Slu12 | exon9 | SluCas9 | + | ATAGCTCTTGAATCGAGGCTTA | GGGG |
| 4111 | E9Slu20 | exon9 | SluCas9 | − | TACACGGAGCCCATTTCCTTCA | CAGG |
| 4112 | E9Slu21 | exon9 | SluCas9 | − | ATGTCACCACCTCTGACCCTAC | ACGG |
| 4113 | E9Slu22 | exon9 | SluCas9 | − | ATTCAAGAGCTATGCCTACACA | CAGG |
| 4114 | E9Slu23 | exon9 | SluCas9 | − | CAGATCACGGTCAGTCTAGCAC | AGGG |
| 4115 | E9Slu24 | exon9 | SluCas9 | − | GCAGATCACGGTCAGTCTAGCA | CAGG |
| 4116 | E9Slu25 | exon9 | SluCas9 | − | CCCAAACCCTTCTCTGCAGATC | ACGG |
| 4117 | E10Slu1 | exon10 | SluCas9 | + | AGTTTACCTCATGAGTATGAAA | CTGG |
| 4118 | E10Slu2 | exon10 | SluCas9 | + | CTTCTTCTAAAGCTGTTTGATA | ACGG |
| 4119 | E10Slu3 | exon10 | SluCas9 | + | CTAAAGCTGTTTGATAACGGTC | CAGG |
| 4120 | E10Slu4 | exon10 | SluCas9 | + | GAACTGCCAAATGACTTGTCTT | CAGG |
| 4121 | E10Slu7 | exon10 | SluCas9 | − | GAAAGACCAGTTTCATACTCAT | GAGG |
| 4122 | E10Slu8 | exon10 | SluCas9 | − | AGAGATTTCTAATGATGTGGAA | GTGG |
| 4123 | E10Slu9 | exon10 | SluCas9 | − | ACAAGGAGAGATTTCTAATGAT | GTGG |
| 4124 | E10Slu10 | exon10 | SluCas9 | − | GCTGAGGACACATTGCAAGCAC | AAGG |
| 4125 | E10Slu11 | exon10 | SluCas9 | − | ATTATCGTGGCTTCTTTCTGCT | GAGG |
| 4126 | E10Slu12 | exon10 | SluCas9 | − | CAGCTTTAGAAGAAGTATTATC | GTGG |
| 4127 | E10Slu13 | exon10 | SluCas9 | − | ATTGATGGAGAGTGAAGTAAAC | CTGG |
| 4128 | E10Slu14 | exon10 | SluCas9 | − | CAAGTCATTTGGCAGTTCATTG | ATGG |
| 4129 | E10Slu15 | exon10 | SluCas9 | − | GAAGCTCCTGAAGACAAGTCAT | TTGG |
| 4130 | E10Slu16 | exon10 | SluCas9 | − | AATTTATTTTATTGTGCAGCAT | TTGG |
| 4131 | E11Slu2 | exon11 | SluCas9 | + | GTTTTTCCATGCTAGCTACCCT | GAGG |
| 4132 | E11Slu3 | exon11 | SluCas9 | + | TGAGGCATTCCCATCTTGAATT | TAGG |
| 4133 | E11Slu4 | exon11 | SluCas9 | + | CCAATTGTAGAATATTACCAAC | CCGG |
| 4134 | E11Slu5 | exon11 | SluCas9 | + | GAATATTACCAACCCGGCCCTG | ATGG |
| 4135 | E11Slu6 | exon11 | SluCas9 | + | AATATTACCAACCCGGCCCTGA | TGGG |
| 4136 | E11Slu8 | exon11 | SluCas9 | − | GGAATGCCTCAGGGTAGCTAGC | ATGG |
| 4137 | E11Slu9 | exon11 | SluCas9 | − | AAATTCAAGATGGGAATGCCTC | AGGG |
| 4138 | E11Slu10 | exon11 | SluCas9 | − | TAAATTCAAGATGGGAATGCCT | CAGG |
| 4139 | E11Slu11 | exon11 | SluCas9 | − | GATGAATCTCCTAAATTCAAGA | TGGG |
| 4140 | E11Slu12 | exon11 | SluCas9 | − | AGATGAATCTCCTAAATTCAAG | ATGG |
| 4141 | E11Slu13 | exon11 | SluCas9 | − | TTGGGAAGTAAGCTGATTGGAA | CAGG |
| 4142 | E11Slu14 | exon11 | SluCas9 | − | CTACAATTGGGAAGTAAGCTGA | TTGG |
| 4143 | E11Slu15 | exon11 | SluCas9 | − | CGGGTTGGTAATATTCTACAAT | TGGG |
| 4144 | E11Slu16 | exon11 | SluCas9 | − | CCGGGTTGGTAATATTCTACAA | TTGG |
| 4145 | E11Slu17 | exon11 | SluCas9 | − | TTGACAGCCCATCAGGGCCGGG | TTGG |
| 4146 | E11Slu18 | exon11 | SluCas9 | − | GGATTTGACAGCCCATCAGGGC | CGGG |
| 4147 | E11Slu19 | exon11 | SluCas9 | − | TGGATTTGACAGCCCATCAGGG | CCGG |
| 4148 | E11Slu20 | exon11 | SluCas9 | − | ATGATGGATTTGACAGCCCATC | AGGG |
| 4149 | E11Slu21 | exon11 | SluCas9 | − | CATGATGGATTTGACAGCCCAT | CAGG |
| 4150 | E11Slu22 | exon11 | SluCas9 | − | CCTTCTTTGTCAGGGGTACATG | ATGG |
| 4151 | E12Slu1 | exon12 | SluCas9 | + | CACCTACCTTATGTTGTTGTAC | TTGG |
| 4152 | E12Slu2 | exon12 | SluCas9 | + | TATGTTGTTGTACTTGGCGTTT | TAGG |
| 4153 | E12Slu3 | exon12 | SluCas9 | + | TGGCGTTTTAGGTCTTCAAGAT | CAGG |
| 4154 | E12Slu4 | exon12 | SluCas9 | + | AGGTCTTCAAGATCAGGTCCAA | GAGG |
| 4155 | E12Slu5 | exon12 | SluCas9 | + | TCAACTCTTTCAGTTTCTGATT | CTGG |
| 4156 | E12Slu6 | exon12 | SluCas9 | − | CGCCAAGTACAACAACATAAGG | TAGG |
| 4157 | E12Slu7 | exon12 | SluCas9 | − | AAAACGCCAAGTACAACAACAT | AAGG |
| 4158 | E12Slu8 | exon12 | SluCas9 | − | AGGAAAATGGAGGAAGAGCCTC | TTGG |
| 4159 | E12Slu9 | exon12 | SluCas9 | − | AGAAGAAAGAACAAGGAAAATG | GAGG |
| 4160 | E12Slu10 | exon12 | SluCas9 | − | AACAGAAGAAAGAACAAGGAAA | ATGG |
| 4161 | E12Slu11 | exon12 | SluCas9 | − | TAACAAAAACAGAAGAAAGAAC | AAGG |
| 4162 | E12Slu12 | exon12 | SluCas9 | − | AGAAACTGAAAGAGTTGAATGA | CTGG |
| 4163 | E12Slu13 | exon12 | SluCas9 | − | TTTCAGTTTACATAGAGTTTTA | ATGG |
| 4164 | E13Slu2 | exon13 | SluCas9 | − | TGCTGCTTTGGAAGAACAACTT | AAGG |
| 4165 | E13Slu3 | exon13 | SluCas9 | − | TGGAGATCACGCAACTGCTGCT | TTGG |
| 4166 | E13Slu4 | exon13 | SluCas9 | − | GTGGTGGTAGTTGATGAATCTA | GTGG |
| 4167 | E13Slu5 | exon13 | SluCas9 | − | CAATTCTCTCACTCACATGGTG | GTGG |
| 4168 | E13Slu6 | exon13 | SluCas9 | − | GGTCAATTCTCTCACTCACATG | GTGG |
| 4169 | E13Slu7 | exon13 | SluCas9 | − | CAGGGTCAATTCTCTCACTCAC | ATGG |
| 4170 | E13Slu8 | exon13 | SluCas9 | − | AGATCTAGAACAAGAACAAGTC | AGGG |
| 4171 | E13Slu9 | exon13 | SluCas9 | − | AAGATCTAGAACAAGAACAAGT | CAGG |
| 4172 | E14Slu1 | exon14 | SluCas9 | + | CAGTAAGACGTTGCCATTTGAG | AAGG |
| 4173 | E14Slu2 | exon14 | SluCas9 | + | GGATGTCTTGTAAAAGAACCCA | GCGG |
| 4174 | E14Slu3 | exon14 | SluCas9 | + | TTGCCCATCGATCTCCCAATAC | CTGG |
| 4175 | E14Slu4 | exon14 | SluCas9 | − | ATGGCAACGTCTTACTGAAGAA | CAGG |
| 4176 | E14Slu5 | exon14 | SluCas9 | − | TTTTACAAGACATCCTTCTCAA | ATGG |
| 4177 | E14Slu6 | exon14 | SluCas9 | − | CTGTAGATGGACAGAAGACCGC | TGGG |
| 4178 | E14Slu7 | exon14 | SluCas9 | − | TCTGTAGATGGACAGAAGACCG | CTGG |
| 4179 | E14Slu8 | exon14 | SluCas9 | − | ATCGATGGGCAAACATCTGTAG | ATGG |
| 4180 | E14Slu9 | exon14 | SluCas9 | − | TCTCCAGGTATTGGGAGATCGA | TGGG |
| 4181 | E14Slu10 | exon14 | SluCas9 | − | TTCTCCAGGTATTGGGAGATCG | ATGG |
| 4182 | E14Slu11 | exon14 | SluCas9 | − | TGATTGTCTCTTCTCCAGGTAT | TGGG |
| 4183 | E14Slu12 | exon14 | SluCas9 | − | TTGATTGTCTCTTCTCCAGGTA | TTGG |
| 4184 | E15Slu2 | exon15 | SluCas9 | + | TTTCTGAAAGCCATGCACTAAA | AAGG |
| 4185 | E15Slu5 | exon15 | SluCas9 | − | AATGTTATCAAGTCTTCAAAAA | CTGG |
| 4186 | E15Slu6 | exon15 | SluCas9 | − | GCAGTGAACAAGATTCACACAA | CTGG |
| 4187 | E15Slu7 | exon15 | SluCas9 | − | TCTTGCAGTGCCTTTTTAGTGC | ATGG |
| 4188 | E16Slu1 | exon16 | SluCas9 | + | GTGCTGTACTCTTTTCAAGTTT | TTGG |
| 4189 | E16Slu2 | exon16 | SluCas9 | + | TTTGGACTAAATTATCCCAACA | CCGG |
| 4190 | E16Slu3 | exon16 | SluCas9 | + | TTGGACTAAATTATCCCAACAC | CGGG |
| 4191 | E16Slu4 | exon16 | SluCas9 | + | TATCCAGCCATGCTTCCGTCTT | CTGG |
| 4192 | E16Slu5 | exon16 | SluCas9 | + | ATCCAGCCATGCTTCCGTCTTC | TGGG |
| 4193 | E16Slu6 | exon16 | SluCas9 | + | TTTGAGTGAATACAGTTTGCCC | ATGG |
| 4194 | E16Slu7 | exon16 | SluCas9 | − | AAAACTTGAAAAGAGTACAGCA | CAGG |
| 4195 | E16Slu8 | exon16 | SluCas9 | − | GCTGGATAACTTTGCCCGGTGT | TGGG |
| 4196 | E16Slu9 | exon16 | SluCas9 | − | GGCTGGATAACTTTGCCCGGTG | TTGG |
| 4197 | E16Slu10 | exon16 | SluCas9 | − | AAGCATGGCTGGATAACTTTGC | CCGG |
| 4198 | E16Slu11 | exon16 | SluCas9 | − | GACCCAGAAGACGGAAGCATGG | CTGG |
| 4199 | E16Slu12 | exon16 | SluCas9 | − | CAGTGACCCAGAAGACGGAAGC | ATGG |
| 4200 | E16Slu13 | exon16 | SluCas9 | − | GAATAAGTCAGTGACCCAGAAG | ACGG |
| 4201 | E16Slu14 | exon16 | SluCas9 | − | CTAGAAAAGAAAAAGCAATCCA | TGGG |
| 4202 | E16Slu15 | exon16 | SluCas9 | − | TCTAGAAAAGAAAAAGCAATCC | ATGG |
| 4203 | E16Slu16 | exon16 | SluCas9 | − | CTTGTTTTAACAGGTTTTAAAA | GCGG |
| 4204 | E17Slu1 | exon17 | SluCas9 | + | CCACAGTAATCTGCCTCTTCTT | TTGG |
| 4205 | E17Slu2 | exon17 | SluCas9 | + | CACAGTAATCTGCCTCTTCTTT | TGGG |
| 4206 | E17Slu3 | exon17 | SluCas9 | + | ACAGTAATCTGCCTCTTCTTTT | GGGG |
| 4207 | E17Slu4 | exon17 | SluCas9 | + | GTAATCTGCCTCTTCTTTTGGG | GAGG |
| 4208 | E17Slu5 | exon17 | SluCas9 | + | ATCTGCCTCTTCTTTTGGGGAG | GTGG |
| 4209 | E17Slu6 | exon17 | SluCas9 | + | TGCCTCTTCTTTTGGGGAGGTG | GTGG |
| 4210 | E17Slu7 | exon17 | SluCas9 | + | CTCTTCTTTTGGGGAGGTGGTG | GTGG |
| 4211 | E17Slu8 | exon17 | SluCas9 | + | GTTCCTCTTGAGCATGCTTTAC | CAGG |
| 4212 | E17Slu9 | exon17 | SluCas9 | + | CTTTACCAGGATCTGTTCCCTT | GTGG |
| 4213 | E17Slu10 | exon17 | SluCas9 | + | ATTACAGTTGTCTGTGTTAGTG | ATGG |
| 4214 | E17Slu11 | exon17 | SluCas9 | + | TGTCTGTGTTAGTGATGGCTGA | GTGG |
| 4215 | E17Slu12 | exon17 | SluCas9 | + | CTGTGTTAGTGATGGCTGAGTG | GTGG |
| 4216 | E17Slu14 | exon17 | SluCas9 | − | CTGTGGATTCTGAAATTAGGAA | AAGG |
| 4217 | E17Slu15 | exon17 | SluCas9 | − | AGATTACTGTGGATTCTGAAAT | TAGG |
| 4218 | E17Slu16 | exon17 | SluCas9 | − | CCAAAAGAAGAGGCAGATTACT | GTGG |
| 4219 | E17Slu17 | exon17 | SluCas9 | − | CACCACCACCTCCCCAAAAGAA | GAGG |
| 4220 | E17Slu18 | exon17 | SluCas9 | − | GATCCTGGTAAAGCATGCTCAA | GAGG |
| 4221 | E17Slu19 | exon17 | SluCas9 | − | GGTGACCACAAGGGAACAGATC | CTGG |
| 4222 | E17Slu20 | exon17 | SluCas9 | − | AACAGTAACTACGGTGACCACA | AGGG |
| 4223 | E17Slu21 | exon17 | SluCas9 | − | AAACAGTAACTACGGTGACCAC | AAGG |
| 4224 | E17Slu22 | exon17 | SluCas9 | − | AACTGTAATGGAAACAGTAACT | ACGG |
| 4225 | E17Slu23 | exon17 | SluCas9 | − | ATCACTAACACAGACAACTGTA | ATGG |
| 4226 | E17Slu24 | exon17 | SluCas9 | − | TCAATACTTCTCACAGATTTCA | CAGG |
| 4227 | E18Slu1 | exon18 | SluCas9 | + | TCCTTCCGAAAGATTGCAAATT | CAGG |
| 4228 | E18Slu2 | exon18 | SluCas9 | − | GACTTAAAAGAAAAAGTCAATG | TAGG |
| 4229 | E18Slu3 | exon18 | SluCas9 | − | GAATTTGCAATCTTTCGGAAGG | AAGG |
| 4230 | E18Slu4 | exon18 | SluCas9 | − | TCCTGAATTTGCAATCTTTCGG | AAGG |
| 4231 | E18Slu5 | exon18 | SluCas9 | − | AGAGTCCTGAATTTGCAATCTT | TCGG |
| 4232 | E18Slu6 | exon18 | SluCas9 | − | TTGATATAACTGAACTTCACAG | CTGG |
| 4233 | E19Slu1 | exon19 | SluCas9 | + | TACCATTCACCATCTGTTCCAC | CAGG |
| 4234 | E19Slu2 | exon19 | SluCas9 | + | ACCATTCACCATCTGTTCCACC | AGGG |
| 4235 | E19Slu3 | exon19 | SluCas9 | + | CACCAGGGCCTGAGCTGATCTG | CTGG |
| 4236 | E19Slu4 | exon19 | SluCas9 | + | CTTCTCAGCTTTTTCTCGCTCT | ATGG |
| 4237 | E19Slu5 | exon19 | SluCas9 | − | GCCCTGGTGGAACAGATGGTGA | ATGG |
| 4238 | E19Slu6 | exon19 | SluCas9 | − | AGCTCAGGCCCTGGTGGAACAG | ATGG |
| 4239 | E19Slu7 | exon19 | SluCas9 | − | CAGCAGATCAGCTCAGGCCCTG | GTGG |
| 4240 | E19Slu8 | exon19 | SluCas9 | − | TGCCAGCAGATCAGCTCAGGCC | CTGG |
| 4241 | E19Slu9 | exon19 | SluCas9 | − | GCAAGATGCCAGCAGATCAGCT | CAGG |
| 4242 | E20Slu1 | exon20 | SluCas9 | + | TTTAACTGACTTTTAATTGCTG | TTGG |
| 4243 | E20Slu2 | exon20 | SluCas9 | + | CTTTTAATTGCTGTTGGCTCTG | ATGG |
| 4244 | E20Slu3 | exon20 | SluCas9 | + | TTTTAATTGCTGTTGGCTCTGA | TGGG |
| 4245 | E20Slu4 | exon20 | SluCas9 | + | TTTAATTGCTGTTGGCTCTGAT | GGGG |
| 4246 | E20Slu5 | exon20 | SluCas9 | + | AATTGCTGTTGGCTCTGATGGG | GTGG |
| 4247 | E20Slu6 | exon20 | SluCas9 | + | TGCTGTTGGCTCTGATGGGGTG | GTGG |
| 4248 | E20Slu7 | exon20 | SluCas9 | + | GCTGTTGGCTCTGATGGGGTGG | TGGG |
| 4249 | E20Slu8 | exon20 | SluCas9 | + | TTGGCTCTGATGGGGTGGTGGG | TTGG |
| 4250 | E20Slu9 | exon20 | SluCas9 | + | AGTTAAGTCTCTCACTTAGCAA | CTGG |
| 4251 | E20Slu10 | exon20 | SluCas9 | + | GCAACTGGCAGAATTCGATCCA | CCGG |
| 4252 | E20Slu11 | exon20 | SluCas9 | + | CCACCGGCTGTTCAGTTGTTCT | GAGG |
| 4253 | E20Slu13 | exon20 | SluCas9 | − | TAAAAGTCAGTTAAAAATTTGT | AAGG |
| 4254 | E20Slu14 | exon20 | SluCas9 | − | AGATGACAACTACTGCTGAAAA | CTGG |
| 4255 | E20Slu15 | exon20 | SluCas9 | − | TTTCTATAATCAGCTACAACAA | TTGG |
| 4256 | E20Slu16 | exon20 | SluCas9 | − | GCTAAGTGAGAGACTTAACTGG | CTGG |
| 4257 | E20Slu17 | exon20 | SluCas9 | − | AGTTGCTAAGTGAGAGACTTAA | CTGG |
| 4258 | E20Slu18 | exon20 | SluCas9 | − | CCTCAGAACAACTGAACAGCCG | GTGG |
| 4259 | E20Slu19 | exon20 | SluCas9 | − | AAGCCTCAGAACAACTGAACAG | CCGG |
| 4260 | E21Slu1 | exon21 | SluCas9 | + | ACTTGTCTGTAGCTCTTTCTCT | CTGG |
| 4261 | E21Slu2 | exon21 | SluCas9 | + | GACTTGCTTAAAATGATTTGTA | AAGG |
| 4262 | E21Slu3 | exon21 | SluCas9 | + | TAAAGGCCACAAAGTCTGCATC | CAGG |
| 4263 | E21Slu4 | exon21 | SluCas9 | + | CACAAAGTCTGCATCCAGGAAC | ATGG |
| 4264 | E21Slu5 | exon21 | SluCas9 | + | ACAAAGTCTGCATCCAGGAACA | TGGG |
| 4265 | E21Slu6 | exon21 | SluCas9 | + | TGGGTCCTTGTCCTTTCTCTTT | CAGG |
| 4266 | E21Slu7 | exon21 | SluCas9 | + | GGGTCCTTGTCCTTTCTCTTTC | AGGG |
| 4267 | E21Slu8 | exon21 | SluCas9 | + | TGAATTTTTAATCGTTCAATTT | GAGG |
| 4268 | E21Slu9 | exon21 | SluCas9 | + | TTTGAGGTTGAAGATCTGATAG | CCGG |
| 4269 | E21Slu12 | exon21 | SluCas9 | − | TAAGCAAGTCTTTTCTGATGTG | CAGG |
| 4270 | E21Slu13 | exon21 | SluCas9 | − | CATGTTCCTGGATGCAGACTTT | GTGG |
| 4271 | E21Slu14 | exon21 | SluCas9 | − | GAAAGGACAAGGACCCATGTTC | CTGG |
| 4272 | E21Slu15 | exon21 | SluCas9 | − | ATAGCCCTGAAAGAGAAAGGAC | AAGG |
| 4273 | E21Slu16 | exon21 | SluCas9 | − | CAAAGCATAGCCCTGAAAGAGA | AAGG |
| 4274 | E21Slu17 | exon21 | SluCas9 | − | CTATGGCACAGGATGAAGTCAA | CCGG |
| 4275 | E22Slu1 | exon22 | SluCas9 | + | TCTCTGCTCCATGATTTCATAG | TCGG |
| 4276 | E22Slu2 | exon22 | SluCas9 | + | TCATAGTCGGTGACACTAAGTT | GAGG |
| 4277 | E22Slu3 | exon22 | SluCas9 | + | GTCGGTGACACTAAGTTGAGGT | ATGG |
| 4278 | E22Slu4 | exon22 | SluCas9 | + | ACTAAGTTGAGGTATGGAGAGT | TTGG |
| 4279 | E22Slu5 | exon22 | SluCas9 | + | TGGAGAGTTTGGTTTCTGACTG | CTGG |
| 4280 | E22Slu6 | exon22 | SluCas9 | + | TGACTGCTGGACCCATGTCCTG | ATGG |
| 4281 | E22Slu7 | exon22 | SluCas9 | + | GACCCATGTCCTGATGGCACTC | ATGG |
| 4282 | E22Slu8 | exon22 | SluCas9 | + | CTCATGGTCTCCTGATAGCGCA | TTGG |
| 4283 | E22Slu9 | exon22 | SluCas9 | + | ATGGTCTCCTGATAGCGCATTG | GTGG |
| 4284 | E22Slu12 | exon22 | SluCas9 | − | GGAGCAGAGACTCGGGGAATTG | CAGG |
| 4285 | E22Slu13 | exon22 | SluCas9 | − | TGAAATCATGGAGCAGAGACTC | GGGG |
| 4286 | E22Slu14 | exon22 | SluCas9 | − | ATGAAATCATGGAGCAGAGACT | CGGG |
| 4287 | E22Slu15 | exon22 | SluCas9 | − | TATGAAATCATGGAGCAGAGAC | TCGG |
| 4288 | E22Slu16 | exon22 | SluCas9 | − | TAGTGTCACCGACTATGAAATC | ATGG |
| 4289 | E22Slu17 | exon22 | SluCas9 | − | GACCATGAGTGCCATCAGGACA | TGGG |
| 4290 | E22Slu18 | exon22 | SluCas9 | − | AGACCATGAGTGCCATCAGGAC | ATGG |
| 4291 | E22Slu19 | exon22 | SluCas9 | − | ATCAGGAGACCATGAGTGCCAT | CAGG |
| 4292 | E22Slu20 | exon22 | SluCas9 | − | CACTTTGCCACCAATGCGCTAT | CAGG |
| 4293 | E23Slu1 | exon23 | SluCas9 | + | AATCTTGAATTACCTGAATTTT | TCGG |
| 4294 | E23Slu2 | exon23 | SluCas9 | + | GCTTTTGACAATGCTCAACCAG | CTGG |
| 4295 | E23Slu3 | exon23 | SluCas9 | + | CTTTTGACAATGCTCAACCAGC | TGGG |
| 4296 | E23Slu4 | exon23 | SluCas9 | + | TTGACAATGCTCAACCAGCTGG | GAGG |
| 4297 | E23Slu5 | exon23 | SluCas9 | + | CTTCAAATTCTGATTGATATTT | CCGG |
| 4298 | E23Slu6 | exon23 | SluCas9 | + | TTGATATTTCCGGCTAATTTCA | GAGG |
| 4299 | E23Slu7 | exon23 | SluCas9 | + | TGATATTTCCGGCTAATTTCAG | AGGG |
| 4300 | E23Slu8 | exon23 | SluCas9 | + | TTTCTTCGACATCTCTTTCACA | GTGG |
| 4301 | E23Slu9 | exon23 | SluCas9 | + | TCACAGTGGTGCTGAGATAGTA | TAGG |
| 4302 | E23Slu10 | exon23 | SluCas9 | − | AATGAATAAACTCCGAAAAATT | CAGG |
| 4303 | E23Slu11 | exon23 | SluCas9 | − | GGTTGAGCATTGTCAAAAGCTA | GAGG |
| 4304 | E23Slu12 | exon23 | SluCas9 | − | CTGGAAGAAGCTCTCCTCCCAG | CTGG |
| 4305 | E23Slu13 | exon23 | SluCas9 | − | AATTTGAAGAAATTGAGGGACG | CTGG |
| 4306 | E23Slu14 | exon23 | SluCas9 | − | CAATCAGAATTTGAAGAAATTG | AGGG |
| 4307 | E23Slu15 | exon23 | SluCas9 | − | TCAATCAGAATTTGAAGAAATT | GAGG |
| 4308 | E23Slu16 | exon23 | SluCas9 | − | AGAAAGCGCCCTCTGAAATTAG | CCGG |
| 4309 | E23Slu17 | exon23 | SluCas9 | − | AGTTCTCTGCAAGAGCAACAAA | GTGG |
| 4310 | E24Slu1 | exon24 | SluCas9 | + | TTTTTAGAATTTCTGAATCCCC | AAGG |
| 4311 | E24Slu2 | exon24 | SluCas9 | + | TTTTAGAATTTCTGAATCCCCA | AGGG |
| 4312 | E24Slu3 | exon24 | SluCas9 | + | AGAATTTCTGAATCCCCAAGGG | CAGG |
| 4313 | E24Slu4 | exon24 | SluCas9 | + | CAACTTCAGCCATCCATTTCTT | CAGG |
| 4314 | E24Slu5 | exon24 | SluCas9 | + | AACTTCAGCCATCCATTTCTTC | AGGG |
| 4315 | E24Slu8 | exon24 | SluCas9 | − | GAAGGAGGAATGGCCTGCCCTT | GGGG |
| 4316 | E24Slu9 | exon24 | SluCas9 | − | TGAAGGAGGAATGGCCTGCCCT | TGGG |
| 4317 | E24Slu10 | exon24 | SluCas9 | − | CTGAAGGAGGAATGGCCTGCCC | TTGG |
| 4318 | E24Slu11 | exon24 | SluCas9 | − | TTGATGTTTTTCTGAAGGAGGA | ATGG |
| 4319 | E24Slu12 | exon24 | SluCas9 | − | TGAAGTTGATGTTTTTCTGAAG | GAGG |
| 4320 | E24Slu13 | exon24 | SluCas9 | − | GGCTGAAGTTGATGTTTTTCTG | AAGG |
| 4321 | E24Slu14 | exon24 | SluCas9 | − | CATACAAACCCTGAAGAAATGG | ATGG |
| 4322 | E24Slu15 | exon24 | SluCas9 | − | ATCACATACAAACCCTGAAGAA | ATGG |
| 4323 | E25Slu1 | exon25 | SluCas9 | + | TGAAAGAGATTGTCTATACCTG | TTGG |
| 4324 | E25Slu2 | exon25 | SluCas9 | + | GTCTCAAGTCTCGAAGCAAACT | CTGG |
| 4325 | E25Slu3 | exon25 | SluCas9 | + | ACCTTCATTGACACTGTTTAGA | CTGG |
| 4326 | E25Slu4 | exon25 | SluCas9 | + | CCTTCATTGACACTGTTTAGAC | TGGG |
| 4327 | E25Slu6 | exon25 | SluCas9 | − | TCAGTGGGATCACATGTGCCAA | CAGG |
| 4328 | E25Slu7 | exon25 | SluCas9 | − | ACTCAAAGAACTTAACACTCAG | TGGG |
| 4329 | E25Slu8 | exon25 | SluCas9 | − | AACTCAAAGAACTTAACACTCA | GTGG |
| 4330 | E25Slu9 | exon25 | SluCas9 | − | GTCTAAACAGTGTCAATGAAGG | TGGG |
| 4331 | E25Slu10 | exon25 | SluCas9 | − | AGTCTAAACAGTGTCAATGAAG | GTGG |
| 4332 | E25Slu11 | exon25 | SluCas9 | − | CCCAGTCTAAACAGTGTCAATG | AAGG |
| 4333 | E26Slu1 | exon26 | SluCas9 | + | TCAACTGCTTTCTGTAATTCAT | CTGG |
| 4334 | E26Slu2 | exon26 | SluCas9 | + | CGTGCATCTCTGATAGATCTTT | CTGG |
| 4335 | E26Slu3 | exon26 | SluCas9 | + | GCATCTCTGATAGATCTTTCTG | GAGG |
| 4336 | E26Slu4 | exon26 | SluCas9 | + | AGTTTTCTCCAAACCTCCCTTC | AAGG |
| 4337 | E26Slu5 | exon26 | SluCas9 | + | ACCTCCCTTCAAGGCCTCCTTT | CTGG |
| 4338 | E26Slu6 | exon26 | SluCas9 | − | ACAGAAAGCAGTTGAAGAGATG | AAGG |
| 4339 | E26Slu7 | exon26 | SluCas9 | − | AAGATCTATCAGAGATGCACGA | ATGG |
| 4340 | E26Slu8 | exon26 | SluCas9 | − | AAAGGAGGCCTTGAAGGGAGGT | TTGG |
| 4341 | E26Slu9 | exon26 | SluCas9 | − | GCCAGAAAGGAGGCCTTGAAGG | GAGG |
| 4342 | E26Slu10 | exon26 | SluCas9 | − | TATGCCAGAAAGGAGGCCTTGA | AGGG |
| 4343 | E26Slu11 | exon26 | SluCas9 | − | CTATGCCAGAAAGGAGGCCTTG | AAGG |
| 4344 | E26Slu12 | exon26 | SluCas9 | − | GTGGAAGGTCTATGCCAGAAAG | GAGG |
| 4345 | E26Slu13 | exon26 | SluCas9 | − | TTTGTGGAAGGTCTATGCCAGA | AAGG |
| 4346 | E27Slu1 | exon27 | SluCas9 | + | CATTCAGCCTAGTGCAGAGCCA | CTGG |
| 4347 | E27Slu2 | exon27 | SluCas9 | + | CCTAGTGCAGAGCCACTGGTAG | TTGG |
| 4348 | E27Slu3 | exon27 | SluCas9 | + | AGTGCAGAGCCACTGGTAGTTG | GTGG |
| 4349 | E27Slu4 | exon27 | SluCas9 | + | TAGAGTTTCAAGTTCCTTTTTT | AAGG |
| 4350 | E27Slu5 | exon27 | SluCas9 | + | TTTTTTAAGGCCTCTTGTGCTA | CAGG |
| 4351 | E27Slu6 | exon27 | SluCas9 | + | TTTAAGGCCTCTTGTGCTACAG | GTGG |
| 4352 | E27Slu7 | exon27 | SluCas9 | + | TGACACTATTTACAGACTCAGT | AAGG |
| 4353 | E27Slu8 | exon27 | SluCas9 | + | GTTTCACTTTCGCTTCTTTTTG | TTGG |
| 4354 | E27Slu9 | exon27 | SluCas9 | + | TTTCACTTTCGCTTCTTTTTGT | TGGG |
| 4355 | E27Slu11 | exon27 | SluCas9 | − | GCTGAATGGGAAATGCAAGACT | TTGG |
| 4356 | E27Slu12 | exon27 | SluCas9 | − | AGTGGCTCTGCACTAGGCTGAA | TGGG |
| 4357 | E27Slu13 | exon27 | SluCas9 | − | CAGTGGCTCTGCACTAGGCTGA | ATGG |
| 4358 | E27Slu14 | exon27 | SluCas9 | − | CCAACTACCAGTGGCTCTGCAC | TAGG |
| 4359 | E27Slu15 | exon27 | SluCas9 | − | AAACTCTAACCACCAACTACCA | GTGG |
| 4360 | E27Slu16 | exon27 | SluCas9 | − | TGTAGCACAAGAGGCCTTAAAA | AAGG |
| 4361 | E27Slu17 | exon27 | SluCas9 | − | TCAAGCTCCACCTGTAGCACAA | GAGG |
| 4362 | E27Slu18 | exon27 | SluCas9 | − | TATTTTTCAGAGAGCTAAAGAA | GAGG |
| 4363 | E28Slu1 | exon28 | SluCas9 | + | TCAGAGATTTCCTCAGCTCCGC | CAGG |
| 4364 | E28Slu2 | exon28 | SluCas9 | + | AGCTCCGCCAGGAATGTTTTCA | GTGG |
| 4365 | E28Slu3 | exon28 | SluCas9 | − | TGGCGGAGCTGAGGAAATCTCT | GAGG |
| 4366 | E28Slu4 | exon28 | SluCas9 | − | TGAAAACATTCCTGGCGGAGCT | GAGG |
| 4367 | E28Slu5 | exon28 | SluCas9 | − | AAAACCACTGAAAACATTCCTG | GCGG |
| 4368 | E28Slu6 | exon28 | SluCas9 | − | CTTAAAACCACTGAAAACATTC | CTGG |
| 4369 | E28Slu7 | exon28 | SluCas9 | − | CATACTTGGAGAAAGCAAACAA | GTGG |
| 4370 | E28Slu8 | exon28 | SluCas9 | − | TTGGCATGAGTTATTGTCATAC | TTGG |
| 4371 | E28Slu9 | exon28 | SluCas9 | − | TTTCTTAGGAAGTTTGGGCATG | TTGG |
| 4372 | E28Slu10 | exon28 | SluCas9 | − | TATATTTCTTTCTTAGGAAGTT | TGGG |
| 4373 | E28Slu11 | exon28 | SluCas9 | − | ATATATTTCTTTCTTAGGAAGT | TTGG |
| 4374 | E29Slu1 | exon29 | SluCas9 | + | CATCCATGACTCCGCCATCTGT | TAGG |
| 4375 | E29Slu2 | exon29 | SluCas9 | + | ATCCATGACTCCGCCATCTGTT | AGGG |
| 4376 | E29Slu3 | exon29 | SluCas9 | + | TGTGCCAATATGCGAATCTGAT | TTGG |
| 4377 | E29Slu4 | exon29 | SluCas9 | + | GTGCCAATATGCGAATCTGATT | TGGG |
| 4378 | E29Slu5 | exon29 | SluCas9 | − | TCGTTGGAGGGAACTACATGAA | GAGG |
| 4379 | E29Slu6 | exon29 | SluCas9 | − | TGAGACATTTAATTCTCGTTGG | AGGG |
| 4380 | E29Slu7 | exon29 | SluCas9 | − | TTGAGACATTTAATTCTCGTTG | GAGG |
| 4381 | E29Slu8 | exon29 | SluCas9 | − | AACTTGAGACATTTAATTCTCG | TTGG |
| 4382 | E29Slu9 | exon29 | SluCas9 | − | AGTCATGGATGAGCTAATCAAT | GAGG |
| 4383 | E29Slu10 | exon29 | SluCas9 | − | GACCCTAACAGATGGCGGAGTC | ATGG |
| 4384 | E29Slu11 | exon29 | SluCas9 | − | TTGGCACAGACCCTAACAGATG | GCGG |
| 4385 | E29Slu12 | exon29 | SluCas9 | − | ATATTGGCACAGACCCTAACAG | ATGG |
| 4386 | E29Slu13 | exon29 | SluCas9 | − | TAACCCAAATCAGATTCGCATA | TTGG |
| 4387 | E29Slu14 | exon29 | SluCas9 | − | TGAAAATTTGATGCGACATTCA | GAGG |
| 4388 | E30Slu3 | exon30 | SluCas9 | + | GATGTACTTGCCTGGGCTTCCT | GAGG |
| 4389 | E30Slu4 | exon30 | SluCas9 | + | CCAACTGCTTGTCAATGAATGT | GAGG |
| 4390 | E30Slu5 | exon30 | SluCas9 | + | CAACTGCTTGTCAATGAATGTG | AGGG |
| 4391 | E30Slu6 | exon30 | SluCas9 | + | TGTCAATGAATGTGAGGGACTC | CTGG |
| 4392 | E30Slu7 | exon30 | SluCas9 | + | GAGGGACTCCTGGATTAAGTGT | AAGG |
| 4393 | E30Slu8 | exon30 | SluCas9 | + | AGTGTAAGGATTTTTCAGTCTC | CTGG |
| 4394 | E30Slu9 | exon30 | SluCas9 | + | GTGTAAGGATTTTTCAGTCTCC | TGGG |
| 4395 | E30Slu10 | exon30 | SluCas9 | + | ATTTTTCAGTCTCCTGGGCAGA | CTGG |
| 4396 | E30Slu14 | exon30 | SluCas9 | − | AGCTCAAATGCCTCAGGAAGCC | CAGG |
| 4397 | E30Slu15 | exon30 | SluCas9 | − | GGTGGACGCAGCTCAAATGCCT | CAGG |
| 4398 | E30Slu16 | exon30 | SluCas9 | − | GGCAGCTTATATTGCAGACAAG | GTGG |
| 4399 | E30Slu17 | exon30 | SluCas9 | − | GTTGGCAGCTTATATTGCAGAC | AAGG |
| 4400 | E30Slu18 | exon30 | SluCas9 | − | CCTCACATTCATTGACAAGCAG | TTGG |
| 4401 | E30Slu19 | exon30 | SluCas9 | − | TGAAAAATCCTTACACTTAATC | CAGG |
| 4402 | E30Slu20 | exon30 | SluCas9 | − | TGAACAGAGCATCCAGTCTGCC | CAGG |
| 4403 | E30Slu21 | exon30 | SluCas9 | − | TTCCTTCTTTTTAGGCTGTAAG | GAGG |
| 4404 | E30Slu22 | exon30 | SluCas9 | − | TTATTCCTTCTTTTTAGGCTGT | AAGG |
| 4405 | E31Slu1 | exon31 | SluCas9 | + | CCTGTGCAACATCAATCTGAGA | CAGG |
| 4406 | E31Slu2 | exon31 | SluCas9 | + | CATCAATCTGAGACAGGACTCT | TTGG |
| 4407 | E31Slu3 | exon31 | SluCas9 | + | ATCAATCTGAGACAGGACTCTT | TGGG |
| 4408 | E31Slu4 | exon31 | SluCas9 | + | TCTCATGACTTGTCAAATCAGA | TTGG |
| 4409 | E31Slu5 | exon31 | SluCas9 | + | TCAAATCAGATTGGATTTTCTG | TTGG |
| 4410 | E31Slu9 | exon31 | SluCas9 | − | CCTGTCTCAGATTGATGTTGCA | CAGG |
| 4411 | E31Slu10 | exon31 | SluCas9 | − | GAAGAAACATAATCAGGGGAAG | GAGG |
| 4412 | E31Slu11 | exon31 | SluCas9 | − | AATGAAGAAACATAATCAGGGG | AAGG |
| 4413 | E31Slu12 | exon31 | SluCas9 | − | AAGAAATGAAGAAACATAATCA | GGGG |
| 4414 | E31Slu13 | exon31 | SluCas9 | − | GAAGAAATGAAGAAACATAATC | AGGG |
| 4415 | E31Slu14 | exon31 | SluCas9 | − | AGAAGAAATGAAGAAACATAAT | CAGG |
| 4416 | E32Slu1 | exon32 | SluCas9 | + | TCCACACTCTTTGTTTCCAATG | CAGG |
| 4417 | E32Slu2 | exon32 | SluCas9 | + | TTCTTGTAGACGCTGCTCAAAA | TTGG |
| 4418 | E32Slu3 | exon32 | SluCas9 | + | TGTAGACGCTGCTCAAAATTGG | CTGG |
| 4419 | E32Slu4 | exon32 | SluCas9 | + | GCTGCTCAAAATTGGCTGGTTT | CTGG |
| 4420 | E32Slu5 | exon32 | SluCas9 | + | TTTCTGGAATAATCGAAACTTC | ATGG |
| 4421 | E32Slu11 | exon32 | SluCas9 | − | ACAGTCACAGCTAAATCATTGT | GTGG |
| 4422 | E32Slu12 | exon32 | SluCas9 | − | ATTGGAAACAAAGAGTGTGGAA | CAGG |
| 4423 | E32Slu13 | exon32 | SluCas9 | − | GCCTGCATTGGAAACAAAGAGT | GTGG |
| 4424 | E32Slu14 | exon32 | SluCas9 | − | AGTGAAGATGCACTTGCCTGCA | TTGG |
| 4425 | E33Slu1 | exon33 | SluCas9 | + | TGTTACTCTTTCATCAAGTTCT | TTGG |
| 4426 | E33Slu2 | exon33 | SluCas9 | + | GTTACTCTTTCATCAAGTTCTT | TGGG |
| 4427 | E33Slu3 | exon33 | SluCas9 | − | GCATTATAATGAGCTGGGAGCA | AAGG |
| 4428 | E33Slu4 | exon33 | SluCas9 | − | TTGAAATTGCATTATAATGAGC | TGGG |
| 4429 | E33Slu5 | exon33 | SluCas9 | − | TTTGAAATTGCATTATAATGAG | CTGG |
| 4430 | E33Slu6 | exon33 | SluCas9 | − | TCAGATTGTACAGAAAAAGCAG | ACGG |
| 4431 | E33Slu7 | exon33 | SluCas9 | − | GAAGTGGAAATGGTGATAAAGA | CTGG |
| 4432 | E33Slu8 | exon33 | SluCas9 | − | TGAAGTGAAGTCTGAAGTGGAA | ATGG |
| 4433 | E33Slu9 | exon33 | SluCas9 | − | TCTGAGTGAAGTGAAGTCTGAA | GTGG |
| 4434 | E34Slu2 | exon34 | SluCas9 | + | TGATATAGGTTTTACCTTTCCC | CAGG |
| 4435 | E34Slu3 | exon34 | SluCas9 | + | GCAACTTCAGAATCCAAATTAC | TAGG |
| 4436 | E34Slu4 | exon34 | SluCas9 | + | CATTCATTTCCTTTCGCATCTT | ACGG |
| 4437 | E34Slu5 | exon34 | SluCas9 | + | ATTCATTTCCTTTCGCATCTTA | CGGG |
| 4438 | E34Slu7 | exon34 | SluCas9 | − | GGATTCTGAAGTTGCCTGGGGA | AAGG |
| 4439 | E34Slu8 | exon34 | SluCas9 | − | AATTTGGATTCTGAAGTTGCCT | GGGG |
| 4440 | E34Slu9 | exon34 | SluCas9 | − | TAATTTGGATTCTGAAGTTGCC | TGGG |
| 4441 | E34Slu10 | exon34 | SluCas9 | − | GTAATTTGGATTCTGAAGTTGC | CTGG |
| 4442 | E34Slu11 | exon34 | SluCas9 | − | AGTTGAAGGAATGCCTAGTAAT | TTGG |
| 4443 | E34Slu12 | exon34 | SluCas9 | − | TTGACAAAGAGATCAGCAGTTG | AAGG |
| 4444 | E34Slu13 | exon34 | SluCas9 | − | AGAATGGCTGGCAGCTACAGAT | ATGG |
| 4445 | E34Slu14 | exon34 | SluCas9 | − | AATGAATGTCTTGACAGAATGG | CTGG |
| 4446 | E34Slu15 | exon34 | SluCas9 | − | AGGAAATGAATGTCTTGACAGA | ATGG |
| 4447 | E34Slu16 | exon34 | SluCas9 | − | GAAATTGTCCCGTAAGATGCGA | AAGG |
| 4448 | E34Slu17 | exon34 | SluCas9 | − | GGTAACAGAAAGAAAGCAACAG | TTGG |
| 4449 | E35Slu1 | exon35 | SluCas9 | + | AAAGATTTAACCACTCTTCTGC | TCGG |
| 4450 | E35Slu2 | exon35 | SluCas9 | + | AAGATTTAACCACTCTTCTGCT | CGGG |
| 4451 | E35Slu3 | exon35 | SluCas9 | + | ATTTAACCACTCTTCTGCTCGG | GAGG |
| 4452 | E35Slu4 | exon35 | SluCas9 | + | CTTCTTGCCCAAAACTGTTTTC | AAGG |
| 4453 | E35Slu5 | exon35 | SluCas9 | + | CTCCTACCTCTGTGATACTCTT | CAGG |
| 4454 | E35Slu8 | exon35 | SluCas9 | − | AGAAGAGTGGTTAAATCTTTTG | TTGG |
| 4455 | E35Slu9 | exon35 | SluCas9 | − | CTGTCACCTCCCGAGCAGAAGA | GTGG |
| 4456 | E35Slu10 | exon35 | SluCas9 | − | AACTCAGTCTTCTGAATAGTAA | CTGG |
| 4457 | E35Slu11 | exon35 | SluCas9 | − | TTTGGGCAAGAAGGAGACGTTG | GTGG |
| 4458 | E35Slu12 | exon35 | SluCas9 | − | AGTTTTGGGCAAGAAGGAGACG | TTGG |
| 4459 | E35Slu13 | exon35 | SluCas9 | − | CTTGAAAACAGTTTTGGGCAAG | AAGG |
| 4460 | E35Slu14 | exon35 | SluCas9 | − | GGAGAGGCCTTGAAAACAGTTT | TGGG |
| 4461 | E35Slu15 | exon35 | SluCas9 | − | AGGAGAGGCCTTGAAAACAGTT | TTGG |
| 4462 | E35Slu16 | exon35 | SluCas9 | − | GAAGAGTATCACAGAGGTAGGA | GAGG |
| 4463 | E35Slu17 | exon35 | SluCas9 | − | CACCTGAAGAGTATCACAGAGG | TAGG |
| 4464 | E35Slu18 | exon35 | SluCas9 | − | GGTGCACCTGAAGAGTATCACA | GAGG |
| 4465 | E35Slu19 | exon35 | SluCas9 | − | TCAAAAAGAGATTGAGAAACAG | AAGG |
| 4466 | E36Slu1 | exon36 | SluCas9 | + | CCTTAAGCACGTCTTCTTTTTG | CTGG |
| 4467 | E36Slu2 | exon36 | SluCas9 | + | CTTAAGCACGTCTTCTTTTTGC | TGGG |
| 4468 | E36Slu3 | exon36 | SluCas9 | + | TTAAGCACGTCTTCTTTTTGCT | GGGG |
| 4469 | E36Slu4 | exon36 | SluCas9 | + | CCTGAATGATCCACTTTGTGAT | GTGG |
| 4470 | E36Slu5 | exon36 | SluCas9 | + | ACTTTGTGATGTGGTCCACATT | CTGG |
| 4471 | E36Slu6 | exon36 | SluCas9 | + | GGTCAAAAGTTTCCATGTGTTT | CTGG |
| 4472 | E36Slu8 | exon36 | SluCas9 | − | CCAGCAAAAAGAAGACGTGCTT | AAGG |
| 4473 | E36Slu9 | exon36 | SluCas9 | − | GATCATTCAGGCTGACACACTT | TTGG |
| 4474 | E36Slu10 | exon36 | SluCas9 | − | CCACATCACAAAGTGGATCATT | CAGG |
| 4475 | E36Slu11 | exon36 | SluCas9 | − | AGAATGTGGACCACATCACAAA | GTGG |
| 4476 | E36Slu12 | exon36 | SluCas9 | − | CATGGAAACTTTTGACCAGAAT | GTGG |
| 4477 | E36Slu13 | exon36 | SluCas9 | − | AATTAAGGAATACCAGAAACAC | ATGG |
| 4478 | E37Slu1 | exon37 | SluCas9 | + | TCCAGTCTTAATTCTGTGTGAA | ATGG |
| 4479 | E37Slu2 | exon37 | SluCas9 | + | TGTGTGAAATGGCTGCAAATCG | ATGG |
| 4480 | E37Slu3 | exon37 | SluCas9 | + | ATCGATGGTTGAGCTCTGAGAT | TTGG |
| 4481 | E37Slu4 | exon37 | SluCas9 | + | TCGATGGTTGAGCTCTGAGATT | TGGG |
| 4482 | E37Slu5 | exon37 | SluCas9 | + | CGATGGTTGAGCTCTGAGATTT | GGGG |
| 4483 | E37Slu6 | exon37 | SluCas9 | + | GGGGCTCTACTAATTTCCTGCA | GTGG |
| 4484 | E37Slu7 | exon37 | SluCas9 | + | CTAATTTCCTGCAGTGGTCACC | GCGG |
| 4485 | E37Slu8 | exon37 | SluCas9 | + | GGTTTGCCATCAAGTTTGCTGC | TTGG |
| 4486 | E37Slu9 | exon37 | SluCas9 | + | TGGTCACGTGTAGAGTCCACCT | TTGG |
| 4487 | E37Slu10 | exon37 | SluCas9 | + | GGTCACGTGTAGAGTCCACCTT | TGGG |
| 4488 | E37Slu14 | exon37 | SluCas9 | − | CACAGAATTAAGACTGGAAAGG | TAGG |
| 4489 | E37Slu15 | exon37 | SluCas9 | − | TTCACACAGAATTAAGACTGGA | AAGG |
| 4490 | E37Slu16 | exon37 | SluCas9 | − | GCCATTTCACACAGAATTAAGA | CTGG |
| 4491 | E37Slu17 | exon37 | SluCas9 | − | TGGCAAACCGCGGTGACCACTG | CAGG |
| 4492 | E37Slu18 | exon37 | SluCas9 | − | GCAGCAAACTTGATGGCAAACC | GCGG |
| 4493 | E37Slu19 | exon37 | SluCas9 | − | ACGTGACCAAGCAGCAAACTTG | ATGG |
| 4494 | E37Slu20 | exon37 | SluCas9 | − | ACTGAATGACATACGCCCAAAG | GTGG |
| 4495 | E37Slu21 | exon37 | SluCas9 | − | AGAACTGAATGACATACGCCCA | AAGG |
| 4496 | E37Slu22 | exon37 | SluCas9 | − | TGCTCATGGAATATAGCGTTTA | AAGG |
| 4497 | E38Slu1 | exon38 | SluCas9 | + | CCCTGCTGAATTTCAGCCTCCA | GTGG |
| 4498 | E38Slu2 | exon38 | SluCas9 | + | TTAAACTGCTCCAATTCCTTCA | AAGG |
| 4499 | E38Slu3 | exon38 | SluCas9 | + | CTGCTCCAATTCCTTCAAAGGA | ATGG |
| 4500 | E38Slu4 | exon38 | SluCas9 | + | CTCCAATTCCTTCAAAGGAATG | GAGG |
| 4501 | E38Slu6 | exon38 | SluCas9 | − | AGAGGAAGACTTCAATAAAGAT | ATGG |
| 4502 | E38Slu7 | exon38 | SluCas9 | − | TCAGCAGGGGGTGAATCTGAAA | GAGG |
| 4503 | E38Slu8 | exon38 | SluCas9 | − | ACTGGAGGCTGAAATTCAGCAG | GGGG |
| 4504 | E38Slu9 | exon38 | SluCas9 | − | CACTGGAGGCTGAAATTCAGCA | GGGG |
| 4505 | E38Slu10 | exon38 | SluCas9 | − | CCACTGGAGGCTGAAATTCAGC | AGGG |
| 4506 | E38Slu11 | exon38 | SluCas9 | − | ACCACTGGAGGCTGAAATTCAG | CAGG |
| 4507 | E38Slu12 | exon38 | SluCas9 | − | ACAAAAATTGCTTGAACCACTG | GAGG |
| 4508 | E38Slu13 | exon38 | SluCas9 | − | TATACAAAAATTGCTTGAACCA | CTGG |
| 4509 | E38Slu14 | exon38 | SluCas9 | − | GGCCTCCATTCCTTTGAAGGAA | TTGG |
| 4510 | E38Slu15 | exon38 | SluCas9 | − | TTTTTAGGCCTCCATTCCTTTG | AAGG |
| 4511 | E39Slu1 | exon39 | SluCas9 | − | ACAGACAAAACATAATGCTCTC | AAGG |
| 4512 | E39Slu2 | exon39 | SluCas9 | − | AATCACAGATGAGAGAAAGCGA | GAGG |
| 4513 | E39Slu3 | exon39 | SluCas9 | − | ACTGTAAAAGAATTGTTGCAAA | GAGG |
| 4514 | E39Slu4 | exon39 | SluCas9 | − | TTTGATCAGAATGAAGACAATG | AGGG |
| 4515 | E39Slu5 | exon39 | SluCas9 | − | TTTTGATCAGAATGAAGACAAT | GAGG |
| 4516 | E40Slu1 | exon40 | SluCas9 | + | TACCTTTATTTTCCTTTCATCT | CTGG |
| 4517 | E40Slu2 | exon40 | SluCas9 | + | ACCTTTATTTTCCTTTCATCTC | TGGG |
| 4518 | E40Slu3 | exon40 | SluCas9 | + | ATTTTCCTTTCATCTCTGGGCT | CAGG |
| 4519 | E40Slu4 | exon40 | SluCas9 | + | TCCTTTCATCTCTGGGCTCAGG | TAGG |
| 4520 | E40Slu5 | exon40 | SluCas9 | + | TTCATCTCTGGGCTCAGGTAGG | CTGG |
| 4521 | E40Slu6 | exon40 | SluCas9 | + | TTTCAATGTCATCCAAGCATTT | CAGG |
| 4522 | E40Slu8 | exon40 | SluCas9 | − | GCCCAGAGATGAAAGGAAAATA | AAGG |
| 4523 | E40Slu9 | exon40 | SluCas9 | − | GCCTACCTGAGCCCAGAGATGA | AAGG |
| 4524 | E40Slu10 | exon40 | SluCas9 | − | GGCTGATGATCTCCTGAAATGC | TTGG |
| 4525 | E40Slu11 | exon40 | SluCas9 | − | TCAGTGGTATCAGTACAAGAGG | CAGG |
| 4526 | E40Slu12 | exon40 | SluCas9 | − | CTCATCAGTGGTATCAGTACAA | GAGG |
| 4527 | E40Slu13 | exon40 | SluCas9 | − | AGGCTCTAGAAATTTCTCATCA | GTGG |
| 4528 | E40Slu14 | exon40 | SluCas9 | − | TTTGAGGTCTCAAAGAAGAAAA | AAGG |
| 4529 | E40Slu15 | exon40 | SluCas9 | − | GTCTGCACCATGAACAGGATTT | GAGG |
| 4530 | E41Slu1 | exon41 | SluCas9 | + | TTTCCCGCCAGCGCTTGCTGAG | CTGG |
| 4531 | E41Slu2 | exon41 | SluCas9 | + | CGCTTGCTGAGCTGGATCTGAG | TTGG |
| 4532 | E41Slu3 | exon41 | SluCas9 | + | CTGAGTTGGCTCCACTGCCATT | GCGG |
| 4533 | E41Slu9 | exon41 | SluCas9 | − | GATCCAGCTCAGCAAGCGCTGG | CGGG |
| 4534 | E41Slu10 | exon41 | SluCas9 | − | AGATCCAGCTCAGCAAGCGCTG | GCGG |
| 4535 | E41Slu11 | exon41 | SluCas9 | − | CTCAGATCCAGCTCAGCAAGCG | CTGG |
| 4536 | E41Slu12 | exon41 | SluCas9 | − | TGAGGATGGGGCCGCAATGGCA | GTGG |
| 4537 | E41Slu13 | exon41 | SluCas9 | − | CTTGTCTGAGGATGGGGCCGCA | ATGG |
| 4538 | E41Slu14 | exon41 | SluCas9 | − | AGCTGAGGGCTTGTCTGAGGAT | GGGG |
| 4539 | E41Slu15 | exon41 | SluCas9 | − | AAGCTGAGGGCTTGTCTGAGGA | TGGG |
| 4540 | E41Slu16 | exon41 | SluCas9 | − | CAAGCTGAGGGCTTGTCTGAGG | ATGG |
| 4541 | E41Slu17 | exon41 | SluCas9 | − | TAGGCAAGCTGAGGGCTTGTCT | GAGG |
| 4542 | E41Slu18 | exon41 | SluCas9 | − | AATGCAGTGCGTAGGCAAGCTG | AGGG |
| 4543 | E41Slu19 | exon41 | SluCas9 | − | GAATGCAGTGCGTAGGCAAGCT | GAGG |
| 4544 | E41Slu20 | exon41 | SluCas9 | − | AAGAGGAGCTGAATGCAGTGCG | TAGG |
| 4545 | E41Slu21 | exon41 | SluCas9 | − | TCGGGAATTGCAGAAGAAGAAA | GAGG |
| 4546 | E41Slu22 | exon41 | SluCas9 | − | TTTTGCTCAATAGGAAATTGAT | CGGG |
| 4547 | E41Slu23 | exon41 | SluCas9 | − | GTTTTGCTCAATAGGAAATTGA | TCGG |
| 4548 | E42Slu2 | exon42 | SluCas9 | + | GATCTTCAAAGTCCTTAGCACA | GAGG |
| 4549 | E42Slu3 | exon42 | SluCas9 | + | TCAAAGTCCTTAGCACAGAGGT | CAGG |
| 4550 | E42Slu4 | exon42 | SluCas9 | + | TGAGAAGTTGTTCCACTTCTAA | TAGG |
| 4551 | E42Slu5 | exon42 | SluCas9 | + | GAGAAGTTGTTCCACTTCTAAT | AGGG |
| 4552 | E42Slu6 | exon42 | SluCas9 | + | GTGATTTCAGTCAAATAAGTAG | AAGG |
| 4553 | E42Slu7 | exon42 | SluCas9 | + | GAAGGCACATAAGAAATTTCCA | AAGG |
| 4554 | E42Slu8 | exon42 | SluCas9 | + | TCATCACCATCATCGTTTCTTC | ACGG |
| 4555 | E42Slu9 | exon42 | SluCas9 | + | TCGTTTCTTCACGGACAGTGTG | CTGG |
| 4556 | E42Slu10 | exon42 | SluCas9 | − | CTTTAAGCAAGAGGAGTCTCTG | AAGG |
| 4557 | E42Slu11 | exon42 | SluCas9 | − | CTTTGAAGATCTCTTTAAGCAA | GAGG |
| 4558 | E42Slu12 | exon42 | SluCas9 | − | CAATGCTCCTGACCTCTGTGCT | AAGG |
| 4559 | E42Slu13 | exon42 | SluCas9 | − | TGTCTCACAAGCCCTATTAGAA | GTGG |
| 4560 | E42Slu14 | exon42 | SluCas9 | − | GGTGATGACTGAAGACATGCCT | TTGG |
| 4561 | E42Slu15 | exon42 | SluCas9 | − | CACTGTCCGTGAAGAAACGATG | ATGG |
| 4562 | E43Slu1 | exon43 | SluCas9 | + | ATATATGTGTTACCTACCCTTG | TCGG |
| 4563 | E43Slu2 | exon43 | SluCas9 | + | ACATTTTGTTAACTTTTTCCCA | TTGG |
| 4564 | E43Slu3 | exon43 | SluCas9 | + | CTTTTTCCCATTGGAAATCAAG | CTGG |
| 4565 | E43Slu4 | exon43 | SluCas9 | + | TTTTTCCCATTGGAAATCAAGC | TGGG |
| 4566 | E43Slu5 | exon43 | SluCas9 | + | TCCTGTAGCTTCACCCTTTCCA | CAGG |
| 4567 | E43Slu6 | exon43 | SluCas9 | − | AATGTACAAGGACCGACAAGGG | TAGG |
| 4568 | E43Slu7 | exon43 | SluCas9 | − | ACAAAATGTACAAGGACCGACA | AGGG |
| 4569 | E43Slu8 | exon43 | SluCas9 | − | AACAAAATGTACAAGGACCGAC | AAGG |
| 4570 | E43Slu9 | exon43 | SluCas9 | − | GGAAAAAGTTAACAAAATGTAC | AAGG |
| 4571 | E43Slu10 | exon43 | SluCas9 | − | TCTCTCCCAGCTTGATTTCCAA | TGGG |
| 4572 | E43Slu11 | exon43 | SluCas9 | − | CTCTCTCCCAGCTTGATTTCCA | ATGG |
| 4573 | E43Slu12 | exon43 | SluCas9 | − | GCCTGTGGAAAGGGTGAAGCTA | CAGG |
| 4574 | E43Slu13 | exon43 | SluCas9 | − | GCAAAGTGCAACGCCTGTGGAA | AGGG |
| 4575 | E43Slu14 | exon43 | SluCas9 | − | TGCAAAGTGCAACGCCTGTGGA | AAGG |
| 4576 | E43Slu15 | exon43 | SluCas9 | − | AGCATTGCAAAGTGCAACGCCT | GTGG |
| 4577 | E43Slu16 | exon43 | SluCas9 | − | ATAGTCTACAACAAAGCTCAGG | TCGG |
| 4578 | E43Slu17 | exon43 | SluCas9 | − | AAAGATAGTCTACAACAAAGCT | CAGG |
| 4579 | E46Slu1 | exon46 | SluCas9 | + | TCTTTTAGTTGCTGCTCTTTTC | CAGG |
| 4580 | E46Slu2 | exon46 | SluCas9 | + | TGCTGCTCTTTTCCAGGTTCAA | GTGG |
| 4581 | E46Slu3 | exon46 | SluCas9 | + | GCTGCTCTTTTCCAGGTTCAAG | TGGG |
| 4582 | E46Slu4 | exon46 | SluCas9 | + | GATATTCTTTTGTTCTTCTAGC | CTGG |
| 4583 | E46Slu7 | exon46 | SluCas9 | − | AAAAGAAAAGCTTGAGCAAGTC | AAGG |
| 4584 | E46Slu8 | exon46 | SluCas9 | − | ATTGCTAGTATCCCACTTGAAC | CTGG |
| 4585 | E46Slu9 | exon46 | SluCas9 | − | AAATGAATTTGTTTTATGGTTG | GAGG |
| 4586 | E46Slu10 | exon46 | SluCas9 | − | TTTAAATGAATTTGTTTTATGG | TTGG |
| 4587 | E46Slu11 | exon46 | SluCas9 | − | GAGATTTAAATGAATTTGTTTT | ATGG |
| 4588 | E47Slu1 | exon47 | SluCas9 | + | GTTAATGTCTAACCTTTATCCA | CTGG |
| 4589 | E47Slu2 | exon47 | SluCas9 | + | TTTTCAAGTTTATCTTGCTCTT | CTGG |
| 4590 | E47Slu3 | exon47 | SluCas9 | + | TTTCAAGTTTATCTTGCTCTTC | TGGG |
| 4591 | E47Slu4 | exon47 | SluCas9 | + | TTTATCTTGCTCTTCTGGGCTT | ATGG |
| 4592 | E47Slu5 | exon47 | SluCas9 | + | TTATCTTGCTCTTCTGGGCTTA | TGGG |
| 4593 | E47Slu6 | exon47 | SluCas9 | + | GCTTATGGGAGCACTTACAAGC | ACGG |
| 4594 | E47Slu7 | exon47 | SluCas9 | + | CTTATGGGAGCACTTACAAGCA | CGGG |
| 4595 | E47Slu8 | exon47 | SluCas9 | + | CATTTAATTGTTTGAGAATTCC | CTGG |
| 4596 | E47Slu9 | exon47 | SluCas9 | + | ATTGTTTGAGAATTCCCTGGCG | CAGG |
| 4597 | E47Slu10 | exon47 | SluCas9 | + | TTGTTTGAGAATTCCCTGGCGC | AGGG |
| 4598 | E47Slu11 | exon47 | SluCas9 | + | TGTTTGAGAATTCCCTGGCGCA | GGGG |
| 4599 | E47Slu12 | exon47 | SluCas9 | − | GCAGACAAATCTCCAGTGGATA | AAGG |
| 4600 | E47Slu13 | exon47 | SluCas9 | − | AGCTCAAGCAGACAAATCTCCA | GTGG |
| 4601 | E47Slu14 | exon47 | SluCas9 | − | CTCAAACAATTAAATGAAACTG | GAGG |
| 4602 | E47Slu15 | exon47 | SluCas9 | − | ATTCTCAAACAATTAAATGAAA | CTGG |
| 4603 | E47Slu16 | exon47 | SluCas9 | − | GTGGAAGAGTTGCCCCTGCGCC | AGGG |
| 4604 | E47Slu17 | exon47 | SluCas9 | − | GGTGGAAGAGTTGCCCCTGCGC | CAGG |
| 4605 | E47Slu18 | exon47 | SluCas9 | − | CATTTGTCTGTTTCAGTTACTG | GTGG |
| 4606 | E47Slu19 | exon47 | SluCas9 | − | TTGCATTTGTCTGTTTCAGTTA | CTGG |
| 4607 | E48Slu1 | exon48 | SluCas9 | + | AAGTTCCCTACCTTAACGTCAA | ATGG |
| 4608 | E48Slu2 | exon48 | SluCas9 | + | CCTTAACGTCAAATGGTCCTTC | TTGG |
| 4609 | E48Slu3 | exon48 | SluCas9 | + | ACGTCAAATGGTCCTTCTTGGT | TTGG |
| 4610 | E48Slu4 | exon48 | SluCas9 | + | CAAATGGTCCTTCTTGGTTTGG | TTGG |
| 4611 | E48Slu5 | exon48 | SluCas9 | + | TAAATTTCCAACTGATTCCTAA | TAGG |
| 4612 | E48Slu6 | exon48 | SluCas9 | + | GCAGATGATTTAACTGCTCTTC | AAGG |
| 4613 | E48Slu7 | exon48 | SluCas9 | + | CAAGCTTTTTTTCAAGCTGCCC | AAGG |
| 4614 | E48Slu8 | exon48 | SluCas9 | + | GCTTCAATTTCTCCTTGTTTCT | CAGG |
| 4615 | E48Slu9 | exon48 | SluCas9 | + | TCCTTGTTTCTCAGGTAAAGCT | CTGG |
| 4616 | E48Slu12 | exon48 | SluCas9 | − | AAGGACCATTTGACGTTAAGGT | AGGG |
| 4617 | E48Slu13 | exon48 | SluCas9 | − | GAAGGACCATTTGACGTTAAGG | TAGG |
| 4618 | E48Slu14 | exon48 | SluCas9 | − | CCAAGAAGGACCATTTGACGTT | AAGG |
| 4619 | E48Slu15 | exon48 | SluCas9 | − | ATTTATAACCAACCAAACCAAG | AAGG |
| 4620 | E48Slu16 | exon48 | SluCas9 | − | GTTATCTCCTATTAGGAATCAG | TTGG |
| 4621 | E48Slu17 | exon48 | SluCas9 | − | TGCTGCTGTGGTTATCTCCTAT | TAGG |
| 4622 | E48Slu18 | exon48 | SluCas9 | − | AGCAGTTAAATCATCTGCTGCT | GTGG |
| 4623 | E48Slu19 | exon48 | SluCas9 | − | TTGAAGCTCAAATAAAAGACCT | TGGG |
| 4624 | E48Slu20 | exon48 | SluCas9 | − | ATTGAAGCTCAAATAAAAGACC | TTGG |
| 4625 | E48Slu21 | exon48 | SluCas9 | − | TCCAGAGCTTTACCTGAGAAAC | AAGG |
| 4626 | E49Slu1 | exon49 | SluCas9 | + | AGAGGTTGCTTCATTACCTTCA | CTGG |
| 4627 | E49Slu2 | exon49 | SluCas9 | + | CTTCATTACCTTCACTGGCTGA | GTGG |
| 4628 | E49Slu3 | exon49 | SluCas9 | + | ATTACCTTCACTGGCTGAGTGG | CTGG |
| 4629 | E49Slu4 | exon49 | SluCas9 | + | TTAGACAAAATCTCTTCCACAT | CCGG |
| 4630 | E49Slu5 | exon49 | SluCas9 | + | CTTGAACTGCTATTTCAGTTTC | CTGG |
| 4631 | E49Slu6 | exon49 | SluCas9 | + | TTGAACTGCTATTTCAGTTTCC | TGGG |
| 4632 | E49Slu7 | exon49 | SluCas9 | + | TGAACTGCTATTTCAGTTTCCT | GGGG |
| 4633 | E49Slu8 | exon49 | SluCas9 | − | AAAACCAGCCACTCAGCCAGTG | AAGG |
| 4634 | E49Slu9 | exon49 | SluCas9 | − | GTCTAAAGGGCAGCATTTGTAC | AAGG |
| 4635 | E49Slu10 | exon49 | SluCas9 | − | ATGTGGAAGAGATTTTGTCTAA | AGGG |
| 4636 | E49Slu11 | exon49 | SluCas9 | − | GATGTGGAAGAGATTTTGTCTA | AAGG |
| 4637 | E49Slu12 | exon49 | SluCas9 | − | AGTTCAAGCTAAACAACCGGAT | GTGG |
| 4638 | E49Slu13 | exon49 | SluCas9 | − | AATAGCAGTTCAAGCTAAACAA | CCGG |
| 4639 | E52Slu1 | exon52 | SluCas9 | + | GATTGTTCTAGCCTCTTGATTG | CTGG |
| 4640 | E52Slu2 | exon52 | SluCas9 | + | TGCTGGTCTTGTTTTTCAAATT | TTGG |
| 4641 | E52Slu3 | exon52 | SluCas9 | + | GCTGGTCTTGTTTTTCAAATTT | TGGG |
| 4642 | E52Slu4 | exon52 | SluCas9 | + | CTTGTTTTTCAAATTTTGGGCA | GCGG |
| 4643 | E52Slu5 | exon52 | SluCas9 | + | CAGCGGTAATGAGTTCTTCCAA | CTGG |
| 4644 | E52Slu6 | exon52 | SluCas9 | + | AGCGGTAATGAGTTCTTCCAAC | TGGG |
| 4645 | E52Slu7 | exon52 | SluCas9 | + | GCGGTAATGAGTTCTTCCAACT | GGGG |
| 4646 | E52Slu12 | exon52 | SluCas9 | − | TCAAGAGGCTAGAACAATCATT | ACGG |
| 4647 | E52Slu13 | exon52 | SluCas9 | − | GAAAAACAAGACCAGCAATCAA | GAGG |
| 4648 | E52Slu14 | exon52 | SluCas9 | − | TTTGGAACAGAGGCGTCCCCAG | TTGG |
| 4649 | E52Slu15 | exon52 | SluCas9 | − | CAACAATGCAGGATTTGGAACA | GAGG |
| 4650 | E52Slu16 | exon52 | SluCas9 | − | CTTACAGGCAACAATGCAGGAT | TTGG |
| 4651 | E52Slu17 | exon52 | SluCas9 | − | TTTGTTCTTACAGGCAACAATG | CAGG |
| 4652 | E54Slu1 | exon54 | SluCas9 | + | CCTTTTATGAATGCTTCTCCAA | GAGG |
| 4653 | E54Slu2 | exon54 | SluCas9 | + | CATTGATATTCTCTGTTATCAT | GTGG |
| 4654 | E54Slu3 | exon54 | SluCas9 | + | CTCTGTTATCATGTGGACTTTT | CTGG |
| 4655 | E54Slu4 | exon54 | SluCas9 | + | TGGTATCATCTGCAGAATAATC | CCGG |
| 4656 | E54Slu5 | exon54 | SluCas9 | + | CAGAATAATCCCGGAGAAGTTT | CAGG |
| 4657 | E54Slu6 | exon54 | SluCas9 | + | AGAATAATCCCGGAGAAGTTTC | AGGG |
| 4658 | E54Slu7 | exon54 | SluCas9 | + | CCACATCTACATTTGTCTGCCA | CTGG |
| 4659 | E54Slu8 | exon54 | SluCas9 | + | CATCTACATTTGTCTGCCACTG | GCGG |
| 4660 | E54Slu9 | exon54 | SluCas9 | + | CTACATTTGTCTGCCACTGGCG | GAGG |
| 4661 | E54Slu10 | exon54 | SluCas9 | + | TGTCTGCCACTGGCGGAGGTCT | TTGG |
| 4662 | E54Slu11 | exon54 | SluCas9 | − | CCTCTTGGAGAAGCATTCATAA | AAGG |
| 4663 | E54Slu12 | exon54 | SluCas9 | − | TAACAGAGAATATCAATGCCTC | TTGG |
| 4664 | E54Slu13 | exon54 | SluCas9 | − | TGACTTGGCCCTGAAACTTCTC | CGGG |
| 4665 | E54Slu14 | exon54 | SluCas9 | − | ATGACTTGGCCCTGAAACTTCT | CCGG |
| 4666 | E54Slu15 | exon54 | SluCas9 | − | AAATGTAGATGTGGCAAATGAC | TTGG |
| 4667 | E54Slu16 | exon54 | SluCas9 | − | CCAGTGGCAGACAAATGTAGAT | GTGG |
| 4668 | E54Slu17 | exon54 | SluCas9 | − | AGTTGGCCAAAGACCTCCGCCA | GTGG |
| 4669 | E54Slu18 | exon54 | SluCas9 | − | TTCTCATAAAAATCTATAGCAG | TTGG |
| 4670 | E55Slu1 | exon55 | SluCas9 | + | TTTCATCAGCTCTTTTACTCCC | TTGG |
| 4671 | E55Slu2 | exon55 | SluCas9 | + | CTTTTACTCCCTTGGAGTCTTC | TAGG |
| 4672 | E55Slu3 | exon55 | SluCas9 | + | AGTCTTCTAGGAGCCTTTCCTT | ACGG |
| 4673 | E55Slu4 | exon55 | SluCas9 | + | GTCTTCTAGGAGCCTTTCCTTA | CGGG |
| 4674 | E55Slu5 | exon55 | SluCas9 | + | TTTCCTTACGGGTAGCATCCTG | TAGG |
| 4675 | E55Slu6 | exon55 | SluCas9 | + | ACGGGTAGCATCCTGTAGGACA | TTGG |
| 4676 | E55Slu7 | exon55 | SluCas9 | + | AGTTGTTTCAGCTTCTGTAAGC | CAGG |
| 4677 | E55Slu8 | exon55 | SluCas9 | + | TAAGCCAGGCAAGAAACTTTTC | CAGG |
| 4678 | E55Slu9 | exon55 | SluCas9 | + | AGGCAAGAAACTTTTCCAGGTC | CAGG |
| 4679 | E55Slu10 | exon55 | SluCas9 | + | GGCAAGAAACTTTTCCAGGTCC | AGGG |
| 4680 | E55Slu11 | exon55 | SluCas9 | + | GCAAGAAACTTTTCCAGGTCCA | GGGG |
| 4681 | E55Slu12 | exon55 | SluCas9 | + | CAAGAAACTTTTCCAGGTCCAG | GGGG |
| 4682 | E55Slu15 | exon55 | SluCas9 | − | GAGTAAAAGAGCTGATGAAACA | ATGG |
| 4683 | E55Slu16 | exon55 | SluCas9 | − | GAAAGGCTCCTAGAAGACTCCA | AGGG |
| 4684 | E55Slu17 | exon55 | SluCas9 | − | GGAAAGGCTCCTAGAAGACTCC | AAGG |
| 4685 | E55Slu18 | exon55 | SluCas9 | − | TACAGGATGCTACCCGTAAGGA | AAGG |
| 4686 | E55Slu19 | exon55 | SluCas9 | − | TGTCCTACAGGATGCTACCCGT | AAGG |
| 4687 | E55Slu20 | exon55 | SluCas9 | − | TGAAACAACTGCCAATGTCCTA | CAGG |
| 4688 | E55Slu21 | exon55 | SluCas9 | − | TGGACCTGGAAAAGTTTCTTGC | CTGG |
| 4689 | E55Slu22 | exon55 | SluCas9 | − | ACTGCAACAGTTCCCCCTGGAC | CTGG |
| 4690 | E55Slu23 | exon55 | SluCas9 | − | TAGATTACTGCAACAGTTCCCC | CTGG |
| 4691 | E55Slu24 | exon55 | SluCas9 | − | GGTGAGTGAGCGAGAGGCTGCT | TTGG |
| 4692 | E55Slu25 | exon55 | SluCas9 | − | CCTTTGCAGGGTGAGTGAGCGA | GAGG |
| 4693 | E56Slu1 | exon56 | SluCas9 | + | TGTTATCCAAACGTCTTTGTAA | CAGG |
| 4694 | E56Slu2 | exon56 | SluCas9 | + | TCTTTGTAACAGGACTGCATCA | TCGG |
| 4695 | E56Slu3 | exon56 | SluCas9 | + | GGACTGCATCATCGGAACCTTC | CAGG |
| 4696 | E56Slu4 | exon56 | SluCas9 | + | GACTGCATCATCGGAACCTTCC | AGGG |
| 4697 | E56Slu5 | exon56 | SluCas9 | + | CATCGGAACCTTCCAGGGATCT | CAGG |
| 4698 | E56Slu6 | exon56 | SluCas9 | + | CTTCCAGGGATCTCAGGATTTT | TTGG |
| 4699 | E56Slu7 | exon56 | SluCas9 | + | GGATTTTTTGGCTGTTTTCATC | CAGG |
| 4700 | E56Slu8 | exon56 | SluCas9 | + | CTGTGTGAGCTTCAATTTCACC | TTGG |
| 4701 | E56Slu9 | exon56 | SluCas9 | + | TGTGAGCTTCAATTTCACCTTG | GAGG |
| 4702 | E56Slu10 | exon56 | SluCas9 | + | CAATTTCACCTTGGAGGTCCTA | CAGG |
| 4703 | E56Slu14 | exon56 | SluCas9 | − | GAAAAAGTCTCTCAACATTAGG | TAGG |
| 4704 | E56Slu15 | exon56 | SluCas9 | − | TTCGGAAAAAGTCTCTCAACAT | TAGG |
| 4705 | E56Slu16 | exon56 | SluCas9 | − | TGAACTTCAAGTGGAGTGAACT | TCGG |
| 4706 | E56Slu17 | exon56 | SluCas9 | − | GTTTGGATAACATGAACTTCAA | GTGG |
| 4707 | E56Slu18 | exon56 | SluCas9 | − | TGCAGTCCTGTTACAAAGACGT | TTGG |
| 4708 | E56Slu19 | exon56 | SluCas9 | − | CAAAAAATCCTGAGATCCCTGG | AAGG |
| 4709 | E56Slu20 | exon56 | SluCas9 | − | CAGCCAAAAAATCCTGAGATCC | CTGG |
| 4710 | E56Slu21 | exon56 | SluCas9 | − | TCACACAGATGTTTATCACAAC | CTGG |
| 4711 | E56Slu22 | exon56 | SluCas9 | − | CCTGCTGTCCTGTAGGACCTCC | AAGG |
| 4712 | E57Slu1 | exon57 | SluCas9 | + | ACATCGTTCTGCTTCTGAACTG | CTGG |
| 4713 | E57Slu2 | exon57 | SluCas9 | + | ACTGCTGGAAAGTCGCCTCCAA | TAGG |
| 4714 | E57Slu3 | exon57 | SluCas9 | + | AGTCGCCTCCAATAGGTGCCTG | CCGG |
| 4715 | E57Slu4 | exon57 | SluCas9 | + | ACACCAGAAGTTCCTGCAGAGA | AAGG |
| 4716 | E57Slu5 | exon57 | SluCas9 | + | GAGAAAGGTGCAGACGCTTCCA | CTGG |
| 4717 | E57Slu6 | exon57 | SluCas9 | + | CAGACGCTTCCACTGGTCAGAA | CTGG |
| 4718 | E57Slu7 | exon57 | SluCas9 | + | ACTGGTCAGAACTGGCTTCCAA | ATGG |
| 4719 | E57Slu8 | exon57 | SluCas9 | + | CTGGTCAGAACTGGCTTCCAAA | TGGG |
| 4720 | E57Slu9 | exon57 | SluCas9 | − | AAGCAGAACGATGTACATAGGG | TAGG |
| 4721 | E57Slu10 | exon57 | SluCas9 | − | TCAGAAGCAGAACGATGTACAT | AGGG |
| 4722 | E57Slu11 | exon57 | SluCas9 | − | TTCAGAAGCAGAACGATGTACA | TAGG |
| 4723 | E57Slu12 | exon57 | SluCas9 | − | TTAAGCCGGCAGGCACCTATTG | GAGG |
| 4724 | E57Slu13 | exon57 | SluCas9 | − | GAATTAAGCCGGCAGGCACCTA | TTGG |
| 4725 | E57Slu14 | exon57 | SluCas9 | − | GAAAGATGATGAATTAAGCCGG | CAGG |
| 4726 | E57Slu15 | exon57 | SluCas9 | − | AGCTGAAAGATGATGAATTAAG | CCGG |
| 4727 | E57Slu16 | exon57 | SluCas9 | − | TTTCTCTGCAGGAACTTCTGGT | GTGG |
| 4728 | E57Slu17 | exon57 | SluCas9 | − | GCACCTTTCTCTGCAGGAACTT | CTGG |
| 4729 | E57Slu18 | exon57 | SluCas9 | − | GAAGCGTCTGCACCTTTCTCTG | CAGG |
| 4730 | E57Slu19 | exon57 | SluCas9 | − | ATTTGGAAGCCAGTTCTGACCA | GTGG |
| 4731 | E57Slu20 | exon57 | SluCas9 | − | GCTGTTCTTTTTCAGGTCCCAT | TTGG |
| 4732 | E58Slu2 | exon58 | SluCas9 | + | TAGTTCCACATTCAATTACCTC | TGGG |
| 4733 | E58Slu3 | exon58 | SluCas9 | + | ACATTCAATTACCTCTGGGCTC | CTGG |
| 4734 | E58Slu4 | exon58 | SluCas9 | + | TAGAGTTTCTCTAGTCCTTCCA | AAGG |
| 4735 | E58Slu5 | exon58 | SluCas9 | + | GTCTCAAGAGTACTCATGATTA | CAGG |
| 4736 | E58Slu6 | exon58 | SluCas9 | + | TTTAGTTTTCAATTCCCTCTTG | AAGG |
| 4737 | E58Slu8 | exon58 | SluCas9 | − | GAGAAACTCTACCAGGAGCCCA | GAGG |
| 4738 | E58Slu9 | exon58 | SluCas9 | − | GGAAGGACTAGAGAAACTCTAC | CAGG |
| 4739 | E58Slu10 | exon58 | SluCas9 | − | TTTCTGACAGAGCAGCCTTTGG | AAGG |
| 4740 | E58Slu11 | exon58 | SluCas9 | − | AATATTTCTGACAGAGCAGCCT | TTGG |
| 4741 | E58Slu12 | exon58 | SluCas9 | − | TCTCATTTCACAGGCCTTCAAG | AGGG |
| 4742 | E58Slu13 | exon58 | SluCas9 | − | ATCTCATTTCACAGGCCTTCAA | GAGG |
| 4743 | E59Slu2 | exon59 | SluCas9 | + | TAGACGGTACCTTGACTTTCTC | GAGG |
| 4744 | E59Slu3 | exon59 | SluCas9 | + | CCTTGACTTTCTCGAGGTGATC | TTGG |
| 4745 | E59Slu4 | exon59 | SluCas9 | + | GGTGATCTTGGAGAGAGTCAAT | GAGG |
| 4746 | E59Slu5 | exon59 | SluCas9 | + | AGAGTCAATGAGGAGATCGCCC | ACGG |
| 4747 | E59Slu6 | exon59 | SluCas9 | + | GAGTCAATGAGGAGATCGCCCA | CGGG |
| 4748 | E59Slu7 | exon59 | SluCas9 | + | GAGGAGATCGCCCACGGGCTGC | CAGG |
| 4749 | E59Slu8 | exon59 | SluCas9 | + | AGGATCCCTTGATCACCTCAGC | TTGG |
| 4750 | E59Slu9 | exon59 | SluCas9 | + | TCACCTCAGCTTGGCGCAGCTT | GAGG |
| 4751 | E59Slu10 | exon59 | SluCas9 | + | CAGCTTGAGGTCCAGCTCATCC | GTGG |
| 4752 | E59Slu11 | exon59 | SluCas9 | + | CATCCGTGGCCTCTTGAAGTTC | CCGG |
| 4753 | E59Slu12 | exon59 | SluCas9 | + | CTTGAAGTTCCCGGAGTCTTTC | AAGG |
| 4754 | E59Slu13 | exon59 | SluCas9 | + | TTGAAGTTCCCGGAGTCTTTCA | AGGG |
| 4755 | E59Slu14 | exon59 | SluCas9 | + | ATCTATTTTTCTCTGCCAGTCA | GCGG |
| 4756 | E59Slu15 | exon59 | SluCas9 | + | TTCTCTGCCAGTCAGCGGAGTG | CAGG |
| 4757 | E59Slu16 | exon59 | SluCas9 | + | TTCGTAGAAGCCGAGTGACATT | CTGG |
| 4758 | E59Slu17 | exon59 | SluCas9 | + | TCGTAGAAGCCGAGTGACATTC | TGGG |
| 4759 | E59Slu18 | exon59 | SluCas9 | + | GTGACATTCTGGGCTCTCTCCT | CAGG |
| 4760 | E59Slu19 | exon59 | SluCas9 | + | ACATTCTGGGCTCTCTCCTCAG | GAGG |
| 4761 | E59Slu25 | exon59 | SluCas9 | − | CCAAGATCACCTCGAGAAAGTC | AAGG |
| 4762 | E59Slu26 | exon59 | SluCas9 | − | ATCAAGGGATCCTGGCAGCCCG | TGGG |
| 4763 | E59Slu27 | exon59 | SluCas9 | − | GATCAAGGGATCCTGGCAGCCC | GTGG |
| 4764 | E59Slu28 | exon59 | SluCas9 | − | AAGCTGAGGTGATCAAGGGATC | CTGG |
| 4765 | E59Slu29 | exon59 | SluCas9 | − | CTGCGCCAAGCTGAGGTGATCA | AGGG |
| 4766 | E59Slu30 | exon59 | SluCas9 | − | GCTGCGCCAAGCTGAGGTGATC | AAGG |
| 4767 | E59Slu31 | exon59 | SluCas9 | − | GGACCTCAAGCTGCGCCAAGCT | GAGG |
| 4768 | E59Slu32 | exon59 | SluCas9 | − | ACTTCAAGAGGCCACGGATGAG | CTGG |
| 4769 | E59Slu33 | exon59 | SluCas9 | − | ACTCCGGGAACTTCAAGAGGCC | ACGG |
| 4770 | E59Slu34 | exon59 | SluCas9 | | TGAAAGACTCCGGGAACTTCAA | GAGG |
| 4771 | E59Slu35 | exon59 | SluCas9 | − | AGATGAGACCCTTGAAAGACTC | CGGG |
| 4772 | E59Slu36 | exon59 | SluCas9 | − | TAGATGAGACCCTTGAAAGACT | CCGG |
| 4773 | E59Slu37 | exon59 | SluCas9 | − | AATTGAACCTGCACTCCGCTGA | CTGG |
| 4774 | E59Slu38 | exon59 | SluCas9 | − | GGCTGAGGAGGTCAATACTGAG | TGGG |
| 4775 | E59Slu39 | exon59 | SluCas9 | − | AGGCTGAGGAGGTCAATACTGA | GTGG |
| 4776 | E59Slu40 | exon59 | SluCas9 | − | GCTTCTACGAAAGCAGGCTGAG | GAGG |
| 4777 | E59Slu41 | exon59 | SluCas9 | − | TCGGCTTCTACGAAAGCAGGCT | GAGG |
| 4778 | E59Slu42 | exon59 | SluCas9 | − | TGTCACTCGGCTTCTACGAAAG | CAGG |
| 4779 | E59Slu43 | exon59 | SluCas9 | − | AGGAGAGAGCCCAGAATGTCAC | TCGG |
| 4780 | E59Slu44 | exon59 | SluCas9 | − | TGCCAATTTAGAGCTGCCTCCT | GAGG |
| 4781 | E60Slu1 | exon60 | SluCas9 | + | GCTTACCTGCAGAAGCTTCCAT | CTGG |
| 4782 | E60Slu2 | exon60 | SluCas9 | + | GCAGAAGCTTCCATCTGGTGTT | CAGG |
| 4783 | E60Slu3 | exon60 | SluCas9 | + | TGTTCAGGTCTTCCAGAGTGCT | GAGG |
| 4784 | E60Slu4 | exon60 | SluCas9 | + | TCTTCCAGAGTGCTGAGGTTAT | ACGG |
| 4785 | E60Slu5 | exon60 | SluCas9 | + | CGGTGAGAGCTGAATGCCCAAA | GTGG |
| 4786 | E60Slu6 | exon60 | SluCas9 | + | GCTGAATGCCCAAAGTGGTAAG | CTGG |
| 4787 | E60Slu7 | exon60 | SluCas9 | + | CCAAAGTGGTAAGCTGGCGAGC | AAGG |
| 4788 | E60Slu8 | exon60 | SluCas9 | + | GCTGGCGAGCAAGGTCATTGAC | GTGG |
| 4789 | E60Slu9 | exon60 | SluCas9 | + | ACGTGGCTCACGTTCTCTTTCA | GAGG |
| 4790 | E60Slu11 | exon60 | SluCas9 | − | GAACACCAGATGGAAGCTTCTG | CAGG |
| 4791 | E60Slu12 | exon60 | SluCas9 | − | CTCTGGAAGACCTGAACACCAG | ATGG |
| 4792 | E60Slu13 | exon60 | SluCas9 | − | CTCACCGTATAACCTCAGCACT | CTGG |
| 4793 | E60Slu14 | exon60 | SluCas9 | − | CTTGCTCGCCAGCTTACCACTT | TGGG |
| 4794 | E60Slu15 | exon60 | SluCas9 | − | CCTTGCTCGCCAGCTTACCACT | TTGG |
| 4795 | E60Slu16 | exon60 | SluCas9 | − | GACTATTGCACACAGGCACTTC | GAGG |
| 4796 | E61Slu2 | exon61 | SluCas9 | + | CTGGAAAGAAAGTGCTGAGATG | CTGG |
| 4797 | E61Slu3 | exon61 | SluCas9 | + | GAGATGCTGGACCAAAGTCCCT | GTGG |
| 4798 | E61Slu4 | exon61 | SluCas9 | + | AGATGCTGGACCAAAGTCCCTG | TGGG |
| 4799 | E61Slu5 | exon61 | SluCas9 | + | GGGCTTCATGCAGCTGCCTGAC | TCGG |
| 4800 | E61Slu6 | exon61 | SluCas9 | + | CAGCTGCCTGACTCGGTCCTCG | ACGG |
| 4801 | E61Slu7 | exon61 | SluCas9 | + | TGACTCGGTCCTCGACGGCCAC | CTGG |
| 4802 | E61Slu8 | exon61 | SluCas9 | + | GACTCGGTCCTCGACGGCCACC | TGGG |
| 4803 | E61Slu11 | exon61 | SluCas9 | − | CTGCATGAAGCCCACAGGGACT | TTGG |
| 4804 | E61Slu12 | exon61 | SluCas9 | − | CAGGCAGCTGCATGAAGCCCAC | AGGG |
| 4805 | E61Slu13 | exon61 | SluCas9 | − | TCAGGCAGCTGCATGAAGCCCA | CAGG |
| 4806 | E61Slu14 | exon61 | SluCas9 | − | AGGTGGCCGTCGAGGACCGAGT | CAGG |
| 4807 | E61Slu15 | exon61 | SluCas9 | − | CTTTTCCTCCCAGGTGGCCGTC | GAGG |
| 4808 | E62Slu1 | exon62 | SluCas9 | + | ACTTCCAACTTACTTGATATAG | TAGG |
| 4809 | E62Slu2 | exon62 | SluCas9 | + | CTTCCAACTTACTTGATATAGT | AGGG |
| 4810 | E62Slu3 | exon62 | SluCas9 | + | TTGATATAGTAGGGCACTTTGT | TTGG |
| 4811 | E62Slu4 | exon62 | SluCas9 | + | GTAGGGCACTTTGTTTGGCGAG | ATGG |
| 4812 | E62Slu5 | exon62 | SluCas9 | + | GTTTGGCGAGATGGCTCTCTCC | CAGG |
| 4813 | E62Slu6 | exon62 | SluCas9 | + | TTTGGCGAGATGGCTCTCTCCC | AGGG |
| 4814 | E62Slu7 | exon62 | SluCas9 | + | AGATGGCTCTCTCCCAGGGACC | CTGG |
| 4815 | E62Slu13 | exon62 | SluCas9 | − | TTCAGCGTCTGTCCAGGGTCCC | TGGG |
| 4816 | E62Slu14 | exon62 | SluCas9 | − | TTTCAGCGTCTGTCCAGGGTCC | CTGG |
| 4817 | E62Slu15 | exon62 | SluCas9 | − | CCCTTCTTTTCAGCGTCTGTCC | AGGG |
| 4818 | E62Slu16 | exon62 | SluCas9 | − | TCCCTTCTTTTCAGCGTCTGTC | CAGG |
| 4819 | E63Slu1 | exon63 | SluCas9 | + | TGGCCATGTCCTTACCTAAAGA | CTGG |
| 4820 | E63Slu2 | exon63 | SluCas9 | + | AGACTGGTAGAGCTCTGTCATT | TTGG |
| 4821 | E63Slu3 | exon63 | SluCas9 | + | GACTGGTAGAGCTCTGTCATTT | TGGG |
| 4822 | E63Slu4 | exon63 | SluCas9 | + | GGTAGAGCTCTGTCATTTTGGG | ATGG |
| 4823 | E63Slu5 | exon63 | SluCas9 | + | CCCAGCAAGTTGTTTGAGTCTC | GTGG |
| 4824 | E63Slu7 | exon63 | SluCas9 | − | AGAGCTCTACCAGTCTTTAGGT | AAGG |
| 4825 | E63Slu8 | exon63 | SluCas9 | − | ATGACAGAGCTCTACCAGTCTT | TAGG |
| 4826 | E63Slu9 | exon63 | SluCas9 | − | CCACGAGACTCAAACAACTTGC | TGGG |
| 4827 | E63Slu10 | exon63 | SluCas9 | − | GCCACGAGACTCAAACAACTTG | CTGG |
| 4828 | E64Slu1 | exon64 | SluCas9 | + | ATACTGGCCAATACTTACAGCA | AAGG |
| 4829 | E64Slu2 | exon64 | SluCas9 | + | TACTGGCCAATACTTACAGCAA | AGGG |
| 4830 | E64Slu3 | exon64 | SluCas9 | + | AGCAAAGGGCCTTCTGCAGTCT | TCGG |
| 4831 | E64Slu4 | exon64 | SluCas9 | + | CTTCTGCAGTCTTCGGAGTTTC | ATGG |
| 4832 | E64Slu5 | exon64 | SluCas9 | + | AAGCTGAGAATCTGACATTATT | CAGG |
| 4833 | E64Slu9 | exon64 | SluCas9 | − | CATGAAACTCCGAAGACTGCAG | AAGG |
| 4834 | E64Slu10 | exon64 | SluCas9 | − | ATAATGTCAGATTCTCAGCTTA | TAGG |
| 4835 | E65Slu1 | exon65 | SluCas9 | + | TTCAGACACATATCCACGCAGA | GAGG |
| 4836 | E65Slu2 | exon65 | SluCas9 | + | TCAGACACATATCCACGCAGAG | AGGG |
| 4837 | E65Slu3 | exon65 | SluCas9 | + | CCAAATTGTTGTGCTCTTGCTC | CAGG |
| 4838 | E65Slu4 | exon65 | SluCas9 | + | AATTGTTGTGCTCTTGCTCCAG | GCGG |
| 4839 | E65Slu5 | exon65 | SluCas9 | + | TTGCTCCAGGCGGTCATAAATA | GTGG |
| 4840 | E65Slu6 | exon65 | SluCas9 | + | TGGTCAAACAATTAATAATCTG | CAGG |
| 4841 | E65Slu7 | exon65 | SluCas9 | + | ATTAATAATCTGCAGGATATCC | ATGG |
| 4842 | E65Slu8 | exon65 | SluCas9 | + | TTAATAATCTGCAGGATATCCA | TGGG |
| 4843 | E65Slu9 | exon65 | SluCas9 | + | TAATCTGCAGGATATCCATGGG | CTGG |
| 4844 | E65Slu10 | exon65 | SluCas9 | + | CCATGGGCTGGTCATTTTGCTT | GAGG |
| 4845 | E65Slu11 | exon65 | SluCas9 | + | GGTCATTTTGCTTGAGGTTGTG | CTGG |
| 4846 | E65Slu12 | exon65 | SluCas9 | + | TTGCTTGAGGTTGTGCTGGTCC | AAGG |
| 4847 | E65Slu13 | exon65 | SluCas9 | + | CCAAGGCATCACATGCAGCTGA | CAGG |
| 4848 | E65Slu17 | exon65 | SluCas9 | − | TCTGCGTGGATATGTGTCTGAA | CTGG |
| 4849 | E65Slu18 | exon65 | SluCas9 | − | TTTGGTCAACGTCCCTCTCTGC | GTGG |
| 4850 | E65Slu19 | exon65 | SluCas9 | − | CCTGGAGCAAGAGCACAACAAT | TTGG |
| 4851 | E65Slu20 | exon65 | SluCas9 | − | TTTGACCACTATTTATGACCGC | CTGG |
| 4852 | E65Slu21 | exon65 | SluCas9 | − | CCTCAAGCAAAATGACCAGCCC | ATGG |
| 4853 | E65Slu22 | exon65 | SluCas9 | − | CCTGTCAGCTGCATGTGATGCC | TTGG |
| 4854 | E66Slu1 | exon66 | SluCas9 | + | TGTCTTCCAAATGTGCTTTACA | CAGG |
| 4855 | E66Slu2 | exon66 | SluCas9 | + | GTCTTCCAAATGTGCTTTACAC | AGGG |
| 4856 | E66Slu3 | exon66 | SluCas9 | + | AAATGATGCCAGTTTTAAAAGA | CAGG |
| 4857 | E66Slu4 | exon66 | SluCas9 | + | TGCCAGTTTTAAAAGACAGGAC | ACGG |
| 4858 | E66Slu5 | exon66 | SluCas9 | − | CATTTCCCTGTGTAAAGCACAT | TTGG |
| 4859 | E66Slu6 | exon66 | SluCas9 | − | ATCCGTGTCCTGTCTTTTAAAA | CTGG |
| 4860 | E66Slu7 | exon66 | SluCas9 | − | TTCATAATAGGGGACGAACAGG | GAGG |
| 4861 | E66Slu8 | exon66 | SluCas9 | − | TTCTTCATAATAGGGGACGAAC | AGGG |
| 4862 | E66Slu9 | exon66 | SluCas9 | − | ATTCTTCATAATAGGGGACGAA | CAGG |
| 4863 | E67Slu1 | exon67 | SluCas9 | + | TAGAAGGTGAATAACTTACAAA | TTGG |
| 4864 | E67Slu2 | exon67 | SluCas9 | + | AACTTACAAATTGGAAGCAGCT | CCGG |
| 4865 | E67Slu3 | exon67 | SluCas9 | + | AATTGGAAGCAGCTCCGGACAC | TTGG |
| 4866 | E67Slu4 | exon67 | SluCas9 | + | TGGCTCAATGTTACTGCCCCCA | AAGG |
| 4867 | E67Slu5 | exon67 | SluCas9 | + | GATGCAACTTCACCCAACTGTC | TTGG |
| 4868 | E67Slu6 | exon67 | SluCas9 | + | CTTCACCCAACTGTCTTGGAAT | TTGG |
| 4869 | E67Slu7 | exon67 | SluCas9 | + | GAATTTGGATAGAATCATGCAG | AAGG |
| 4870 | E67Slu8 | exon67 | SluCas9 | + | TTTGGATAGAATCATGCAGAAG | GAGG |
| 4871 | E67Slu9 | exon67 | SluCas9 | + | GCAGAAGGAGGCCCAGCCTGCG | CTGG |
| 4872 | E67Slu10 | exon67 | SluCas9 | + | TTGAACTTGCCACTTGCTTGAA | AAGG |
| 4873 | E67Slu13 | exon67 | SluCas9 | − | GCAGTAACATTGAGCCAAGTGT | CCGG |
| 4874 | E67Slu14 | exon67 | SluCas9 | − | TTGGGTGAAGTTGCATCCTTTG | GGGG |
| 4875 | E67Slu15 | exon67 | SluCas9 | − | GTTGGGTGAAGTTGCATCCTTT | GGGG |
| 4876 | E67Slu16 | exon67 | SluCas9 | − | AGTTGGGTGAAGTTGCATCCTT | TGGG |
| 4877 | E67Slu17 | exon67 | SluCas9 | − | CAGTTGGGTGAAGTTGCATCCT | TTGG |
| 4878 | E67Slu18 | exon67 | SluCas9 | − | TCTATCCAAATTCCAAGACAGT | TGGG |
| 4879 | E67Slu19 | exon67 | SluCas9 | − | TTCTATCCAAATTCCAAGACAG | TTGG |
| 4880 | E67Slu20 | exon67 | SluCas9 | − | GGATTTTGTGACCAGCGCAGGC | TGGG |
| 4881 | E67Slu21 | exon67 | SluCas9 | − | AGGATTTTGTGACCAGCGCAGG | CTGG |
| 4882 | E67Slu22 | exon67 | SluCas9 | − | CAACAGGATTTTGTGACCAGCG | CAGG |
| 4883 | E67Slu23 | exon67 | SluCas9 | − | TTCAAGCAAGTGGCAAGTTCAA | CAGG |
| 4884 | E67Slu24 | exon67 | SluCas9 | − | CTTTGTAGACCTTTTCAAGCAA | GTGG |
| 4885 | E68Slu1 | exon68 | SluCas9 | + | TCCTAATACCTGAATCCAATGA | TTGG |
| 4886 | E68Slu2 | exon68 | SluCas9 | + | ACACTCTTTGCAGATGTTACAT | TTGG |
| 4887 | E68Slu3 | exon68 | SluCas9 | + | GATGTTACATTTGGCCTGATGC | TTGG |
| 4888 | E68Slu4 | exon68 | SluCas9 | + | TTTCTGCAGCAGCCACTCTGTG | CAGG |
| 4889 | E68Slu5 | exon68 | SluCas9 | + | TGCAGCAGCCACTCTGTGCAGG | ACGG |
| 4890 | E68Slu6 | exon68 | SluCas9 | + | GCAGCAGCCACTCTGTGCAGGA | CGGG |
| 4891 | E68Slu7 | exon68 | SluCas9 | + | GTGCAGGACGGGCAGCCACACC | ATGG |
| 4892 | E68Slu8 | exon68 | SluCas9 | + | GGACGGGCAGCCACACCATGGA | CTGG |
| 4893 | E68Slu9 | exon68 | SluCas9 | + | GACGGGCAGCCACACCATGGAC | TGGG |
| 4894 | E68Slu10 | exon68 | SluCas9 | + | ACGGGCAGCCACACCATGGACT | GGGG |
| 4895 | E68Slu11 | exon68 | SluCas9 | + | GGGGTTCCAGTCTCATCCAGTC | TAGG |
| 4896 | E68Slu12 | exon68 | SluCas9 | + | CCAGTCTCATCCAGTCTAGGAA | GAGG |
| 4897 | E68Slu13 | exon68 | SluCas9 | + | CAGTCTCATCCAGTCTAGGAAG | AGGG |
| 4898 | E68Slu14 | exon68 | SluCas9 | + | AGGAAGAGGGCCGCTTCGATCT | CTGG |
| 4899 | E68Slu22 | exon68 | SluCas9 | − | AAGAGTGTCCAATCATTGGATT | CAGG |
| 4900 | E68Slu23 | exon68 | SluCas9 | − | ATCTGCAAAGAGTGTCCAATCA | TTGG |
| 4901 | E68Slu24 | exon68 | SluCas9 | − | TGCTGCAGAAACTGCCAAGCAT | CAGG |
| 4902 | E68Slu25 | exon68 | SluCas9 | − | GTGGCTGCCCGTCCTGCACAGA | GTGG |
| 4903 | E68Slu26 | exon68 | SluCas9 | − | GACTGGAACCCCAGTCCATGGT | GTGG |
| 4904 | E68Slu27 | exon68 | SluCas9 | − | GATGAGACTGGAACCCCAGTCC | ATGG |
| 4905 | E68Slu28 | exon68 | SluCas9 | − | CCTCTTCCTAGACTGGATGAGA | CTGG |
| 4906 | E68Slu29 | exon68 | SluCas9 | − | TCGAAGCGGCCCTCTTCCTAGA | CTGG |
| 4907 | E68Slu30 | exon68 | SluCas9 | − | TAATAATAAGCCAGAGATCGAA | GCGG |
| 4908 | E69Slu2 | exon69 | SluCas9 | + | TACCGGAGTGCAATATTCCACC | ATGG |
| 4909 | E69Slu3 | exon69 | SluCas9 | + | ACCGGAGTGCAATATTCCACCA | TGGG |
| 4910 | E69Slu4 | exon69 | SluCas9 | + | CCACCATGGGATAGTGCATTTT | ATGG |
| 4911 | E69Slu5 | exon69 | SluCas9 | + | CTCGACCAGAAAAAAAGCAGCT | TTGG |
| 4912 | E69Slu6 | exon69 | SluCas9 | − | TCCCATGGTGGAATATTGCACT | CCGG |
| 4913 | E69Slu7 | exon69 | SluCas9 | − | CCATAAAATGCACTATCCCATG | GTGG |
| 4914 | E69Slu8 | exon69 | SluCas9 | − | AGGCCATAAAATGCACTATCCC | ATGG |
| 4915 | E69Slu9 | exon69 | SluCas9 | − | TTTTTTTCTGGTCGAGTTGCAA | AAGG |
| 4916 | E69Slu10 | exon69 | SluCas9 | − | ATCTGCCAAAGCTGCTTTTTTT | CTGG |
| 4917 | E69Slu11 | exon69 | SluCas9 | − | TGTTTTTGCTCTTTATCAGGTA | CAGG |
| 4918 | E70Slu1 | exon70 | SluCas9 | + | TCCCCCTCTAAGACAGTCTGCA | CTGG |
| 4919 | E70Slu2 | exon70 | SluCas9 | + | CCTCTAAGACAGTCTGCACTGG | CAGG |
| 4920 | E70Slu3 | exon70 | SluCas9 | + | TCTGCACTGGCAGGTAGCCCAT | TCGG |
| 4921 | E70Slu4 | exon70 | SluCas9 | + | CTGCACTGGCAGGTAGCCCATT | CGGG |
| 4922 | E70Slu5 | exon70 | SluCas9 | + | TGCACTGGCAGGTAGCCCATTC | GGGG |
| 4923 | E70Slu6 | exon70 | SluCas9 | + | GGGATGCTTCGCAAAATACCTT | TTGG |
| 4924 | E70Slu7 | exon70 | SluCas9 | + | TCGAAATTTGTTTTTTAGTACC | TTGG |
| 4925 | E70Slu11 | exon70 | SluCas9 | − | GACTGTCTTAGAGGGGGACAAC | ATGG |
| 4926 | E70Slu12 | exon70 | SluCas9 | − | GCCAGTGCAGACTGTCTTAGAG | GGGG |
| 4927 | E70Slu13 | exon70 | SluCas9 | − | TGCCAGTGCAGACTGTCTTAGA | GGGG |
| 4928 | E70Slu14 | exon70 | SluCas9 | − | CTGCCAGTGCAGACTGTCTTAG | AGGG |
| 4929 | E70Slu15 | exon70 | SluCas9 | − | CCTGCCAGTGCAGACTGTCTTA | GAGG |
| 4930 | E70Slu16 | exon70 | SluCas9 | − | TATTTTGCGAAGCATCCCCGAA | TGGG |
| 4931 | E70Slu17 | exon70 | SluCas9 | − | GTATTTTGCGAAGCATCCCCGA | ATGG |
| 4932 | E70Slu18 | exon70 | SluCas9 | − | TAAAAAACAAATTTCGAACCAA | AAGG |
| 4933 | E70Slu19 | exon70 | SluCas9 | − | AGAAGATGTTCGAGACTTTGCC | AAGG |
| 4934 | E70Slu20 | exon70 | SluCas9 | − | TGATCTCCCTTTTAGACTACAT | CAGG |
| 4935 | E71Slu1 | exon71 | SluCas9 | + | AAAAGTACTCACGCAGAATCTA | CTGG |
| 4936 | E71Slu2 | exon71 | SluCas9 | + | TGGCCAGAAGTTGATCAGAGTA | ACGG |
| 4937 | E71Slu3 | exon71 | SluCas9 | + | GGCCAGAAGTTGATCAGAGTAA | CGGG |
| 4938 | E71Slu9 | exon71 | SluCas9 | − | GTCCCGTTACTCTGATCAACTT | CTGG |
| 4939 | E72Slu1 | exon72 | SluCas9 | + | TGAGTATCATCGTGTGAAAGCT | GAGG |
| 4940 | E72Slu2 | exon72 | SluCas9 | + | GAGTATCATCGTGTGAAAGCTG | AGGG |
| 4941 | E72Slu3 | exon72 | SluCas9 | + | AGTATCATCGTGTGAAAGCTGA | GGGG |
| 4942 | E72Slu4 | exon72 | SluCas9 | + | ATCGTGTGAAAGCTGAGGGGAC | GAGG |
| 4943 | E72Slu5 | exon72 | SluCas9 | + | TGTGAAAGCTGAGGGGACGAGG | CAGG |
| 4944 | E72Slu8 | exon72 | SluCas9 | − | CACGCATTGAACATTATGCTAG | CAGG |
| 4945 | E73Slu1 | exon73 | SluCas9 | + | TGGGATACTTACATGCTCTCAT | TAGG |
| 4946 | E73Slu2 | exon73 | SluCas9 | − | CTAGCAGAAATGGAAAACAGCA | ATGG |
| 4947 | E73Slu3 | exon73 | SluCas9 | − | GTTTTTTATCAGGCTAGCAGAA | ATGG |
| 4948 | E74Slu1 | exon74 | SluCas9 | + | TGTTTTCTTCCTCAAGATCTGC | TAGG |
| 4949 | E74Slu2 | exon74 | SluCas9 | + | CTCCCCTCTTTCCTCACTCTCT | AAGG |
| 4950 | E74Slu3 | exon74 | SluCas9 | + | CACTCTCTAAGGAAATCAAGAT | CTGG |
| 4951 | E74Slu4 | exon74 | SluCas9 | + | ACTCTCTAAGGAAATCAAGATC | TGGG |
| 4952 | E74Slu5 | exon74 | SluCas9 | + | TCTAAGGAAATCAAGATCTGGG | CAGG |
| 4953 | E74Slu6 | exon74 | SluCas9 | + | ATCAAGATCTGGGCAGGACTAC | GAGG |
| 4954 | E74Slu7 | exon74 | SluCas9 | + | AGATCTGGGCAGGACTACGAGG | CTGG |
| 4955 | E74Slu8 | exon74 | SluCas9 | + | GGGCAGGACTACGAGGCTGGCT | CAGG |
| 4956 | E74Slu9 | exon74 | SluCas9 | + | GGCAGGACTACGAGGCTGGCTC | AGGG |
| 4957 | E74Slu10 | exon74 | SluCas9 | + | GCAGGACTACGAGGCTGGCTCA | GGGG |
| 4958 | E74Slu11 | exon74 | SluCas9 | + | CAGGACTACGAGGCTGGCTCAG | GGGG |
| 4959 | E74Slu12 | exon74 | SluCas9 | + | AGGACTACGAGGCTGGCTCAGG | GGGG |
| 4960 | E74Slu13 | exon74 | SluCas9 | + | GAGGCTGGCTCAGGGGGGAGTC | CTGG |
| 4961 | E74Slu14 | exon74 | SluCas9 | + | GGGGGGAGTCCTGGTTCAAACT | TTGG |
| 4962 | E74Slu15 | exon74 | SluCas9 | + | GGTTCAAACTTTGGCAGTAATG | CTGG |
| 4963 | E74Slu16 | exon74 | SluCas9 | + | GATTAACAAATGTTCATCATCT | CTGG |
| 4964 | E74Slu20 | exon74 | SluCas9 | − | TAGCAGATCTTGAGGAAGAAAA | CAGG |
| 4965 | E74Slu21 | exon74 | SluCas9 | − | AGAGAGAATCCTAGCAGATCTT | GAGG |
| 4966 | E74Slu22 | exon74 | SluCas9 | − | TTCCTTAGAGAGTGAGGAAAGA | GGGG |
| 4967 | E74S1u23 | exon74 | SluCas9 | − | TTTCCTTAGAGAGTGAGGAAAG | AGGG |
| 4968 | E74Slu24 | exon74 | SluCas9 | − | ATTTCCTTAGAGAGTGAGGAAA | GAGG |
| 4969 | E74Slu25 | exon74 | SluCas9 | − | GATCTTGATTTCCTTAGAGAGT | GAGG |
| 4970 | E74Slu26 | exon74 | SluCas9 | − | GCATTACTGCCAAAGTTTGAAC | CAGG |
| 4971 | E75Slu1 | exon75 | SluCas9 | + | ACTGTGACTCCAGCTGTTTATT | GTGG |
| 4972 | E75Slu2 | exon75 | SluCas9 | + | CCAGCTGTTTATTGTGGTCTTC | CAGG |
| 4973 | E75Slu3 | exon75 | SluCas9 | + | GTGGTCTTCCAGGATTTGCATC | CTGG |
| 4974 | E75Slu4 | exon75 | SluCas9 | + | CCAGGATTTGCATCCTGGCTTC | CAGG |
| 4975 | E75Slu5 | exon75 | SluCas9 | + | GGATTTGCATCCTGGCTTCCAG | GCGG |
| 4976 | E75Slu6 | exon75 | SluCas9 | + | GCCTTTGTGTTGACGCAGTAGC | TTGG |
| 4977 | E75Slu7 | exon75 | SluCas9 | + | CCTCAGCAATGAGCTCAGCATC | CCGG |
| 4978 | E75Slu8 | exon75 | SluCas9 | + | CTCAGCAATGAGCTCAGCATCC | CGGG |
| 4979 | E75Slu9 | exon75 | SluCas9 | + | TCAGCAATGAGCTCAGCATCCC | GGGG |
| 4980 | E75Slu10 | exon75 | SluCas9 | + | TGAGCTCAGCATCCCGGGGACT | CTGG |
| 4981 | E75Slu11 | exon75 | SluCas9 | + | GAGCTCAGCATCCCGGGGACTC | TGGG |
| 4982 | E75Slu12 | exon75 | SluCas9 | + | AGCTCAGCATCCCGGGGACTCT | GGGG |
| 4983 | E75Slu13 | exon75 | SluCas9 | + | AGCATCCCGGGGACTCTGGGGA | GAGG |
| 4984 | E75Slu14 | exon75 | SluCas9 | + | ATCCCGGGGACTCTGGGGAGAG | GTGG |
| 4985 | E75Slu15 | exon75 | SluCas9 | + | TCCCGGGGACTCTGGGGAGAGG | TGGG |
| 4986 | E75Slu16 | exon75 | SluCas9 | + | TGGGGAGAGGTGGGCATCATTT | CAGG |
| 4987 | E75Slu17 | exon75 | SluCas9 | + | GGAGAGGTGGGCATCATTTCAG | GAGG |
| 4988 | E75Slu18 | exon75 | SluCas9 | + | GAGAGGTGGGCATCATTTCAGG | AGGG |
| 4989 | E75Slu19 | exon75 | SluCas9 | + | AGAGGTGGGCATCATTTCAGGA | GGGG |
| 4990 | E75Slu20 | exon75 | SluCas9 | + | GTGGGCATCATTTCAGGAGGGG | ACGG |
| 4991 | E75Slu21 | exon75 | SluCas9 | + | ATCATTTCAGGAGGGGACGGCA | GTGG |
| 4992 | E75Slu22 | exon75 | SluCas9 | + | TCATTTCAGGAGGGGACGGCAG | TGGG |
| 4993 | E75Slu23 | exon75 | SluCas9 | + | CATTTCAGGAGGGGACGGCAGT | GGGG |
| 4994 | E75Slu24 | exon75 | SluCas9 | + | CAGGAGGGGACGGCAGTGGGGA | CAGG |
| 4995 | E75Slu25 | exon75 | SluCas9 | + | TATGTTCGTGCTGCTGCTTTAG | ACGG |
| 4996 | E75Slu26 | exon75 | SluCas9 | + | ATATTCTGCTTGCAGATTCCTA | TTGG |
| 4997 | E75Slu29 | exon75 | SluCas9 | − | TAAGGCAGCTGCTGGAGCAAGT | GAGG |
| 4998 | E75Slu30 | exon75 | SluCas9 | − | GTTACACAGGCTAAGGCAGCTG | CTGG |
| 4999 | E75Slu31 | exon75 | SluCas9 | − | TGGAGTCACAGTTACACAGGCT | AAGG |
| 5000 | E75Slu32 | exon75 | SluCas9 | − | AACAGCTGGAGTCACAGTTACA | CAGG |
| 5001 | E75Slu33 | exon75 | SluCas9 | − | CCTGGAAGACCACAATAAACAG | CTGG |
| 5002 | E75Slu34 | exon75 | SluCas9 | − | CCTGGAAGCCAGGATGCAAATC | CTGG |
| 5003 | E75Slu35 | exon75 | SluCas9 | − | AACACAAAGGCCGCCTGGAAGC | CAGG |
| 5004 | E75Slu36 | exon75 | SluCas9 | − | ACTGCGTCAACACAAAGGCCGC | CTGG |
| 5005 | E75Slu37 | exon75 | SluCas9 | − | GCCAAGCTACTGCGTCAACACA | AAGG |
| 5006 | E75Slu38 | exon75 | SluCas9 | − | CCGGGATGCTGAGCTCATTGCT | GAGG |
| 5007 | E75Slu39 | exon75 | SluCas9 | − | GCCCACCTCTCCCCAGAGTCCC | CGGG |
| 5008 | E75Slu40 | exon75 | SluCas9 | − | TGCCCACCTCTCCCCAGAGTCC | CCGG |
| 5009 | E75Slu41 | exon75 | SluCas9 | − | CTAAAGCAGCAGCACGAACATA | AAGG |
| 5010 | E76Slu2 | exon76 | SluCas9 | + | CCGAAGTTTGACTGCCAACCAC | TCGG |
| 5011 | E76Slu3 | exon76 | SluCas9 | + | CTGCCAACCACTCGGAGCAGCA | TAGG |
| 5012 | E76Slu4 | exon76 | SluCas9 | + | GAGCAGCATAGGCTGACTGCTG | TCGG |
| 5013 | E76Slu5 | exon76 | SluCas9 | + | ACTGCTGTCGGACCTCTGTAGA | GAGG |
| 5014 | E76Slu6 | exon76 | SluCas9 | + | TCGGACCTCTGTAGAGAGGTAG | AAGG |
| 5015 | E76Slu7 | exon76 | SluCas9 | + | CCTCTGTAGAGAGGTAGAAGGA | GAGG |
| 5016 | E76Slu8 | exon76 | SluCas9 | + | GGACACCGTTGTGCCATTCACT | TTGG |
| 5017 | E76Slu9 | exon76 | SluCas9 | + | TGCCATTCACTTTGGCCTCTGC | CTGG |
| 5018 | E76Slu10 | exon76 | SluCas9 | + | GCCATTCACTTTGGCCTCTGCC | TGGG |
| 5019 | E76Slu11 | exon76 | SluCas9 | + | CCATTCACTTTGGCCTCTGCCT | GGGG |
| 5020 | E76Slu12 | exon76 | SluCas9 | − | GGCAGTCAAACTTCGGACTCCA | TGGG |
| 5021 | E76Slu13 | exon76 | SluCas9 | − | TGGCAGTCAAACTTCGGACTCC | ATGG |
| 5022 | E76Slu14 | exon76 | SluCas9 | − | CCGAGTGGTTGGCAGTCAAACT | TCGG |
| 5023 | E76Slu15 | exon76 | SluCas9 | − | CAGCCTATGCTGCTCCGAGTGG | TTGG |
| 5024 | E76Slu16 | exon76 | SluCas9 | − | CAGTCAGCCTATGCTGCTCCGA | GTGG |
| 5025 | E76Slu17 | exon76 | SluCas9 | − | CCTCTCCTTCTACCTCTCTACA | GAGG |
| 5026 | E76Slu18 | exon76 | SluCas9 | − | AGAGGCCAAAGTGAATGGCACA | ACGG |
| 5027 | E76Slu19 | exon76 | SluCas9 | − | CCCCAGGCAGAGGCCAAAGTGA | ATGG |
| 5028 | E76Slu20 | exon76 | SluCas9 | − | TTGGATGACTTAGCCCCAGGCA | GAGG |
| 5029 | E76Slu21 | exon76 | SluCas9 | − | TTTCTTTTGGATGACTTAGCCC | CAGG |
| 5030 | E77Slu1 | exon77 | SluCas9 | + | TATTGGAGCTTACCTCTTGAAC | TAGG |
| 5031 | E77Slu2 | exon77 | SluCas9 | + | ATTGGAGCTTACCTCTTGAACT | AGGG |
| 5032 | E77Slu3 | exon77 | SluCas9 | + | GAGCTTACCTCTTGAACTAGGG | AAGG |
| 5033 | E77Slu4 | exon77 | SluCas9 | + | CCTCTAACCCTGTGCTTGTGTC | CTGG |
| 5034 | E77Slu5 | exon77 | SluCas9 | + | CTCTAACCCTGTGCTTGTGTCC | TGGG |
| 5035 | E77Slu6 | exon77 | SluCas9 | + | TCTAACCCTGTGCTTGTGTCCT | GGGG |
| 5036 | E77Slu7 | exon77 | SluCas9 | + | AACCCTGTGCTTGTGTCCTGGG | GAGG |
| 5037 | E77Slu11 | exon77 | SluCas9 | − | AACAACTCCTTCCCTAGTTCAA | GAGG |
| 5038 | E77Slu12 | exon77 | SluCas9 | − | AAGCACAGGGTTAGAGGAGGTG | ATGG |
| 5039 | E77Slu13 | exon77 | SluCas9 | − | GGACACAAGCACAGGGTTAGAG | GAGG |
| 5040 | E77Slu14 | exon77 | SluCas9 | − | CCAGGACACAAGCACAGGGTTA | GAGG |
| 5041 | E77Slu15 | exon77 | SluCas9 | − | GTCCTCCCCAGGACACAAGCAC | AGGG |
| 5042 | E77Slu16 | exon77 | SluCas9 | − | AGTCCTCCCCAGGACACAAGCA | CAGG |
| 5043 | E77Slu17 | exon77 | SluCas9 | − | GGAAGATCTTCTCAGTCCTCCC | CAGG |
| 5044 | E78Slu1 | exon78 | SluCas9 | + | TGAATCTCACTAACCTCTCTCA | TTGG |
| 5045 | E78Slu2 | exon78 | SluCas9 | + | CTAACCTCTCTCATTGGCTTTC | CAGG |
| 5046 | E78Slu3 | exon78 | SluCas9 | + | TAACCTCTCTCATTGGCTTTCC | AGGG |
| 5047 | E78Slu4 | exon78 | SluCas9 | + | AACCTCTCTCATTGGCTTTCCA | GGGG |
| 5048 | E78Slu10 | exon78 | SluCas9 | − | TACCCCTGGAAAGCCAATGAGA | GAGG |
| 5049 | E78Slu11 | exon78 | SluCas9 | − | ATTATTAAAGGAAGAAATACCC | CTGG |
| 5050 | E79Slu1 | exon79 | SluCas9 | + | AGCAGTACTATAATACAATAGT | AAGG |
| 5051 | E79Slu2 | exon79 | SluCas9 | + | AATACAATAGTAAGGCATATAT | TTGG |
| 5052 | E79Slu3 | exon79 | SluCas9 | + | ATAATTCGTAAATGTTACAGTG | TTGG |
| 5053 | E79Slu4 | exon79 | SluCas9 | + | ATTATGATTTACAGTTTAATAC | TTGG |
| 5054 | E79Slu5 | exon79 | SluCas9 | + | ATGATTTACAGTTTAATACTTG | GTGG |
| 5055 | E79Slu6 | exon79 | SluCas9 | + | CTTACTTACTTAAACTTCTTAG | TAGG |
| 5056 | E79Slu7 | exon79 | SluCas9 | + | TAAATCTGAGTTTTAAAAATCC | TTGG |
| 5057 | E79Slu8 | exon79 | SluCas9 | + | AAATCTGAGTTTTAAAAATCCT | TGGG |
| 5058 | E79Slu9 | exon79 | SluCas9 | + | TTAAAAATCCTTGGGTAAAGAA | AAGG |
| 5059 | E79Slu10 | exon79 | SluCas9 | + | AGAAAAGGTCCAGCGTCACATA | AAGG |
| 5060 | E79Slu11 | exon79 | SluCas9 | + | AAAAACCAAATCTTCATGTAAT | TTGG |
| 5061 | E79Slu12 | exon79 | SluCas9 | + | GGTAATTTGTTACCTTAGAGCT | TTGG |
| 5062 | E79Slu13 | exon79 | SluCas9 | + | GTAATTTGTTACCTTAGAGCTT | TGGG |
| 5063 | E79Slu14 | exon79 | SluCas9 | + | GGGTTTTCTTTTGAAAATTATG | AAGG |
| 5064 | E79Slu15 | exon79 | SluCas9 | + | GAAGGAAAAAGAAAGAATTATA | AAGG |
| 5065 | E79Slu16 | exon79 | SluCas9 | + | AAAGGAAAAAGAAAATAACGCA | ATGG |
| 5066 | E79Slu17 | exon79 | SluCas9 | + | AAGAAAATAACGCAATGGACAA | GTGG |
| 5067 | E79Slu18 | exon79 | SluCas9 | + | ACAAGTGGTGAAGCTGTGAACT | CAGG |
| 5068 | E79Slu19 | exon79 | SluCas9 | + | GAACTCAGGTGTGCACAATTAT | CAGG |
| 5069 | E79Slu20 | exon79 | SluCas9 | + | AGGAACACCCCAAAACCAAAGT | GAGG |
| 5070 | E79Slu21 | exon79 | SluCas9 | + | GTTAATTAATTAATTATTAATA | ATGG |
| 5071 | E79Slu22 | exon79 | SluCas9 | + | AAGCTAATTACACTTGATGTCA | GAGG |
| 5072 | E79Slu23 | exon79 | SluCas9 | + | AGGTAACAGATTTGCAAAATTA | TAGG |
| 5073 | E79Slu24 | exon79 | SluCas9 | + | GATTTGCAAAATTATAGGTCAC | ACGG |
| 5074 | E79Slu25 | exon79 | SluCas9 | + | TTGCAAAATTATAGGTCACACG | GTGG |
| 5075 | E79Slu26 | exon79 | SluCas9 | + | TTAAAAATCAAAAAGAAATAAA | ATGG |
| 5076 | E79Slu27 | exon79 | SluCas9 | + | TTAACCTGTCTAATCCACCAAG | AAGG |
| 5077 | E79Slu28 | exon79 | SluCas9 | + | TAACCTGTCTAATCCACCAAGA | AGGG |
| 5078 | E79Slu29 | exon79 | SluCas9 | + | TCTTACTTACTTACAAACCTGT | GTGG |
| 5079 | E79Slu30 | exon79 | SluCas9 | + | GAACTACTCGCAGAGAAATGCA | AAGG |
| 5080 | E79Slu31 | exon79 | SluCas9 | + | TACTCGCAGAGAAATGCAAAGG | ATGG |
| 5081 | E79Slu32 | exon79 | SluCas9 | + | GCAAAGGATGGAAACACAGTTC | ATGG |
| 5082 | E79Slu33 | exon79 | SluCas9 | + | CAAAGGATGGAAACACAGTTCA | TGGG |
| 5083 | E79Slu34 | exon79 | SluCas9 | + | TGGAAACACAGTTCATGGGCTT | CTGG |
| 5084 | E79Slu35 | exon79 | SluCas9 | + | GGAAACACAGTTCATGGGCTTC | TGGG |
| 5085 | E79Slu36 | exon79 | SluCas9 | + | ACCTGTCAGTATCACAAATGTG | ATGG |
| 5086 | E79Slu37 | exon79 | SluCas9 | + | CCTGTCAGTATCACAAATGTGA | TGGG |
| 5087 | E79Slu38 | exon79 | SluCas9 | + | CTGTCAGTATCACAAATGTGAT | GGGG |
| 5088 | E79Slu39 | exon79 | SluCas9 | + | TATTTATTTTAAATTATGTCTT | GTGG |
| 5089 | E79Slu40 | exon79 | SluCas9 | + | GGCATTTAAAAACTCATCCCAC | ATGG |
| 5090 | E79Slu41 | exon79 | SluCas9 | + | GCATTTAAAAACTCATCCCACA | TGGG |
| 5091 | E79Slu42 | exon79 | SluCas9 | + | CAGTTCCTTTTGACTGTGAGAA | GAGG |
| 5092 | E79Slu43 | exon79 | SluCas9 | + | AGTTCCTTTTGACTGTGAGAAG | AGGG |
| 5093 | E79Slu44 | exon79 | SluCas9 | + | CATCCAATCCTTCACTTAAAGA | GTGG |
| 5094 | E79Slu45 | exon79 | SluCas9 | + | TAAAGAGTGGCCTACTCCTTCA | CAGG |
| 5095 | E79Slu46 | exon79 | SluCas9 | + | AAAGAGTGGCCTACTCCTTCAC | AGGG |
| 5096 | E79Slu47 | exon79 | SluCas9 | + | AGTGGCCTACTCCTTCACAGGG | ATGG |
| 5097 | E79Slu48 | exon79 | SluCas9 | + | GTGGCCTACTCCTTCACAGGGA | TGGG |
| 5098 | E79Slu49 | exon79 | SluCas9 | + | CCTACTCCTTCACAGGGATGGG | CTGG |
| 5099 | E79Slu50 | exon79 | SluCas9 | + | CTACTCCTTCACAGGGATGGGC | TGGG |
| 5100 | E79Slu51 | exon79 | SluCas9 | + | CCTATCATCGTCCTGCTACTGC | TTGG |
| 5101 | E79Slu52 | exon79 | SluCas9 | + | CTATCATCGTCCTGCTACTGCT | TGGG |
| 5102 | E79Slu53 | exon79 | SluCas9 | + | AAAAATACCTTCTGATGTTCAC | AAGG |
| 5103 | E79Slu54 | exon79 | SluCas9 | + | GAAGCATCATTAAATCTAAATC | GTGG |
| 5104 | E79Slu55 | exon79 | SluCas9 | + | TAAATCGTGGCATTGCTAGCAG | CAGG |
| 5105 | E79Slu56 | exon79 | SluCas9 | + | CTTTACTTTACTTTCGTTGTCA | GTGG |
| 5106 | E79Slu57 | exon79 | SluCas9 | + | AGTTGTTTAAAATGAGAAACAT | CTGG |
| 5107 | E79Slu58 | exon79 | SluCas9 | + | GAGCTGTAGACAATGTTTTAGT | GTGG |
| 5108 | E79Slu59 | exon79 | SluCas9 | 1 | TTTAGTGTGGTGTAGTTTCCTC | CTGG |
| 5109 | E79Slu60 | exon79 | SluCas9 | + | TGGTGTAGTTTCCTCCTGGCTT | CCGG |
| 5110 | E79Slu61 | exon79 | SluCas9 | + | AGTTTCCTCCTGGCTTCCGGCT | CCGG |
| 5111 | E79Slu62 | exon79 | SluCas9 | + | GTTTCCTCCTGGCTTCCGGCTC | CGGG |
| 5112 | E79Slu63 | exon79 | SluCas9 | + | GAAAAATCCATTCTAGACCAAG | CAGG |
| 5113 | E79Slu64 | exon79 | SluCas9 | + | ATTCTAGACCAAGCAGGTAAGC | CTGG |
| 5114 | E79Slu65 | exon79 | SluCas9 | + | ACTAGAAGTAATTTCTTTCTAT | TAGG |
| 5115 | E79Slu66 | exon79 | SluCas9 | + | ATGTGACATGAACATTTAAAAA | ATGG |
| 5116 | E79Slu67 | exon79 | SluCas9 | + | ATGAGCAGTGTGTAGTAGTCAT | TTGG |
| 5117 | E79Slu68 | exon79 | SluCas9 | + | CAGTGTGTAGTAGTCATTTGGT | GTGG |
| 5118 | E79Slu69 | exon79 | SluCas9 | + | TGTGTAGTAGTCATTTGGTGTG | GTGG |
| 5119 | E79Slu70 | exon79 | SluCas9 | + | GTAGTCATTTGGTGTGGTGGTA | GAGG |
| 5120 | E79Slu71 | exon79 | SluCas9 | + | AAGAAAAAGCCATGAATTTCAT | ATGG |
| 5121 | E79Slu72 | exon79 | SluCas9 | + | TTTCATATGGATATCACGCCAA | AAGG |
| 5122 | E79Slu73 | exon79 | SluCas9 | + | GTGTGTGTGTTTGTTTTGTTTT | TAGG |
| 5123 | E79Slu74 | exon79 | SluCas9 | + | TGTGTGTGTTTGTTTTGTTTTT | AGGG |
| 5124 | E79Slu75 | exon79 | SluCas9 | + | GTGTGTGTTTGTTTTGTTTTTA | GGGG |
| 5125 | E79Slu76 | exon79 | SluCas9 | + | AAAATGCGTTCCATATAAAGAA | ATGG |
| 5126 | E79Slu77 | exon79 | SluCas9 | + | GTATGAATATTATAAAAACCAT | GCGG |
| 5127 | E79Slu78 | exon79 | SluCas9 | + | TATGAATATTATAAAAACCATG | CGGG |
| 5128 | E79Slu79 | exon79 | SluCas9 | + | ATTATAAAAACCATGCGGGAAT | CAGG |
| 5129 | E79Slu80 | exon79 | SluCas9 | + | TTCATCTGTCATGACTGATACT | AAGG |
| 5130 | E79Slu81 | exon79 | SluCas9 | + | CTCTGCCCAAATCATCTGCCAT | GTGG |
| 5131 | E79Slu82 | exon79 | SluCas9 | + | AAAAGACTTCCTACATTGTGTC | CTGG |
| 5132 | E79Slu84 | exon79 | SluCas9 | − | TGGCATGTTTTGTCATTGTTTT | CAGG |
| 5133 | E79Slu85 | exon79 | SluCas9 | − | GTAACTGAAGCATAAACATCAC | ATGG |
| 5134 | E79Slu86 | exon79 | SluCas9 | − | CTGCATTTTAAGCATAATTAAT | TTGG |
| 5135 | E79Slu87 | exon79 | SluCas9 | − | TTTTCTTTTAGACACATTAGCT | CTGG |
| 5136 | E79Slu88 | exon79 | SluCas9 | − | AGTAAGTTTCATTCTAAAATCA | GAGG |
| 5137 | E79Slu89 | exon79 | SluCas9 | − | TTAAAACTCAGATTTAAAACAA | GGGG |
| 5138 | E79Slu90 | exon79 | SluCas9 | − | TTTAAAACTCAGATTTAAAACA | AGGG |
| 5139 | E79Slu91 | exon79 | SluCas9 | − | TTTTAAAACTCAGATTTAAAAC | AAGG |
| 5140 | E79Slu92 | exon79 | SluCas9 | − | ACGCTGGACCTTTTCTTTACCC | AAGG |
| 5141 | E79Slu93 | exon79 | SluCas9 | − | CATTTTTTTCCTTTATGTGACG | CTGG |
| 5142 | E79Slu94 | exon79 | SluCas9 | − | AATTACCAAATTACATGAAGAT | TTGG |
| 5143 | E79Slu95 | exon79 | SluCas9 | − | TCAAAAGAAAACCCAAAGCTCT | AAGG |
| 5144 | E79Slu96 | exon79 | SluCas9 | − | ATTTCTACCTCACTTTGGTTTT | GGGG |
| 5145 | E79Slu97 | exon79 | SluCas9 | − | TATTTCTACCTCACTTTGGTTT | TGGG |
| 5146 | E79Slu98 | exon79 | SluCas9 | − | CTATTTCTACCTCACTTTGGTT | TTGG |
| 5147 | E79Slu99 | exon79 | SluCas9 | − | CTCATGCTATTTCTACCTCACT | TTGG |
| 5148 | E79Slu100 | exon79 | SluCas9 | − | AATTAATTAATTAACATCAAAC | ACGG |
| 5149 | E79Slu101 | exon79 | SluCas9 | − | AGTGTAATTAGCTTTTGGAGAG | TGGG |
| 5150 | E79Slu102 | exon79 | SluCas9 | − | AAGTGTAATTAGCTTTTGGAGA | GTGG |
| 5151 | E79Slu103 | exon79 | SluCas9 | − | TGACATCAAGTGTAATTAGCTT | TTGG |
| 5152 | E79Slu104 | exon79 | SluCas9 | − | AGTGTAATTAGCTTTTGGAGAG | TGGG |
| 5153 | E79Slu105 | exon79 | SluCas9 | − | AAGTGTAATTAGCTTTTGGAGA | GTGG |
| 5154 | E79Slu106 | exon79 | SluCas9 | − | TGACATCAAGTGTAATTAGCTT | TTGG |
| 5155 | E79Slu107 | exon79 | SluCas9 | − | AAGAGGAAAAGCTAAGGACTGG | TAGG |
| 5156 | E79Slu108 | exon79 | SluCas9 | − | GCTCAAGAGGAAAAGCTAAGGA | CTGG |
| 5157 | E79Slu109 | exon79 | SluCas9 | − | GAAAAGCTCAAGAGGAAAAGCT | AAGG |
| 5158 | E79Slu110 | exon79 | SluCas9 | − | AAAAAAGAGGAGAAAAGCTCAA | GAGG |
| 5159 | E79Slu111 | exon79 | SluCas9 | − | CAAACAAAAATTGCTCAAAAAA | GAGG |
| 5160 | E79Slu112 | exon79 | SluCas9 | − | AAACCCTTCTTGGTGGATTAGA | CAGG |
| 5161 | E79Slu113 | exon79 | SluCas9 | − | TGTTACAAAAAAACCCTTCTTG | GTGG |
| 5162 | E79Slu114 | exon79 | SluCas9 | − | AAATGTTACAAAAAAACCCTTC | TTGG |
| 5163 | E79Slu115 | exon79 | SluCas9 | − | GGTTTGTAAGTAAGTAAGAAAG | AAGG |
| 5164 | E79Slu116 | exon79 | SluCas9 | − | TTTCTCTGCGAGTAGTTCCACA | CAGG |
| 5165 | E79Slu117 | exon79 | SluCas9 | − | CCCATCACATTTGTGATACTGA | CAGG |
| 5166 | E79Slu118 | exon79 | SluCas9 | − | TAAAATAAATAAACTTTGGGAA | AAGG |
| 5167 | E79Slu119 | exon79 | SluCas9 | − | ATAATTTAAAATAAATAAACTT | TGGG |
| 5168 | E79Slu120 | exon79 | SluCas9 | − | CATAATTTAAAATAAATAAACT | TTGG |
| 5169 | E79Slu121 | exon79 | SluCas9 | − | TTTTTAGATTTATTGTCCCATG | TGGG |
| 5170 | E79Slu122 | exon79 | SluCas9 | − | TTTTTTAGATTTATTGTCCCAT | GTGG |
| 5171 | E79Slu123 | exon79 | SluCas9 | − | CAGTCAAAAGGAACTGGGTGGT | TTGG |
| 5172 | E79Slu124 | exon79 | SluCas9 | − | TCTCACAGTCAAAAGGAACTGG | GTGG |
| 5173 | E79Slu125 | exon79 | SluCas9 | − | TCTTCTCACAGTCAAAAGGAAC | TGGG |
| 5174 | E79Slu126 | exon79 | SluCas9 | − | CTCTTCTCACAGTCAAAAGGAA | CTGG |
| 5175 | E79Slu127 | exon79 | SluCas9 | − | TATGCCCTCTTCTCACAGTCAA | AAGG |
| 5176 | E79Slu128 | exon79 | SluCas9 | − | AGGCCACTCTTTAAGTGAAGGA | TTGG |
| 5177 | E79Slu129 | exon79 | SluCas9 | − | GGAGTAGGCCACTCTTTAAGTG | AAGG |
| 5178 | E79Slu130 | exon79 | SluCas9 | − | CCAGCCCATCCCTGTGAAGGAG | TAGG |
| 5179 | E79Slu131 | exon79 | SluCas9 | − | GGATTCCCAGCCCATCCCTGTG | AAGG |
| 5180 | E79Slu132 | exon79 | SluCas9 | − | ACGATGATAGGGCTGGAGGGCT | ATGG |
| 5181 | E79Slu133 | exon79 | SluCas9 | − | AGCAGGACGATGATAGGGCTGG | AGGG |
| 5182 | E79Slu134 | exon79 | SluCas9 | − | TAGCAGGACGATGATAGGGCTG | GAGG |
| 5183 | E79Slu135 | exon79 | SluCas9 | − | CAGTAGCAGGACGATGATAGGG | CTGG |
| 5184 | E79Slu136 | exon79 | SluCas9 | − | CAAGCAGTAGCAGGACGATGAT | AGGG |
| 5185 | E79Slu137 | exon79 | SluCas9 | − | CCAAGCAGTAGCAGGACGATGA | TAGG |
| 5186 | E79Slu138 | exon79 | SluCas9 | − | TTTTTTAACTCCCAAGCAGTAG | CAGG |
| 5187 | E79Slu139 | exon79 | SluCas9 | − | ATTCTGACCTTGTGAACATCAG | AAGG |
| 5188 | E79Slu140 | exon79 | SluCas9 | − | TGCTTCAGTGGAAATCAATCAG | AAGG |
| 5189 | E79Slu141 | exon79 | SluCas9 | − | ATTTAGATTTAATGATGCTTCA | GTGG |
| 5190 | E79Slu142 | exon79 | SluCas9 | − | ATCAGAGTGAGTAATCGGTTGG | TTGG |
| 5191 | E79Slu143 | exon79 | SluCas9 | − | TTATATCAGAGTGAGTAATCGG | TTGG |
| 5192 | E79Slu144 | exon79 | SluCas9 | − | CTTATTATATCAGAGTGAGTAA | TCGG |
| 5193 | E79Slu145 | exon79 | SluCas9 | − | AGGGAACATGTGAATGAATACA | CAGG |
| 5194 | E79Slu146 | exon79 | SluCas9 | − | GTAAAGTATTGGATTTTTTTAA | AGGG |
| 5195 | E79Slu147 | exon79 | SluCas9 | − | AGTAAAGTATTGGATTTTTTTA | AAGG |
| 5196 | E79Slu148 | exon79 | SluCas9 | − | ACAACGAAAGTAAAGTAAAGTA | TTGG |
| 5197 | E79Slu149 | exon79 | SluCas9 | − | TTTTCCCGGAGCCGGAAGCCAG | GAGG |
| 5198 | E79Slu150 | exon79 | SluCas9 | − | GATTTTTCCCGGAGCCGGAAGC | CAGG |
| 5199 | E79Slu151 | exon79 | SluCas9 | − | CTAGAATGGATTTTTCCCGGAG | CCGG |
| 5200 | E79Slu152 | exon79 | SluCas9 | − | CTTGGTCTAGAATGGATTTTTC | CCGG |
| 5201 | E79Slu153 | exon79 | SluCas9 | − | AGGCTTACCTGCTTGGTCTAGA | ATGG |
| 5202 | E79Slu154 | exon79 | SluCas9 | − | TCAGTCATCCAGGCTTACCTGC | TTGG |
| 5203 | E79Slu155 | exon79 | SluCas9 | − | AAATTACTTCTAGTCAGTCATC | CAGG |
| 5204 | E79Slu156 | exon79 | SluCas9 | − | GAGAACTGTCAGCTGAGTGGGG | CAGG |
| 5205 | E79Slu157 | exon79 | SluCas9 | − | ATTTGAGAACTGTCAGCTGAGT | GGGG |
| 5206 | E79Slu158 | exon79 | SluCas9 | − | CATTTGAGAACTGTCAGCTGAG | TGGG |
| 5207 | E79Slu159 | exon79 | SluCas9 | − | TCATTTGAGAACTGTCAGCTGA | GTGG |
| 5208 | E79Slu160 | exon79 | SluCas9 | − | GCGTGATATCCATATGAAATTC | ATGG |
| 5209 | E79Slu161 | exon79 | SluCas9 | − | CAGCGCATTGTTTTGCATCCTT | TTGG |
| 5210 | E79Slu162 | exon79 | SluCas9 | − | ACACACACACACACAAAACTTT | GAGG |
| 5211 | E79Slu163 | exon79 | SluCas9 | − | TTTCTTTATATGGAACGCATTT | TGGG |
| 5212 | E79Slu164 | exon79 | SluCas9 | − | ATTTCTTTATATGGAACGCATT | TTGG |
| 5213 | E79Slu165 | exon79 | SluCas9 | − | AAATAACTTGCCATTTCTTTAT | ATGG |
| 5214 | E79Slu166 | exon79 | SluCas9 | − | GTTATTGTTTTGTTAACAATGG | CAGG |
| 5215 | E79Slu167 | exon79 | SluCas9 | − | GTCTGTTATTGTTTTGTTAACA | ATGG |
| 5216 | E79Slu168 | exon79 | SluCas9 | − | ATCTATATTTTTGTGAAGGGTA | GTGG |
| 5217 | E79Slu169 | exon79 | SluCas9 | − | AAATAAATCTATATTTTTGTGA | AGGG |
| 5218 | E79Slu170 | exon79 | SluCas9 | − | GAAATAAATCTATATTTTTGTG | AAGG |
| 5219 | E79Slu171 | exon79 | SluCas9 | − | TTTATAATATTCATACAACAAA | GAGG |
| 5220 | E79Slu172 | exon79 | SluCas9 | − | TTTTACAACTCCTGATTCCCGC | ATGG |
| 5221 | E79Slu173 | exon79 | SluCas9 | − | GTATCAGTCATGACAGATGAAG | AAGG |
| 5222 | E79Slu174 | exon79 | SluCas9 | − | GGCAGATGATTTGGGCAGAGCG | ATGG |
| 5223 | E79Slu175 | exon79 | SluCas9 | − | CTTTTCCACATGGCAGATGATT | TGGG |
| 5224 | E79Slu176 | exon79 | SluCas9 | − | TCTTTTCCACATGGCAGATGAT | TTGG |
| 5225 | E79Slu177 | exon79 | SluCas9 | − | CAATGTAGGAAGTCTTTTCCAC | ATGG |
| 5226 | E79Slu178 | exon79 | SluCas9 | − | CTTTGTTTTCCAGGACACAATG | TAGG |

### Compositions

In some embodiments, a composition is provided comprising one or more guide RNAs, or one or more nucleic acids encoding one or more guide RNAs, comprising or consisting of one or more guide sequence of Table 1A (SEQ ID NOs: 1000-3081 for SaCas9) or Table 1B (SEQ ID NOs: 4000-5226 for SluCas9). Tables 1A-B provide the endonuclease associated with each guide sequence such that for each guide sequence described herein the type of endonuclease to be paired with the guide (for compositions) or used with the guide (for methods/uses) can be determined.

In some embodiments, a composition is provided comprising one or more guide RNAs, or one or more nucleic acids encoding one or more guide RNAs, wherein the guide RNA comprises at least 16, 17, 18, 19, or 20 contiguous nucleotides of a guide sequence of Tables 1A-B, or is at least 70%, at least 75%, at least 80%, at least 85%, or at least 90% identical to a guide sequence comprising at least 16, 17, 18, 19, or 20 nucleotides of a guide sequence of Tables 1A-B. In particular embodiments, a composition is provided comprising one or more guide RNAs, or one or more nucleic acids encoding one or more guide RNAs, wherein the guide RNA comprises at least 20 contiguous nucleotides of a guide sequence of Tables 1A-B, or is at least 70%, at least 75%, at least 80%, at least 85%, or at least 90% identical to guide sequence comprising at least 20 nucleotides of a guide sequence of Tables 1A-B. In other particular embodiments, a composition is provided comprising one or more guide RNAs, or one or more nucleic acids encoding one or more guide RNAs, wherein the guide RNA comprises no more than 16, no more than 17, no more than 18, no more than 19, no more than 20, no more than 21, or no more than 22 contiguous nucleotides of a guide sequence of Tables 1A-B, or is at least 70%, at least 75%, at least 80%, at least 85%, or at least 90% identical to guide sequence comprising no more than 16, no more than 17, no more than 18, no more than 19, no more than 20, no more than 21, or no more than 22 contiguous nucleotides of a guide sequence of Tables 1A-B.

In some embodiments, a composition is provided comprising two or more guide RNAs, or nucleic acid encoding two or more guide RNAs, wherein each guide RNA comprises a guide sequence of Tables 1A-B, or at least 16, 17, 18, 19, or 20 contiguous nucleotides of a guide sequence of Tables 1A-B, or is at least 70%, at least 75%, at least 80%, at least 85%, or at least 90% identical to a guide sequence comprising at least 16, 17, 18, 19, or 20 nucleotides of a guide sequence selected from Tables 1A-B. In particular embodiments, a composition is provided comprising two or more guide RNAs, or nucleic acid encoding two or more guide RNAs, wherein each guide RNA comprises at least 20 contiguous nucleotides of a guide sequence of Tables 1A-B, or is at least 70%, at least 75%, at least 80%, at least 85%, or at least 90% identical to a guide sequence comprising at least 20 nucleotides of a guide sequence selected from Tables 1A-B. In other particular embodiments, a composition is provided comprising two or more guide RNAs, or nucleic acid encoding two or more guide RNAs, wherein at least one of the two or more guide RNAs, optionally each guide RNA, comprises no more than 16, no more than 17, no more than 18, no more than 19, no more than 20, no more than 21, or no more than 22 contiguous nucleotides of a guide sequence of Tables 1A-B, or is at least 70%, at least 75%, at least 80%, at least 85%, or at least 90% identical to a guide sequence comprising no more than 16, no more than 17, no more than 18, no more than 19, no more than 20, no more than 21, or no more than 22 nucleotides of a guide sequence selected from Tables 1A-B.

In some embodiments, a composition is provided comprising one or more nucleic acid molecules, wherein at least one of the molecules comprises nucleic acid encoding: a) a SaCas9 or SluCas9; and b) a first and a second guide RNA comprising a first and a second guide sequence, wherein the first and second guide sequence are selected from any one of the guide sequences of Tables 1A-B.

In some embodiments, a composition is provided comprising two nucleic acid molecules, wherein at least one of the molecules comprises a nucleic acid encoding a first and a second guide RNA comprising a first and a second guide sequence, wherein the first and second guide sequence are selected from any one of the guide sequences of Tables 1A-B, optionally wherein the nucleic acid does not comprise a nucleic acid encoding an endonuclease.

In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon 1. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon 2. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon 3. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 4. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 5. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 6. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 7. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 8. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 9. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 10. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 11. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 12. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 13. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 14. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 15. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 16. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 17. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 18. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 19. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 20. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 21. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 22. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 23. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 24. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 25. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 26. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 27. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 28. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 29. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 30. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 31. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 32. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 33. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 34. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 35. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 36. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 37. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 38. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 39. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 40. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 41. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 42. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 43. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 46. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 47. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 48. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 49. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 52. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 54. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 55. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 56. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 57. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 58. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 59. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 60. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 61. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 62. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 63. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 64. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 65. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 66. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 67. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 68. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 69. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 70. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 71. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 72. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 73. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 74. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 75. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 76. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 77. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 78. In some embodiments, a composition is provided comprising a pair of guide RNAs, or nucleic acid encoding a pair of guide RNAs, wherein the pair of guide RNAs comprises or consists of any two of the guide sequences of Tables 1A (for SaCas9) or 1B (for SluCas9) for exon: 79. For clarity, a guide sequence of Tables 1A or 1B is “for exon: XX” if the guide targets a region in the recited exon.

In some embodiments, a composition is provided comprising: i) an SaCas9 or a nucleic acid encoding a SaCas9, and ii) a first and a second guide RNA, or a nucleic acid encoding a first and a second guide RNA, wherein the first and second guide RNA comprise any two spacer sequences of SEQ ID NOs: 1000-1353. In particular embodiments, the first and second guide RNAs each comprise sequences that target the same exon. In particular embodiments, the first and second guide RNAs each comprise sequences that target a different site in the same exon.

In some embodiments, a composition is provided comprising: i) an SluCas9 or a nucleic acid encoding a SluCas9, and ii) a first and a second guide RNA, or a nucleic acid encoding a first and a second guide RNA, wherein the first and second guide RNA comprises any two spacer sequences of SEQ ID NOs: 4000-5226. In particular embodiments, the first and second guide RNAs each comprise sequences that target the same exon. In particular embodiments, the first and second guide RNAs each comprise sequences that target a different site in the same exon.

In some embodiments, a composition is provided comprising: i) an SaCas9-KKH or a nucleic acid encoding a SaCas9-KKH, and ii) a first and a second guide RNA, or a nucleic acid encoding a first and a second guide RNA, wherein the first and second guide RNA comprises any two spacer sequences of SEQ ID NOs 1354-3081. In particular embodiments, the first and second guide RNAs each comprise sequences that target the same exon. In particular embodiments, the first and second guide RNAs each comprise sequences that target a different site in the same exon. In some embodiments, the composition further comprises an endonuclease or a nucleic acid encoding an endonuclease. An exemplary endonuclease for use with each of the guide RNAs or pairs of guide RNAs is provided herein, for example, in Tables 1A-B, at column “enzyme,” or in the “Guide ID” name as “Sa” for SaCas9, “SaCas9KKH” for SaCas9, or “SL” for SluCas9.

In some embodiments, a composition is provided comprising a single nucleic acid molecule comprising a nucleic acid encoding any of the guide RNAs disclosed herein, or any of the pairs of guide RNAs disclosed herein, and optionally a nucleic acid encoding an endonuclease.

In some embodiments, a composition is provided comprising at least two nucleic acid molecules comprising a nucleic acid encoding any of the guide RNAs disclosed herein, or any of the pairs of guide RNAs disclosed herein, and optionally a nucleic acid encoding an endonuclease, wherein at least one nucleic acid molecule does not comprise a nucleic acid encoding an endonuclease.

In some embodiments, a composition is provided comprising or consisting of a single nucleic acid molecule comprising: i) a nucleic acid encoding Staphylococcus aureus Cas9 (SaCas9) or Staphylococcus lugdunensis (SluCas9) and at least one, at least two, or at least three guide RNAs; or ii) a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or iii) a nucleic acid encoding Staphylococcus aureus Cas9 (SaCas9) or Staphylococcus lugdunensis (SluCas9) and one to three guide RNAs, wherein in each instance, the single nucleic acid molecule comprises a nucleic acid encoding at least one, at least two, or two guide sequence(s) of Table 1A or Table 1B.

In some embodiments, a composition is provided comprising or consisting of at least two nucleic acid molecules comprising a first nucleic acid encoding Staphylococcus aureus Cas9 (SaCas9) or Staphylococcus lugdunensis (SluCas9) and i) a nucleic acid encoding at least one, at least two, or at least three guide RNAs; or ii) a nucleic acid encoding from one to n guide RNAs, wherein n is no more than the maximum number of guide RNAs that can be expressed from said nucleic acid; or iii) a nucleic acid encoding one to three guide RNAs; and a second nucleic acid that does not encode a SaCas9 or SluCas9, optionally wherein the second nucleic acid comprises any one of i) at least one, at least two, at least three, at least four, at least five, or at least six guide RNAs; or ii) from one to n guide RNAs, wherein n is no more than the maximum number of guide RNAs that can be expressed from said nucleic acid; or iii) from one to six guide RNAs, wherein in each instance, the guide RNAs comprises nucleic acid encoding at least one, at least two, or two guide sequence of Table 1A or Table 1B. The disclosure herein may reference a “first and a second spacer” or a “first and a second guide RNA, gRNA, or sgRNA” followed by one or more pairs of specific sequences. It should be noted that the order of the sequences in the pair is not intended to be restricted to the order in which they are presented/described. For example, the phrase “the first sgRNA and the second sgRNA comprise the sequences of SEQ ID NOs: 1000 and 1015” could mean that the first sgRNA comprises the sequence of SEQ ID NO: 1000 and the second sgRNA sequence comprises the sequence of SEQ ID NO: 1015, or this phrase could mean that the first sgRNA comprises the sequence of SEQ ID NO: 1015 and the second sgRNA sequence comprises the sequence of SEQ ID NO: 1000.

In some embodiments, the first and/or the second nucleic acid, if present, comprises at least two guide RNAs. In some embodiments, the first and/or the second nucleic acid, if present, comprises at least three guide RNAs. In some embodiments, the first and/or the second nucleic acid, if present, comprises at least four guide RNAs. In some embodiments, the first and/or the second nucleic acid, if present, comprises at least five guide RNAs. In some embodiments, the first and/or the second nucleic acid, if present, comprises at least six guide RNAs. In some embodiments, the first nucleic acid encodes an endonuclease and at least one, at least two, or at least three guide RNAs. In some embodiments, the first nucleic acid comprises an endonuclease and from one to n guide RNAs, wherein n is no more than the maximum number of guide RNAs that can be expressed from said nucleic acid. In some embodiments, the first nucleic acid encodes an endonuclease and from one to three guide RNAs. In some embodiments, the first nucleic acid comprises 1, 2, 3, 4, 5, or 6 guide RNAs, but does not encode an endonuclease.

In some embodiments, the second nucleic acid, if present, encodes at least one, at least two, at least three, at least four, at least five, or at least six guide RNAs. In some embodiments, the second nucleic acid, if present, encodes from one to n guide RNAs, wherein n is no more than the maximum number of guide RNAs that can be expressed from said nucleic acid In some embodiments, the second nucleic acid, if present, encodes from one to six guide RNAs. In some embodiments, the second nucleic acid, if present, encodes 2-10, 2-9, 2-8, 2-7, 2-6, 2-5, 2-4, or 2-3 guide RNAs. In some embodiments, the second nucleic acid, if present, encodes 2, 3, 4, 5, or 6 guide RNAs. In some embodiments, the second nucleic acid comprises 1, 2, 3, 4, 5, or 6 guide RNAs, but does not encode an endonuclease.

In some embodiments, the disclosure provides for a composition comprising at least two nucleic acid molecules, wherein the first nucleic acid comprises a guide RNA and the second nucleic acid comprises a guide RNA, wherein the guide RNA encoded by the first nucleic acid and the second nucleic acid are the same. In some embodiments, the disclosure provides for a composition comprising at least two nucleic acid molecules, wherein the first nucleic acid comprises a guide RNA and the second nucleic acid comprises a guide RNA, wherein the guide RNA encoded by the first nucleic acid and the second nucleic acid are different. In some embodiments, the disclosure provides for a composition comprising at least two nucleic acid molecules, wherein the first nucleic acid comprises a guide RNA and the second nucleic acid comprises a guide RNA, wherein at least one guide RNA binds to a target sequence within an exon in the DMD gene that is upstream of a premature stop codon, and wherein at least one guide RNA binds to a target sequence within an exon in the DMD gene that is downstream of a premature stop codon. In some embodiments, the disclosure provides for a composition comprising at least two nucleic acid molecules, wherein the first nucleic acid comprises a guide RNA and the second nucleic acid comprises a guide RNA, wherein the same guide RNA is encoded by the nucleic acid of the first and second nucleic acid molecule. In some embodiments, the disclosure provides for a composition comprising at least two nucleic acid molecules, wherein the first nucleic acid comprises a guide RNA and the second nucleic acid comprises a guide RNA, wherein the second nucleic acid molecule encodes a guide RNA that binds to the same target sequence as the guide RNA in the first nucleic acid molecule. In some embodiments, the disclosure provides for a composition comprising at least two nucleic acid molecules, wherein the second nucleic acid molecule encodes at least 2, at least 3, at least 4, at least 5, or at least 6 guide RNAs, wherein the guide RNAs in the second nucleic acid molecule bind to the same target sequence as the guide RNA in the first nucleic acid molecule.

In some embodiments, the disclosure provides for a composition comprising at least two guide RNAs, i) each guide RNA targets the same genomic target sequence; ii) each guide RNA targets a different target sequence; or iii) at least one guide RNA targets one sequence and at least one guide RNA targets a different sequence.

In some embodiments, the disclosure provides for a composition comprising a guide RNA that binds to an exon of the DMD gene, wherein the exon is selected from exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 46, 47, 48, 49, 52, 54, 55, 56, 57, 58, 59, 60, 61, 62, 63, 64, 65, 66, 67, 68, 69, 70, 71, 72, 73, 74, 75, 76, 77, 78, and 79.

In some embodiments, the disclosure provides for a composition comprising at least two guide RNAs that bind to an exon of the DMD gene, wherein at least one guide RNA binds to a target sequence within an exon in the DMD gene, and wherein at least one guide RNA binds to a different target sequence within the same exon in the DMD gene.

In some embodiments, the disclosure provides for a composition comprising at least two guide RNAs, wherein at least one guide RNA binds to a target sequence within an exon that is upstream of a premature stop codon, and wherein at least one guide RNA binds to a target sequence within an exon that is downstream of a premature stop codon.

In some embodiments, the disclosure provides for a composition comprising at least two guide RNAs, wherein at least one guide RNA binds to a target sequence within an exon in the DMD gene, and wherein at least one guide RNA binds to a different target sequence within the same exon in the DMD gene, wherein when expressed in vitro or in vivo in the presence of an appropriate endonuclease (e.g., SaCas9 or SluCas9), a portion of the exon is excised. In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon.

In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, and wherein the portion of the exon remaining after excision are rejoined with a one nucleotide insertion. In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, wherein the portion of the exon remaining after excision is rejoined without a nucleotide insertion.

In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of a dystrophin gene (e.g., an exon). In some embodiments, the portions of the exon remaining after excision are rejoined with a one nucleotide insertion. In some embodiments, the portions of the gene remaining after excision are rejoined without a nucleotide insertion or a two nucleotide insertion. In some embodiments, the portions of the exon remaining after excision are rejoined without any nucleotide insertion. s“Precise segmental deletion” or “precise deletion” means that the dual cut process takes out a specific deletion. This precise deletion can lead to exon refraining. In some embodiments, the size of the excised portion is between 5 and 250, 5 and 225, 5 and 200, 5 and 190, 5 and 180, 5 and 170, 5 and 160, 5 and 150, 5 and 125, 5 and 120, 5 and 115, 5 and 110, 5 and 100, 5 and 95, 5 and 90, 5 and 85, 5 and 80, 5 and 75, 5 and 70, 5 and 65, 5 and 60, 5 and 55, 5 and 50, 5 and 45, 5 and 40, 5 and 35, 5 and 30, 5 and 25, 5 and 20, 5 and 15, and 5-10 nucleotides. In some embodiments, the size of excised portion is between 20 and 250, 20 and 225, 20 and 200, 20 and 190, 20 and 180, 20 and 170, 20 and 160, 20 and 150, 20 and 125, 20 and 120, 20 and 115, 20 and 110, 20 and 100, 20 and 95, 20 and 90, 20 and 85, 20 and 80, 20 and 75, 20 and 70, 20 and 65, 20 and 60, 20 and 55, 20 and 50, 20 and 45, 20 and 40, 20 and 35, 20 and 30, and 20 and 25 nucleotides. In some embodiments, the size of excised portion is between 50 and 250, 50 and 225, 50 and 200, 50 and 190, 50 and 180, 50 and 170, 50 and 160, 50 and 150, 50 and 125, 50 and 120, 50 and 115, 50 and 110, and 50 and 100 nucleotides. In some embodiments, the size of excised portion of the exon is between 8 and 167 nucleotides.

In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, wherein the size of the excised portion of the exon is between 5, 6, 7, 8, 9, 10, 15, or 20 and 250 nucleotides in length. In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, wherein the size of excised portion of the exon is between 150 and 250, 100 and 250, 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, and 50 and 75 nucleotides.

In some embodiments, the disclosure provides for a composition comprising at least two guide RNAs, wherein once expressed in vitro or in vivo in the presence of an appropriate endonuclease (e.g., SaCas9 or SluCas9), the endonuclease excises a portion of the exon, wherein the size of excised portion of the exon is between 8 and 167 nucleotides.

In some embodiments, a guide RNA and a Cas9 are encoded on a single nucleic acid molecule. In some embodiments, the single nucleic acid molecule comprises a nucleic acid encoding a guide RNA and a nucleic acid encoding a SaCas9 or SluCas9. In some embodiments, two guide RNAs and a Cas9 are encoded on a single nucleic acid molecule. In some embodiments, the single nucleic acid molecule comprises a nucleic acid encoding a first guide RNA, a nucleic acid encoding a second guide RNA, and a nucleic acid encoding a SaCas9 or SluCas9. In some embodiments, the spacer sequences of the first and second guide RNAs are identical. In some embodiments, the spacer sequences of the first and second guide RNAs are not identical.

In some embodiments, a single nucleic acid molecule comprises a nucleic acid encoding a Cas9 and a nucleic acid encoding two guide RNAs, wherein the nucleic acid molecule encodes no more than two guide RNAs

In some embodiments, the single nucleic acid molecule is a single vector or comprised in a single vector. In some embodiments, the single vector expresses the two guide RNAs and Cas9. In some embodiments, a pair of guide RNAs and a Cas9 are provided on a single vector. In some embodiments, the single vector comprises a nucleic acid encoding a pair of guide RNAs and a nucleic acid encoding a SaCas9 or SluCas9. In some embodiments, two guide RNAs and a Cas9 are encoded on a single vector. In some embodiments, the single vector comprises a nucleic acid encoding a first guide RNA, a nucleic acid encoding a second guide RNA, and a nucleic acid encoding a SaCas9 or SluCas9. In some embodiments, the spacer sequences of the first and second guide RNAs are identical. In some embodiments, the spacer sequences of the first and second guide RNAs are not identical.

In some embodiments, the first nucleic acid molecule encodes for a Cas9 molecule and also encodes for one or more copies of a first guide RNA and one or more copies of a second guide RNA. In some embodiments, the first nucleic acid molecule encodes for a Cas9 molecule, but does not encode for any guide RNAs. In some embodiments, the second nucleic acid molecule encodes for one or more copies of a first guide RNA and one or more copies of a second guide RNA, wherein the second nucleic acid molecule does not encode for a Cas9 molecule.

In some embodiments, a composition is provided comprising at least one guide RNA (e.g., a pair of guide RNAs), or nucleic acid encoding at least one guide RNA (e.g., a pair of guide RNAs), wherein the guide RNA comprises or consists of i) any one or more of the following guide sequences; ii) at least 16, 17, 18, 19, or 20 contiguous nucleotides of any one or more of the following guide sequences; or iii) at least 70%, at least 75%, at least 80%, at least 85%, or at least 90% identical to a guide sequence comprising at least 16, 17, 18, 19, or 20 nucleotides of any one or more of the following guide sequences: SEQ ID NO: 1000; SEQ ID NO: 1001; SEQ ID NO: 1002; SEQ ID NO: 1003; SEQ ID NO: 1004; SEQ ID NO: 1005; SEQ ID NO: 1006; SEQ ID NO: 1007; SEQ ID NO: 1008; SEQ ID NO: 1009; SEQ ID NO: 1010; SEQ ID NO: 1011; SEQ ID NO: 1012; SEQ ID NO: 1013; SEQ ID NO: 1014; SEQ ID NO: 1015; SEQ ID NO: 1016; SEQ ID NO: 1017; SEQ ID NO: 1018; SEQ ID NO: 1019; SEQ ID NO: 1020; SEQ ID NO: 1021; SEQ ID NO: 1022; SEQ ID NO: 1023; SEQ ID NO: 1024; SEQ ID NO: 1025; SEQ ID NO: 1026; SEQ ID NO: 1027; SEQ ID NO: 1028; SEQ ID NO: 1029; SEQ ID NO: 1030; SEQ ID NO: 1031; SEQ ID NO: 1032; SEQ ID NO: 1033; SEQ ID NO: 1034; SEQ ID NO: 1035; SEQ ID NO: 1036; SEQ ID NO: 1037; SEQ ID NO: 1038; SEQ ID NO: 1039; SEQ ID NO: 1040; SEQ ID NO: 1041; SEQ ID NO: 1042; SEQ ID NO: 1043; SEQ ID NO: 1044; SEQ ID NO: 1045; SEQ ID NO: 1046; SEQ ID NO: 1047; SEQ ID NO: 1048; SEQ ID NO: 1049; SEQ ID NO: 1050; SEQ ID NO: 1051; SEQ ID NO: 1052; SEQ ID NO: 1053; SEQ ID NO: 1054; SEQ ID NO: 1055; SEQ ID NO: 1056; SEQ ID NO: 1057; SEQ ID NO: 1058; SEQ ID NO: 1059; SEQ ID NO: 1060; SEQ ID NO: 1061; SEQ ID NO: 1062; SEQ ID NO: 1063; SEQ ID NO: 1064; SEQ ID NO: 1065; SEQ ID NO: 1066; SEQ ID NO: 1067; SEQ ID NO: 1068; SEQ ID NO: 1069; SEQ ID NO: 1070; SEQ ID NO: 1071; SEQ ID NO: 1072; SEQ ID NO: 1073; SEQ ID NO: 1074; SEQ ID NO: 1075; SEQ ID NO: 1076; SEQ ID NO: 1077; SEQ ID NO: 1078; SEQ ID NO: 1079; SEQ ID NO: 1080; SEQ ID NO: 1081; SEQ ID NO: 1082; SEQ ID NO: 1083; SEQ ID NO: 1084; SEQ ID NO: 1085; SEQ ID NO: 1086; SEQ ID NO: 1087; SEQ ID NO: 1088; SEQ ID NO: 1089; SEQ ID NO: 1090; SEQ ID NO: 1091; SEQ ID NO: 1092; SEQ ID NO: 1093; SEQ ID NO: 1094; SEQ ID NO: 1095; SEQ ID NO: 1096; SEQ ID NO: 1097; SEQ ID NO: 1098; SEQ ID NO: 1099; SEQ ID NO: 1100; SEQ ID NO: 1101; SEQ ID NO: 1102; SEQ ID NO: 1103; SEQ ID NO: 1104; SEQ ID NO: 1105; SEQ ID NO: 1106; SEQ ID NO: 1107; SEQ ID NO: 1108; SEQ ID NO: 1109; SEQ ID NO: 1110; SEQ ID NO: 1111; SEQ ID NO: 1112; SEQ ID NO: 1113; SEQ ID NO: 1114; SEQ ID NO: 1115; SEQ ID NO: 1116; SEQ ID NO: 1117; SEQ ID NO: 1118; SEQ ID NO: 1119; SEQ ID NO: 1120; SEQ ID NO: 1121; SEQ ID NO: 1122; SEQ ID NO: 1123; SEQ ID NO: 1124; SEQ ID NO: 1125; SEQ ID NO: 1126; SEQ ID NO: 1127; SEQ ID NO: 1128; SEQ ID NO: 1129; SEQ ID NO: 1130; SEQ ID NO: 1131; SEQ ID NO: 1132; SEQ ID NO: 1133; SEQ ID NO: 1134; SEQ ID NO: 1135; SEQ ID NO: 1136; SEQ ID NO: 1137; SEQ ID NO: 1138; SEQ ID NO: 1139; SEQ ID NO: 1140; SEQ ID NO: 1141; SEQ ID NO: 1142; SEQ ID NO: 1143; SEQ ID NO: 1144; SEQ ID NO: 1145; SEQ ID NO: 1146; SEQ ID NO: 1147; SEQ ID NO: 1148; SEQ ID NO: 1149; SEQ ID NO: 1150; SEQ ID NO: 1151; SEQ ID NO: 1152; SEQ ID NO: 1153; SEQ ID NO: 1154; SEQ ID NO: 1155; SEQ ID NO: 1156; SEQ ID NO: 1157; SEQ ID NO: 1158; SEQ ID NO: 1159; SEQ ID NO: 1160; SEQ ID NO: 1161; SEQ ID NO: 1162; SEQ ID NO: 1163; SEQ ID NO: 1164; SEQ ID NO: 1165; SEQ ID NO: 1166; SEQ ID NO: 1167; SEQ ID NO: 1168; SEQ ID NO: 1169; SEQ ID NO: 1170; SEQ ID NO: 1171; SEQ ID NO: 1172; SEQ ID NO: 1173; SEQ ID NO: 1174; SEQ ID NO: 1175; SEQ ID NO: 1176; SEQ ID NO: 1177; SEQ ID NO: 1178; SEQ ID NO: 1179; SEQ ID NO: 1180; SEQ ID NO: 1181; SEQ ID NO: 1182; SEQ ID NO: 1183; SEQ ID NO: 1184; SEQ ID NO: 1185; SEQ ID NO: 1186; SEQ ID NO: 1187; SEQ ID NO: 1188; SEQ ID NO: 1189; SEQ ID NO: 1190; SEQ ID NO: 1191; SEQ ID NO: 1192; SEQ ID NO: 1193; SEQ ID NO: 1194; SEQ ID NO: 1195; SEQ ID NO: 1196; SEQ ID NO: 1197; SEQ ID NO: 1198; SEQ ID NO: 1199; SEQ ID NO: 1200; SEQ ID NO: 1201; SEQ ID NO: 1202; SEQ ID NO: 1203; SEQ ID NO: 1204; SEQ ID NO: 1205; SEQ ID NO: 1206; SEQ ID NO: 1207; SEQ ID NO: 1208; SEQ ID NO: 1209; SEQ ID NO: 1210; SEQ ID NO: 1211; SEQ ID NO: 1212; SEQ ID NO: 1213; SEQ ID NO: 1214; SEQ ID NO: 1215; SEQ ID NO: 1216; SEQ ID NO: 1217; SEQ ID NO: 1218; SEQ ID NO: 1219; SEQ ID NO: 1220; SEQ ID NO: 1221; SEQ ID NO: 1222; SEQ ID NO: 1223; SEQ ID NO: 1224; SEQ ID NO: 1225; SEQ ID NO: 1226; SEQ ID NO: 1227; SEQ ID NO: 1228; SEQ ID NO: 1229; SEQ ID NO: 1230; SEQ ID NO: 1231; SEQ ID NO: 1232; SEQ ID NO: 1233; SEQ ID NO: 1234; SEQ ID NO: 1235; SEQ ID NO: 1236; SEQ ID NO: 1237; SEQ ID NO: 1238; SEQ ID NO: 1239; SEQ ID NO: 1240; SEQ ID NO: 1241; SEQ ID NO: 1242; SEQ ID NO: 1243; SEQ ID NO: 1244; SEQ ID NO: 1245; SEQ ID NO: 1246; SEQ ID NO: 1247; SEQ ID NO: 1248; SEQ ID NO: 1249; SEQ ID NO: 1250; SEQ ID NO: 1251; SEQ ID NO: 1252; SEQ ID NO: 1253; SEQ ID NO: 1254; SEQ ID NO: 1255; SEQ ID NO: 1256; SEQ ID NO: 1257; SEQ ID NO: 1258; SEQ ID NO: 1259; SEQ ID NO: 1260; SEQ ID NO: 1261; SEQ ID NO: 1262; SEQ ID NO: 1263; SEQ ID NO: 1264; SEQ ID NO: 1265; SEQ ID NO: 1266; SEQ ID NO: 1267; SEQ ID NO: 1268; SEQ ID NO: 1269; SEQ ID NO: 1270; SEQ ID NO: 1271; SEQ ID NO: 1272; SEQ ID NO: 1273; SEQ ID NO: 1274; SEQ ID NO: 1275; SEQ ID NO: 1276; SEQ ID NO: 1277; SEQ ID NO: 1278; SEQ ID NO: 1279; SEQ ID NO: 1280; SEQ ID NO: 1281; SEQ ID NO: 1282; SEQ ID NO: 1283; SEQ ID NO: 1284; SEQ ID NO: 1285; SEQ ID NO: 1286; SEQ ID NO: 1287; SEQ ID NO: 1288; SEQ ID NO: 1289; SEQ ID NO: 1290; SEQ ID NO: 1291; SEQ ID NO: 1292; SEQ ID NO: 1293; SEQ ID NO: 1294; SEQ ID NO: 1295; SEQ ID NO: 1296; SEQ ID NO: 1297; SEQ ID NO: 1298; SEQ ID NO: 1299; SEQ ID NO: 1300; SEQ ID NO: 1301; SEQ ID NO: 1302; SEQ ID NO: 1303; SEQ ID NO: 1304; SEQ ID NO: 1305; SEQ ID NO: 1306; SEQ ID NO: 1307; SEQ ID NO: 1308; SEQ ID NO: 1309; SEQ ID NO: 1310; SEQ ID NO: 1311; SEQ ID NO: 1312; SEQ ID NO: 1313; SEQ ID NO: 1314; SEQ ID NO: 1315; SEQ ID NO: 1316; SEQ ID NO: 1317; SEQ ID NO: 1318; SEQ ID NO: 1319; SEQ ID NO: 1320; SEQ ID NO: 1321; SEQ ID NO: 1322; SEQ ID NO: 1323; SEQ ID NO: 1324; SEQ ID NO: 1325; SEQ ID NO: 1326; SEQ ID NO: 1327; SEQ ID NO: 1328; SEQ ID NO: 1329; SEQ ID NO: 1330; SEQ ID NO: 1331; SEQ ID NO: 1332; SEQ ID NO: 1333; SEQ ID NO: 1334; SEQ ID NO: 1335; SEQ ID NO: 1336; SEQ ID NO: 1337; SEQ ID NO: 1338; SEQ ID NO: 1339; SEQ ID NO: 1340; SEQ ID NO: 1341; SEQ ID NO: 1342; SEQ ID NO: 1343; SEQ ID NO: 1344; SEQ ID NO: 1345; SEQ ID NO: 1346; SEQ ID NO: 1347; SEQ ID NO: 1348; SEQ ID NO: 1349; SEQ ID NO: 1350; SEQ ID NO: 1351; SEQ ID NO: 1352; SEQ ID NO: 1353; SEQ ID NO: 1354; SEQ ID NO: 1355; SEQ ID NO: 1356; SEQ ID NO: 1357; SEQ ID NO: 1358; SEQ ID NO: 1359; SEQ ID NO: 1360; SEQ ID NO: 1361; SEQ ID NO: 1362; SEQ ID NO: 1363; SEQ ID NO: 1364; SEQ ID NO: 1365; SEQ ID NO: 1366; SEQ ID NO: 1367; SEQ ID NO: 1368; SEQ ID NO: 1369; SEQ ID NO: 1370; SEQ ID NO: 1371; SEQ ID NO: 1372; SEQ ID NO: 1373; SEQ ID NO: 1374; SEQ ID NO: 1375; SEQ ID NO: 1376; SEQ ID NO: 1377; SEQ ID NO: 1378; SEQ ID NO: 1379; SEQ ID NO: 1380; SEQ ID NO: 1381; SEQ ID NO: 1382; SEQ ID NO: 1383; SEQ ID NO: 1384; SEQ ID NO: 1385; SEQ ID NO: 1386; SEQ ID NO: 1387; SEQ ID NO: 1388; SEQ ID NO: 1389; SEQ ID NO: 1390; SEQ ID NO: 1391; SEQ ID NO: 1392; SEQ ID NO: 1393; SEQ ID NO: 1394; SEQ ID NO: 1395; SEQ ID NO: 1396; SEQ ID NO: 1397; SEQ ID NO: 1398; SEQ ID NO: 1399; SEQ ID NO: 1400; SEQ ID NO: 1401; SEQ ID NO: 1402; SEQ ID NO: 1403; SEQ ID NO: 1404; SEQ ID NO: 1405; SEQ ID NO: 1406; SEQ ID NO: 1407; SEQ ID NO: 1408; SEQ ID NO: 1409; SEQ ID NO: 1410; SEQ ID NO: 1411; SEQ ID NO: 1412; SEQ ID NO: 1413; SEQ ID NO: 1414; SEQ ID NO: 1415; SEQ ID NO: 1416; SEQ ID NO: 1417; SEQ ID NO: 1418; SEQ ID NO: 1419; SEQ ID NO: 1420; SEQ ID NO: 1421; SEQ ID NO: 1422; SEQ ID NO: 1423; SEQ ID NO: 1424; SEQ ID NO: 1425; SEQ ID NO: 1426; SEQ ID NO: 1427; SEQ ID NO: 1428; SEQ ID NO: 1429; SEQ ID NO: 1430; SEQ ID NO: 1431; SEQ ID NO: 1432; SEQ ID NO: 1433; SEQ ID NO: 1434; SEQ ID NO: 1435; SEQ ID NO: 1436; SEQ ID NO: 1437; SEQ ID NO: 1438; SEQ ID NO: 1439; SEQ ID NO: 1440; SEQ ID NO: 1441; SEQ ID NO: 1442; SEQ ID NO: 1443; SEQ ID NO: 1444; SEQ ID NO: 1445; SEQ ID NO: 1446; SEQ ID NO: 1447; SEQ ID NO: 1448; SEQ ID NO: 1449; SEQ ID NO: 1450; SEQ ID NO: 1451; SEQ ID NO: 1452; SEQ ID NO: 1453; SEQ ID NO: 1454; SEQ ID NO: 1455; SEQ ID NO: 1456; SEQ ID NO: 1457; SEQ ID NO: 1458; SEQ ID NO: 1459; SEQ ID NO: 1460; SEQ ID NO: 1461; SEQ ID NO: 1462; SEQ ID NO: 1463; SEQ ID NO: 1464; SEQ ID NO: 1465; SEQ ID NO: 1466; SEQ ID NO: 1467; SEQ ID NO: 1468; SEQ ID NO: 1469; SEQ ID NO: 1470; SEQ ID NO: 1471; SEQ ID NO: 1472; SEQ ID NO: 1473; SEQ ID NO: 1474; SEQ ID NO: 1475; SEQ ID NO: 1476; SEQ ID NO: 1477; SEQ ID NO: 1478; SEQ ID NO: 1479; SEQ ID NO: 1480; SEQ ID NO: 1481; SEQ ID NO: 1482; SEQ ID NO: 1483; SEQ ID NO: 1484; SEQ ID NO: 1485; SEQ ID NO: 1486; SEQ ID NO: 1487; SEQ ID NO: 1488; SEQ ID NO: 1489; SEQ ID NO: 1490; SEQ ID NO: 1491; SEQ ID NO: 1492; SEQ ID NO: 1493; SEQ ID NO: 1494; SEQ ID NO: 1495; SEQ ID NO: 1496; SEQ ID NO: 1497; SEQ ID NO: 1498; SEQ ID NO: 1499; SEQ ID NO: 1500; SEQ ID NO: 1501; SEQ ID NO: 1502; SEQ ID NO: 1503; SEQ ID NO: 1504; SEQ ID NO: 1505; SEQ ID NO: 1506; SEQ ID NO: 1507; SEQ ID NO: 1508; SEQ ID NO: 1509; SEQ ID NO: 1510; SEQ ID NO: 1511; SEQ ID NO: 1512; SEQ ID NO: 1513; SEQ ID NO: 1514; SEQ ID NO: 1515; SEQ ID NO: 1516; SEQ ID NO: 1517; SEQ ID NO: 1518; SEQ ID NO: 1519; SEQ ID NO: 1520; SEQ ID NO: 1521; SEQ ID NO: 1522; SEQ ID NO: 1523; SEQ ID NO: 1524; SEQ ID NO: 1525; SEQ ID NO: 1526; SEQ ID NO: 1527; SEQ ID NO: 1528; SEQ ID NO: 1529; SEQ ID NO: 1530; SEQ ID NO: 1531; SEQ ID NO: 1532; SEQ ID NO: 1533; SEQ ID NO: 1534; SEQ ID NO: 1535; SEQ ID NO: 1536; SEQ ID NO: 1537; SEQ ID NO: 1538; SEQ ID NO: 1539; SEQ ID NO: 1540; SEQ ID NO: 1541; SEQ ID NO: 1542; SEQ ID NO: 1543; SEQ ID NO: 1544; SEQ ID NO: 1545; SEQ ID NO: 1546; SEQ ID NO: 1547; SEQ ID NO: 1548; SEQ ID NO: 1549; SEQ ID NO: 1550; SEQ ID NO: 1551; SEQ ID NO: 1552; SEQ ID NO: 1553; SEQ ID NO: 1554; SEQ ID NO: 1555; SEQ ID NO: 1556; SEQ ID NO: 1557; SEQ ID NO: 1558; SEQ ID NO: 1559; SEQ ID NO: 1560; SEQ ID NO: 1561; SEQ ID NO: 1562; SEQ ID NO: 1563; SEQ ID NO: 1564; SEQ ID NO: 1565; SEQ ID NO: 1566; SEQ ID NO: 1567; SEQ ID NO: 1568; SEQ ID NO: 1569; SEQ ID NO: 1570; SEQ ID NO: 1571; SEQ ID NO: 1572; SEQ ID NO: 1573; SEQ ID NO: 1574; SEQ ID NO: 1575; SEQ ID NO: 1576; SEQ ID NO: 1577; SEQ ID NO: 1578; SEQ ID NO: 1579; SEQ ID NO: 1580; SEQ ID NO: 1581; SEQ ID NO: 1582; SEQ ID NO: 1583; SEQ ID NO: 1584; SEQ ID NO: 1585; SEQ ID NO: 1586; SEQ ID NO: 1587; SEQ ID NO: 1588; SEQ ID NO: 1589; SEQ ID NO: 1590; SEQ ID NO: 1591; SEQ ID NO: 1592; SEQ ID NO: 1593; SEQ ID NO: 1594; SEQ ID NO: 1595; SEQ ID NO: 1596; SEQ ID NO: 1597; SEQ ID NO: 1598; SEQ ID NO: 1599; SEQ ID NO: 1600; SEQ ID NO: 1601; SEQ ID NO: 1602; SEQ ID NO: 1603; SEQ ID NO: 1604; SEQ ID NO: 1605; SEQ ID NO: 1606; SEQ ID NO: 1607; SEQ ID NO: 1608; SEQ ID NO: 1609; SEQ ID NO: 1610; SEQ ID NO: 1611; SEQ ID NO: 1612; SEQ ID NO: 1613; SEQ ID NO: 1614; SEQ ID NO: 1615; SEQ ID NO: 1616; SEQ ID NO: 1617; SEQ ID NO: 1618; SEQ ID NO: 1619; SEQ ID NO: 1620; SEQ ID NO: 1621; SEQ ID NO: 1622; SEQ ID NO: 1623; SEQ ID NO: 1624; SEQ ID NO: 1625; SEQ ID NO: 1626; SEQ ID NO: 1627; SEQ ID NO: 1628; SEQ ID NO: 1629; SEQ ID NO: 1630; SEQ ID NO: 1631; SEQ ID NO: 1632; SEQ ID NO: 1633; SEQ ID NO: 1634; SEQ ID NO: 1635; SEQ ID NO: 1636; SEQ ID NO: 1637; SEQ ID NO: 1638; SEQ ID NO: 1639; SEQ ID NO: 1640; SEQ ID NO: 1641; SEQ ID NO: 1642; SEQ ID NO: 1643; SEQ ID NO: 1644; SEQ ID NO: 1645; SEQ ID NO: 1646; SEQ ID NO: 1647; SEQ ID NO: 1648; SEQ ID NO: 1649; SEQ ID NO: 1650; SEQ ID NO: 1651; SEQ ID NO: 1652; SEQ ID NO: 1653; SEQ ID NO: 1654; SEQ ID NO: 1655; SEQ ID NO: 1656; SEQ ID NO: 1657; SEQ ID NO: 1658; SEQ ID NO: 1659; SEQ ID NO: 1660; SEQ ID NO: 1661; SEQ ID NO: 1662; SEQ ID NO: 1663; SEQ ID NO: 1664; SEQ ID NO: 1665; SEQ ID NO: 1666; SEQ ID NO: 1667; SEQ ID NO: 1668; SEQ ID NO: 1669; SEQ ID NO: 1670; SEQ ID NO: 1671; SEQ ID NO: 1672; SEQ ID NO: 1673; SEQ ID NO: 1674; SEQ ID NO: 1675; SEQ ID NO: 1676; SEQ ID NO: 1677; SEQ ID NO: 1678; SEQ ID NO: 1679; SEQ ID NO: 1680; SEQ ID NO: 1681; SEQ ID NO: 1682; SEQ ID NO: 1683; SEQ ID NO: 1684; SEQ ID NO: 1685; SEQ ID NO: 1686; SEQ ID NO: 1687; SEQ ID NO: 1688; SEQ ID NO: 1689; SEQ ID NO: 1690; SEQ ID NO: 1691; SEQ ID NO: 1692; SEQ ID NO: 1693; SEQ ID NO: 1694; SEQ ID NO: 1695; SEQ ID NO: 1696; SEQ ID NO: 1697; SEQ ID NO: 1698; SEQ ID NO: 1699; SEQ ID NO: 1700; SEQ ID NO: 1701; SEQ ID NO: 1702; SEQ ID NO: 1703; SEQ ID NO: 1704; SEQ ID NO: 1705; SEQ ID NO: 1706; SEQ ID NO: 1707; SEQ ID NO: 1708; SEQ ID NO: 1709; SEQ ID NO: 1710; SEQ ID NO: 1711; SEQ ID NO: 1712; SEQ ID NO: 1713; SEQ ID NO: 1714; SEQ ID NO: 1715; SEQ ID NO: 1716; SEQ ID NO: 1717; SEQ ID NO: 1718; SEQ ID NO: 1719; SEQ ID NO: 1720; SEQ ID NO: 1721; SEQ ID NO: 1722; SEQ ID NO: 1723; SEQ ID NO: 1724; SEQ ID NO: 1725; SEQ ID NO: 1726; SEQ ID NO: 1727; SEQ ID NO: 1728; SEQ ID NO: 1729; SEQ ID NO: 1730; SEQ ID NO: 1731; SEQ ID NO: 1732; SEQ ID NO: 1733; SEQ ID NO: 1734; SEQ ID NO: 1735; SEQ ID NO: 1736; SEQ ID NO: 1737; SEQ ID NO: 1738; SEQ ID NO: 1739; SEQ ID NO: 1740; SEQ ID NO: 1741; SEQ ID NO: 1742; SEQ ID NO: 1743; SEQ ID NO: 1744; SEQ ID NO: 1745; SEQ ID NO: 1746; SEQ ID NO: 1747; SEQ ID NO: 1748; SEQ ID NO: 1749; SEQ ID NO: 1750; SEQ ID NO: 1751; SEQ ID NO: 1752; SEQ ID NO: 1753; SEQ ID NO: 1754; SEQ ID NO: 1755; SEQ ID NO: 1756; SEQ ID NO: 1757; SEQ ID NO: 1758; SEQ ID NO: 1759; SEQ ID NO: 1760; SEQ ID NO: 1761; SEQ ID NO: 1762; SEQ ID NO: 1763; SEQ ID NO: 1764; SEQ ID NO: 1765; SEQ ID NO: 1766; SEQ ID NO: 1767; SEQ ID NO: 1768; SEQ ID NO: 1769; SEQ ID NO: 1770; SEQ ID NO: 1771; SEQ ID NO: 1772; SEQ ID NO: 1773; SEQ ID NO: 1774; SEQ ID NO: 1775; SEQ ID NO: 1776; SEQ ID NO: 1777; SEQ ID NO: 1778; SEQ ID NO: 1779; SEQ ID NO: 1780; SEQ ID NO: 1781; SEQ ID NO: 1782; SEQ ID NO: 1783; SEQ ID NO: 1784; SEQ ID NO: 1785; SEQ ID NO: 1786; SEQ ID NO: 1787; SEQ ID NO: 1788; SEQ ID NO: 1789; SEQ ID NO: 1790; SEQ ID NO: 1791; SEQ ID NO: 1792; SEQ ID NO: 1793; SEQ ID NO: 1794; SEQ ID NO: 1795; SEQ ID NO: 1796; SEQ ID NO: 1797; SEQ ID NO: 1798; SEQ ID NO: 1799; SEQ ID NO: 1800; SEQ ID NO: 1801; SEQ ID NO: 1802; SEQ ID NO: 1803; SEQ ID NO: 1804; SEQ ID NO: 1805; SEQ ID NO: 1806; SEQ ID NO: 1807; SEQ ID NO: 1808; SEQ ID NO: 1809; SEQ ID NO: 1810; SEQ ID NO: 1811; SEQ ID NO: 1812; SEQ ID NO: 1813; SEQ ID NO: 1814; SEQ ID NO: 1815; SEQ ID NO: 1816; SEQ ID NO: 1817; SEQ ID NO: 1818; SEQ ID NO: 1819; SEQ ID NO: 1820; SEQ ID NO: 1821; SEQ ID NO: 1822; SEQ ID NO: 1823; SEQ ID NO: 1824; SEQ ID NO: 1825; SEQ ID NO: 1826; SEQ ID NO: 1827; SEQ ID NO: 1828; SEQ ID NO: 1829; SEQ ID NO: 1830; SEQ ID NO: 1831; SEQ ID NO: 1832; SEQ ID NO: 1833; SEQ ID NO: 1834; SEQ ID NO: 1835; SEQ ID NO: 1836; SEQ ID NO: 1837; SEQ ID NO: 1838; SEQ ID NO: 1839; SEQ ID NO: 1840; SEQ ID NO: 1841; SEQ ID NO: 1842; SEQ ID NO: 1843; SEQ ID NO: 1844; SEQ ID NO: 1845; SEQ ID NO: 1846; SEQ ID NO: 1847; SEQ ID NO: 1848; SEQ ID NO: 1849; SEQ ID NO: 1850; SEQ ID NO: 1851; SEQ ID NO: 1852; SEQ ID NO: 1853; SEQ ID NO: 1854; SEQ ID NO: 1855; SEQ ID NO: 1856; SEQ ID NO: 1857; SEQ ID NO: 1858; SEQ ID NO: 1859; SEQ ID NO: 1860; SEQ ID NO: 1861; SEQ ID NO: 1862; SEQ ID NO: 1863; SEQ ID NO: 1864; SEQ ID NO: 1865; SEQ ID NO: 1866; SEQ ID NO: 1867; SEQ ID NO: 1868; SEQ ID NO: 1869; SEQ ID NO: 1870; SEQ ID NO: 1871; SEQ ID NO: 1872; SEQ ID NO: 1873; SEQ ID NO: 1874; SEQ ID NO: 1875; SEQ ID NO: 1876; SEQ ID NO: 1877; SEQ ID NO: 1878; SEQ ID NO: 1879; SEQ ID NO: 1880; SEQ ID NO: 1881; SEQ ID NO: 1882; SEQ ID NO: 1883; SEQ ID NO: 1884; SEQ ID NO: 1885; SEQ ID NO: 1886; SEQ ID NO: 1887; SEQ ID NO: 1888; SEQ ID NO: 1889; SEQ ID NO: 1890; SEQ ID NO: 1891; SEQ ID NO: 1892; SEQ ID NO: 1893; SEQ ID NO: 1894; SEQ ID NO: 1895; SEQ ID NO: 1896; SEQ ID NO: 1897; SEQ ID NO: 1898; SEQ ID NO: 1899; SEQ ID NO: 1900; SEQ ID NO: 1901; SEQ ID NO: 1902; SEQ ID NO: 1903; SEQ ID NO: 1904; SEQ ID NO: 1905; SEQ ID NO: 1906; SEQ ID NO: 1907; SEQ ID NO: 1908; SEQ ID NO: 1909; SEQ ID NO: 1910; SEQ ID NO: 1911; SEQ ID NO: 1912; SEQ ID NO: 1913; SEQ ID NO: 1914; SEQ ID NO: 1915; SEQ ID NO: 1916; SEQ ID NO: 1917; SEQ ID NO: 1918; SEQ ID NO: 1919; SEQ ID NO: 1920; SEQ ID NO: 1921; SEQ ID NO: 1922; SEQ ID NO: 1923; SEQ ID NO: 1924; SEQ ID NO: 1925; SEQ ID NO: 1926; SEQ ID NO: 1927; SEQ ID NO: 1928; SEQ ID NO: 1929; SEQ ID NO: 1930; SEQ ID NO: 1931; SEQ ID NO: 1932; SEQ ID NO: 1933; SEQ ID NO: 1934; SEQ ID NO: 1935; SEQ ID NO: 1936; SEQ ID NO: 1937; SEQ ID NO: 1938; SEQ ID NO: 1939; SEQ ID NO: 1940; SEQ ID NO: 1941; SEQ ID NO: 1942; SEQ ID NO: 1943; SEQ ID NO: 1944; SEQ ID NO: 1945; SEQ ID NO: 1946; SEQ ID NO: 1947; SEQ ID NO: 1948; SEQ ID NO: 1949; SEQ ID NO: 1950; SEQ ID NO: 1951; SEQ ID NO: 1952; SEQ ID NO: 1953; SEQ ID NO: 1954; SEQ ID NO: 1955; SEQ ID NO: 1956; SEQ ID NO: 1957; SEQ ID NO: 1958; SEQ ID NO: 1959; SEQ ID NO: 1960; SEQ ID NO: 1961; SEQ ID NO: 1962; SEQ ID NO: 1963; SEQ ID NO: 1964; SEQ ID NO: 1965; SEQ ID NO: 1966; SEQ ID NO: 1967; SEQ ID NO: 1968; SEQ ID NO: 1969; SEQ ID NO: 1970; SEQ ID NO: 1971; SEQ ID NO: 1972; SEQ ID NO: 1973; SEQ ID NO: 1974; SEQ ID NO: 1975; SEQ ID NO: 1976; SEQ ID NO: 1977; SEQ ID NO: 1978; SEQ ID NO: 1979; SEQ ID NO: 1980; SEQ ID NO: 1981; SEQ ID NO: 1982; SEQ ID NO: 1983; SEQ ID NO: 1984; SEQ ID NO: 1985; SEQ ID NO: 1986; SEQ ID NO: 1987; SEQ ID NO: 1988; SEQ ID NO: 1989; SEQ ID NO: 1990; SEQ ID NO: 1991; SEQ ID NO: 1992; SEQ ID NO: 1993; SEQ ID NO: 1994; SEQ ID NO: 1995; SEQ ID NO: 1996; SEQ ID NO: 1997; SEQ ID NO: 1998; SEQ ID NO: 1999; SEQ ID NO: 2000; SEQ ID NO: 2001; SEQ ID NO: 2002; SEQ ID NO: 2003; SEQ ID NO: 2004; SEQ ID NO: 2005; SEQ ID NO: 2006; SEQ ID NO: 2007; SEQ ID NO: 2008; SEQ ID NO: 2009; SEQ ID NO: 2010; SEQ ID NO: 2011; SEQ ID NO: 2012; SEQ ID NO: 2013; SEQ ID NO: 2014; SEQ ID NO: 2015; SEQ ID NO: 2016; SEQ ID NO: 2017; SEQ ID NO: 2018; SEQ ID NO: 2019; SEQ ID NO: 2020; SEQ ID NO: 2021; SEQ ID NO: 2022; SEQ ID NO: 2023; SEQ ID NO: 2024; SEQ ID NO: 2025; SEQ ID NO: 2026; SEQ ID NO: 2027; SEQ ID NO: 2028; SEQ ID NO: 2029; SEQ ID NO: 2030; SEQ ID NO: 2031; SEQ ID NO: 2032; SEQ ID NO: 2033; SEQ ID NO: 2034; SEQ ID NO: 2035; SEQ ID NO: 2036; SEQ ID NO: 2037; SEQ ID NO: 2038; SEQ ID NO: 2039; SEQ ID NO: 2040; SEQ ID NO: 2041; SEQ ID NO: 2042; SEQ ID NO: 2043; SEQ ID NO: 2044; SEQ ID NO: 2045; SEQ ID NO: 2046; SEQ ID NO: 2047; SEQ ID NO: 2048; SEQ ID NO: 2049; SEQ ID NO: 2050; SEQ ID NO: 2051; SEQ ID NO: 2052; SEQ ID NO: 2053; SEQ ID NO: 2054; SEQ ID NO: 2055; SEQ ID NO: 2056; SEQ ID NO: 2057; SEQ ID NO: 2058; SEQ ID NO: 2059; SEQ ID NO: 2060; SEQ ID NO: 2061; SEQ ID NO: 2062; SEQ ID NO: 2063; SEQ ID NO: 2064; SEQ ID NO: 2065; SEQ ID NO: 2066; SEQ ID NO: 2067; SEQ ID NO: 2068; SEQ ID NO: 2069; SEQ ID NO: 2070; SEQ ID NO: 2071; SEQ ID NO: 2072; SEQ ID NO: 2073; SEQ ID NO: 2074; SEQ ID NO: 2075; SEQ ID NO: 2076; SEQ ID NO: 2077; SEQ ID NO: 2078; SEQ ID NO: 2079; SEQ ID NO: 2080; SEQ ID NO: 2081; SEQ ID NO: 2082; SEQ ID NO: 2083; SEQ ID NO: 2084; SEQ ID NO: 2085; SEQ ID NO: 2086; SEQ ID NO: 2087; SEQ ID NO: 2088; SEQ ID NO: 2089; SEQ ID NO: 2090; SEQ ID NO: 2091; SEQ ID NO: 2092; SEQ ID NO: 2093; SEQ ID NO: 2094; SEQ ID NO: 2095; SEQ ID NO: 2096; SEQ ID NO: 2097; SEQ ID NO: 2098; SEQ ID NO: 2099; SEQ ID NO: 2100; SEQ ID NO: 2101; SEQ ID NO: 2102; SEQ ID NO: 2103; SEQ ID NO: 2104; SEQ ID NO: 2105; SEQ ID NO: 2106; SEQ ID NO: 2107; SEQ ID NO: 2108; SEQ ID NO: 2109; SEQ ID NO: 2110; SEQ ID NO: 2111; SEQ ID NO: 2112; SEQ ID NO: 2113; SEQ ID NO: 2114; SEQ ID NO: 2115; SEQ ID NO: 2116; SEQ ID NO: 2117; SEQ ID NO: 2118; SEQ ID NO: 2119; SEQ ID NO: 2120; SEQ ID NO: 2121; SEQ ID NO: 2122; SEQ ID NO: 2123; SEQ ID NO: 2124; SEQ ID NO: 2125; SEQ ID NO: 2126; SEQ ID NO: 2127; SEQ ID NO: 2128; SEQ ID NO: 2129; SEQ ID NO: 2130; SEQ ID NO: 2131; SEQ ID NO: 2132; SEQ ID NO: 2133; SEQ ID NO: 2134; SEQ ID NO: 2135; SEQ ID NO: 2136; SEQ ID NO: 2137; SEQ ID NO: 2138; SEQ ID NO: 2139; SEQ ID NO: 2140; SEQ ID NO: 2141; SEQ ID NO: 2142; SEQ ID NO: 2143; SEQ ID NO: 2144; SEQ ID NO: 2145; SEQ ID NO: 2146; SEQ ID NO: 2147; SEQ ID NO: 2148; SEQ ID NO: 2149; SEQ ID NO: 2150; SEQ ID NO: 2151; SEQ ID NO: 2152; SEQ ID NO: 2153; SEQ ID NO: 2154; SEQ ID NO: 2155; SEQ ID NO: 2156; SEQ ID NO: 2157; SEQ ID NO: 2158; SEQ ID NO: 2159; SEQ ID NO: 2160; SEQ ID NO: 2161; SEQ ID NO: 2162; SEQ ID NO: 2163; SEQ ID NO: 2164; SEQ ID NO: 2165; SEQ ID NO: 2166; SEQ ID NO: 2167; SEQ ID NO: 2168; SEQ ID NO: 2169; SEQ ID NO: 2170; SEQ ID NO: 2171; SEQ ID NO: 2172; SEQ ID NO: 2173; SEQ ID NO: 2174; SEQ ID NO: 2175; SEQ ID NO: 2176; SEQ ID NO: 2177; SEQ ID NO: 2178; SEQ ID NO: 2179; SEQ ID NO: 2180; SEQ ID NO: 2181; SEQ ID NO: 2182; SEQ ID NO: 2183; SEQ ID NO: 2184; SEQ ID NO: 2185; SEQ ID NO: 2186; SEQ ID NO: 2187; SEQ ID NO: 2188; SEQ ID NO: 2189; SEQ ID NO: 2190; SEQ ID NO: 2191; SEQ ID NO: 2192; SEQ ID NO: 2193; SEQ ID NO: 2194; SEQ ID NO: 2195; SEQ ID NO: 2196; SEQ ID NO: 2197; SEQ ID NO: 2198; SEQ ID NO: 2199; SEQ ID NO: 2200; SEQ ID NO: 2201; SEQ ID NO: 2202; SEQ ID NO: 2203; SEQ ID NO: 2204; SEQ ID NO: 2205; SEQ ID NO: 2206; SEQ ID NO: 2207; SEQ ID NO: 2208; SEQ ID NO: 2209; SEQ ID NO: 2210; SEQ ID NO: 2211; SEQ ID NO: 2212; SEQ ID NO: 2213; SEQ ID NO: 2214; SEQ ID NO: 2215; SEQ ID NO: 2216; SEQ ID NO: 2217; SEQ ID NO: 2218; SEQ ID NO: 2219; SEQ ID NO: 2220; SEQ ID NO: 2221; SEQ ID NO: 2222; SEQ ID NO: 2223; SEQ ID NO: 2224; SEQ ID NO: 2225; SEQ ID NO: 2226; SEQ ID NO: 2227; SEQ ID NO: 2228; SEQ ID NO: 2229; SEQ ID NO: 2230; SEQ ID NO: 2231; SEQ ID NO: 2232; SEQ ID NO: 2233; SEQ ID NO: 2234; SEQ ID NO: 2235; SEQ ID NO: 2236; SEQ ID NO: 2237; SEQ ID NO: 2238; SEQ ID NO: 2239; SEQ ID NO: 2240; SEQ ID NO: 2241; SEQ ID NO: 2242; SEQ ID NO: 2243; SEQ ID NO: 2244; SEQ ID NO: 2245; SEQ ID NO: 2246; SEQ ID NO: 2247; SEQ ID NO: 2248; SEQ ID NO: 2249; SEQ ID NO: 2250; SEQ ID NO: 2251; SEQ ID NO: 2252; SEQ ID NO: 2253; SEQ ID NO: 2254; SEQ ID NO: 2255; SEQ ID NO: 2256; SEQ ID NO: 2257; SEQ ID NO: 2258; SEQ ID NO: 2259; SEQ ID NO: 2260; SEQ ID NO: 2261; SEQ ID NO: 2262; SEQ ID NO: 2263; SEQ ID NO: 2264; SEQ ID NO: 2265; SEQ ID NO: 2266; SEQ ID NO: 2267; SEQ ID NO: 2268; SEQ ID NO: 2269; SEQ ID NO: 2270; SEQ ID NO: 2271; SEQ ID NO: 2272; SEQ ID NO: 2273; SEQ ID NO: 2274; SEQ ID NO: 2275; SEQ ID NO: 2276; SEQ ID NO: 2277; SEQ ID NO: 2278; SEQ ID NO: 2279; SEQ ID NO: 2280; SEQ ID NO: 2281; SEQ ID NO: 2282; SEQ ID NO: 2283; SEQ ID NO: 2284; SEQ ID NO: 2285; SEQ ID NO: 2286; SEQ ID NO: 2287; SEQ ID NO: 2288; SEQ ID NO: 2289; SEQ ID NO: 2290; SEQ ID NO: 2291; SEQ ID NO: 2292; SEQ ID NO: 2293; SEQ ID NO: 2294; SEQ ID NO: 2295; SEQ ID NO: 2296; SEQ ID NO: 2297; SEQ ID NO: 2298; SEQ ID NO: 2299; SEQ ID NO: 2300; SEQ ID NO: 2301; SEQ ID NO: 2302; SEQ ID NO: 2303; SEQ ID NO: 2304; SEQ ID NO: 2305; SEQ ID NO: 2306; SEQ ID NO: 2307; SEQ ID NO: 2308; SEQ ID NO: 2309; SEQ ID NO: 2310; SEQ ID NO: 2311; SEQ ID NO: 2312; SEQ ID NO: 2313; SEQ ID NO: 2314; SEQ ID NO: 2315; SEQ ID NO: 2316; SEQ ID NO: 2317; SEQ ID NO: 2318; SEQ ID NO: 2319; SEQ ID NO: 2320; SEQ ID NO: 2321; SEQ ID NO: 2322; SEQ ID NO: 2323; SEQ ID NO: 2324; SEQ ID NO: 2325; SEQ ID NO: 2326; SEQ ID NO: 2327; SEQ ID NO: 2328; SEQ ID NO: 2329; SEQ ID NO: 2330; SEQ ID NO: 2331; SEQ ID NO: 2332; SEQ ID NO: 2333; SEQ ID NO: 2334; SEQ ID NO: 2335; SEQ ID NO: 2336; SEQ ID NO: 2337; SEQ ID NO: 2338; SEQ ID NO: 2339; SEQ ID NO: 2340; SEQ ID NO: 2341; SEQ ID NO: 2342; SEQ ID NO: 2343; SEQ ID NO: 2344; SEQ ID NO: 2345; SEQ ID NO: 2346; SEQ ID NO: 2347; SEQ ID NO: 2348; SEQ ID NO: 2349; SEQ ID NO: 2350; SEQ ID NO: 2351; SEQ ID NO: 2352; SEQ ID NO: 2353; SEQ ID NO: 2354; SEQ ID NO: 2355; SEQ ID NO: 2356; SEQ ID NO: 2357; SEQ ID NO: 2358; SEQ ID NO: 2359; SEQ ID NO: 2360; SEQ ID NO: 2361; SEQ ID NO: 2362; SEQ ID NO: 2363; SEQ ID NO: 2364; SEQ ID NO: 2365; SEQ ID NO: 2366; SEQ ID NO: 2367; SEQ ID NO: 2368; SEQ ID NO: 2369; SEQ ID NO: 2370; SEQ ID NO: 2371; SEQ ID NO: 2372; SEQ ID NO: 2373; SEQ ID NO: 2374; SEQ ID NO: 2375; SEQ ID NO: 2376; SEQ ID NO: 2377; SEQ ID NO: 2378; SEQ ID NO: 2379; SEQ ID NO: 2380; SEQ ID NO: 2381; SEQ ID NO: 2382; SEQ ID NO: 2383; SEQ ID NO: 2384; SEQ ID NO: 2385; SEQ ID NO: 2386; SEQ ID NO: 2387; SEQ ID NO: 2388; SEQ ID NO: 2389; SEQ ID NO: 2390; SEQ ID NO: 2391; SEQ ID NO: 2392; SEQ ID NO: 2393; SEQ ID NO: 2394; SEQ ID NO: 2395; SEQ ID NO: 2396; SEQ ID NO: 2397; SEQ ID NO: 2398; SEQ ID NO: 2399; SEQ ID NO: 2400; SEQ ID NO: 2401; SEQ ID NO: 2402; SEQ ID NO: 2403; SEQ ID NO: 2404; SEQ ID NO: 2405; SEQ ID NO: 2406; SEQ ID NO: 2407; SEQ ID NO: 2408; SEQ ID NO: 2409; SEQ ID NO: 2410; SEQ ID NO: 2411; SEQ ID NO: 2412; SEQ ID NO: 2413; SEQ ID NO: 2414; SEQ ID NO: 2415; SEQ ID NO: 2416; SEQ ID NO: 2417; SEQ ID NO: 2418; SEQ ID NO: 2419; SEQ ID NO: 2420; SEQ ID NO: 2421; SEQ ID NO: 2422; SEQ ID NO: 2423; SEQ ID NO: 2424; SEQ ID NO: 2425; SEQ ID NO: 2426; SEQ ID NO: 2427; SEQ ID NO: 2428; SEQ ID NO: 2429; SEQ ID NO: 2430; SEQ ID NO: 2431; SEQ ID NO: 2432; SEQ ID NO: 2433; SEQ ID NO: 2434; SEQ ID NO: 2435; SEQ ID NO: 2436; SEQ ID NO: 2437; SEQ ID NO: 2438; SEQ ID NO: 2439; SEQ ID NO: 2440; SEQ ID NO: 2441; SEQ ID NO: 2442; SEQ ID NO: 2443; SEQ ID NO: 2444; SEQ ID NO: 2445; SEQ ID NO: 2446; SEQ ID NO: 2447; SEQ ID NO: 2448; SEQ ID NO: 2449; SEQ ID NO: 2450; SEQ ID NO: 2451; SEQ ID NO: 2452; SEQ ID NO: 2453; SEQ ID NO: 2454; SEQ ID NO: 2455; SEQ ID NO: 2456; SEQ ID NO: 2457; SEQ ID NO: 2458; SEQ ID NO: 2459; SEQ ID NO: 2460; SEQ ID NO: 2461; SEQ ID NO: 2462; SEQ ID NO: 2463; SEQ ID NO: 2464; SEQ ID NO: 2465; SEQ ID NO: 2466; SEQ ID NO: 2467; SEQ ID NO: 2468; SEQ ID NO: 2469; SEQ ID NO: 2470; SEQ ID NO: 2471; SEQ ID NO: 2472; SEQ ID NO: 2473; SEQ ID NO: 2474; SEQ ID NO: 2475; SEQ ID NO: 2476; SEQ ID NO: 2477; SEQ ID NO: 2478; SEQ ID NO: 2479; SEQ ID NO: 2480; SEQ ID NO: 2481; SEQ ID NO: 2482; SEQ ID NO: 2483; SEQ ID NO: 2484; SEQ ID NO: 2485; SEQ ID NO: 2486; SEQ ID NO: 2487; SEQ ID NO: 2488; SEQ ID NO: 2489; SEQ ID NO: 2490; SEQ ID NO: 2491; SEQ ID NO: 2492; SEQ ID NO: 2493; SEQ ID NO: 2494; SEQ ID NO: 2495; SEQ ID NO: 2496; SEQ ID NO: 2497; SEQ ID NO: 2498; SEQ ID NO: 2499; SEQ ID NO: 2500; SEQ ID NO: 2501; SEQ ID NO: 2502; SEQ ID NO: 2503; SEQ ID NO: 2504; SEQ ID NO: 2505; SEQ ID NO: 2506; SEQ ID NO: 2507; SEQ ID NO: 2508; SEQ ID NO: 2509; SEQ ID NO: 2510; SEQ ID NO: 2511; SEQ ID NO: 2512; SEQ ID NO: 2513; SEQ ID NO: 2514; SEQ ID NO: 2515; SEQ ID NO: 2516; SEQ ID NO: 2517; SEQ ID NO: 2518; SEQ ID NO: 2519; SEQ ID NO: 2520; SEQ ID NO: 2521; SEQ ID NO: 2522; SEQ ID NO: 2523; SEQ ID NO: 2524; SEQ ID NO: 2525; SEQ ID NO: 2526; SEQ ID NO: 2527; SEQ ID NO: 2528; SEQ ID NO: 2529; SEQ ID NO: 2530; SEQ ID NO: 2531; SEQ ID NO: 2532; SEQ ID NO: 2533; SEQ ID NO: 2534; SEQ ID NO: 2535; SEQ ID NO: 2536; SEQ ID NO: 2537; SEQ ID NO: 2538; SEQ ID NO: 2539; SEQ ID NO: 2540; SEQ ID NO: 2541; SEQ ID NO: 2542; SEQ ID NO: 2543; SEQ ID NO: 2544; SEQ ID NO: 2545; SEQ ID NO: 2546; SEQ ID NO: 2547; SEQ ID NO: 2548; SEQ ID NO: 2549; SEQ ID NO: 2550; SEQ ID NO: 2551; SEQ ID NO: 2552; SEQ ID NO: 2553; SEQ ID NO: 2554; SEQ ID NO: 2555; SEQ ID NO: 2556; SEQ ID NO: 2557; SEQ ID NO: 2558; SEQ ID NO: 2559; SEQ ID NO: 2560; SEQ ID NO: 2561; SEQ ID NO: 2562; SEQ ID NO: 2563; SEQ ID NO: 2564; SEQ ID NO: 2565; SEQ ID NO: 2566; SEQ ID NO: 2567; SEQ ID NO: 2568; SEQ ID NO: 2569; SEQ ID NO: 2570; SEQ ID NO: 2571; SEQ ID NO: 2572; SEQ ID NO: 2573; SEQ ID NO: 2574; SEQ ID NO: 2575; SEQ ID NO: 2576; SEQ ID NO: 2577; SEQ ID NO: 2578; SEQ ID NO: 2579; SEQ ID NO: 2580; SEQ ID NO: 2581; SEQ ID NO: 2582; SEQ ID NO: 2583; SEQ ID NO: 2584; SEQ ID NO: 2585; SEQ ID NO: 2586; SEQ ID NO: 2587; SEQ ID NO: 2588; SEQ ID NO: 2589; SEQ ID NO: 2590; SEQ ID NO: 2591; SEQ ID NO: 2592; SEQ ID NO: 2593; SEQ ID NO: 2594; SEQ ID NO: 2595; SEQ ID NO: 2596; SEQ ID NO: 2597; SEQ ID NO: 2598; SEQ ID NO: 2599; SEQ ID NO: 2600; SEQ ID NO: 2601; SEQ ID NO: 2602; SEQ ID NO: 2603; SEQ ID NO: 2604; SEQ ID NO: 2605; SEQ ID NO: 2606; SEQ ID NO: 2607; SEQ ID NO: 2608; SEQ ID NO: 2609; SEQ ID NO: 2610; SEQ ID NO: 2611; SEQ ID NO: 2612; SEQ ID NO: 2613; SEQ ID NO: 2614; SEQ ID NO: 2615; SEQ ID NO: 2616; SEQ ID NO: 2617; SEQ ID NO: 2618; SEQ ID NO: 2619; SEQ ID NO: 2620; SEQ ID NO: 2621; SEQ ID NO: 2622; SEQ ID NO: 2623; SEQ ID NO: 2624; SEQ ID NO: 2625; SEQ ID NO: 2626; SEQ ID NO: 2627; SEQ ID NO: 2628; SEQ ID NO: 2629; SEQ ID NO: 2630; SEQ ID NO: 2631; SEQ ID NO: 2632; SEQ ID NO: 2633; SEQ ID NO: 2634; SEQ ID NO: 2635; SEQ ID NO: 2636; SEQ ID NO: 2637; SEQ ID NO: 2638; SEQ ID NO: 2639; SEQ ID NO: 2640; SEQ ID NO: 2641; SEQ ID NO: 2642; SEQ ID NO: 2643; SEQ ID NO: 2644; SEQ ID NO: 2645; SEQ ID NO: 2646; SEQ ID NO: 2647; SEQ ID NO: 2648; SEQ ID NO: 2649; SEQ ID NO: 2650; SEQ ID NO: 2651; SEQ ID NO: 2652; SEQ ID NO: 2653; SEQ ID NO: 2654; SEQ ID NO: 2655; SEQ ID NO: 2656; SEQ ID NO: 2657; SEQ ID NO: 2658; SEQ ID NO: 2659; SEQ ID NO: 2660; SEQ ID NO: 2661; SEQ ID NO: 2662; SEQ ID NO: 2663; SEQ ID NO: 2664; SEQ ID NO: 2665; SEQ ID NO: 2666; SEQ ID NO: 2667; SEQ ID NO: 2668; SEQ ID NO: 2669; SEQ ID NO: 2670; SEQ ID NO: 2671; SEQ ID NO: 2672; SEQ ID NO: 2673; SEQ ID NO: 2674; SEQ ID NO: 2675; SEQ ID NO: 2676; SEQ ID NO: 2677; SEQ ID NO: 2678; SEQ ID NO: 2679; SEQ ID NO: 2680; SEQ ID NO: 2681; SEQ ID NO: 2682; SEQ ID NO: 2683; SEQ ID NO: 2684; SEQ ID NO: 2685; SEQ ID NO: 2686; SEQ ID NO: 2687; SEQ ID NO: 2688; SEQ ID NO: 2689; SEQ ID NO: 2690; SEQ ID NO: 2691; SEQ ID NO: 2692; SEQ ID NO: 2693; SEQ ID NO: 2694; SEQ ID NO: 2695; SEQ ID NO: 2696; SEQ ID NO: 2697; SEQ ID NO: 2698; SEQ ID NO: 2699; SEQ ID NO: 2700; SEQ ID NO: 2701; SEQ ID NO: 2702; SEQ ID NO: 2703; SEQ ID NO: 2704; SEQ ID NO: 2705; SEQ ID NO: 2706; SEQ ID NO: 2707; SEQ ID NO: 2708; SEQ ID NO: 2709; SEQ ID NO: 2710; SEQ ID NO: 2711; SEQ ID NO: 2712; SEQ ID NO: 2713; SEQ ID NO: 2714; SEQ ID NO: 2715; SEQ ID NO: 2716; SEQ ID NO: 2717; SEQ ID NO: 2718; SEQ ID NO: 2719; SEQ ID NO: 2720; SEQ ID NO: 2721; SEQ ID NO: 2722; SEQ ID NO: 2723; SEQ ID NO: 2724; SEQ ID NO: 2725; SEQ ID NO: 2726; SEQ ID NO: 2727; SEQ ID NO: 2728; SEQ ID NO: 2729; SEQ ID NO: 2730; SEQ ID NO: 2731; SEQ ID NO: 2732; SEQ ID NO: 2733; SEQ ID NO: 2734; SEQ ID NO: 2735; SEQ ID NO: 2736; SEQ ID NO: 2737; SEQ ID NO: 2738; SEQ ID NO: 2739; SEQ ID NO: 2740; SEQ ID NO: 2741; SEQ ID NO: 2742; SEQ ID NO: 2743; SEQ ID NO: 2744; SEQ ID NO: 2745; SEQ ID NO: 2746; SEQ ID NO: 2747; SEQ ID NO: 2748; SEQ ID NO: 2749; SEQ ID NO: 2750; SEQ ID NO: 2751; SEQ ID NO: 2752; SEQ ID NO: 2753; SEQ ID NO: 2754; SEQ ID NO: 2755; SEQ ID NO: 2756; SEQ ID NO: 2757; SEQ ID NO: 2758; SEQ ID NO: 2759; SEQ ID NO: 2760; SEQ ID NO: 2761; SEQ ID NO: 2762; SEQ ID NO: 2763; SEQ ID NO: 2764; SEQ ID NO: 2765; SEQ ID NO: 2766; SEQ ID NO: 2767; SEQ ID NO: 2768; SEQ ID NO: 2769; SEQ ID NO: 2770; SEQ ID NO: 2771; SEQ ID NO: 2772; SEQ ID NO: 2773; SEQ ID NO: 2774; SEQ ID NO: 2775; SEQ ID NO: 2776; SEQ ID NO: 2777; SEQ ID NO: 2778; SEQ ID NO: 2779; SEQ ID NO: 2780; SEQ ID NO: 2781; SEQ ID NO: 2782; SEQ ID NO: 2783; SEQ ID NO: 2784; SEQ ID NO: 2785; SEQ ID NO: 2786; SEQ ID NO: 2787; SEQ ID NO: 2788; SEQ ID NO: 2789; SEQ ID NO: 2790; SEQ ID NO: 2791; SEQ ID NO: 2792; SEQ ID NO: 2793; SEQ ID NO: 2794; SEQ ID NO: 2795; SEQ ID NO: 2796; SEQ ID NO: 2797; SEQ ID NO: 2798; SEQ ID NO: 2799; SEQ ID NO: 2800; SEQ ID NO: 2801; SEQ ID NO: 2802; SEQ ID NO: 2803; SEQ ID NO: 2804; SEQ ID NO: 2805; SEQ ID NO: 2806; SEQ ID NO: 2807; SEQ ID NO: 2808; SEQ ID NO: 2809; SEQ ID NO: 2810; SEQ ID NO: 2811; SEQ ID NO: 2812; SEQ ID NO: 2813; SEQ ID NO: 2814; SEQ ID NO: 2815; SEQ ID NO: 2816; SEQ ID NO: 2817; SEQ ID NO: 2818; SEQ ID NO: 2819; SEQ ID NO: 2820; SEQ ID NO: 2821; SEQ ID NO: 2822; SEQ ID NO: 2823; SEQ ID NO: 2824; SEQ ID NO: 2825; SEQ ID NO: 2826; SEQ ID NO: 2827; SEQ ID NO: 2828; SEQ ID NO: 2829; SEQ ID NO: 2830; SEQ ID NO: 2831; SEQ ID NO: 2832; SEQ ID NO: 2833; SEQ ID NO: 2834; SEQ ID NO: 2835; SEQ ID NO: 2836; SEQ ID NO: 2837; SEQ ID NO: 2838; SEQ ID NO: 2839; SEQ ID NO: 2840; SEQ ID NO: 2841; SEQ ID NO: 2842; SEQ ID NO: 2843; SEQ ID NO: 2844; SEQ ID NO: 2845; SEQ ID NO: 2846; SEQ ID NO: 2847; SEQ ID NO: 2848; SEQ ID NO: 2849; SEQ ID NO: 2850; SEQ ID NO: 2851; SEQ ID NO: 2852; SEQ ID NO: 2853; SEQ ID NO: 2854; SEQ ID NO: 2855; SEQ ID NO: 2856; SEQ ID NO: 2857; SEQ ID NO: 2858; SEQ ID NO: 2859; SEQ ID NO: 2860; SEQ ID NO: 2861; SEQ ID NO: 2862; SEQ ID NO: 2863; SEQ ID NO: 2864; SEQ ID NO: 2865; SEQ ID NO: 2866; SEQ ID NO: 2867; SEQ ID NO: 2868; SEQ ID NO: 2869; SEQ ID NO: 2870; SEQ ID NO: 2871; SEQ ID NO: 2872; SEQ ID NO: 2873; SEQ ID NO: 2874; SEQ ID NO: 2875; SEQ ID NO: 2876; SEQ ID NO: 2877; SEQ ID NO: 2878; SEQ ID NO: 2879; SEQ ID NO: 2880; SEQ ID NO: 2881; SEQ ID NO: 2882; SEQ ID NO: 2883; SEQ ID NO: 2884; SEQ ID NO: 2885; SEQ ID NO: 2886; SEQ ID NO: 2887; SEQ ID NO: 2888; SEQ ID NO: 2889; SEQ ID NO: 2890; SEQ ID NO: 2891; SEQ ID NO: 2892; SEQ ID NO: 2893; SEQ ID NO: 2894; SEQ ID NO: 2895; SEQ ID NO: 2896; SEQ ID NO: 2897; SEQ ID NO: 2898; SEQ ID NO: 2899; SEQ ID NO: 2900; SEQ ID NO: 2901; SEQ ID NO: 2902; SEQ ID NO: 2903; SEQ ID NO: 2904; SEQ ID NO: 2905; SEQ ID NO: 2906; SEQ ID NO: 2907; SEQ ID NO: 2908; SEQ ID NO: 2909; SEQ ID NO: 2910; SEQ ID NO: 2911; SEQ ID NO: 2912; SEQ ID NO: 2913; SEQ ID NO: 2914; SEQ ID NO: 2915; SEQ ID NO: 2916; SEQ ID NO: 2917; SEQ ID NO: 2918; SEQ ID NO: 2919; SEQ ID NO: 2920; SEQ ID NO: 2921; SEQ ID NO: 2922; SEQ ID NO: 2923; SEQ ID NO: 2924; SEQ ID NO: 2925; SEQ ID NO: 2926; SEQ ID NO: 2927; SEQ ID NO: 2928; SEQ ID NO: 2929; SEQ ID NO: 2930; SEQ ID NO: 2931; SEQ ID NO: 2932; SEQ ID NO: 2933; SEQ ID NO: 2934; SEQ ID NO: 2935; SEQ ID NO: 2936; SEQ ID NO: 2937; SEQ ID NO: 2938; SEQ ID NO: 2939; SEQ ID NO: 2940; SEQ ID NO: 2941; SEQ ID NO: 2942; SEQ ID NO: 2943; SEQ ID NO: 2944; SEQ ID NO: 2945; SEQ ID NO: 2946; SEQ ID NO: 2947; SEQ ID NO: 2948; SEQ ID NO: 2949; SEQ ID NO: 2950; SEQ ID NO: 2951; SEQ ID NO: 2952; SEQ ID NO: 2953; SEQ ID NO: 2954; SEQ ID NO: 2955; SEQ ID NO: 2956; SEQ ID NO: 2957; SEQ ID NO: 2958; SEQ ID NO: 2959; SEQ ID NO: 2960; SEQ ID NO: 2961; SEQ ID NO: 2962; SEQ ID NO: 2963; SEQ ID NO: 2964; SEQ ID NO: 2965; SEQ ID NO: 2966; SEQ ID NO: 2967; SEQ ID NO: 2968; SEQ ID NO: 2969; SEQ ID NO: 2970; SEQ ID NO: 2971; SEQ ID NO: 2972; SEQ ID NO: 2973; SEQ ID NO: 2974; SEQ ID NO: 2975; SEQ ID NO: 2976; SEQ ID NO: 2977; SEQ ID NO: 2978; SEQ ID NO: 2979; SEQ ID NO: 2980; SEQ ID NO: 2981; SEQ ID NO: 2982; SEQ ID NO: 2983; SEQ ID NO: 2984; SEQ ID NO: 2985; SEQ ID NO: 2986; SEQ ID NO: 2987; SEQ ID NO: 2988; SEQ ID NO: 2989; SEQ ID NO: 2990; SEQ ID NO: 2991; SEQ ID NO: 2992; SEQ ID NO: 2993; SEQ ID NO: 2994; SEQ ID NO: 2995; SEQ ID NO: 2996; SEQ ID NO: 2997; SEQ ID NO: 2998; SEQ ID NO: 2999; SEQ ID NO: 3000; SEQ ID NO: 3001; SEQ ID NO: 3002; SEQ ID NO: 3003; SEQ ID NO: 3004; SEQ ID NO: 3005; SEQ ID NO: 3006; SEQ ID NO: 3007; SEQ ID NO: 3008; SEQ ID NO: 3009; SEQ ID NO: 3010; SEQ ID NO: 3011; SEQ ID NO: 3012; SEQ ID NO: 3013; SEQ ID NO: 3014; SEQ ID NO: 3015; SEQ ID NO: 3016; SEQ ID NO: 3017; SEQ ID NO: 3018; SEQ ID NO: 3019; SEQ ID NO: 3020; SEQ ID NO: 3021; SEQ ID NO: 3022; SEQ ID NO: 3023; SEQ ID NO: 3024; SEQ ID NO: 3025; SEQ ID NO: 3026; SEQ ID NO: 3027; SEQ ID NO: 3028; SEQ ID NO: 3029; SEQ ID NO: 3030; SEQ ID NO: 3031; SEQ ID NO: 3032; SEQ ID NO: 3033; SEQ ID NO: 3034; SEQ ID NO: 3035; SEQ ID NO: 3036; SEQ ID NO: 3037; SEQ ID NO: 3038; SEQ ID NO: 3039; SEQ ID NO: 3040; SEQ ID NO: 3041; SEQ ID NO: 3042; SEQ ID NO: 3043; SEQ ID NO: 3044; SEQ ID NO: 3045; SEQ ID NO: 3046; SEQ ID NO: 3047; SEQ ID NO: 3048; SEQ ID NO: 3049; SEQ ID NO: 3050; SEQ ID NO: 3051; SEQ ID NO: 3052; SEQ ID NO: 3053; SEQ ID NO: 3054; SEQ ID NO: 3055; SEQ ID NO: 3056; SEQ ID NO: 3057; SEQ ID NO: 3058; SEQ ID NO: 3059; SEQ ID NO: 3060; SEQ ID NO: 3061; SEQ ID NO: 3062; SEQ ID NO: 3063; SEQ ID NO: 3064; SEQ ID NO: 3065; SEQ ID NO: 3066; SEQ ID NO: 3067; SEQ ID NO: 3068; SEQ ID NO: 3069; SEQ ID NO: 3070; SEQ ID NO: 3071; SEQ ID NO: 3072; SEQ ID NO: 3073; SEQ ID NO: 3074; SEQ ID NO: 3075; SEQ ID NO: 3076; SEQ ID NO: 3077; SEQ ID NO: 3078; SEQ ID NO: 3079; SEQ ID NO: 3080; SEQ ID NO: 3081; SEQ ID NO: 4000; SEQ ID NO: 4001; SEQ ID NO: 4002; SEQ ID NO: 4003; SEQ ID NO: 4004; SEQ ID NO: 4005; SEQ ID NO: 4006; SEQ ID NO: 4007; SEQ ID NO: 4008; SEQ ID NO: 4009; SEQ ID NO: 4010; SEQ ID NO: 4011; SEQ ID NO: 4012; SEQ ID NO: 4013; SEQ ID NO: 4014; SEQ ID NO: 4015; SEQ ID NO: 4016; SEQ ID NO: 4017; SEQ ID NO: 4018; SEQ ID NO: 4019; SEQ ID NO: 4020; SEQ ID NO: 4021; SEQ ID NO: 4022; SEQ ID NO: 4023; SEQ ID NO: 4024; SEQ ID NO: 4025; SEQ ID NO: 4026; SEQ ID NO: 4027; SEQ ID NO: 4028; SEQ ID NO: 4029; SEQ ID NO: 4030; SEQ ID NO: 4031; SEQ ID NO: 4032; SEQ ID NO: 4033; SEQ ID NO: 4034; SEQ ID NO: 4035; SEQ ID NO: 4036; SEQ ID NO: 4037; SEQ ID NO: 4038; SEQ ID NO: 4039; SEQ ID NO: 4040; SEQ ID NO: 4041; SEQ ID NO: 4042; SEQ ID NO: 4043; SEQ ID NO: 4044; SEQ ID NO: 4045; SEQ ID NO: 4046; SEQ ID NO: 4047; SEQ ID NO: 4048; SEQ ID NO: 4049; SEQ ID NO: 4050; SEQ ID NO: 4051; SEQ ID NO: 4052; SEQ ID NO: 4053; SEQ ID NO: 4054; SEQ ID NO: 4055; SEQ ID NO: 4056; SEQ ID NO: 4057; SEQ ID NO: 4058; SEQ ID NO: 4059; SEQ ID NO: 4060; SEQ ID NO: 4061; SEQ ID NO: 4062; SEQ ID NO: 4063; SEQ ID NO: 4064; SEQ ID NO: 4065; SEQ ID NO: 4066; SEQ ID NO: 4067; SEQ ID NO: 4068; SEQ ID NO: 4069; SEQ ID NO: 4070; SEQ ID NO: 4071; SEQ ID NO: 4072; SEQ ID NO: 4073; SEQ ID NO: 4074; SEQ ID NO: 4075; SEQ ID NO: 4076; SEQ ID NO: 4077; SEQ ID NO: 4078; SEQ ID NO: 4079; SEQ ID NO: 4080; SEQ ID NO: 4081; SEQ ID NO: 4082; SEQ ID NO: 4083; SEQ ID NO: 4084; SEQ ID NO: 4085; SEQ ID NO: 4086; SEQ ID NO: 4087; SEQ ID NO: 4088; SEQ ID NO: 4089; SEQ ID NO: 4090; SEQ ID NO: 4091; SEQ ID NO: 4092; SEQ ID NO: 4093; SEQ ID NO: 4094; SEQ ID NO: 4095; SEQ ID NO: 4096; SEQ ID NO: 4097; SEQ ID NO: 4098; SEQ ID NO: 4099; SEQ ID NO: 4100; SEQ ID NO: 4101; SEQ ID NO: 4102; SEQ ID NO: 4103; SEQ ID NO: 4104; SEQ ID NO: 4105; SEQ ID NO: 4106; SEQ ID NO: 4107; SEQ ID NO: 4108; SEQ ID NO: 4109; SEQ ID NO: 4110; SEQ ID NO: 4111; SEQ ID NO: 4112; SEQ ID NO: 4113; SEQ ID NO: 4114; SEQ ID NO: 4115; SEQ ID NO: 4116; SEQ ID NO: 4117; SEQ ID NO: 4118; SEQ ID NO: 4119; SEQ ID NO: 4120; SEQ ID NO: 4121; SEQ ID NO: 4122; SEQ ID NO: 4123; SEQ ID NO: 4124; SEQ ID NO: 4125; SEQ ID NO: 4126; SEQ ID NO: 4127; SEQ ID NO: 4128; SEQ ID NO: 4129; SEQ ID NO: 4130; SEQ ID NO: 4131; SEQ ID NO: 4132; SEQ ID NO: 4133; SEQ ID NO: 4134; SEQ ID NO: 4135; SEQ ID NO: 4136; SEQ ID NO: 4137; SEQ ID NO: 4138; SEQ ID NO: 4139; SEQ ID NO: 4140; SEQ ID NO: 4141; SEQ ID NO: 4142; SEQ ID NO: 4143; SEQ ID NO: 4144; SEQ ID NO: 4145; SEQ ID NO: 4146; SEQ ID NO: 4147; SEQ ID NO: 4148; SEQ ID NO: 4149; SEQ ID NO: 4150; SEQ ID NO: 4151; SEQ ID NO: 4152; SEQ ID NO: 4153; SEQ ID NO: 4154; SEQ ID NO: 4155; SEQ ID NO: 4156; SEQ ID NO: 4157; SEQ ID NO: 4158; SEQ ID NO: 4159; SEQ ID NO: 4160; SEQ ID NO: 4161; SEQ ID NO: 4162; SEQ ID NO: 4163; SEQ ID NO: 4164; SEQ ID NO: 4165; SEQ ID NO: 4166; SEQ ID NO: 4167; SEQ ID NO: 4168; SEQ ID NO: 4169; SEQ ID NO: 4170; SEQ ID NO: 4171; SEQ ID NO: 4172; SEQ ID NO: 4173; SEQ ID NO: 4174; SEQ ID NO: 4175; SEQ ID NO: 4176; SEQ ID NO: 4177; SEQ ID NO: 4178; SEQ ID NO: 4179; SEQ ID NO: 4180; SEQ ID NO: 4181; SEQ ID NO: 4182; SEQ ID NO: 4183; SEQ ID NO: 4184; SEQ ID NO: 4185; SEQ ID NO: 4186; SEQ ID NO: 4187; SEQ ID NO: 4188; SEQ ID NO: 4189; SEQ ID NO: 4190; SEQ ID NO: 4191; SEQ ID NO: 4192; SEQ ID NO: 4193; SEQ ID NO: 4194; SEQ ID NO: 4195; SEQ ID NO: 4196; SEQ ID NO: 4197; SEQ ID NO: 4198; SEQ ID NO: 4199; SEQ ID NO: 4200; SEQ ID NO: 4201; SEQ ID NO: 4202; SEQ ID NO: 4203; SEQ ID NO: 4204; SEQ ID NO: 4205; SEQ ID NO: 4206; SEQ ID NO: 4207; SEQ ID NO: 4208; SEQ ID NO: 4209; SEQ ID NO: 4210; SEQ ID NO: 4211; SEQ ID NO: 4212; SEQ ID NO: 4213; SEQ ID NO: 4214; SEQ ID NO: 4215; SEQ ID NO: 4216; SEQ ID NO: 4217; SEQ ID NO: 4218; SEQ ID NO: 4219; SEQ ID NO: 4220; SEQ ID NO: 4221; SEQ ID NO: 4222; SEQ ID NO: 4223; SEQ ID NO: 4224; SEQ ID NO: 4225; SEQ ID NO: 4226; SEQ ID NO: 4227; SEQ ID NO: 4228; SEQ ID NO: 4229; SEQ ID NO: 4230; SEQ ID NO: 4231; SEQ ID NO: 4232; SEQ ID NO: 4233; SEQ ID NO: 4234; SEQ ID NO: 4235; SEQ ID NO: 4236; SEQ ID NO: 4237; SEQ ID NO: 4238; SEQ ID NO: 4239; SEQ ID NO: 4240; SEQ ID NO: 4241; SEQ ID NO: 4242; SEQ ID NO: 4243; SEQ ID NO: 4244; SEQ ID NO: 4245; SEQ ID NO: 4246; SEQ ID NO: 4247; SEQ ID NO: 4248; SEQ ID NO: 4249; SEQ ID NO: 4250; SEQ ID NO: 4251; SEQ ID NO: 4252; SEQ ID NO: 4253; SEQ ID NO: 4254; SEQ ID NO: 4255; SEQ ID NO: 4256; SEQ ID NO: 4257; SEQ ID NO: 4258; SEQ ID NO: 4259; SEQ ID NO: 4260; SEQ ID NO: 4261; SEQ ID NO: 4262; SEQ ID NO: 4263; SEQ ID NO: 4264; SEQ ID NO: 4265; SEQ ID NO: 4266; SEQ ID NO: 4267; SEQ ID NO: 4268; SEQ ID NO: 4269; SEQ ID NO: 4270; SEQ ID NO: 4271; SEQ ID NO: 4272; SEQ ID NO: 4273; SEQ ID NO: 4274; SEQ ID NO: 4275; SEQ ID NO: 4276; SEQ ID NO: 4277; SEQ ID NO: 4278; SEQ ID NO: 4279; SEQ ID NO: 4280; SEQ ID NO: 4281; SEQ ID NO: 4282; SEQ ID NO: 4283; SEQ ID NO: 4284; SEQ ID NO: 4285; SEQ ID NO: 4286; SEQ ID NO: 4287; SEQ ID NO: 4288; SEQ ID NO: 4289; SEQ ID NO: 4290; SEQ ID NO: 4291; SEQ ID NO: 4292; SEQ ID NO: 4293; SEQ ID NO: 4294; SEQ ID NO: 4295; SEQ ID NO: 4296; SEQ ID NO: 4297; SEQ ID NO: 4298; SEQ ID NO: 4299; SEQ ID NO: 4300; SEQ ID NO: 4301; SEQ ID NO: 4302; SEQ ID NO: 4303; SEQ ID NO: 4304; SEQ ID NO: 4305; SEQ ID NO: 4306; SEQ ID NO: 4307; SEQ ID NO: 4308; SEQ ID NO: 4309; SEQ ID NO: 4310; SEQ ID NO: 4311; SEQ ID NO: 4312; SEQ ID NO: 4313; SEQ ID NO: 4314; SEQ ID NO: 4315; SEQ ID NO: 4316; SEQ ID NO: 4317; SEQ ID NO: 4318; SEQ ID NO: 4319; SEQ ID NO: 4320; SEQ ID NO: 4321; SEQ ID NO: 4322; SEQ ID NO: 4323; SEQ ID NO: 4324; SEQ ID NO: 4325; SEQ ID NO: 4326; SEQ ID NO: 4327; SEQ ID NO: 4328; SEQ ID NO: 4329; SEQ ID NO: 4330; SEQ ID NO: 4331; SEQ ID NO: 4332; SEQ ID NO: 4333; SEQ ID NO: 4334; SEQ ID NO: 4335; SEQ ID NO: 4336; SEQ ID NO: 4337; SEQ ID NO: 4338; SEQ ID NO: 4339; SEQ ID NO: 4340; SEQ ID NO: 4341; SEQ ID NO: 4342; SEQ ID NO: 4343; SEQ ID NO: 4344; SEQ ID NO: 4345; SEQ ID NO: 4346; SEQ ID NO: 4347; SEQ ID NO: 4348; SEQ ID NO: 4349; SEQ ID NO: 4350; SEQ ID NO: 4351; SEQ ID NO: 4352; SEQ ID NO: 4353; SEQ ID NO: 4354; SEQ ID NO: 4355; SEQ ID NO: 4356; SEQ ID NO: 4357; SEQ ID NO: 4358; SEQ ID NO: 4359; SEQ ID NO: 4360; SEQ ID NO: 4361; SEQ ID NO: 4362; SEQ ID NO: 4363; SEQ ID NO: 4364; SEQ ID NO: 4365; SEQ ID NO: 4366; SEQ ID NO: 4367; SEQ ID NO: 4368; SEQ ID NO: 4369; SEQ ID NO: 4370; SEQ ID NO: 4371; SEQ ID NO: 4372; SEQ ID NO: 4373; SEQ ID NO: 4374; SEQ ID NO: 4375; SEQ ID NO: 4376; SEQ ID NO: 4377; SEQ ID NO: 4378; SEQ ID NO: 4379; SEQ ID NO: 4380; SEQ ID NO: 4381; SEQ ID NO: 4382; SEQ ID NO: 4383; SEQ ID NO: 4384; SEQ ID NO: 4385; SEQ ID NO: 4386; SEQ ID NO: 4387; SEQ ID NO: 4388; SEQ ID NO: 4389; SEQ ID NO: 4390; SEQ ID NO: 4391; SEQ ID NO: 4392; SEQ ID NO: 4393; SEQ ID NO: 4394; SEQ ID NO: 4395; SEQ ID NO: 4396; SEQ ID NO: 4397; SEQ ID NO: 4398; SEQ ID NO: 4399; SEQ ID NO: 4400; SEQ ID NO: 4401; SEQ ID NO: 4402; SEQ ID NO: 4403; SEQ ID NO: 4404; SEQ ID NO: 4405; SEQ ID NO: 4406; SEQ ID NO: 4407; SEQ ID NO: 4408; SEQ ID NO: 4409; SEQ ID NO: 4410; SEQ ID NO: 4411; SEQ ID NO: 4412; SEQ ID NO: 4413; SEQ ID NO: 4414; SEQ ID NO: 4415; SEQ ID NO: 4416; SEQ ID NO: 4417; SEQ ID NO: 4418; SEQ ID NO: 4419; SEQ ID NO: 4420; SEQ ID NO: 4421; SEQ ID NO: 4422; SEQ ID NO: 4423; SEQ ID NO: 4424; SEQ ID NO: 4425; SEQ ID NO: 4426; SEQ ID NO: 4427; SEQ ID NO: 4428; SEQ ID NO: 4429; SEQ ID NO: 4430; SEQ ID NO: 4431; SEQ ID NO: 4432; SEQ ID NO: 4433; SEQ ID NO: 4434; SEQ ID NO: 4435; SEQ ID NO: 4436; SEQ ID NO: 4437; SEQ ID NO: 4438; SEQ ID NO: 4439; SEQ ID NO: 4440; SEQ ID NO: 4441; SEQ ID NO: 4442; SEQ ID NO: 4443; SEQ ID NO: 4444; SEQ ID NO: 4445; SEQ ID NO: 4446; SEQ ID NO: 4447; SEQ ID NO: 4448; SEQ ID NO: 4449; SEQ ID NO: 4450; SEQ ID NO: 4451; SEQ ID NO: 4452; SEQ ID NO: 4453; SEQ ID NO: 4454; SEQ ID NO: 4455; SEQ ID NO: 4456; SEQ ID NO: 4457; SEQ ID NO: 4458; SEQ ID NO: 4459; SEQ ID NO: 4460; SEQ ID NO: 4461; SEQ ID NO: 4462; SEQ ID NO: 4463; SEQ ID NO: 4464; SEQ ID NO: 4465; SEQ ID NO: 4466; SEQ ID NO: 4467; SEQ ID NO: 4468; SEQ ID NO: 4469; SEQ ID NO: 4470; SEQ ID NO: 4471; SEQ ID NO: 4472; SEQ ID NO: 4473; SEQ ID NO: 4474; SEQ ID NO: 4475; SEQ ID NO: 4476; SEQ ID NO: 4477; SEQ ID NO: 4478; SEQ ID NO: 4479; SEQ ID NO: 4480; SEQ ID NO: 4481; SEQ ID NO: 4482; SEQ ID NO: 4483; SEQ ID NO: 4484; SEQ ID NO: 4485; SEQ ID NO: 4486; SEQ ID NO: 4487; SEQ ID NO: 4488; SEQ ID NO: 4489; SEQ ID NO: 4490; SEQ ID NO: 4491; SEQ ID NO: 4492; SEQ ID NO: 4493; SEQ ID NO: 4494; SEQ ID NO: 4495; SEQ ID NO: 4496; SEQ ID NO: 4497; SEQ ID NO: 4498; SEQ ID NO: 4499; SEQ ID NO: 4500; SEQ ID NO: 4501; SEQ ID NO: 4502; SEQ ID NO: 4503; SEQ ID NO: 4504; SEQ ID NO: 4505; SEQ ID NO: 4506; SEQ ID NO: 4507; SEQ ID NO: 4508; SEQ ID NO: 4509; SEQ ID NO: 4510; SEQ ID NO: 4511; SEQ ID NO: 4512; SEQ ID NO: 4513; SEQ ID NO: 4514; SEQ ID NO: 4515; SEQ ID NO: 4516; SEQ ID NO: 4517; SEQ ID NO: 4518; SEQ ID NO: 4519; SEQ ID NO: 4520; SEQ ID NO: 4521; SEQ ID NO: 4522; SEQ ID NO: 4523; SEQ ID NO: 4524; SEQ ID NO: 4525; SEQ ID NO: 4526; SEQ ID NO: 4527; SEQ ID NO: 4528; SEQ ID NO: 4529; SEQ ID NO: 4530; SEQ ID NO: 4531; SEQ ID NO: 4532; SEQ ID NO: 4533; SEQ ID NO: 4534; SEQ ID NO: 4535; SEQ ID NO: 4536; SEQ ID NO: 4537; SEQ ID NO: 4538; SEQ ID NO: 4539; SEQ ID NO: 4540; SEQ ID NO: 4541; SEQ ID NO: 4542; SEQ ID NO: 4543; SEQ ID NO: 4544; SEQ ID NO: 4545; SEQ ID NO: 4546; SEQ ID NO: 4547; SEQ ID NO: 4548; SEQ ID NO: 4549; SEQ ID NO: 4550; SEQ ID NO: 4551; SEQ ID NO: 4552; SEQ ID NO: 4553; SEQ ID NO: 4554; SEQ ID NO: 4555; SEQ ID NO: 4556; SEQ ID NO: 4557; SEQ ID NO: 4558; SEQ ID NO: 4559; SEQ ID NO: 4560; SEQ ID NO: 4561; SEQ ID NO: 4562; SEQ ID NO: 4563; SEQ ID NO: 4564; SEQ ID NO: 4565; SEQ ID NO: 4566; SEQ ID NO: 4567; SEQ ID NO: 4568; SEQ ID NO: 4569; SEQ ID NO: 4570; SEQ ID NO: 4571; SEQ ID NO: 4572; SEQ ID NO: 4573; SEQ ID NO: 4574; SEQ ID NO: 4575; SEQ ID NO: 4576; SEQ ID NO: 4577; SEQ ID NO: 4578; SEQ ID NO: 4579; SEQ ID NO: 4580; SEQ ID NO: 4581; SEQ ID NO: 4582; SEQ ID NO: 4583; SEQ ID NO: 4584; SEQ ID NO: 4585; SEQ ID NO: 4586; SEQ ID NO: 4587; SEQ ID NO: 4588; SEQ ID NO: 4589; SEQ ID NO: 4590; SEQ ID NO: 4591; SEQ ID NO: 4592; SEQ ID NO: 4593; SEQ ID NO: 4594; SEQ ID NO: 4595; SEQ ID NO: 4596; SEQ ID NO: 4597; SEQ ID NO: 4598; SEQ ID NO: 4599; SEQ ID NO: 4600; SEQ ID NO: 4601; SEQ ID NO: 4602; SEQ ID NO: 4603; SEQ ID NO: 4604; SEQ ID NO: 4605; SEQ ID NO: 4606; SEQ ID NO: 4607; SEQ ID NO: 4608; SEQ ID NO: 4609; SEQ ID NO: 4610; SEQ ID NO: 4611; SEQ ID NO: 4612; SEQ ID NO: 4613; SEQ ID NO: 4614; SEQ ID NO: 4615; SEQ ID NO: 4616; SEQ ID NO: 4617; SEQ ID NO: 4618; SEQ ID NO: 4619; SEQ ID NO: 4620; SEQ ID NO: 4621; SEQ ID NO: 4622; SEQ ID NO: 4623; SEQ ID NO: 4624; SEQ ID NO: 4625; SEQ ID NO: 4626; SEQ ID NO: 4627; SEQ ID NO: 4628; SEQ ID NO: 4629; SEQ ID NO: 4630; SEQ ID NO: 4631; SEQ ID NO: 4632; SEQ ID NO: 4633; SEQ ID NO: 4634; SEQ ID NO: 4635; SEQ ID NO: 4636; SEQ ID NO: 4637; SEQ ID NO: 4638; SEQ ID NO: 4639; SEQ ID NO: 4640; SEQ ID NO: 4641; SEQ ID NO: 4642; SEQ ID NO: 4643; SEQ ID NO: 4644; SEQ ID NO: 4645; SEQ ID NO: 4646; SEQ ID NO: 4647; SEQ ID NO: 4648; SEQ ID NO: 4649; SEQ ID NO: 4650; SEQ ID NO: 4651; SEQ ID NO: 4652; SEQ ID NO: 4653; SEQ ID NO: 4654; SEQ ID NO: 4655; SEQ ID NO: 4656; SEQ ID NO: 4657; SEQ ID NO: 4658; SEQ ID NO: 4659; SEQ ID NO: 4660; SEQ ID NO: 4661; SEQ ID NO: 4662; SEQ ID NO: 4663; SEQ ID NO: 4664; SEQ ID NO: 4665; SEQ ID NO: 4666; SEQ ID NO: 4667; SEQ ID NO: 4668; SEQ ID NO: 4669; SEQ ID NO: 4670; SEQ ID NO: 4671; SEQ ID NO: 4672; SEQ ID NO: 4673; SEQ ID NO: 4674; SEQ ID NO: 4675; SEQ ID NO: 4676; SEQ ID NO: 4677; SEQ ID NO: 4678; SEQ ID NO: 4679; SEQ ID NO: 4680; SEQ ID NO: 4681; SEQ ID NO: 4682; SEQ ID NO: 4683; SEQ ID NO: 4684; SEQ ID NO: 4685; SEQ ID NO: 4686; SEQ ID NO: 4687; SEQ ID NO: 4688; SEQ ID NO: 4689; SEQ ID NO: 4690; SEQ ID NO: 4691; SEQ ID NO: 4692; SEQ ID NO: 4693; SEQ ID NO: 4694; SEQ ID NO: 4695; SEQ ID NO: 4696; SEQ ID NO: 4697; SEQ ID NO: 4698; SEQ ID NO: 4699; SEQ ID NO: 4700; SEQ ID NO: 4701; SEQ ID NO: 4702; SEQ ID NO: 4703; SEQ ID NO: 4704; SEQ ID NO: 4705; SEQ ID NO: 4706; SEQ ID NO: 4707; SEQ ID NO: 4708; SEQ ID NO: 4709; SEQ ID NO: 4710; SEQ ID NO: 4711; SEQ ID NO: 4712; SEQ ID NO: 4713; SEQ ID NO: 4714; SEQ ID NO: 4715; SEQ ID NO: 4716; SEQ ID NO: 4717; SEQ ID NO: 4718; SEQ ID NO: 4719; SEQ ID NO: 4720; SEQ ID NO: 4721; SEQ ID NO: 4722; SEQ ID NO: 4723; SEQ ID NO: 4724; SEQ ID NO: 4725; SEQ ID NO: 4726; SEQ ID NO: 4727; SEQ ID NO: 4728; SEQ ID NO: 4729; SEQ ID NO: 4730; SEQ ID NO: 4731; SEQ ID NO: 4732; SEQ ID NO: 4733; SEQ ID NO: 4734; SEQ ID NO: 4735; SEQ ID NO: 4736; SEQ ID NO: 4737; SEQ ID NO: 4738; SEQ ID NO: 4739; SEQ ID NO: 4740; SEQ ID NO: 4741; SEQ ID NO: 4742; SEQ ID NO: 4743; SEQ ID NO: 4744; SEQ ID NO: 4745; SEQ ID NO: 4746; SEQ ID NO: 4747; SEQ ID NO: 4748; SEQ ID NO: 4749; SEQ ID NO: 4750; SEQ ID NO: 4751; SEQ ID NO: 4752; SEQ ID NO: 4753; SEQ ID NO: 4754; SEQ ID NO: 4755; SEQ ID NO: 4756; SEQ ID NO: 4757; SEQ ID NO: 4758; SEQ ID NO: 4759; SEQ ID NO: 4760; SEQ ID NO: 4761; SEQ ID NO: 4762; SEQ ID NO: 4763; SEQ ID NO: 4764; SEQ ID NO: 4765; SEQ ID NO: 4766; SEQ ID NO: 4767; SEQ ID NO: 4768; SEQ ID NO: 4769; SEQ ID NO: 4770; SEQ ID NO: 4771; SEQ ID NO: 4772; SEQ ID NO: 4773; SEQ ID NO: 4774; SEQ ID NO: 4775; SEQ ID NO: 4776; SEQ ID NO: 4777; SEQ ID NO: 4778; SEQ ID NO: 4779; SEQ ID NO: 4780; SEQ ID NO: 4781; SEQ ID NO: 4782; SEQ ID NO: 4783; SEQ ID NO: 4784; SEQ ID NO: 4785; SEQ ID NO: 4786; SEQ ID NO: 4787; SEQ ID NO: 4788; SEQ ID NO: 4789; SEQ ID NO: 4790; SEQ ID NO: 4791; SEQ ID NO: 4792; SEQ ID NO: 4793; SEQ ID NO: 4794; SEQ ID NO: 4795; SEQ ID NO: 4796; SEQ ID NO: 4797; SEQ ID NO: 4798; SEQ ID NO: 4799; SEQ ID NO: 4800; SEQ ID NO: 4801; SEQ ID NO: 4802; SEQ ID NO: 4803; SEQ ID NO: 4804; SEQ ID NO: 4805; SEQ ID NO: 4806; SEQ ID NO: 4807; SEQ ID NO: 4808; SEQ ID NO: 4809; SEQ ID NO: 4810; SEQ ID NO: 4811; SEQ ID NO: 4812; SEQ ID NO: 4813; SEQ ID NO: 4814; SEQ ID NO: 4815; SEQ ID NO: 4816; SEQ ID NO: 4817; SEQ ID NO: 4818; SEQ ID NO: 4819; SEQ ID NO: 4820; SEQ ID NO: 4821; SEQ ID NO: 4822; SEQ ID NO: 4823; SEQ ID NO: 4824; SEQ ID NO: 4825; SEQ ID NO: 4826; SEQ ID NO: 4827; SEQ ID NO: 4828; SEQ ID NO: 4829; SEQ ID NO: 4830; SEQ ID NO: 4831; SEQ ID NO: 4832; SEQ ID NO: 4833; SEQ ID NO: 4834; SEQ ID NO: 4835; SEQ ID NO: 4836; SEQ ID NO: 4837; SEQ ID NO: 4838; SEQ ID NO: 4839; SEQ ID NO: 4840; SEQ ID NO: 4841; SEQ ID NO: 4842; SEQ ID NO: 4843; SEQ ID NO: 4844; SEQ ID NO: 4845; SEQ ID NO: 4846; SEQ ID NO: 4847; SEQ ID NO: 4848; SEQ ID NO: 4849; SEQ ID NO: 4850; SEQ ID NO: 4851; SEQ ID NO: 4852; SEQ ID NO: 4853; SEQ ID NO: 4854; SEQ ID NO: 4855; SEQ ID NO: 4856; SEQ ID NO: 4857; SEQ ID NO: 4858; SEQ ID NO: 4859; SEQ ID NO: 4860; SEQ ID NO: 4861; SEQ ID NO: 4862; SEQ ID NO: 4863; SEQ ID NO: 4864; SEQ ID NO: 4865; SEQ ID NO: 4866; SEQ ID NO: 4867; SEQ ID NO: 4868; SEQ ID NO: 4869; SEQ ID NO: 4870; SEQ ID NO: 4871; SEQ ID NO: 4872; SEQ ID NO: 4873; SEQ ID NO: 4874; SEQ ID NO: 4875; SEQ ID NO: 4876; SEQ ID NO: 4877; SEQ ID NO: 4878; SEQ ID NO: 4879; SEQ ID NO: 4880; SEQ ID NO: 4881; SEQ ID NO: 4882; SEQ ID NO: 4883; SEQ ID NO: 4884; SEQ ID NO: 4885; SEQ ID NO: 4886; SEQ ID NO: 4887; SEQ ID NO: 4888; SEQ ID NO: 4889; SEQ ID NO: 4890; SEQ ID NO: 4891; SEQ ID NO: 4892; SEQ ID NO: 4893; SEQ ID NO: 4894; SEQ ID NO: 4895; SEQ ID NO: 4896; SEQ ID NO: 4897; SEQ ID NO: 4898; SEQ ID NO: 4899; SEQ ID NO: 4900; SEQ ID NO: 4901; SEQ ID NO: 4902; SEQ ID NO: 4903; SEQ ID NO: 4904; SEQ ID NO: 4905; SEQ ID NO: 4906; SEQ ID NO: 4907; SEQ ID NO: 4908; SEQ ID NO: 4909; SEQ ID NO: 4910; SEQ ID NO: 4911; SEQ ID NO: 4912; SEQ ID NO: 4913; SEQ ID NO: 4914; SEQ ID NO: 4915; SEQ ID NO: 4916; SEQ ID NO: 4917; SEQ ID NO: 4918; SEQ ID NO: 4919; SEQ ID NO: 4920; SEQ ID NO: 4921; SEQ ID NO: 4922; SEQ ID NO: 4923; SEQ ID NO: 4924; SEQ ID NO: 4925; SEQ ID NO: 4926; SEQ ID NO: 4927; SEQ ID NO: 4928; SEQ ID NO: 4929; SEQ ID NO: 4930; SEQ ID NO: 4931; SEQ ID NO: 4932; SEQ ID NO: 4933; SEQ ID NO: 4934; SEQ ID NO: 4935; SEQ ID NO: 4936; SEQ ID NO: 4937; SEQ ID NO: 4938; SEQ ID NO: 4939; SEQ ID NO: 4940; SEQ ID NO: 4941; SEQ ID NO: 4942; SEQ ID NO: 4943; SEQ ID NO: 4944; SEQ ID NO: 4945; SEQ ID NO: 4946; SEQ ID NO: 4947; SEQ ID NO: 4948; SEQ ID NO: 4949; SEQ ID NO: 4950; SEQ ID NO: 4951; SEQ ID NO: 4952; SEQ ID NO: 4953; SEQ ID NO: 4954; SEQ ID NO: 4955; SEQ ID NO: 4956; SEQ ID NO: 4957; SEQ ID NO: 4958; SEQ ID NO: 4959; SEQ ID NO: 4960; SEQ ID NO: 4961; SEQ ID NO: 4962; SEQ ID NO: 4963; SEQ ID NO: 4964; SEQ ID NO: 4965; SEQ ID NO: 4966; SEQ ID NO: 4967; SEQ ID NO: 4968; SEQ ID NO: 4969; SEQ ID NO: 4970; SEQ ID NO: 4971; SEQ ID NO: 4972; SEQ ID NO: 4973; SEQ ID NO: 4974; SEQ ID NO: 4975; SEQ ID NO: 4976; SEQ ID NO: 4977; SEQ ID NO: 4978; SEQ ID NO: 4979; SEQ ID NO: 4980; SEQ ID NO: 4981; SEQ ID NO: 4982; SEQ ID NO: 4983; SEQ ID NO: 4984; SEQ ID NO: 4985; SEQ ID NO: 4986; SEQ ID NO: 4987; SEQ ID NO: 4988; SEQ ID NO: 4989; SEQ ID NO: 4990; SEQ ID NO: 4991; SEQ ID NO: 4992; SEQ ID NO: 4993; SEQ ID NO: 4994; SEQ ID NO: 4995; SEQ ID NO: 4996; SEQ ID NO: 4997; SEQ ID NO: 4998; SEQ ID NO: 4999; SEQ ID NO: 5000; SEQ ID NO: 5001; SEQ ID NO: 5002; SEQ ID NO: 5003; SEQ ID NO: 5004; SEQ ID NO: 5005; SEQ ID NO: 5006; SEQ ID NO: 5007; SEQ ID NO: 5008; SEQ ID NO: 5009; SEQ ID NO: 5010; SEQ ID NO: 5011; SEQ ID NO: 5012; SEQ ID NO: 5013; SEQ ID NO: 5014; SEQ ID NO: 5015; SEQ ID NO: 5016; SEQ ID NO: 5017; SEQ ID NO: 5018; SEQ ID NO: 5019; SEQ ID NO: 5020; SEQ ID NO: 5021; SEQ ID NO: 5022; SEQ ID NO: 5023; SEQ ID NO: 5024; SEQ ID NO: 5025; SEQ ID NO: 5026; SEQ ID NO: 5027; SEQ ID NO: 5028; SEQ ID NO: 5029; SEQ ID NO: 5030; SEQ ID NO: 5031; SEQ ID NO: 5032; SEQ ID NO: 5033; SEQ ID NO: 5034; SEQ ID NO: 5035; SEQ ID NO: 5036; SEQ ID NO: 5037; SEQ ID NO: 5038; SEQ ID NO: 5039; SEQ ID NO: 5040; SEQ ID NO: 5041; SEQ ID NO: 5042; SEQ ID NO: 5043; SEQ ID NO: 5044; SEQ ID NO: 5045; SEQ ID NO: 5046; SEQ ID NO: 5047; SEQ ID NO: 5048; SEQ ID NO: 5049; SEQ ID NO: 5050; SEQ ID NO: 5051; SEQ ID NO: 5052; SEQ ID NO: 5053; SEQ ID NO: 5054; SEQ ID NO: 5055; SEQ ID NO: 5056; SEQ ID NO: 5057; SEQ ID NO: 5058; SEQ ID NO: 5059; SEQ ID NO: 5060; SEQ ID NO: 5061; SEQ ID NO: 5062; SEQ ID NO: 5063; SEQ ID NO: 5064; SEQ ID NO: 5065; SEQ ID NO: 5066; SEQ ID NO: 5067; SEQ ID NO: 5068; SEQ ID NO: 5069; SEQ ID NO: 5070; SEQ ID NO: 5071; SEQ ID NO: 5072; SEQ ID NO: 5073; SEQ ID NO: 5074; SEQ ID NO: 5075; SEQ ID NO: 5076; SEQ ID NO: 5077; SEQ ID NO: 5078; SEQ ID NO: 5079; SEQ ID NO: 5080; SEQ ID NO: 5081; SEQ ID NO: 5082; SEQ ID NO: 5083; SEQ ID NO: 5084; SEQ ID NO: 5085; SEQ ID NO: 5086; SEQ ID NO: 5087; SEQ ID NO: 5088; SEQ ID NO: 5089; SEQ ID NO: 5090; SEQ ID NO: 5091; SEQ ID NO: 5092; SEQ ID NO: 5093; SEQ ID NO: 5094; SEQ ID NO: 5095; SEQ ID NO: 5096; SEQ ID NO: 5097; SEQ ID NO: 5098; SEQ ID NO: 5099; SEQ ID NO: 5100; SEQ ID NO: 5101; SEQ ID NO: 5102; SEQ ID NO: 5103; SEQ ID NO: 5104; SEQ ID NO: 5105; SEQ ID NO: 5106; SEQ ID NO: 5107; SEQ ID NO: 5108; SEQ ID NO: 5109; SEQ ID NO: 5110; SEQ ID NO: 5111; SEQ ID NO: 5112; SEQ ID NO: 5113; SEQ ID NO: 5114; SEQ ID NO: 5115; SEQ ID NO: 5116; SEQ ID NO: 5117; SEQ ID NO: 5118; SEQ ID NO: 5119; SEQ ID NO: 5120; SEQ ID NO: 5121; SEQ ID NO: 5122; SEQ ID NO: 5123; SEQ ID NO: 5124; SEQ ID NO: 5125; SEQ ID NO: 5126; SEQ ID NO: 5127; SEQ ID NO: 5128; SEQ ID NO: 5129; SEQ ID NO: 5130; SEQ ID NO: 5131; SEQ ID NO: 5132; SEQ ID NO: 5133; SEQ ID NO: 5134; SEQ ID NO: 5135; SEQ ID NO: 5136; SEQ ID NO: 5137; SEQ ID NO: 5138; SEQ ID NO: 5139; SEQ ID NO: 5140; SEQ ID NO: 5141; SEQ ID NO: 5142; SEQ ID NO: 5143; SEQ ID NO: 5144; SEQ ID NO: 5145; SEQ ID NO: 5146; SEQ ID NO: 5147; SEQ ID NO: 5148; SEQ ID NO: 5149; SEQ ID NO: 5150; SEQ ID NO: 5151; SEQ ID NO: 5152; SEQ ID NO: 5153; SEQ ID NO: 5154; SEQ ID NO: 5155; SEQ ID NO: 5156; SEQ ID NO: 5157; SEQ ID NO: 5158; SEQ ID NO: 5159; SEQ ID NO: 5160; SEQ ID NO: 5161; SEQ ID NO: 5162; SEQ ID NO: 5163; SEQ ID NO: 5164; SEQ ID NO: 5165; SEQ ID NO: 5166; SEQ ID NO: 5167; SEQ ID NO: 5168; SEQ ID NO: 5169; SEQ ID NO: 5170; SEQ ID NO: 5171; SEQ ID NO: 5172; SEQ ID NO: 5173; SEQ ID NO: 5174; SEQ ID NO: 5175; SEQ ID NO: 5176; SEQ ID NO: 5177; SEQ ID NO: 5178; SEQ ID NO: 5179; SEQ ID NO: 5180; SEQ ID NO: 5181; SEQ ID NO: 5182; SEQ ID NO: 5183; SEQ ID NO: 5184; SEQ ID NO: 5185; SEQ ID NO: 5186; SEQ ID NO: 5187; SEQ ID NO: 5188; SEQ ID NO: 5189; SEQ ID NO: 5190; SEQ ID NO: 5191; SEQ ID NO: 5192; SEQ ID NO: 5193; SEQ ID NO: 5194; SEQ ID NO: 5195; SEQ ID NO: 5196; SEQ ID NO: 5197; SEQ ID NO: 5198; SEQ ID NO: 5199; SEQ ID NO: 5200; SEQ ID NO: 5201; SEQ ID NO: 5202; SEQ ID NO: 5203; SEQ ID NO: 5204; SEQ ID NO: 5205; SEQ ID NO: 5206; SEQ ID NO: 5207; SEQ ID NO: 5208; SEQ ID NO: 5209; SEQ ID NO: 5210; SEQ ID NO: 5211; SEQ ID NO: 5212; SEQ ID NO: 5213; SEQ ID NO: 5214; SEQ ID NO: 5215; SEQ ID NO: 5216; SEQ ID NO: 5217; SEQ ID NO: 5218; SEQ ID NO: 5219; SEQ ID NO: 5220; SEQ ID NO: 5221; SEQ ID NO: 5222; SEQ ID NO: 5223; SEQ ID NO: 5224; SEQ ID NO: 5225; SEQ ID NO: 5226.

In some embodiments, a composition is provided comprising: a) one or more nucleic acid molecules encoding a SaCas9 or SluCas9 and b) a pair of guide sequences comprising a first guide sequence and a second guide sequence, wherein the first and second guide sequences are selected from any two of the guide sequences of Table 1A (for SaCas9) or Table 1B (for SluCas9). In particular embodiments, the first and second guide sequences each comprise sequences that target the same exon. In particular embodiments, the first and second guide sequences each comprise sequences that target a different site in the same exon. In some embodiments, a composition is provided comprising a single nucleic acid molecule encoding, or two nucleic acid molecules where one molecule encodes, 1) one or more guide RNA that comprises a guide sequence selected from any one of SEQ ID NOs: 1000-3081; and 2) a SaCas9. In one aspect, a composition is provided comprising a single nucleic acid molecule encoding, or two nucleic acid molecules where one molecule encodes, 1) one or more guide RNA that comprises a guide sequence selected from any one of SEQ ID NOs: 4000-5226; and 2) a SluCas9. In particular embodiments, if the composition comprises one or more nucleic acid molecules encoding more than one guide RNAs, each guide RNA targets the same exon. In particular embodiments, each guide RNA targets a different site in the same exon.

In one aspect, a composition is provided comprising a single nucleic acid molecule encoding, or two nucleic acid molecules where one molecule encodes, a) one or more guide RNA that comprises a guide sequence that is at least 99%, 98%, 97%, 96%, 95%, 94%, 93%, 92%, 91%, or 90% identical to any one of SEQ ID NOs: 1000-3081 and a SaCas9; or b) one or more guide RNA that comprises a guide sequence that is at least 99%, 98%, 97%, 96%, 95%, 94%, 93%, 92%, 91%, or 90% identical to any one of SEQ ID NOs: 4000-5226; and a SluCas9. In one aspect, a composition is provided comprising a single nucleic acid molecule encoding a) one or more guide RNA that comprises a guide sequence that is at least 99%, 98%, 97%, 96%, 95%, 94%, 93%, 92%, 91%, or 90% identical to any one of 1) SEQ ID NOs: 4000-5226, and a SluCas9; or b) one or more guide RNA that comprises a guide sequence that is at least 99%, 98%, 97%, 96%, 95%, 94%, 93%, 92%, 91%, or 90% identical to any one of SEQ ID NOs: 1000-3081, and a SaCas9. In particular embodiments, if the composition comprises one or more nucleic acid molecules encoding more than one guide RNAs, each guide RNA targets the same exon. In particular embodiments, each guide RNA targets a different site in the same exon.

In another aspect, a composition is provided comprising a single nucleic acid molecule encoding, or two nucleic acid molecules where one molecule encodes, a) one or more guide RNA that comprises a guide sequence comprising at least 16, 17, 18, 19, or 20 contiguous nucleotides of a spacer sequence selected from any one of SEQ ID NOs: 1000-3081 and a SaCas9; or b) one or more guide RNA that comprises a guide sequence comprising at least 16, 17, 18, 19, or 20 contiguous nucleotides of a spacer sequence selected from any one of SEQ ID NOs: 4000-5226 and a SluCas9. In another aspect, a composition is provided comprising a single nucleic acid molecule encoding, or two nucleic acid molecules where one molecule encodes, a) one or more guide RNA that comprises a guide sequence comprising at least 16, 17, 18, 19, or 20 contiguous nucleotides of a spacer sequence selected from any one of SEQ ID NOs: 1000-3081 and a SaCas9; or b) one or more guide RNA that comprises a guide sequence comprising at least 16, 17, 18, 19, or 20 contiguous nucleotides of a spacer sequence selected from any one of SEQ ID NOs: 4000-5226 and a SluCas9. In particular embodiments, if the composition comprises one or more nucleic acid molecules encoding more than one guide RNAs, each guide RNA targets the same exon. In particular embodiments, each guide RNA targets a different site in the same exon.

In some embodiments, any of the guides disclosed herein may be used as a research tool, e.g., to study trafficking, expression, and processing by cells.

### Scaffold Sequences

Each of the guide sequences shown in Table 1A and Table 1B may further comprise additional nucleotides to form or encode a crRNA, e.g., using any known sequence appropriate for the Cas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

The guide RNA can be considered to comprise a scaffold sequence necessary for endonuclease binding and a spacer sequence required to bind to the genomic target sequence. In some embodiments, the guide RNA comprises any of the scaffold sequences disclosed herein and any of the spacer sequences disclosed herein. In some embodiments, the guide RNA comprises any of the scaffold sequences disclosed herein and any of the spacer sequences disclosed herein without any nucleotides between the scaffold sequence and the spacer sequence. An exemplary scaffold sequence suitable for use with SaCas9 to follow the guide sequence at its 3′ end is: GTTTAAGTACTCTGTGCTGGAAACAGCACAGAATCTACTTAAACAAGGCAAAATGCCGT GTTTATCTCGTCAACTTGTTGGCGAGA (SEQ ID NO: 500) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 500, or a sequence that differs from SEQ ID NO: 500 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV1” and is: GTTTTAGTACTCTGGAAACAGAATCTACTAAAACAAGGCAAAATGCCGTGTTTATCTCGT CAACTTGTTGGCGAGAT (SEQ ID NO: 501) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 501, or a sequence that differs from SEQ ID NO: 501 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV2” and is: GTTTAAGTACTCTGTGCTGGAAACAGCACAGAATCTACTTAAACAAGGCAAAATGCCGT GTTTATCTCGTCAACTTGTTGGCGAGAT (SEQ ID NO: 502) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 502, or a sequence that differs from SEQ ID NO: 502 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV3” and is: GTTTAAGTACTCTGGAAACAGAATCTACTTAAACAAGGCAAAATGCCGTGTTTATCTCGT CAACTTGTTGGCGAGAT (SEQ ID NO: 503) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 503, or a sequence that differs from SEQ ID NO: 503 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV5” and is: GTTTCAGTACTCTGGAAACAGAATCTACTGAAACAAGGCAAAATGCCGTGTTTATCTCGT CAACTTGTTGGCGAGAT (SEQ ID NO: 504) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 504, or a sequence that differs from SEQ ID NO: 504 by no more than 1, 2, 3, 4, 5, 10, 15, 20, or 25 nucleotides.

Two exemplary scaffold sequences suitable for use with SluCas9 to follow the guide sequence at its 3′ end are: GTTTTAGTACTCTGGAAACAGAATCTACTGAAACAAGACAATATGTCGTGTTTATCCCAT CAATTTATTGGTGGGA (SEQ ID NO: 600), orGTTTAAGTACTCTGTGCTGGAAACAGCACAGAATCTACTGAAACAAGACAATATGTCG TGTTTATCCCATCAATTTATTGGTGGGA (SEQ ID NO: 601) in 5′ to 3′ orientation. In some embodiments, an exemplary sequence for use with SluCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 600 or SEQ ID NO: 601, or a sequence that differs from SEQ ID NO: 600 or SEQ ID NO: 601 by no more than 1, 2, 3, 4, 5, 10, 15, 20, or 25 nucleotides.

Exemplary scaffold sequences suitable for use with SluCas9 to follow the guide sequence at its 3′ end are also shown below in the 5′ to 3′ orientation.

TABLE 2

| | | | | Streak of |
|---|---|---|---|---|
| | | | | Homology to |
| Scaffold | SEQ | | Homology | Slu v5 |
| ID | ID NO | Scaffold Sequence (5' to 3') | to Slu v5 | (# nucleotides) |
| Wildtype | 900 | GTTTTAGTACTCTGGAAACAGAATCTACTGAA | N/A | N/A |
| | | ACAAGACAATATGTCGTGTTTATCCCATCAAT | | |
| | | TTATTGGTGGGAT | | |
| Slu- | 601 | GTTTAAGTACTCTGTGCTGGAAACAGCACAG | N/A | N/A |
| VCGT- | | AATCTACTGAAACAAGACAATATGTCGTGTTT | | |
| 4.5 | | ATCCCATCAATTTATTGGTGGGA | | |
| Slu_v5 | 901 | GTTTCAGTACTCTGGAAACAGAATCTACTGAA | 100.00% | 77 |
| | | ACAAGACAATATGTCGTGTTTATCCCATCAAT | | |
| | | TTATTGGTGGGAT | | |
| Slu_v5-1 | 902 | GTTTggTaACcTaGGAAACTagATCTTaccAAACA | 87.50% | 47 |
| | | AGACAATATGTCGTGTTTATCCCATCAATTTA | | |
| | | TTGGTGGGAT | | |
| Slu_v5-2 | 903 | GTTTCAGTACTCTGGAAACAGAATCTACTGAA | 96.10% | 37 |
| | | ACAAGgCAAaATGcCGTGTTTATCCCATCAATT | | |
| | | TATTGGTGGGAT | | |
| Slu_v5-3 | 904 | GTTTCAGTACTCTGGAAACAGAATCTACTGAA | 94.81% | 48 |
| | | ACAAGACAATATGTCGcgcccaTCCCATCAATTT | | |
| | | ATTGGTGGGAT | | |
| Slu_v5-4 | 905 | GTTTCAGTACTCTGGAAACAGAATCTACTGAA | 91.55% | 55 |
| | | ACAAGACAATATGTCGTGTTTATgggTTgAATT | | |
| | | TATTcGacccAT | | |
| Slu_v5-5 | 906 | GTTTggTaACcTaGGAAACTagATCTTaccAAACA | 83.75% | 31 |
| | | AGgCAAaATGcCGTGTTTATCCCATCAATTTAT | | |
| | | TGGTGGGAT | | |
| Slu_v5-6 | 907 | GTTTggTaACcTaGGAAACTagATCTTaccAAACA | 82.50% | 23 |
| | | AGACAATATGTCGcgcccaTCCCATCAATTTATT | | |
| | | GGTGGGAT | | |
| Slu_v5-7 | 908 | GTTTggTaACcTaGGAAACTagATCTTaccAAACA | 78.38% | 25 |
| | | AGACAATATGTCGTGTTTATgggTTgAATTTAT | | |
| | | TcGacccAT | | |
| Slu_v5-8 | 909 | GTTTCAGTACTCTGGAAACAGAATCTACTGAA | 90.91% | 37 |
| | | ACAAGgCAAaATGcCGcgcccaTCCCATCAATTTA | | |
| | | TTGGTGGGAT | | |
| Slu_v5-9 | 910 | GTTTCAGTACTCTGGAAACAGAATCTACTGAA | 87.32% | 37 |
| | | ACAAGgCAAaATGcCGTGTTTATgggTTgAATTT | | |
| | | ATTcGacccAT | | |
| Slu_v5- | 911 | GTTTCAGTACTCTGGAAACAGAATCTACTGAA | 82.89% | 48 |
| 10 | | ACAAGACAATATGTCGcgcccaTgggTTgAATTTA | | |
| | | TTcGacccAT | | |
| Slu_v5- | 912 | GTTTggTaACcTaGGAAACTagATCTTaccAAACA | 78.75% | 23 |
| 11 | | AGgCAAaATGcCGcgcccaTCCCATCAATTTATTG | | |
| | | GTGGGAT | | |
| Slu_v5- | 913 | GTTTggTaACcTaGGAAACTagATCTTaccAAACA | 74.32% | 9 |
| 12 | | AGgCAAaATGcCGTGTTTATgggTTgAATTTATTc | | |
| | | GacccAT | | |
| Slu_v5- | 914 | GTTTggTaACcTaGGAAACTagATCTTaccAAACA | 70.89% | 18 |
| 13 | | AGACAATATGTCGcgcccaTgggTTgAATTTATTc | | |
| | | GacccAT | | |
| Slu_v5- | 915 | GTTTCAGTACTCTGGAAACAGAATCTACTGAA | 78.95% | 37 |
| 14 | | ACAAGgCAAaATGcCGcgcccaTgggTTgAATTTAT | | |
| | | TcGacccAT | | |
| Slu_v5- | 916 | GTTTggTaACcTaGGAAACTagATCTTaccAAACA | 67.09% | 8 |
| 15 | | AGgCAAaATGcCGcgcccaTgggTTgAATTTATTcGa | | |
| | | cccAT | | |
| Slu_v4 | 917 | GTTTCAGTACTCTGTGCTGGAAACAGCACAGA | N/A | N/A |
| | | ATCTACTGAAACAAGACAATATGTCGTGTTTA | | |
| | | TCCCATCAATTTATTGGTGGGAT | | |

In some embodiments, the scaffold sequence suitable for use with SaCas9 to follow the guide sequence at its 3′ end is selected from any one of SEQ ID NOs: 500-504 in 5′ to 3 orientation. In some embodiments, an exemplary sequence for use with SaCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 500-504, or a sequence that differs from any one of SEQ ID NOs: 500-504 by no more than 1, 2, 3, 4, 5, 10, 15, 20, or 25 nucleotides.

In some embodiments, the scaffold sequence suitable for use with SluCas9 to follow the guide sequence at its 3′ end is selected from any one of SEQ ID NOs: 900 or 601, or 901-917 in 5′ to 3 orientation. In some embodiments, an exemplary sequence for use with SluCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 900 or 601, or 901-917, or a sequence that differs from any one of SEQ ID NOs: 900 or 601, or 901-917 by no more than 1, 2, 3, 4, 5, 10, 15, 20, or 25 nucleotides.

In some embodiments, any scaffold sequence (e.g., any of the scaffold sequences disclosed herein) is suitable for use with any sRGN disclosed herein, such as any one of sRGN1, sRGN2, sRGN3, sRGN3.1, sRGN3.2, sRGN3.3, sRGN4, or a nucleic acid molecule encoding the same. In some embodiments, a scaffold sequence suitable for use with a sRGN, such as at the 3′ end of a guide RNA sequence within an expression vector comprising the sRGN (e.g., an expression vector as shown in FIG. 5) is selected from any one of SEQ ID NOs: 500-504, 601, or 900-917. In some embodiments, a scaffold sequence suitable for use with a sRGN, such as at the 3′ end of a guide RNA sequence within an expression vector comprising the sRGN (e.g., an expression vector as shown in FIG. 5) is a sequence that is at least 65%, 70%, 75%, 80%, 85%, 90%, 91%, 92%, 93%, 94%, 95%, 96%, 97%, 98%, 99% or 100% identical to any one of SEQ ID NOs: 500-504, 601, or 900-917, or a sequence that differs from any one of SEQ ID NOs: 500-504, 601, or 900-917 by no more than 1, 2, 3, 4, 5, 10, 15, 20, or 25 nucleotides.

In some embodiments, the nucleic acid encoding the gRNA comprises a sequence comprising SEQ ID NO: 500. In some embodiments, the nucleic acid encoding the gRNA comprises a sequence comprising SEQ ID NO: 501. In some embodiments, the nucleic acid encoding the gRNA comprises a sequence comprising SEQ ID NO: 502. In some embodiments, the nucleic acid encoding the gRNA comprises a sequence comprising SEQ ID NO: 503. In some embodiments, the nucleic acid encoding the gRNA comprises a sequence comprising SEQ ID NO: 504. In some embodiments, comprising a pair of gRNAs, one of the gRNAs comprises a sequence selected from any one of SEQ ID NOs: 500-504. In some embodiments, comprising a pair of gRNAs, both of the gRNAs comprise a sequence selected from any one of SEQ ID NOs: 500-504. In some embodiments, comprising a pair of gRNAs, the nucleotides 3′ of the guide sequence of the gRNAs are the same sequence. In some embodiments, comprising a pair of gRNAs, the nucleotides 3′ of the guide sequence of the gRNAs are different sequences.

In some embodiments, the nucleic acid encoding the gRNA comprises a sequence comprising SEQ ID NO: 900. In some embodiments, the nucleic acid encoding the gRNA comprises a sequence comprising SEQ ID NO: 601. In some embodiments, the nucleic acid encoding the gRNA comprises a sequence comprising SEQ ID NO: 900. In some embodiments, the nucleic acid encoding the gRNA comprises a sequence comprising SEQ ID NO: 901. In some embodiments, the nucleic acid encoding the gRNA comprises a sequence comprising SEQ ID NO: 902. In some embodiments, the nucleic acid encoding the gRNA comprises a sequence comprising SEQ ID NO: 903. In some embodiments, the nucleic acid encoding the gRNA comprises a sequence comprising SEQ ID NO: 904. In some embodiments, the nucleic acid encoding the gRNA comprises a sequence comprising SEQ ID NO: 905. In some embodiments, the nucleic acid encoding the gRNA comprises a sequence comprising SEQ ID NO: 906. In some embodiments, the nucleic acid encoding the gRNA comprises a sequence comprising SEQ ID NO: 907. In some embodiments, the nucleic acid encoding the gRNA comprises a sequence comprising SEQ ID NO: 908. In some embodiments, the nucleic acid encoding the gRNA comprises a sequence comprising SEQ ID NO: 909. In some embodiments, the nucleic acid encoding the gRNA comprises a sequence comprising SEQ ID NO: 910. In some embodiments, the nucleic acid encoding the gRNA comprises a sequence comprising SEQ ID NO: 911. In some embodiments, the nucleic acid encoding the gRNA comprises a sequence comprising SEQ ID NO: 912. In some embodiments, the nucleic acid encoding the gRNA comprises a sequence comprising SEQ ID NO: 913. In some embodiments, the nucleic acid encoding the gRNA comprises a sequence comprising SEQ ID NO: 914. In some embodiments, the nucleic acid encoding the gRNA comprises a sequence comprising SEQ ID NO: 915. In some embodiments, the nucleic acid encoding the gRNA comprises a sequence comprising SEQ ID NO: 916. In some embodiments, the nucleic acid encoding the gRNA comprises a sequence comprising SEQ ID NO: 917. In some embodiments, comprising a pair of gRNAs, one of the gRNAs comprises a sequence selected from any one of SEQ ID NOs: 900 or 601, or 901-917. In some embodiments, comprising a pair of gRNAs, both of the gRNAs comprise a sequence selected from any one of SEQ ID NOs: 900 or 601, or 901-917. In some embodiments, comprising a pair of gRNAs, the nucleotides 3′ of the guide sequence of the gRNAs are the same sequence. In some embodiments, comprising a pair of gRNAs, the nucleotides 3′ of the guide sequence of the gRNAs are different sequences.

In some embodiments, the scaffold sequence comprises one or more alterations in the stem loop 1 as compared to the stem loop 1 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the stem loop 2 as compared to the stem loop 2 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the tetraloop as compared to the tetraloop of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the repeat region as compared to the repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the anti-repeat region as compared to the anti-repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the linker region as compared to the linker region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). See, e.g., Nishimasu et al., 2015, Cell, 162:1113-1126 for description of regions of a scaffold.

Where a tracrRNA is used, in some embodiments, it comprises (5′ to 3′) a second complementary domain and a proximal domain. In the case of a sgRNA, guide sequences together with additional nucleotides (e.g., SEQ ID Nos: 500-504 (for SaCas9), and 900 or 601, or 901-917 (for SluCas9)) form or encode a sgRNA. In some embodiments, an sgRNA comprises (5′ to 3′) at least a spacer sequence, a first complementary domain, a linking domain, a second complementary domain, and a proximal domain. A sgRNA or tracrRNA may further comprise a tail domain. The linking domain may be hairpin-forming. See, e.g., US 2017/0007679 for detailed discussion and examples of crRNA and gRNA domains, including second complementarity domains, linking domains, proximal domains, and tail domains.

In some embodiments, the disclosure provides for specific nucleic acid sequences encoding one or more guide RNA components (e.g., any of the spacer and or scaffold sequences disclosed herein). The disclosure contemplates RNA equivalents of any of the DNA sequences provided herein (i.e., in which “T”s are replaced with “U”s), or DNA equivalents of any of the RNA sequences provided herein (i.e., in which “U”s are replaced with “T”s), as well as complements (including reverse complements) of any of the sequences disclosed herein.

In some embodiments, a composition is provided comprising a guide RNA, or nucleic acid encoding a guide RNA, wherein the guide RNA further comprises a trRNA. In each composition and method embodiment described herein, the crRNA (comprising the spacer sequence) and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

### Vectors

In any embodiment comprising a nucleic acid molecule encoding a guide RNA and/or a Cas9, the nucleic acid molecule may be a vector. In some embodiments, a composition is provided comprising a single nucleic acid molecule encoding at least one guide RNA and Cas9, wherein the nucleic acid molecule is a vector. In some embodiments, a composition is provided comprising more than one nucleic acid molecule encoding a guide RNA and Cas9, wherein the nucleic acid molecule is a vector. In some embodiments, a composition is provided comprising more than one nucleic acid molecule wherein one molecule encodes one or more guide RNA, and the other molecule encodes Cas9 plus or minus at least one guide RNA, wherein the nucleic acid molecule is a vector.

Any type of vector, such as any of those described herein, may be used. In some embodiments, the vector is a lipid nanoparticle. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector (AAV).

In some embodiment, the vector is a lipid nanoparticle comprising an endonuclease (e.g., any of the endonucleases disclosed herein) and one or more of any of the guide RNAs disclosed herein. In some embodiments, the vector is a lipid nanoparticle comprising a nucleic acid encoding an endonuclease (e.g., any of the endonucleases disclosed herein) and one or more of any of the guide RNAs disclosed herein. In some embodiments, the vector is a lipid nanoparticle comprising a nucleic acid encoding an endonuclease (e.g., any of the endonucleases disclosed herein) and a nucleic acid encoding one or more of any of the guide RNAs disclosed herein.

Where a vector is used, it may be a viral vector, such as a non-integrating viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. In some embodiments, the AAV vector size is measured in length of nucleotides from ITR to ITR, inclusive of both ITRs. In some embodiments, the AAV vector is less than 5 kb in size from ITR to ITR, inclusive of both ITRs. In particular embodiments, the AAV vector is less than 4.9 kb from ITR to ITR in size, inclusive of both ITRs. In further embodiments, the AAV vector is less than 4.85 kb in size from ITR to ITR, inclusive of both ITRs. In further embodiments, the AAV vector is less than 4.8 kb in size from ITR to ITR, inclusive of both ITRs. In further embodiments, the AAV vector is less than 4.75 kb in size from ITR to ITR, inclusive of both ITRs. In further embodiments, the AAV vector is less than 4.7 kb in size from ITR to ITR, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb in size from ITR to ITR, inclusive of both ITRs. In some embodiments, the vector is an AAV9 vector.

In some embodiments, the vector (e.g., viral vector, such as an adeno-associated viral vector) comprises a tissue-specific (e.g., muscle-specific) promoter, e.g., which is operatively linked to a sequence encoding the guide RNA and/or the Cas protein. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016; 3:16081, and references cited therein for detailed discussion of tissue-specific promoters including neuron-specific promoters.

In some embodiments, in addition to guide RNA and Cas9 sequences, the vectors further comprise nucleic acids that do not encode guide RNAs. Nucleic acids that do not encode guide RNA and Cas9 include, but are not limited to, promoters, enhancers, and regulatory sequences. In some embodiments, the vector comprises one or more nucleotide sequence(s) encoding a crRNA, a trRNA, or a crRNA and trRNA. In some embodiments, the muscle specific promoter is the CK8 promoter. The CK8 promoter has the following sequence (SEQ ID NO. 700):

| | |
|---|---|
| 1 | CTAGACTAGC ATGCTGCCCA TGTAAGGAGG CAAGGCCTGG |
| | GGACACCCGA GATGCCTGGT |
| 61 | TATAATTAAC CCAGACATGT GGCTGCCCCC CCCCCCCCAA |
| | CACCTGCTGC CTCTAAAAAT |
| 121 | AACCCTGCAT GCCATGTTCC CGGCGAAGGG CCAGCTGTCC |
| | CCCGCCAGCT AGACTCAGCA |
| 181 | CTTAGTTTAG GAACCAGTGA GCAAGTCAGC CCTTGGGGCA |
| | GCCCATACAA GGCCATGGGG |
| 241 | CTGGGCAAGC TGCACGCCTG GGTCCGGGGT GGGCACGGTG |
| | CCCGGGCAAC GAGCTGAAAG |
| 301 | CTCATCTGCT CTCAGGGGCC CCTCCCTGGG GACAGCCCCT |
| | CCTGGCTAGT CACACCCTGT |
| 361 | AGGCTCCTCT ATATAACCCA GGGGCACAGG GGCTGCCCTC |
| | ATTCTACCAC CACCTCCACA |
| 421 | GCACAGACAG ACACTCAGGA GCCAGCCAGC |

In some embodiments, the muscle-cell cell specific promoter is a variant of the CK8 promoter, called CK8e. In some embodiments, the size of the CK8e promoter is 436 bp. The CK8e promoter has the following sequence (SEQ ID NO. 701):

| | |
|---|---|
| 1 | TGCCCATGTA AGGAGGCAAG GCCTGGGGAC ACCCGAGATG |
| | CCTGGTTATA ATTAACCCAG |
| 61 | ACATGTGGCT GCCCCCCCCC CCCCAACACC TGCTGCCTCT |
| | AAAAATAACC CTGCATGCCA |
| 121 | TGTTCCCGGC GAAGGGCCAG CTGTCCCCCG CCAGCTAGAC |
| | TCAGCACTTA GTTTAGGAAC |
| 181 | CAGTGAGCAA GTCAGCCCTT GGGGCAGCCC ATACAAGGCC |
| | ATGGGGCTGG GCAAGCTGCA |
| 241 | CGCCTGGGTC CGGGGTGGGC ACGGTGCCCG GGCAACGAGC |
| | TGAAAGCTCA TCTGCTCTCA |
| 301 | GGGGCCCCTC CCTGGGGACA GCCCCTCCTG GCTAGTCACA |
| | CCCTGTAGGC TCCTCTATAT |
| 361 | AACCCAGGGG CACAGGGGCT GCCCTCATTC TACCACCACC |
| | TCCACAGCAC AGACAGACAC |
| 421 | TCAGGAGCCA GCCAGC |

In some embodiments, the Ck8e promoter comprises a nucleotide sequence that is at least 90%, 91%, 92%, 93%, 94%, 95%, 96%, 97%, 98%, 99% or 100% identical to the sequence of SEQ ID NO: 701.

In some embodiments, the vector comprises one or more of a U6, H1, or 7SK promoter. In some embodiments, the U6 promoter is the human U6 promoter (e.g., the U6L promoter or U6S promoter). In some embodiments, the promoter is the murine U6 promoter. In some embodiments, the 7SK promoter is a human 7SK promoter. In some embodiments, the 7SK promoter is the 7SK1 promoter. In some embodiments, the 7SK promoter is the 7SK2 promoter. In some embodiments, the H1 promoter is a human H1 promoter (e.g., the H1L promoter or the HIS promoter). In some embodiments, the vector comprises multiple guide sequences, wherein each guide sequence is under the control of a separate promoter. In some embodiments, each of the multiple guide sequences comprises a different sequence. In some embodiments, each of the multiple guide sequences comprise the same sequence (e.g., each of the multiple guide sequences comprise the same spacer sequence). In some embodiments, each of the multiple guide sequences comprises the same spacer sequence and the same scaffold sequence. In some embodiments, each of the multiple guide sequences comprises different spacer sequences and different scaffold sequences. In some embodiments, each of the multiple guide sequences comprises the same spacer sequence, but comprises a different scaffold sequence. In some embodiments, each of the multiple guide sequences comprises different spacer sequences and different scaffold sequences. In some embodiments, each of the separate promoters comprises the same nucleotide sequence (e.g., the U6 promoter sequence). In some embodiments, each of the separate promoters comprises a different nucleotide sequence (e.g., the U6, H1, and/or 7SK promoter sequence).

In some embodiments, the U6 promoter comprises a nucleotide sequence that is at least 90%, 91%, 92%, 93%, 94%, 95%, 96%, 97%, 98%, 99% or 100% identical to the sequence of SEQ ID NO: 702:

| | |
|---|---|
| cgagtccaac acccgtggga atcccatggg caccatggcc cctcgctcca aaaatgcttt | 60 |
| cgcgtcgcgc agacactgct cggtagtttc ggggatcagc gtttgagtaa gagcccgcgt | 120 |
| ctgaaccctc cgcgccgccc cggccccagt ggaaagacgc gcaggcaaaa cgcaccacgt | 180 |
| gacggagcgt gaccgcgcgc cgagcgcgcg ccaaggtcgg gcaggaagag ggcctatttc | 240 |
| ccatgattcc ttcatatttg catatacgat acaaggctgt tagagagata attagaatta | 300 |
| atttgactgt aaacacaaag atattagtac aaaatacgtg acgtagaaag taataatttc | 360 |
| ttgggtagtt tgcagtttta aaattatgtt ttaaaatgga ctatcatatg cttaccgtaa | 420 |
| cttgaaagta tttcgatttc ttggctttat atatcttgtg gaaaggacga aa | 472 |

In some embodiments, the H1 promoter comprises a nucleotide sequence that is at least 90%, 91%, 92%, 93%, 94%, 95%, 96%, 97%, 98%, 99% or 100% identical to the sequence of SEQ ID NO: 703:

| | |
|---|---|
| gctcggcgcg cccatatttg catgtcgcta tgtgttctgg gaaatcacca taaacgtgaa | 60 |
| atgtctttgg atttgggaat cttataagtt ctgtatgaga ccacggta | 108 |

In some embodiments, the 7SK promoter comprises a nucleotide sequence that is at least 90%, 91%, 92%, 93%, 94%, 95%, 96%, 97%, 98%, 99% or 100% identical to the sequence of SEQ ID NO: 704:

| | |
|---|---|
| tgacggcgcg ccctgcagta tttagcatgc cccacccatc tgcaaggcat tctggatagt | 60 |
| gtcaaaacag ccggaaatca agtccgttta tctcaaactt tagcattttg ggaataaatg | 120 |
| atatttgcta tgctggttaa attagatttt agttaaattt cctgctgaag ctctagtacg | 180 |
| ataagtaact tgacctaagt gtaaagttga gatttccttc aggtttatat agcttgtgcg | 240 |
| ccgcctgggt a | 251 |

In some embodiments, the U6 promoter is a hU6c promoter and comprises a nucleotide sequence that is at least 90%, 91%, 92%, 93%, 94%, 95%, 96%, 97%, 98%, 99% or 100% identical to the sequence of SEQ ID NO: 705:

GAGGGCCTATTTCCCATGATTCCTTCATATTTGCATATACGATACAAGGC
TGTTAGAGAGATAATTGGAATTAATTTGACTGTAAACACAAAGATATTAG
TACAAAATACGTGACGTAGAAAGTAATAATTTCTTGGGTAGTTTGCAGTT
TTAAAATTATGTTTTAAAATGGACTATCATATGCTTACCGTAACTTGAAA
GTATTTCGATTTCTTGGCTTTATATATCTTGTGGAAAGGACGAAACACC.

In some embodiments, the U6 promoter is a variant of the hU6c promoter. In some embodiments, the variant of the hU6c promoter comprises alternative nucleotides as compared to the sequence of SEQ ID NO: 705. In some embodiments, the variant of the hU6c promoter comprises fewer nucleotides as compared to the 249 nucleotides of SEQ ID NO: 705. In some embodiments, the variant of the hU6c promoter has fewer nucleotides in the nucleosome binding sequence of the hU6c promoter of SEQ ID NO: 705. In some embodiments, the variant of the hU6c promoter lacks all of or at least a portion of (e.g., at least 5, 10, 15, 20, 25, or 30 nucleotides) the nucleotides corresponding to nucleotides 96-125 of SEQ ID NO: 705. In some embodiments, the variant of the hU6c promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 705. In some embodiments, the variant of the hU6c promoter lacks all of or at least a portion of (e.g., at least 10, 20, 30, 40, 50, 60, 65, 70, 75, 80, or 85 nucleotides) the nucleotides corresponding to nucleotides 66-150 of SEQ ID NO: 705. In some embodiments, the variant of the hU6c promoter lacks all of or at least a portion of (e.g., at least 10, 20, 30, 40, 50, 60, 70, 80, 90, 100, 110, or 120 nucleotides) the nucleotides corresponding to nucleotides 51-170 of SEQ ID NO: 705. In some embodiments, the variant of the hU6c promoter lacks the nucleotides corresponding to nucleotides 96-125 of SEQ ID NO: 705. In some embodiments, the variant of the hU6c promoter comprises 129-219 nucleotides. In some embodiments, the variant of the hU6c promoter comprises 219 nucleotides. In some embodiments, the variant of the hU6c promoter comprises 189 nucleotides. In some embodiments, the variant of the hU6c promoter comprises 159 nucleotides. In some embodiments, the variant of the hU6c promoter comprises 129 nucleotides.

In some embodiments, the U6 promoter is hU6d30 and comprises a nucleotide sequence that is at least 90%, 91%, 92%, 93%, 94%, 95%, 96%, 97%, 98%, 99% or 100% identical to the sequence of SEQ ID NO: 9001:

GAGGGCCTATTTCCCATGATTCCTTCATATTTGCATATACGATACAAGGC
TGTTAGAGAGATAATTGGAATTAATTTGACTGTAAACACAAAGATATAAT
TTCTTGGGTAGTTTGCAGTTTTAAAATTATGTTTTAAAATGGACTATCAT
ATGCTTACCGTAACTTGAAAGTATTTCGATTTCTTGGCTTTATATATCTT
GTGGAAAGGACGAAACACC.

In some embodiments, the U6 promoter is hU6d60 and comprises a nucleotide sequence that is at least 90%, 91%, 92%, 93%, 94%, 95%, 96%, 97%, 98%, 99% or 100% identical to the sequence of SEQ ID NO: 9002:

GAGGGCCTATTTCCCATGATTCCTTCATATTTGCATATACGATACAAGGC
TGTTAGAGAGATAATTGGAATTAATTTGACGTTTGCAGTTTTAAAATTAT
GTTTTAAAATGGACTATCATATGCTTACCGTAACTTGAAAGTATTTCGAT
TTCTTGGCTTTATATATCTTGTGGAAAGGACGAAACACC.

In some embodiments, the U6 promoter is hU6d90 and comprises a nucleotide sequence that is at least 90%, 91%, 92%, 93%, 94%, 95%, 96%, 97%, 98%, 99% or 100% identical to the sequence of SEQ ID NO: 9003:

GAGGGCCTATTTCCCATGATTCCTTCATATTTGCATATACGATACAAGGC
TGTTAGAGAGATAATATTATGTTTTAAAATGGACTATCATATGCTTACCG
TAACTTGAAAGTATTTCGATTTCTTGGCTTTATATATCTTGTGGAAAGGA
CGAAACACC.

In some embodiments, the U6 promoter is hU6d120 and comprises a nucleotide sequence that is at least 90%, 91%, 92%, 93%, 94%, 95%, 96%, 97%, 98%, 99% or 100% identical to the sequence of SEQ ID NO: 9004.

GAGGGCCTATTTCCCATGATTCCTTCATATTTGCATATACGATACAAGGC
GGACTATCATATGCTTACCGTAACTTGAAAGTATTTCGATTTCTTGGCTT
TATATATCTTGTGGAAAGGACGAAACACC.

In some embodiments, the 7SK promoter is a 7SK2 promoter and comprises a nucleotide sequence that is at least 90%, 91%, 92%, 93%, 94%, 95%, 96%, 97%, 98%, 99% or 100% identical to the sequence of SEQ ID NO: 706:

CTGCAGTATTTAGCATGCCCCACCCATCTGCAAGGCATTCTGGATAGTGT
CAAAACAGCCGGAAATCAAGTCCGTTTATCTCAAACTTTAGCATTTTGGG
AATAAATGATATTTGCTATGCTGGTTAAATTAGATTTTAGTTAAATTTCC
TGCTGAAGCTCTAGTACGATAAGCAACTTGACCTAAGTGTAAAGTTGAGA
CTTCCTTCAGGTTTATATAGCTTGTGCGCCGCTTGGGTACCTC.

In some embodiments, the 7SK promoter is a variant of the 7SK2 promoter. In some embodiments, the variant of the 7SK2 promoter comprises alternative nucleotides as compared to the sequence of SEQ ID NO: 706. In some embodiments, the variant of the 7SK2 promoter e.g., comprises fewer nucleotides as compared to the 243 nucleotides of SEQ ID NO: 706. In some embodiments, the variant of the 7SK2 promoter has fewer nucleotides in the nucleosome binding sequence of the 7SK2 promoter of SEQ ID NO: 706. In some embodiments, the variant of the 7SK2 promoter lacks all of or at least a portion of (e.g., at least 5, 10, 15, 20, 25, or 30 nucleotides) the nucleotides corresponding to nucleotides 95-124 of SEQ ID NO: 706. In some embodiments, the variant of the 7SK2 promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 706. In some embodiments, the variant of the 7SK2 promoter lacks all of or at least a portion of (e.g., at least 10, 20, 30, 40, 50, 60, 65, 70, 75, 80, 85 or 90 nucleotides) the nucleotides corresponding to nucleotides 67-156 of SEQ ID NO: 706. In some embodiments, the variant of the 7SK2 promoter lacks all of or at least a portion of (e.g., at least 10, 20, 30, 40, 50, 60, 70, 80, 90, 100, 110, or 120 nucleotides) the nucleotides corresponding to nucleotides 52-171 of SEQ ID NO: 706. In some embodiments, the variant of the 7SK2 promoter comprises 123-213 nucleotides. In some embodiments, the variant of the 7SK2 promoter comprises 213 nucleotides. In some embodiments, the variant of the 7SK2 promoter comprises 183 nucleotides. In some embodiments, the variant of the 7SK2 promoter comprises 153 nucleotides. In some embodiments, the variant of the 7SK2 promoter comprises 123 nucleotides.

In some embodiments, the 7SK promoter is 7SKd30 and comprises a nucleotide sequence that is at least 90%, 91%, 92%, 93%, 94%, 95%, 96%, 97%, 98%, 99% or 100% identical to the sequence of SEQ ID NO: 9006:

CTGCAGTATTTAGCATGCCCCACCCATCTGCAAGGCATTCTGGATAGTGT
CAAAACAGCCGGAAATCAAGTCCGTTTATCTCAAACTTTAGCATTTAAAT
TAGATTTTAGTTAAATTTCCTGCTGAAGCTCTAGTACGATAAGCAACTTG
ACCTAAGTGTAAAGTTGAGACTTCCTTCAGGTTTATATAGCTTGTGCGCC
GCTTGGGTACCTC.

In some embodiments, the 7SK promoter is 7SKd60 and comprises a nucleotide sequence that is at least 90%, 91%, 92%, 93%, 94%, 95%, 96%, 97%, 98%, 99% or 100% identical to the sequence of SEQ ID NO: 9007:

CTGCAGTATTTAGCATGCCCCACCCATCTGCAAGGCATTCTGGATAGTGT
CAAAACAGCCGGAAATCAAGTCCGTTTATCTTAAATTTCCTGCTGAAGCT
CTAGTACGATAAGCAACTTGACCTAAGTGTAAAGTTGAGACTTCCTTCAG
GTTTATATAGCTTGTGCGCCGCTTGGGTACCTC.

In some embodiments, the 7SK promoter is 7SKd90 and comprises a nucleotide sequence that is at least 90%, 91%, 92%, 93%, 94%, 95%, 96%, 97%, 98%, 99% or 100% identical to the sequence of SEQ ID NO: 9008:

CTGCAGTATTTAGCATGCCCCACCCATCTGCAAGGCATTCTGGATAGTGT
CAAAACAGCCGGAAATAGCTCTAGTACGATAAGCAACTTGACCTAAGTGT
AAAGTTGAGACTTCCTTCAGGTTTATATAGCTTGTGCGCCGCTTGGGTAC
CTC.

In some embodiments, the 7SK promoter is 7SKd120 and comprises a nucleotide sequence that is at least 90%, 91%, 92%, 93%, 94%, 95%, 96%, 97%, 98%, 99% or 100% identical to the sequence of SEQ ID NO: 9009:

CTGCAGTATTTAGCATGCCCCACCCATCTGCAAGGCATTCTGGATAGTGTCAG CAACTTGACCTAAGTGTAAAGTTGAGACTTCCTTCAGGTTTATATAGCTTGTGCGCCGCTT GGGTACCTC. In some embodiments, the H1 promoter is a H1m or mH1 promoter and comprises a nucleotide sequence that is at least 90%, 91%, 92%, 93%, 94%, 95%, 96%, 97%, 98%, 99% or 100% identical to the sequence of SEQ ID NO: 707:

AATATTTGCATGTCGCTATGTGTTCTGGGAAATCACCATAAACGTGAAAT
GTCTTTGGATTTGGGAATCTTATAAGTTCTGTATGAGACCACTCTTTCC
C.

In some embodiments, the promoter is an M 11 promoter and comprises a nucleotide sequence that is at least 90%, 91%, 92%, 93%, 94%, 95%, 96%, 97%, 98%, 99% or 100% identical to the sequence of SEQ ID NO: 708:

ATATTTAGCATGTCGCTATGTGTTCTGGGAAACTTGACCTAAGTGTAAA
GTTGAGATTTCCTTCAGGTTTATATAGTTCTGTATGAGACCACTCTTTC
CC.

In some embodiments, the vector comprises multiple inverted terminal repeats (ITRs). These ITRs may be of an AAV1, AAV2, AAV3, AAV4, AAV5, AAV6, AAV7, AAV8, or AAV9 serotype. In some embodiments, the ITRs are of an AAV2 serotype. In some embodiments, the 5′ ITR comprises a sequence that is at least 80%, 85%, 90%, 91%, 92%, 93%, 94%, 95%, 96%, 97%, 98%, 99%, or 100% identical to the sequence of SEQ ID NO: 709:

GGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGC
GAGCGCGCAGAGAGGGAGTGGCCAACTCCATCACTAGGGGTTCCT.

In some embodiments, the 5′ ITR comprises a sequence that is at least 80%, 85%, 90%, 91%, 92%, 93%, 94%, 95%, 96%, 97%, 98%, 99%, or 100% identical to the sequence of SEQ ID NO: 6:

CGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAG
CGAGCGCGCAGAGAGGGAGTGGCCAACTCCATCACTAGGGGTTCCT.

In some embodiments, the 3′ ITR comprises a sequence that is at least 80%, 85%, 90%, 91%, 92%, 93%, 94%, 95%, 96%, 97%, 98%, 99%, or 100% identical to the sequence of SEQ ID NO: 710:

AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGAGAGGGA.

In some embodiments, the 3′ ITR comprises a sequence that is at least 80%, 85%, 90%, 91%, 92%, 93%, 94%, 95%, 96%, 97%, 98%, 99%, or 100% identical to the sequence of SEQ ID NO: 7:

AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGAGAGGGAA.

In some embodiments, a vector comprising a single nucleic acid molecule encoding 1) two or more guide RNA comprising any one or more of the spacer sequences of Table 1A and Table 1B; and 2) a SaCas9 (for any one or more of SEQ ID Nos: 1000-3081) or SluCas9 (for any one or more of SEQ ID NO: 4000-5226) is provided. In particular embodiments, if the two or more guide RNAs target the same exon. In particular embodiments, the two or more guide RNAs each target a different site in the same exon. In some embodiments, the vector is an AAV vector. In some embodiments, the AAV vector is administered to a subject to treat DMD. In some embodiments, only one vector is needed due to the use of a particular guide sequence that is useful in the context of SaCas9 or SluCas9.

In some embodiments, the vector comprises a nucleic acid encoding a Cas9 protein (e.g., an SaCas9 or SluCas9 protein) and further comprises a nucleic acid encoding one or more single guide RNA(s). In some embodiments, the nucleic acid encoding the Cas9 protein is under the control of a CK8e promoter. In some embodiments, the nucleic acid encoding the guide RNA sequence is under the control of a hU6c promoter. In some embodiments, the vector is AAV9.

In some embodiments, the vector comprises multiple nucleic acids encoding more than one guide RNA. In some embodiments, the vector comprises two nucleic acids encoding two different guide RNA sequences.

In some embodiments, the vector comprises a nucleic acid encoding a Cas9 protein (e.g., an SaCas9 protein or SluCas9 protein), a nucleic acid encoding a first guide RNA, and a nucleic acid encoding a second guide RNA. In some embodiments, the vector does not comprise a nucleic acid encoding more than two guide RNAs. In some embodiments, the nucleic acid encoding the first guide RNA is the same as the nucleic acid encoding the second guide RNA. In some embodiments, the nucleic acid encoding the first guide RNA is different from the nucleic acid encoding the second guide RNA. In some embodiments, the vector comprises a single nucleic acid molecule, wherein the single nucleic acid molecule comprises a nucleic acid encoding a Cas9 protein, a nucleic acid encoding a first guide RNA, and a nucleic acid that is the reverse complement to the coding sequence for the second guide RNA. In some embodiments, the vector comprises a single nucleic acid molecule, wherein the single nucleic acid molecule comprises a nucleic acid encoding a Cas9 protein, a nucleic acid that is the reverse complement to the coding sequence for the first guide RNA, and a nucleic acid that is the reverse complement to the coding sequence for the second guide RNA. In some embodiments, the nucleic acid encoding a Cas9 protein (e.g., an SaCas9 or SluCas9 protein) is under the control of the CK8e promoter. In some embodiments, the first guide is under the control of the hU6c promoter and the second guide is under the control of the hU6c promoter. In some embodiments, the first guide is under the control of the 7SK2 promoter, and the second guide is under the control of the H1m promoter. In some embodiments, the first guide is under the control of the H1m promoter, and the second guide is under the control of the 7SK2 promoter. In some embodiments, the first guide is under the control of the hU6c promoter, and the second guide is under the control of the H1m promoter. In some embodiments, the first guide is under the control of the H1m promoter, and the second guide is under the control of the hU6c promoter. In some embodiments, the nucleic acid encoding the Cas9 protein is: a) between the nucleic acids encoding the guide RNAs, b) between the nucleic acids that are the reverse complement to the coding sequences for the guide RNAs, c) between the nucleic acid encoding the first guide RNA and the nucleic acid that is the reverse complement to the coding sequence for the second guide RNA, d) between the nucleic acid encoding the second guide RNA and the nucleic acid that is the reverse complement to the coding sequence for the first guide RNA, e) 5′ to the nucleic acids encoding the guide RNAs, f) 5′ to the nucleic acids that are the reverse complements to the coding sequences for the guide RNAs, g) 5′ to a nucleic acid encoding one of the guide RNAs and 5′ to a nucleic acid that is the reverse complement to the coding sequence for the other guide RNA, h) 3′ to the nucleic acids encoding the guide RNAs, i) 3′ to the nucleic acids that are the reverse complements to the coding sequences for the guide RNAs, or j) 3′ to a nucleic acid encoding one of the guide RNAs and 3′ to a nucleic acid that is the reverse complement to the coding sequence for the other guide RNA. In some embodiments, any of the vectors disclosed herein is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb from ITR to ITR in size, inclusive of both ITRs. In some embodiments, the vector is an AAV9 vector.

In some embodiments, any of the vectors disclosed herein comprises a nucleic acid encoding at least a first guide RNA and a second guide RNA. In some embodiments, the nucleic acid comprises a spacer-encoding sequence for the first guide RNA, a scaffold-encoding sequence for the first guide RNA, a spacer-encoding sequence for the second guide RNA, and a scaffold-encoding sequence of the second guide RNA. In some embodiments, the spacer-encoding sequence (e.g., encoding any of the spacer sequences disclosed herein) for the first guide RNA is identical to the spacer-encoding sequence for the second guide RNA. In some embodiments, the spacer-encoding sequence (e.g., encoding any of the spacer sequences disclosed herein) for the first guide RNA is different from the spacer-encoding sequence for the second guide RNA. In some embodiments, the scaffold-encoding sequence for the first guide RNA is identical to the scaffold-encoding sequence for the second guide RNA. In some embodiments, the scaffold-encoding sequence for the first guide RNA is different from the scaffold-encoding sequence for the nucleic acid encoding the second guide RNA. In some embodiments, the scaffold-encoding sequence for the first guide RNA comprises a sequence selected from the group consisting of SEQ ID Nos: 500-504 (for SaCas9) and 601 or 900-917 (for SluCas9), and the scaffold-encoding sequence for the second guide RNA comprises a different sequence selected from the group consisting of SEQ ID Nos: 500-504 (for SaCas9) and 601 or 900-917 (for SluCas9). In some embodiments, the scaffold-encoding sequence for the first guide RNA comprises the sequence of SEQ ID NO: 500, and the scaffold-encoding sequence for the second guide RNA comprises the sequence of SEQ ID NO: 501. In some embodiments, the scaffold-encoding sequence for the first guide RNA comprises the sequence of SEQ ID NO: 500, and the scaffold-encoding sequence for the second guide RNA comprises the sequence of SEQ ID NO: 502. In some embodiments, the scaffold-encoding sequence for the first guide RNA comprises the sequence of SEQ ID NO: 500, and the scaffold-encoding sequence for the second guide RNA comprises the sequence of SEQ ID NO: 503. In some embodiments, the scaffold-encoding sequence for the first guide RNA comprises the sequence of SEQ ID NO: 500, and the scaffold-encoding sequence for the second guide RNA comprises the sequence of SEQ ID NO: 504. In some embodiments, the scaffold-encoding sequence for the first guide RNA comprises the sequence of SEQ ID NO: 501, and the scaffold-encoding sequence for the second guide RNA comprises the sequence of SEQ ID NO: 502. In some embodiments, the scaffold-encoding sequence for the first guide RNA comprises the sequence of SEQ ID NO: 501, and the scaffold-encoding sequence for the second guide RNA comprises the sequence of SEQ ID NO: 503. In some embodiments, the scaffold-encoding sequence for the first guide RNA comprises the sequence of SEQ ID NO: 501, and the scaffold-encoding sequence for the second guide RNA comprises the sequence of SEQ ID NO: 504. In some embodiments, the scaffold-encoding sequence for the first guide RNA comprises the sequence of SEQ ID NO: 502, and the scaffold-encoding sequence for the second guide RNA comprises the sequence of SEQ ID NO: 503. In some embodiments, the scaffold-encoding sequence for the first guide RNA comprises the sequence of SEQ ID NO: 502, and the scaffold-encoding sequence for the second guide RNA comprises the sequence of SEQ ID NO: 504. In some embodiments, the scaffold-encoding sequence for the first guide RNA comprises the sequence of SEQ ID NO: 503, and the scaffold-encoding sequence for the second guide RNA comprises the sequence of SEQ ID NO: 504. In particular embodiments, the scaffold-encoding sequence for the first guide RNA comprises the sequence of SEQ ID NO: 504, and the scaffold-encoding sequence for the second guide RNA comprises the sequence of SEQ ID NO: 504. In some embodiments, the scaffold-encoding sequence for the first guide RNA comprises the sequence of SEQ ID NO: 600, and the scaffold-encoding sequence for the second guide RNA comprises the sequence of SEQ ID NO: 900. In some embodiments, the scaffold-encoding sequence for the first guide RNA comprises the sequence of SEQ ID NO: 600, and the scaffold-encoding sequence for the second guide RNA comprises the sequence of SEQ ID NO: 901. In some embodiments, the scaffold-encoding sequence for the first guide RNA comprises the sequence of SEQ ID NO: 600, and the scaffold-encoding sequence for the second guide RNA comprises the sequence of SEQ ID NO: 902. In some embodiments, the scaffold-encoding sequence for the first guide RNA comprises the sequence of SEQ ID NO: 600, and the scaffold-encoding sequence for the second guide RNA comprises the sequence of SEQ ID NO: 903. In some embodiments, the scaffold-encoding sequence for the first guide RNA comprises the sequence of SEQ ID NO: 600, and the scaffold-encoding sequence for the second guide RNA comprises the sequence of SEQ ID NO: 904. In some embodiments, the scaffold-encoding sequence for the first guide RNA comprises the sequence of SEQ ID NO: 600, and the scaffold-encoding sequence for the second guide RNA comprises the sequence of SEQ ID NO: 905. In some embodiments, the scaffold-encoding sequence for the first guide RNA comprises the sequence of SEQ ID NO: 600, and the scaffold-encoding sequence for the second guide RNA comprises the sequence of SEQ ID NO: 906. In some embodiments, the scaffold-encoding sequence for the first guide RNA comprises the sequence of SEQ ID NO: 600, and the scaffold-encoding sequence for the second guide RNA comprises the sequence of SEQ ID NO: 907. In some embodiments, the scaffold-encoding sequence for the first guide RNA comprises the sequence of SEQ ID NO: 600, and the scaffold-encoding sequence for the second guide RNA comprises the sequence of SEQ ID NO: 908. In some embodiments, the scaffold-encoding sequence for the first guide RNA comprises the sequence of SEQ ID NO: 600, and the scaffold-encoding sequence for the second guide RNA comprises the sequence of SEQ ID NO: 909. In some embodiments, the scaffold-encoding sequence for the first guide RNA comprises the sequence of SEQ ID NO: 600, and the scaffold-encoding sequence for the second guide RNA comprises the sequence of SEQ ID NO: 910. In some embodiments, the scaffold-encoding sequence for the first guide RNA comprises the sequence of SEQ ID NO: 600, and the scaffold-encoding sequence for the second guide RNA comprises the sequence of SEQ ID NO: 911. In some embodiments, the scaffold-encoding sequence for the first guide RNA comprises the sequence of SEQ ID NO: 600, and the scaffold-encoding sequence for the second guide RNA comprises the sequence of SEQ ID NO: 912. In some embodiments, the scaffold-encoding sequence for the first guide RNA comprises the sequence of SEQ ID NO: 600, and the scaffold-encoding sequence for the second guide RNA comprises the sequence of SEQ ID NO: 913. In some embodiments, the scaffold-encoding sequence for the first guide RNA comprises the sequence of SEQ ID NO: 600, and the scaffold-encoding sequence for the second guide RNA comprises the sequence of SEQ ID NO: 914. In some embodiments, the scaffold-encoding sequence for the first guide RNA comprises the sequence of SEQ ID NO: 600, and the scaffold-encoding sequence for the second guide RNA comprises the sequence of SEQ ID NO: 915. In some embodiments, the scaffold-encoding sequence for the first guide RNA comprises the sequence of SEQ ID NO: 600, and the scaffold-encoding sequence for the second guide RNA comprises the sequence of SEQ ID NO: 916. In some embodiments, the scaffold-encoding sequence for the first guide RNA comprises the sequence of SEQ ID NO: 600, and the scaffold-encoding sequence for the second guide RNA comprises the sequence of SEQ ID NO: 917. In some embodiments, the scaffold-encoding sequence for the first guide RNA comprises the sequence of SEQ ID NO: 901, and the scaffold-encoding sequence for the second guide RNA comprises the sequence of SEQ ID NO: 902. In some embodiments, the scaffold-encoding sequence for the first guide RNA comprises the sequence of SEQ ID NO: 901, and the scaffold-encoding sequence for the second guide RNA comprises the sequence of SEQ ID NO: 903. In some embodiments, the scaffold-encoding sequence for the first guide RNA comprises the sequence of SEQ ID NO: 901, and the scaffold-encoding sequence for the second guide RNA comprises the sequence of SEQ ID NO: 904. In some embodiments, the scaffold-encoding sequence for the first guide RNA comprises the sequence of SEQ ID NO: 901, and the scaffold-encoding sequence for the second guide RNA comprises the sequence of SEQ ID NO: 905. In some embodiments, the scaffold-encoding sequence for the first guide RNA comprises the sequence of SEQ ID NO: 901, and the scaffold-encoding sequence for the second guide RNA comprises the sequence of SEQ ID NO: 906. In some embodiments, the scaffold-encoding sequence for the first guide RNA comprises the sequence of SEQ ID NO: 901, and the scaffold-encoding sequence for the second guide RNA comprises the sequence of SEQ ID NO: 907. In some embodiments, the scaffold-encoding sequence for the first guide RNA comprises the sequence of SEQ ID NO: 901, and the scaffold-encoding sequence for the second guide RNA comprises the sequence of SEQ ID NO: 908. In some embodiments, the scaffold-encoding sequence for the first guide RNA comprises the sequence of SEQ ID NO: 901, and the scaffold-encoding sequence for the second guide RNA comprises the sequence of SEQ ID NO: 909. In some embodiments, the scaffold-encoding sequence for the first guide RNA comprises the sequence of SEQ ID NO: 901, and the scaffold-encoding sequence for the second guide RNA comprises the sequence of SEQ ID NO: 910. In some embodiments, the scaffold-encoding sequence for the first guide RNA comprises the sequence of SEQ ID NO: 901, and the scaffold-encoding sequence for the second guide RNA comprises the sequence of SEQ ID NO: 911. In some embodiments, the scaffold-encoding sequence for the first guide RNA comprises the sequence of SEQ ID NO: 901, and the scaffold-encoding sequence for the second guide RNA comprises the sequence of SEQ ID NO: 912. In some embodiments, the scaffold-encoding sequence for the first guide RNA comprises the sequence of SEQ ID NO: 901, and the scaffold-encoding sequence for the second guide RNA comprises the sequence of SEQ ID NO: 913. In some embodiments, the scaffold-encoding sequence for the first guide RNA comprises the sequence of SEQ ID NO: 901, and the scaffold-encoding sequence for the second guide RNA comprises the sequence of SEQ ID NO: 914. In some embodiments, the scaffold-encoding sequence for the first guide RNA comprises the sequence of SEQ ID NO: 901, and the scaffold-encoding sequence for the second guide RNA comprises the sequence of SEQ ID NO: 915. In some embodiments, the scaffold-encoding sequence for the first guide RNA comprises the sequence of SEQ ID NO: 901, and the scaffold-encoding sequence for the second guide RNA comprises the sequence of SEQ ID NO: 916. In some embodiments, the scaffold-encoding sequence for the first guide RNA comprises the sequence of SEQ ID NO: 901, and the scaffold-encoding sequence for the second guide RNA comprises the sequence of SEQ ID NO: 917. In particular embodiments, the scaffold-encoding sequence for the first guide RNA comprises the sequence of SEQ ID NO: 901, and the scaffold-encoding sequence for the second guide RNA comprises the sequence of SEQ ID NO: 901. In some embodiments, the spacer encoding sequence for the first guide RNA is the same as the spacer-encoding sequence in the second guide RNA, and the scaffold-encoding sequence for the first guide RNA is different from the scaffold-encoding sequence in the nucleic acid encoding the second guide RNA.

In some embodiments, the AAV vector is in a particular configuration. Some examples of these AAV vector configurations are provided herein, and the order of elements in these exemplary vectors are referenced in a 5′ to 3′ manner with respect to the plus strand. For these configurations, it should be understood that the recited elements may not be directly contiguous, and that one or more nucleotides or one or more additional elements may be present between the recited elements. However, in some embodiments, it is possible that no nucleotides or no additional elements are present between the recited elements. Also, unless otherwise stated, “a promoter for expression of element X” means that the promoter is oriented in a manner to facilitate expression of the recited element X. In addition, unless otherwise stated, references to an “sgRNA scaffold sequence” or “a guide RNA scaffold sequence” are synonymous with “a nucleotide sequence/nucleic acid encoding an sgRNA scaffold sequence” or “a nucleotide sequence/nucleic acid encoding a guide RNA scaffold sequence.” In some embodiments, the disclosure provides for a nucleic acid encoding an SaCas9 (e.g., an SaCas9-KKH) or SluCas9. In some embodiments, the nucleic acid encodes for one or more nuclear localization signals (e.g., the SV40 NLS and/or the c-Myc NLS) on the C-terminus of the encoded SaCas9 or SluCas9. In some embodiments, the nucleic acid encodes for one or more NLSs (e.g., the SV40 NLS and/or the c-Myc NLS) on the C-terminus of the encoded SaCas9 or SluCas9, and the nucleic acid does not encode for an NLS on the N-terminus of the encoded SaCas9 or SluCas9. In some embodiments, the nucleic acid encodes for one or more nuclear localization signals (e.g., the SV40 NLS and/or the c-Myc NLS) on the N-terminus of the encoded SaCas9 or SluCas9. In some embodiments, the nucleic acid encodes for one or more NLSs (e.g., the SV40 NLS and/or the c-Myc NLS) on the N-terminus of the encoded SaCas9 or SluCas9, and the nucleic acid does not encode for an NLS on the C-terminus of the encoded SaCas9 or SluCas9. In some embodiments, the nucleic acid encodes for one or more nuclear localization signals (e.g., the SV40 NLS and/or the c-Myc NLS) on the C-terminus of the encoded SaCas9 or SluCas9 and also encodes for one or more NLSs on the N-terminus of the encoded SaCas9 or SluCas9 (e.g., the SV40 NLS and/or the c-Myc NLS). In some embodiments, the nucleic acid encodes one NLS. In some embodiments, the nucleic acid encodes two NLSs. In some embodiments, the nucleic acid encodes three NLSs. The one, two, or three NLS may all be C-terminal, N-terminal, or any combination of C- and N-terminal. The NLS may be fused/attached directly to the C- or N-terminus or to another NLS, or may be fused/attached indirectly attached through a linker. In some embodiments, an additional domain may be: a) fused to the N- or C-terminus of the Cas protein (e.g., a Cas9 protein), b) fused to the N-terminus of an NLS fused to the N-terminus of a Cas protein, or c) fused to the C-terminus of an NLS fused to the C-terminus of a Cas protein, with or without a linker. In some embodiments, an NLS is fused to the N- and/or C-terminus of the Cas protein by means of a linker. In some embodiments, an NLS is fused to the N-terminus of an N-terminally-fused NLS on a Cas protein by means of a linker, and/or an NLS is fused to the C-terminus of a C-terminally fused NLS on a Cas protein by means of a linker. In some embodiments, the linker is GSVD (SEQ ID NO: 550) or GSGS (SEQ ID NO: 551). In some embodiments, the Cas protein comprises a c-Myc NLS fused to the N-terminus of the Cas protein (or to an N-terminally-fused NLS on the Cas protein), optionally by means of a linker. In some embodiments, the Cas protein comprises an SV40 NLS fused to the C-terminus of the Cas protein (or to a C-terminally-fused NLS on the Cas protein), optionally by means of a linker. In some embodiments, the Cas protein comprises a nucleoplasmin NLS fused to the C-terminus of the Cas protein (or to a C-terminally-fused NLS on the Cas protein), optionally by means of a linker. In some embodiments, the Cas protein comprises: a) a c-Myc NLS fused to the N-terminus of the Cas protein, optionally by means of a linker, b) an SV40 NLS fused to the C-terminus of the Cas protein, optionally by means of a linker, and c) a nucleoplasmin NLS fused to the C-terminus of the SV40 NLS, optionally by means of a linker. In some embodiments, the Cas protein comprises: a) a c-Myc NLS fused to the N-terminus of the Cas protein, optionally by means of a linker, b) a nucleoplasmin NLS fused to the C-terminus of the Cas protein, optionally by means of a linker, and c) an SV40 NLS fused to the C-terminus of the nucleoplasmin NLS, optionally by means of a linker. In some embodiments, a c-myc NLS is fused to the N-terminus of the Cas and an SV40 NLS and/or nucleoplasmin NLS is fused to the C-terminus of the Cas. In some embodiments, a c-myc NLS is fused to the N-terminus of the Cas (e.g., by means of a linker such as GSVD), an SV40 NLS is fused to the C-terminus of the Cas (e.g., by means of a linker such as GSGS), and a nucleoplasmin NLS is fused to the C-terminus of the SV-40 NLS (e.g., by means of a linker such as GSGS).

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding Cas9 (e.g., CK8e), a nucleic acid encoding Cas9, a polyadenylation sequence, a promoter for expression of a second sgRNA, a second sgRNA guide sequence, and a second sgRNA scaffold sequence. In some embodiments, the first sgRNA is associated with weaker expression than the second sgRNA when compared individually in a sgRNA expression assay, e.g., in an assay in which each guide is separately assessed (i.e., not in the same construct) using the same promoter, same concentration of genetic material/vector/RNP in substantially the same conditions (e.g., time, pH, temperature, buffer conditions). In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 9001. In some embodiments the promoter for expression of the nucleic acid encoding the first sgRNA comprises SEQ ID NO: 9002. In some embodiments the promoter for expression of the nucleic acid encoding the first sgRNA comprises SEQ ID NO: 9003. In some embodiments the promoter for expression of the nucleic acid encoding the first sgRNA comprises SEQ ID NO: 9004. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 9006. In some embodiments the promoter for expression of the nucleic acid encoding the first sgRNA comprises SEQ ID NO: 9007. In some embodiments the promoter for expression of the nucleic acid encoding the first sgRNA comprises SEQ ID NO: 9008. In some embodiments the promoter for expression of the nucleic acid encoding the first sgRNA comprises SEQ ID NO: 9009. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 9001. In some embodiments the promoter for expression of the nucleic acid encoding the second sgRNA comprises SEQ ID NO: 9002. In some embodiments the promoter for expression of the nucleic acid encoding the second sgRNA comprises SEQ ID NO: 9003. In some embodiments the promoter for expression of the nucleic acid encoding the second sgRNA comprises SEQ ID NO: 9004. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 9006. In some embodiments the promoter for expression of the nucleic acid encoding the second sgRNA comprises SEQ ID NO: 9007. In some embodiments the promoter for expression of the nucleic acid encoding the second sgRNA comprises SEQ ID NO: 9008. In some embodiments the promoter for expression of the nucleic acid encoding the second sgRNA comprises SEQ ID NO: 9009. In some embodiments the promoter for expression of the nucleic acid encoding the second sgRNA is any of the H1m promoters disclosed herein. In some embodiments, the promoter for SaCas9 is the CK8e promoter. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the one or more NLSs is a nucleoplasmin NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding Cas9 (e.g., CK8e), a nucleic acid encoding Cas9, a polyadenylation sequence, an hU6c promoter for expression of a second sgRNA, a second sgRNA guide sequence, and a second sgRNA scaffold sequence. In some embodiments, the first sgRNA and the second sgRNA are selected from Table 1A (for SaCas9) or Table 1B (for SluCas9). In some embodiments, the nucleic acid sequence encoding Cas9 is fused to a nucleic acid sequence encoding a nuclear localization sequence (NLS). In some embodiments, the nucleic acid sequence encoding Cas9 is fused to two nucleic acid sequences each encoding a nuclear localization sequence (NLS). In some embodiments, the nucleic acid sequence encoding Cas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the Cas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding Cas9 (e.g., CK8e), a nucleic acid encoding Cas9, a polyadenylation sequence, an 7SK promoter for expression of a second sgRNA, a second sgRNA guide sequence, and a second sgRNA scaffold sequence. In some embodiments, the first sgRNA and the second sgRNA are selected from Table 1A (for SaCas9) or Table 1B (for SluCas9). In some embodiments, the nucleic acid sequence encoding Cas9 is fused to a nucleic acid sequence encoding a nuclear localization sequence (NLS). In some embodiments, the nucleic acid sequence encoding Cas9 is fused to two nucleic acid sequences each encoding a nuclear localization sequence (NLS). In some embodiments, the nucleic acid sequence encoding Cas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the Cas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding Cas9 (e.g., CK8e), a nucleic acid encoding Cas9, a polyadenylation sequence, an H1m promoter for expression of a second sgRNA, a second sgRNA guide sequence, and a second sgRNA scaffold sequence. In some embodiments, the first sgRNA and the second sgRNA are selected from Table 1A (for SaCas9) or Table 1B (for SluCas9). In some embodiments, the nucleic acid sequence encoding Cas9 is fused to a nucleic acid sequence encoding a nuclear localization sequence (NLS). In some embodiments, the nucleic acid sequence encoding Cas9 is fused to two nucleic acid sequences each encoding a nuclear localization sequence (NLS). In some embodiments, the nucleic acid sequence encoding Cas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the Cas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an 7SK promoter for expression of the nucleic acid encoding the first sgRNA, a promoter for expression of a nucleic acid encoding Cas9 (e.g., CK8e), a nucleic acid encoding Cas9, a polyadenylation sequence, an H1m promoter for expression of a second sgRNA, a second sgRNA guide sequence, and a second sgRNA scaffold sequence. In some embodiments, the first sgRNA and the second sgRNA are selected from Table 1A (for SaCas9) or Table 1B (for SluCas9). In some embodiments, the nucleic acid sequence encoding Cas9 is fused to a nucleic acid sequence encoding a nuclear localization sequence (NLS). In some embodiments, the nucleic acid sequence encoding Cas9 is fused to two nucleic acid sequences each encoding a nuclear localization sequence (NLS). In some embodiments, the nucleic acid sequence encoding Cas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the Cas9 with a linker.

In some embodiments, the disclosure provides for a composition comprising at least two nucleic acids. In some embodiments, the composition comprises at least two nucleic acid molecules, wherein the first nucleic acid molecule comprises a sequence encoding any of the endonucleases disclosed herein (e.g., a SaCas9, SluCas9, or sRGN), wherein the second nucleic acid molecule encodes a first guide RNA and a second guide RNA, wherein the first guide RNA and the second guide RNA are not the same sequence, and wherein the second nucleic acid molecule does not encode an endonuclease. In some embodiments, the first nucleic acid molecule also encodes a copy of the first guide RNA and a copy of the second guide RNA. In some embodiments, the first nucleic acid molecule does not encode any guide RNAs. In some embodiments, the second nucleic acid molecule encodes two copies of the first guide RNA and two copies of the second guide RNA. In some embodiments, the second nucleic molecule encodes two copies of the first guide RNA, and one copy of the second guide RNA. In some embodiments, the second nucleic acid molecule encodes one copy of the first guide RNA, and two copies of the second guide RNA. In some embodiments, the second nucleic acid molecule comprises two copies of the first guide RNA, and three copies of the second guide RNA. In some embodiments, the second nucleic acid molecule comprises three copies of the first guide RNA, and two copies of the second guide RNA. In some embodiments, the second nucleic acid does not encode a Cas protein. In some embodiments, the second nucleic acid molecule encodes three copies of the first guide RNA and three copies of the second guide RNA. In some embodiments, the first nucleic acid molecule comprises from 5′ to 3′ with respect to the plus strand: the reverse complement of a first guide RNA scaffold sequence, the reverse complement of a nucleotide sequence encoding the first guide RNA sequence, the reverse complement of a promoter for expression of the nucleotide sequence encoding the first guide RNA sequence, a promoter for expression of a nucleotide sequence encoding the endonuclease, a nucleotide sequence encoding an endonuclease, a polyadenylation sequence, a promoter for expression of the second guide RNA in the same direction as the promoter for the endonuclease, the second guide RNA sequence, and a second guide RNA scaffold sequence. In some embodiments, the promoter for expression of the nucleotide sequence encoding the first guide RNA sequence in the first nucleic acid molecule is a U6 promoter and the promoter for expression of the nucleotide sequence encoding the second guide RNA in the first nucleic acid molecule is a U6 promoter.

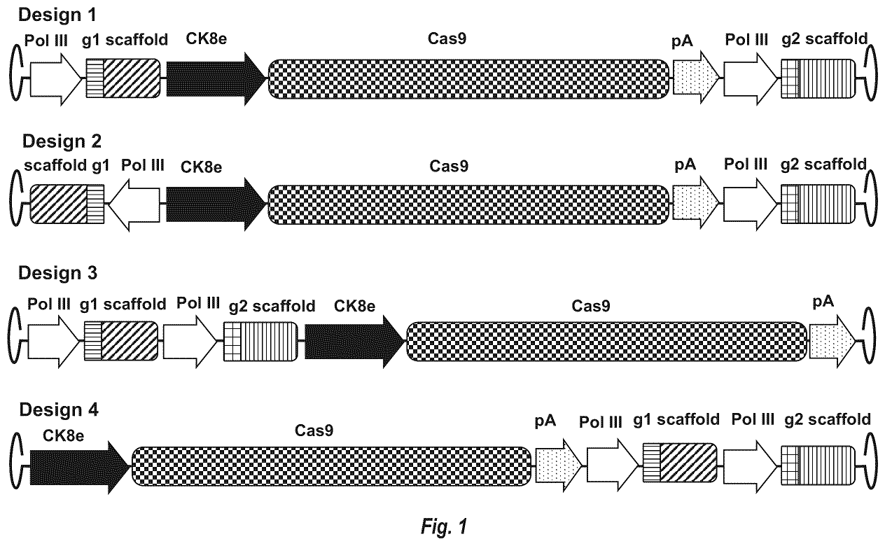

In some embodiments, the AAV vector comprises from 5′ to 3′ with respect to the plus strand: a promoter for expression of a nucleic acid encoding a first sgRNA, a nucleic acid encoding the first sgRNA guide sequence, the first sgRNA scaffold sequence, a promoter for expression of Cas9 (e.g., CK8e), a nucleic acid encoding Cas9, a polyadenylation sequence, a promoter for expression of a second sgRNA, the second sgRNA guide sequence, and a second sgRNA scaffold sequence. See FIG. 1 at “Design 1”.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding Cas9 (e.g., CK8e), a nucleic acid encoding Cas9, a polyadenylation sequence, a promoter for expression of a second sgRNA, a second sgRNA guide sequence, and a second sgRNA scaffold sequence. See FIG. 1 at “Design 2”.

In some embodiments, the AAV vector comprises from 5′ to 3′ with respect to the plus strand: a promoter for expression of a nucleic acid encoding a first sgRNA, a nucleic acid encoding the first sgRNA guide sequence, a first sgRNA scaffold sequence, a promoter for expression of a second sgRNA, a second sgRNA guide sequence, and a second sgRNA scaffold sequence, a promoter for Cas9 (e.g., CK8e), a nucleic acid encoding Cas9, and a polyadenylation sequence. See FIG. 1 at “Design 3”.

In some embodiments, the AAV vector comprises from 5′ to 3′ with respect to the plus strand: a promoter for expression of a nucleic acid encoding Cas9 (e.g., CK8e), a nucleic acid encoding Cas9, a polyadenylation sequence, a promoter for expression of the nucleic acid encoding a first guide RNA, a nucleic acid encoding the first sgRNA guide sequence, a first sgRNA scaffold sequence, a promoter for expression of the second sgRNA, a second sgRNA guide sequence, and a second sgRNA scaffold sequence. See FIG. 1 at “Design 4”.

In some embodiments, the first nucleic acid molecule is in a first vector (e.g., AAV9), and the second nucleic acid is in a separate second vector. In some embodiments, the first vector is AAV9. In some embodiments, the second vector is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the second vector comprises from 5′ to 3′ with respect to the plus strand: a promoter for expression of a first copy of a first guide RNA (e.g., a U6 promoter), a first copy of a nucleotide sequence encoding a first guide RNA, a first copy of a nucleotide sequence encoding a first guide RNA scaffold, a promoter for expression of a second copy of the first guide RNA (e.g., a H1 promoter), a second copy of the nucleotide sequence encoding the first guide RNA, a second copy of the nucleotide sequence encoding the first guide RNA scaffold, a promoter for expression of a second guide RNA (e.g., a 7SK promoter), a nucleotide sequence encoding a second guide RNA, and a nucleotide sequence encoding a second guide RNA scaffold. In some embodiments, the second vector comprises from 5′ to 3′ with respect to the plus strand: a promoter for expression of a first guide RNA (e.g., a U6 promoter), a nucleotide sequence encoding a first guide RNA, a nucleotide sequence encoding a first guide RNA scaffold, a promoter for expression of a second guide RNA (e.g., a 7SK promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the nucleotide sequence encoding the first guide scaffold sequence and the promoter for expression of the second guide sequence. In some embodiments, the second vector comprises from 5′ to 3′ with respect to the plus strand: the reverse complement of a nucleotide sequence encoding a scaffold for a first guide RNA, the reverse complement of a nucleotide sequence encoding a first guide RNA, the reverse complement of a promoter for expression of the first guide RNA (e.g., a U6c promoter), a promoter for expression of a second guide RNA (e.g., a U6c promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the reverse complement of the promoter for expression of the first guide RNA and the promoter for expression of the second guide RNA. In some embodiments, the second vector comprises from 5′ to 3′ with respect to the plus strand: the reverse complement of a nucleotide sequence encoding a first copy of a first guide RNA scaffold, the reverse complement of a nucleotide sequence encoding a first copy of the first guide RNA, the reverse complement of a promoter for expression of the first copy of the first guide RNA (e.g., a 7SK2 promoter), the reverse complement of a second copy of the nucleotide sequence encoding the first guide RNA scaffold, the reverse complement of a second copy of the nucleotide sequence encoding the first guide RNA, the reverse complement of a promoter for expression of the second copy of the nucleotide sequence encoding the first guide RNA (e.g., a hU6c promoter), a promoter for expression of a first copy of a second guide RNA (e.g., a hU6c promoter), a first copy of a nucleotide sequence encoding a second guide RNA, a first copy of a nucleotide sequence encoding a second guide RNA scaffold, a promoter for expression of a second copy of the second guide RNA (e.g., a 7Sk2 promoter), a second copy of the nucleotide sequence encoding the second guide RNA, and a second copy of the nucleotide sequence encoding the second guide RNA scaffold. In some embodiments, the second vector comprises a stuffer sequence (e.g., a 3′UTR desmin sequence) between the reverse complement of the promoter for expression of the second copy of the first guide RNA and the promoter for expression of the first copy of the second guide RNA. In some embodiments, the second vector comprises from 5′ to 3′ with respect to the plus strand: the reverse complement of a nucleotide sequence encoding a first copy of a first guide RNA scaffold, the reverse complement of a first copy of a nucleotide sequence encoding the first guide RNA, the reverse complement of a promoter for expression of the first copy of the first guide RNA (e.g., a 7SK2 promoter), the reverse complement of a first copy of a nucleotide sequence encoding a second guide RNA scaffold, the reverse complement of a nucleotide sequence encoding the first copy of the second guide RNA, the reverse complement of a promoter for expression of the first copy of the second guide RNA (e.g., a hU6c promoter), a promoter for expression of a second copy of the second guide RNA (e.g., a hU6c promoter), a second copy of the nucleotide sequence encoding the second guide RNA, a second copy of the nucleotide sequence encoding the second guide RNA scaffold, a promoter for expression of a second copy of the first guide RNA (e.g., a 7SK2 promoter), a second copy of the nucleotide sequence encoding the first guide RNA, and a second copy of the nucleotide sequence encoding the first guide RNA scaffold. In some embodiments, the second vector comprises a stuffer sequence (e.g., a 3′UTR desmin sequence) between the reverse complement of the promoter for expression of the first copy of the second guide RNA and the promoter for expression of the second copy of the first guide RNA. In some embodiments, the second vector comprises from 5′ to 3′ with respect to the plus strand: the reverse complement of a nucleotide sequence encoding a first guide RNA scaffold, the reverse complement of a nucleotide sequence encoding a first guide RNA, the reverse complement of a promoter for expression of a first guide RNA (e.g., a hU6c promoter), a promoter for expression of a second guide RNA (e.g., a hU6c promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the reverse complement of the promoter for expression of the first guide RNA and the promoter for expression of the second guide RNA. In particular embodiments, the first guide RNA is different from the second guide RNA. In some embodiments, the first guide RNA comprises a sequence of Table 1A (for SaCas9) or Table 1B (for SluCas9) and the second guide RNA comprises a different sequence from Table 1A (for SaCas9) or Table 1B (for SluCas9).

In some embodiments, if the composition comprises one or more nucleic acids encoding an RNA-targeted endonuclease and one or more guide RNAs, the one or more nucleic acids are designed such that they express the one or more guide RNAs at an equivalent or higher level (e.g., a greater number of expressed transgene copies) as compared to the expression level of the RNA-targeted endonuclease. In some embodiments, the one or more nucleic acids are designed such that they express (e.g., on average in 100 cells) the one or more guide RNAs at least a 1.1, 1.2, 1.3, 1.4, or 1.5 times higher level (e.g., a greater number of expressed transgene copies) as compared to the expression level of the RNA-targeted endonuclease. In some embodiments, the one or more nucleic acids are designed such that they express the one or more guide RNAs at 1.01-1.5, 1.01-1.4, 1.01-1.3, 1.01-1.2, 1.01-1.1, 1.1-2.0, 1.1-1.8, 1.1-1.6, 1.1-1.4, 1.1-1.3, 1.2-2.0, 1.2-1.8, 1.2-1.6, 1.2-1.4, 1.4-2.0, 1.4-1.8, 1.4-1.6, 1.6-2.0, 1.6-1.8, or 1.8-2.0 times higher level (e.g., a greater number of expressed transgene copies) as compared to the expression level of the RNA-targeted endonuclease. In some embodiments, the one or more guide RNAs are designed to express a higher level than the RNA-targeted endonuclease by: a) utilizing one or more regulatory elements (e.g., promoters or enhancers) that express the one or more guide RNAs at a higher level as compared to the regulatory elements (e.g., promoters or enhancers) for expression of the RNA-targeted endonuclease; and/or b) expressing more copies of one or more of the guide RNAs as compared to the number of copies of the RNA-targeted endonuclease (e.g., 2× or 3× as many copies of the nucleotide sequences encoding the one or more guide RNAs as compared to the number of copies of the nucleotide sequences encoding the RNA-targeted endonuclease). For example, in some embodiments, the composition comprises multiple nucleic acid molecules (e.g., in multiple vectors), wherein for every nucleotide sequence encoding an RNA-targeted endonuclease in the nucleic acid molecules in the composition, there are two or three copies of the nucleotide sequence encoding the guide RNA in the nucleic acid molecules in the composition. In some embodiments, the composition comprises a first guide RNA and a second guide RNA, wherein the first guide RNA and the second guide RNA are not the same (e.g., any of the guide RNA pairs disclosed herein), and for every nucleotide sequence encoding an RNA-targeted endonuclease in the nucleic acid molecules in the composition, there are two or three copies of the nucleotide sequence encoding the first guide RNA and/or the second guide RNA.

### Endonucleases

In some embodiments, any of the nucleic acids disclosed herein encodes an RNA-targeted endonuclease. In some embodiments, the RNA-targeted endonuclease has cleavase activity, which can also be referred to as double-strand endonuclease activity. In some embodiments, the RNA-targeted endonuclease comprises a Cas nuclease. Examples of Cas9 nucleases include those of the type II CRISPR systems.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 8 (designated herein as SpCas9):

MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGD.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 711:

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

In some embodiments, the nucleic acid encoding SaCas9 comprises the nucleic acid of SEQ ID NO: 9014:

AAGCGCAATTACATCCTGGGCCTGGATATCGGCATCACCTCCGTGGGCTACG GCATCATCGACTATGAGACACGGGATGTGATCGACGCCGGCGTGAGACTGTTCAAGGAG GCCAACGTGGAGAACAATGAGGGCCGGCGGAGCAAGAGGGGAGCAAGGCGCCTGAAGC GGAGAAGGCGCCACAGAATCCAGAGAGTGAAGAAGCTGCTGTTCGATTACAACCTGCTG ACCGACCACTCCGAGCTGTCTGGCATCAATCCTTATGAGGCCCGGGTGAAGGGCCTGTCC CAGAAGCTGTCTGAGGAGGAGTTTTCTGCCGCCCTGCTGCACCTGGCAAAGAGGAGAGG CGTGCACAACGTGAATGAGGTGGAGGAGGACACCGGCAACGAGCTGAGCACAAAGGAG CAGATCAGCCGCAATTCCAAGGCCCTGGAGGAGAAGTATGTGGCCGAGCTGCAGCTGGA GCGGCTGAAGAAGGATGGCGAGGTGAGGGGCTCCATCAATCGCTTCAAGACCTCTGACT ACGTGAAGGAGGCCAAGCAGCTGCTGAAGGTGCAGAAGGCCTACCACCAGCTGGATCAG AGCTTTATCGATACATATATCGACCTGCTGGAGACCAGGCGCACATACTATGAGGGACC AGGAGAGGGCTCCCCCTTCGGCTGGAAGGACATCAAGGAGTGGTACGAGATGCTGATGG GCCACTGCACCTATTTTCCAGAGGAGCTGAGATCCGTGAAGTACGCCTATAACGCCGATC TGTACAACGCCCTGAATGACCTGAACAACCTGGTCATCACCAGGGATGAGAACGAGAAG CTGGAGTACTATGAGAAGTTCCAGATCATCGAGAACGTGTTCAAGCAGAAGAAGAAGCC TACACTGAAGCAGATCGCCAAGGAGATCCTGGTGAACGAGGAGGACATCAAGGGCTACC GCGTGACCAGCACAGGCAAGCCAGAGTTCACCAATCTGAAGGTGTATCACGATATCAAG GACATCACAGCCCGGAAGGAGATCATCGAGAACGCCGAGCTGCTGGATCAGATCGCCAA GATCCTGACCATCTATCAGAGCTCCGAGGACATCCAGGAGGAGCTGACCAACCTGAATA GCGAGCTGACACAGGAGGAGATCGAGCAGATCAGCAATCTGAAGGGCTACACCGGCAC ACACAACCTGTCCCTGAAGGCCATCAATCTGATCCTGGATGAGCTGTGGCACACAAACG ACAATCAGATCGCCATCTTTAACAGGCTGAAGCTGGTGCCAAAGAAGGTGGACCTGAGC CAGCAGAAGGAGATCCCAACCACACTGGTGGACGATTTCATCCTGTCCCCCGTGGTGAA GCGGAGCTTCATCCAGAGCATCAAAGTGATCAACGCCATCATCAAGAAGTACGGCCTGC CCAATGATATCATCATCGAGCTGGCCAGGGAGAAGAACTCTAAGGACGCCCAGAAGATG ATCAATGAGATGCAGAAGAGGAACCGCCAGACCAATGAGCGGATCGAGGAGATCATCA GAACCACAGGCAAGGAGAACGCCAAGTACCTGATCGAGAAGATCAAGCTGCACGATAT GCAGGAGGGCAAGTGTCTGTATAGCCTGGAGGCCATCCCTCTGGAGGACCTGCTGAACA ATCCATTCAACTACGAGGTGGATCACATCATCCCCCGGAGCGTGAGCTTCGACAATTCCT TTAACAATAAGGTGCTGGTGAAGCAGGAGGAGAACTCTAAGAAGGGCAATAGGACCCCT TTCCAGTACCTGTCTAGCTCCGATTCTAAGATCAGCTACGAGACCTTCAAGAAGCACATC CTGAATCTGGCCAAGGGCAAGGGCCGCATCTCTAAGACCAAGAAGGAGTACCTGCTGGA GGAGCGGGACATCAACAGATTCAGCGTGCAGAAGGACTTCATCAACCGGAATCTGGTGG ACACCAGATACGCCACACGCGGCCTGATGAATCTGCTGCGGTCCTATTTCAGAGTGAACA ATCTGGATGTGAAGGTGAAGAGCATCAACGGCGGCTTCACCTCCTTTCTGCGGAGAAAG TGGAAGTTTAAGAAGGAGAGAAACAAGGGCTATAAGCACCACGCCGAGGATGCCCTGAT CATCGCCAATGCCGACTTCATCTTTAAGGAGTGGAAGAAGCTGGACAAGGCCAAGAAAG TGATGGAGAACCAGATGTTCGAGGAGAAGCAGGCCGAGAGCATGCCCGAGATCGAGAC CGAGCAGGAGTACAAGGAGATTTTCATCACACCTCACCAGATCAAGCACATCAAGGACT TCAAGGACTACAAGTATTCCCACAGGGTGGATAAGAAGCCCAACCGCGAGCTGATCAAT GACACCCTGTATTCTACAAGGAAGGACGATAAGGGCAATACCCTGATCGTGAACAATCT GAACGGCCTGTACGACAAGGATAATGACAAGCTGAAGAAGCTGATCAACAAGAGCCCC GAGAAGCTGCTGATGTACCACCACGATCCTCAGACATATCAGAAGCTGAAGCTGATCAT GGAGCAGTACGGCGACGAGAAGAACCCACTGTATAAGTACTATGAGGAGACCGGCAACT ACCTGACAAAGTATTCCAAGAAGGATAATGGCCCCGTGATCAAGAAGATCAAGTACTAT GGCAACAAGCTGAATGCCCACCTGGACATCACCGACGATTACCCCAACAGCCGGAATAA GGTGGTGAAGCTGAGCCTGAAGCCATACAGGTTCGACGTGTACCTGGACAACGGCGTGT ATAAGTTTGTGACAGTGAAGAATCTGGATGTGATCAAGAAGGAGAACTACTATGAAGTG AATAGCAAGTGCTACGAGGAGGCCAAGAAGCTGAAGAAGATCAGCAACCAGGCCGAGT TCATCGCCTCTTTTTACAACAATGACCTGATCAAGATCAATGGCGAGCTGTATAGAGTGA TCGGCGTGAACAATGATCTGCTGAACCGCATCGAAGTGAATATGATCGACATCACCTACC GGGAGTATCTGGAGAACATGAATGATAAGAGGCCCCCTCGCATCATCAAGACCATCGCC TCTAAGACACAGAGCATCAAGAAGTACTCTACAGACATCCTGGGCAACCTGTATGAGGT GAAGAGCAAGAAGCACCCTCAGATCATCAAGAAGGGC. In some embodiments comprising a nucleic acid encoding SaCas9, the SaCas9 comprises an amino acid sequence of SEQ ID NO: 711.

In some embodiments, the SaCas9 is a variant of the amino acid sequence of SEQ ID NO: 711. In some embodiments, the SaCas9 comprises an amino acid other than an E at the position corresponding to position 781 of SEQ ID NO: 711. In some embodiments, the SaCas9 comprises an amino acid other than an N at the position corresponding to position 967 of SEQ ID NO: 711. In some embodiments, the SaCas9 comprises an amino acid other than an R at the position corresponding to position 1014 of SEQ ID NO: 711. In some embodiments, the SaCas9 comprises a K at the position corresponding to position 781 of SEQ ID NO: 711. In some embodiments, the SaCas9 comprises a K at the position corresponding to position 967 of SEQ ID NO: 711. In some embodiments, the SaCas9 comprises an H at the position corresponding to position 1014 of SEQ ID NO: 711. In some embodiments, the SaCas9 comprises an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid other than an R at the position corresponding to position 244 of SEQ ID NO: 711. In some embodiments, the SaCas9 comprises an amino acid other than an N at the position corresponding to position 412 of SEQ ID NO: 711. In some embodiments, the SaCas9 comprises an amino acid other than an N at the position corresponding to position 418 of SEQ ID NO: 711. In some embodiments, the SaCas9 comprises an amino acid other than an R at the position corresponding to position 653 of SEQ ID NO: 711. In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; and an amino acid other than an R at the position corresponding to position 653 of SEQ ID NO: 711. In some embodiments, the SaCas9 comprises an A at the position corresponding to position 244 of SEQ ID NO: 711. In some embodiments, the SaCas9 comprises an A at the position corresponding to position 412 of SEQ ID NO: 711. In some embodiments, the SaCas9 comprises an A at the position corresponding to position 418 of SEQ ID NO: 711. In some embodiments, the SaCas9 comprises an A at the position corresponding to position 653 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; and an A at the position corresponding to position 653 of SEQ ID NO: 711.

In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; an amino acid other than an R at the position corresponding to position 653 of SEQ ID NO: 711; an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; an A at the position corresponding to position 653 of SEQ ID NO: 711; a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 715 (designated herein as SaCas9-KKH or SACAS9KKH):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 716 (designated herein as SaCas9-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELASVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDAQIAIFARLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATAGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 717 (designated herein as SaCas9-KKH-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELASVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDAQIAIFARLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATAGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

In some embodiments, the nucleic acid encoding SluCas9 encodes a SluCas9 comprising an amino acid sequence that is at least 80%, 85%, 90%, 91%, 92%, 93%, 94%, 95%, 96%, 97%, 98%, 99% or 100% identical to the sequence of SEQ ID NO: 712:

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the SluCas9 is a variant of the amino acid sequence of SEQ ID NO: 712. In some embodiments, the SluCas9 comprises an amino acid other than an Q at the position corresponding to position 781 of SEQ ID NO: 712. In some embodiments, the SluCas9 comprises an amino acid other than an R at the position corresponding to position 1013 of SEQ ID NO: 712. In some embodiments, the SluCas9 comprises a K at the position corresponding to position 781 of SEQ ID NO: 712. In some embodiments, the SluCas9 comprises a K at the position corresponding to position 966 of SEQ ID NO: 712. In some embodiments, the SluCas9 comprises an H at the position corresponding to position 1013 of SEQ ID NO: 712. In some embodiments, the SluCas9 comprises an amino acid other than an Q at the position corresponding to position 781 of SEQ ID NO: 712; and an amino acid other than an R at the position corresponding to position 1013 of SEQ ID NO: 712. In some embodiments, the SluCas9 comprises a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid other than an R at the position corresponding to position 246 of SEQ ID NO: 712. In some embodiments, the SluCas9 comprises an amino acid other than an N at the position corresponding to position 414 of SEQ ID NO: 712. In some embodiments, the SluCas9 comprises an amino acid other than a T at the position corresponding to position 420 of SEQ ID NO: 712. In some embodiments, the SluCas9 comprises an amino acid other than an R at the position corresponding to position 655 of SEQ ID NO: 712. In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; and an amino acid other than an R at the position corresponding to position 655 of SEQ ID NO: 712. In some embodiments, the SluCas9 comprises an A at the position corresponding to position 246 of SEQ ID NO: 712. In some embodiments, the SluCas9 comprises an A at the position corresponding to position 414 of SEQ ID NO: 712. In some embodiments, the SluCas9 comprises an A at the position corresponding to position 420 of SEQ ID NO: 712. In some embodiments, the SluCas9 comprises an A at the position corresponding to position 655 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; and an A at the position corresponding to position 655 of SEQ ID NO: 712.

In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; an amino acid other than an R at the position corresponding to position 655 of SEQ ID NO: 712; an amino acid other than an Q at the position corresponding to position 781 of SEQ ID NO: 712; a K at the position corresponding to position 966 of SEQ ID NO: 712; and an amino acid other than an R at the position corresponding to position 1013 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; an A at the position corresponding to position 655 of SEQ ID NO: 712; a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 718 (designated herein as SluCas9-KH or SLUCAS9KH):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRKLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
HIKKTIGKKVNSIEKLTTDVLGNVFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 719 (designated herein as SluCas9-HF):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELASVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRAQMEIFAHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATAELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 720 (designated herein as SluCas9-HF-KH):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELASVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRAQMEIFAHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATAELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRKLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
HIKKTIGKKVNSIEKLTTDVLGNVFTNTQYTKPQLLFKRGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7021 (designated herein as sRGN1):

MNQKFILGLDIGITSVGYGLIDYETKNIIDAGVRLFPEANVENNEGRRSKRGSRRLKRRRIHRL
DRVKHLLAEYDLLDLTNIPKSTNPYQTRVKGLNEKLSKDELVIALLHIAKRRGIHNVDVAAD
KEETASDSLSTKDQINKNAKFLESRYVCELQKERLENEGHVRGVENRFLTKDIVREAKKIIDT
QMQYYPEIDETFKEKYISLVETRREYFEGPGKGSPFGWEGNIKKWFEQMMGHCTYFPEELRS
VKYSYSAELFNALNDLNNLVITRDEDAKLNYGEKFQIIENVFKQKKTPNLKQIAIEIGVHETEI
KGYRVNKSGTPEFTEFKLYHDLKSIVFDKSILENEAILDQIAEILTIYQDEQSIKEELNKLPEILN
EQDKAEIAKLIGYNGTHRLSLKCIHLINEELWQTSRNQMEIFNYLNIKPNKVDLSEQNKIPKD
MVNDFILSPVVKRTFIQSINVINKVIEKYGIPEDIIIELARENNSDDRKKFINNLQKKNEATRKRI
NEIIGQTGNQNAKRIVEKIRLHDQQEGKCLYSLKDIPLEDLLRNPNNYDIDHIIPRSVSFDDSM
HNKVLVRREQNAKKNNQTPYQYLTSGYADIKYSVFKQHVLNLAENKDRMTKKKREYLLEE
RDINKFEVQKEFINRNLVDTRYATRELTNYLKAYFSANNMNVKVKTINGSFTDYLRKVWKF
KKERNHGYKHHAEDALIIANADFLFKENKKLKAVNSVLEKPEIETKQLDIQVDSEDNYSEMFI
IPKQVQDIKDFRNFKYSHRVDKKPNRQLINDTLYSTRKKDNSTYIVQTIKDIYAKDNTTLKKQ
FDKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7022 (designated herein as sRGN2):

MNQKFILGLDIGITSVGYGLIDYETKNIIDAGVRLFPEANVENNEGRRSKRGSRRLKRRRIHRL
ERVKSLLSEYKIISGLAPTNNQPYNIRVKGLTEQLTKDELAVALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPEFTEFKLYHDLKSVLFDQSILENEDVLDQIAEILTIYQDKDSIKSKLTELDILLNEEDK
ENIAQLTGYNGTHRLSLKCIRLVLEEQWYSSRNQMEIFTHLNIKPKKINLTAANKIPKAMIDEF
ILSPVVKRTFIQSINVINKVIEKYGIPEDIIIELARENNSDDRKKFINNLQKKNEATRKRINEIIGQ
TGNQNAKRIVEKIRLHDQQEGKCLYSLESIALMDLLNNPQNYEVDHIIPRSVAFDNSIHNKVL
VKQIENSKKGNRTPYQYLNSSDAKLSYNQFKQHILNLSKSKDRISKKKKDYLLEERDINKFEV
QKEFINRNLVDTRYATRELTSYLKAYFSANNMDVKVKTINGSFTNHLRKVWRFDKYRNHGY
KHHAEDALIIANADFLFKENKKLKAVNSVLEKPEIETKQLDIQVDSEDNYSEMFIIPKQVQDIK
DFRNFKYSHRVDKKPNRQLINDTLYSTRKKDNSTYIVQTIKDIYAKDNTTLKKQFDKSPEKFL
MYQHDPRTFEKLEVIMKQYANEKNPLAKYHEETGEYLTKYSKKNNGPIVKSLKYIGNKLGS
HLDVTHQFKSSTKKLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYKEYCELNNIKGEPRIK
KTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7023 (designated herein as sRGN3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7024 (designated herein as sRGN3.1):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKNYRFDVYLTEKGYKFVTIAYLNVFKKDNYYYIP
KDKYQELKEKKKIKDTDQFIASFYKNDLIKLNGDLYKIIGVNSDDRNIIELDYYDIKYKDYCEI
NNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7025 (designated herein as sRGN3.2):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKFSHRVDKKPNRQLINDTLYSTRMKDEHDYIVQTITDIYGKDNTNLKKQ
FNKNPEKFLMYQNDPKTFEKLSIIMKQYSDEKNPLAKYYEETGEYLTKYSKKNNGPIVKKIK
LLGNKVGNHLDVTNKYENSTKKLVKLSIKNYRFDVYLTEKGYKFVTIAYLNVFKKDNYYYI
PKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is encoded by a nucleic acid molecule at least 80%, 85%, 90%, 91%, 92%, 93%, 94%, 95%, 96%, 97%, 98%, 99% or 100% identical to the sequence of SEQ ID NO: 1 (an exemplary nucleic acid molecule encoding sRGN3.1):

ATGAATCAGAAATTCATCCTGGGACTGGACATCGGCATTACCTCTGTGGGCTACGGCTTG
ATTGACTACGAGACCAAGAACATCATCGACGCCGGCGTTAGACTGTTTCCTGAGGCCAAT
GTGGAAAACAACGAGGGCAGACGGTCTAAGCGGGGCTCTAGACGACTGAAAAGAAGAA
GAATCCACAGACTGGAAAGAGTGAAGCTGCTGCTGACCGAGTACGACCTGATCAATAAG
GAACAGATCCCTACAAGCAACAACCCCTACCAGATTAGAGTGAAGGGCCTGAGCGAGAT
CCTGAGCAAAGACGAGCTGGCCATCGCCCTGCTGCACCTGGCCAAGCGGAGGGGCATCC
ACAACGTGGATGTGGCCGCCGACAAGGAGGAAACCGCCAGCGACTCCCTGAGCACCAAA
GATCAGATCAACAAGAATGCCAAGTTCCTGGAGAGCAGATACGTGTGCGAGCTGCAGAA
GGAGAGATTGGAGAACGAGGGCCACGTGCGGGGCGTGGAGAATAGATTCCTGACAAAA
GATATCGTGCGGGAAGCCAAGAAGATCATCGACACCCAGATGCAGTACTATCCTGAAAT
CGACGAAACCTTCAAGGAGAAGTACATCAGCCTGGTCGAAACCAGACGCGAGTATTTCG
AGGGCCCCGGACAGGGCTCTCCTTTCGGCTGGAACGGCGATCTGAAGAAGTGGTACGAG
ATGCTGATGGGACACTGCACCTACTTCCCTCAAGAGCTGAGATCTGTGAAGTACGCCTAC
AGCGCCGATCTGTTCAACGCCCTGAACGATCTGAACAACCTTATCATCCAGCGGGACAAT
TCTGAGAAGCTGGAATACCACGAGAAATATCATATCATCGAGAACGTCTTTAAACAAAA
GAAAAAGCCTACCCTGAAGCAGATCGCCAAGGAGATCGGAGTGAATCCTGAGGATATCA
AAGGCTACAGAATCACAAAGTCCGGCACCCCTGAGTTCACCAGCTTTAAGCTGTTCCACG
ACCTGAAGAAGGTCGTGAAAGACCACGCCATCCTCGATGACATCGATCTGCTGAACCAG
ATCGCTGAGATCCTGACAATCTACCAGGACAAAGATTCTATCGTGGCTGAACTGGGACA
GCTGGAATACCTTATGAGCGAGGCCGACAAGCAAAGCATCTCCGAACTGACCGGCTACA
CGGGCACCCATAGCCTGAGCCTGAAGTGTATGAACATGATCATCGATGAGCTGTGGCAC
TCTAGCATGAACCAGATGGAAGTTTTCACCTACCTGAACATGCGGCCTAAGAAGTACGA
GCTGAAGGGCTACCAGAGAATCCCCACCGATATGATCGACGACGCCATCCTGAGCCCCG
TGGTGAAAAGAACATTCATCCAGAGCATCAACGTGATCAACAAGGTGATCGAGAAGTAC
GGCATTCCAGAGGACATCATCATCGAGCTGGCCAGAGAAAACAACAGCGACGATAGAA
AGAAGTTTATCAACAACCTGCAGAAAAAAAACGAGGCCACCCGGAAGAGAATTAATGA
GATCATCGGCCAGACAGGCAACCAGAACGCCAAAAGGATCGTGGAAAAAATCAGACTG
CACGACCAGCAGGAGGGCAAGTGCCTGTACTCTCTGGAAAGCATCCCCCTGGAGGACCT
GCTGAACAATCCAAATCACTACGAGGTGGACCACATCATCCCTAGAAGCGTCAGCTTCG
ACAACAGCTACCACAACAAGGTGCTGGTGAAGCAGAGCGAAAACAGTAAGAAATCCAA
CCTGACACCTTACCAGTACTTTAACAGCGGCAAGAGCAAGCTGAGCTACAACCAGTTCA
AGCAGCACATCCTGAACCTGTCCAAATCTCAGGATAGAATCTCCAAGAAAAAGAAGGAA
TACCTGCTGGAGGAGAGAGATATCAACAAGTTCGAAGTCCAAAAGGAGTTCATCAACAG
GAACCTGGTGGACACCCGGTACGCCACAAGAGAGCTGACAAACTACCTGAAAGCCTACT
TCAGCGCTAACAACATGAACGTGAAGGTGAAAACCATCAATGGAAGTTTCACAGACTAC
CTTCGGAAGGTGTGGAAGTTCAAGAAGGAACGGAATCACGGCTACAAGCACCACGCAGA
GGACGCCCTGATTATAGCTAATGCCGATTTCCTGTTCAAAGAAAACAAGAAGCTGAAAG
CCGTGAACAGCGTTCTGGAAAAGCCTGAAATTGAGACCAAGCAACTGGATATACAGGTG
GACAGCGAGGACAACTACTCCGAGATGTTCATCATCCCTAAACAGGTGCAGGACATCAA
AGACTTCAGAAATTTCAAGTACAGCCACAGAGTGGACAAGAAACCCAACCGGCAGCTGA
TCAATGACACCCTGTATAGCACCCGCAAGAAGGATAACAGCACCTACATCGTCCAGACC
ATCAAGGACATCTACGCTAAGGACAACACCACCCTGAAAAAGCAATTTGATAAGTCCCC
CGAAAAGTTTCTGATGTACCAACACGATCCTAGAACCTTCGAAAAGTTGGAGGTGATCAT
GAAGCAATATGCCAACGAGAAGAACCCACTGGCCAAGTACCATGAGGAGACAGGAGAA
TACCTGACCAAGTATTCTAAGAAGAATAACGGCCCCATCGTGAAGTCCCTGAAGTACATT
GGGAACAAACTCGGAAGCCACCTGGACGTGACGCACCAGTTCAAGAGCAGCACCAAGA
AGCTAGTGAAACTGAGCATCAAGAACTACAGATTCGACGTGTACCTGACAGAAAAGGGC
TACAAATTTGTGACCATCGCTTACCTGAATGTGTTCAAAAAGGACAATTATTACTACATC
CCAAAGGACAAGTACCAGGAGCTTAAAGAGAAAAAAAAGATCAAGGATACCGACCAGT
TTATCGCTAGCTTCTACAAGAACGACCTGATTAAGCTGAACGGCGACCTGTACAAGATCA
TCGGCGTGAACTCTGACGACCGGAACATAATAGAGCTGGATTATTATGACATCAAGTAC
AAGGACTACTGCGAGATCAACAACATCAAGGGCGAGCCTAGAATCAAAAAGACCATCG
GGAAGAAAACCGAGTCTATCGAAAAGTTTACAACAGACGTGCTGGGCAACCTGTACCTG
CACAGCACGGAAAAGGCCCCTCAGCTCATCTTCAAGAGAGGCCTG.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7026 (designated herein as sRGN3.3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTSYLKAYFSANNMDVKVKTINGSFTNHLRKVWRFD
KYRNHGYKHHAEDALIIANADFLFKENKKLQNTNKILEKPTIENNTKKVTVEKEEDYNNVFE
TPKLVEDIKQYRDYKFSHRVDKKPNRQLINDTLYSTRMKDEHDYIVQTITDIYGKDNTNLKK
QFNKNPEKFLMYQNDPKTFEKLSIIMKQYSDEKNPLAKYYEETGEYLTKYSKKNNGPIVKKI
KLLGNKVGNHLDVTNKYENSTKKLVKLSIKNYRFDVYLTEKGYKFVTIAYLNVFKKDNYYY
IPKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is encoded by a nucleic acid molecule at least 80%, 85%, 90%, 91%, 92%, 93%, 94%, 95%, 96%, 97%, 98%, 99% or 100% identical to the sequence of SEQ ID NO: 2 (an exemplary nucleic acid molecule encoding sRGN3.3):

AACCAGAAGTTTATCCTGGGCCTGGATATCGGCATCACATCCGTGGGCTACGGCCTGATT
GATTACGAAACCAAGAACATCATTGATGCCGGCGTCCGGCTTTTCCCTGAAGCTAACGTG
GAAAACAATGAGGGCAGACGGAGCAAGAGAGGCAGCAGACGGCTGAAGCGGAGAAGA
ATCCATAGACTCGAACGGGTGAAGCTGCTGCTGACCGAGTACGACCTGATCAACAAGGA
GCAGATCCCCACCAGCAACAACCCATACCAGATCAGAGTGAAAGGCCTTTCTGAGATTC
TGAGCAAGGATGAGCTGGCTATCGCTCTGCTCCACCTGGCCAAGAGAAGGGGAATCCAC
AACGTTGACGTGGCCGCCGATAAGGAAGAGACCGCCAGCGATAGCCTGAGCACCAAGG
ACCAGATCAACAAGAACGCCAAGTTCCTGGAAAGCAGATACGTGTGCGAGCTGCAAAAA
GAGAGACTGGAAAATGAGGGCCATGTGCGGGGCGTTGAGAACAGATTCCTGACCAAAG
ACATCGTCAGAGAGGCCAAGAAAATTATTGACACCCAGATGCAGTACTATCCTGAGATA
GACGAGACCTTTAAGGAAAAGTACATCAGCCTGGTGGAAACAAGAAGAGAATACTTCGA
AGGACCTGGCCAGGGCTCCCCTTTCGGCTGGAACGGCGACCTGAAGAAGTGGTACGAGA
TGCTGATGGGCCACTGCACCTACTTCCCCCAGGAGCTGCGGAGCGTGAAGTACGCTTACA
GCGCCGACCTGTTCAATGCCCTGAACGACCTGAACAATCTTATCATCCAAAGAGATAACA
GCGAGAAATTAGAATACCACGAGAAGTACCACATCATCGAGAATGTGTTCAAGCAAAAG
AAGAAGCCTACCCTGAAGCAGATCGCCAAAGAGATCGGCGTGAACCCTGAGGACATCAA
GGGCTATCGGATCACCAAGTCCGGCACCCCTGAATTCACCAGCTTCAAGCTGTTTCACGA
CCTCAAAAAGGTCGTGAAGGACCACGCCATCCTGGACGACATCGATCTGCTGAATCAGA
TCGCCGAGATCCTGACCATCTACCAGGACAAGGACTCTATCGTGGCCGAGCTTGGACAG
CTGGAGTACCTGATGAGCGAGGCTGACAAGCAGAGCATCAGCGAGCTGACCGGCTACAC
CGGAACCCACAGCCTGTCCCTGAAGTGCATGAACATGATCATCGACGAGCTGTGGCACT
CTAGCATGAACCAGATGGAAGTGTTCACCTACCTGAACATGAGACCTAAGAAGTACGAA
CTGAAGGGTTACCAGAGAATCCCAACCGACATGATCGACGACGCCATCCTGAGCCCCGT
GGTGAAGCGGACCTTTATCCAGAGCATCAATGTGATCAACAAGGTGATCGAGAAATACG
GCATCCCCGAGGACATCATCATCGAACTGGCCAGAGAGAATAACTCTGATGACCGGAAG
AAGTTCATCAACAACCTGCAGAAGAAGAACGAAGCCACCAGAAAGCGCATCAACGAGA
TCATCGGCCAAACAGGGAATCAGAACGCCAAGAGGATCGTGGAAAAGATTCGGCTGCAC
GACCAGCAGGAGGGAAAATGCCTGTACAGCCTGGAAAGCATCCCCCTGGAAGATCTACT
GAACAACCCCAACCACTACGAGGTGGATCACATCATCCCTAGAAGCGTGTCCTTTGACA
ACAGTTACCACAACAAGGTGCTGGTGAAGCAATCCGAGAACTCGAAGAAGAGCAACCTG
ACACCTTACCAGTACTTTAACAGCGGCAAGTCCAAGCTGTCTTATAACCAGTTCAAGCAG
CACATCCTCAACCTGTCAAAGTCTCAGGACAGAATCTCTAAGAAGAAGAAGGAGTATCT
GCTGGAAGAGCGGGACATCAACAAGTTCGAGGTGCAGAAAGAGTTCATTAACAGAAACC
TGGTCGACACCCGGTACGCCACACGCGAACTGACAAGCTACCTGAAGGCCTACTTCTCCG
CCAACAATATGGACGTGAAGGTCAAGACCATCAATGGCAGCTTCACAAATCACCTGAGA
AAGGTCTGGCGGTTCGACAAGTACAGAAACCACGGCTACAAGCACCACGCCGAGGATGC
TCTGATTATCGCCAACGCCGACTTCCTGTTCAAGGAAAACAAAAAACTGCAGAACACCA
ACAAGATCCTGGAAAAACCTACAATCGAGAACAACACAAAGAAAGTGACAGTGGAAAA
AGAGGAAGATTACAACAACGTGTTTGAGACACCTAAGCTGGTTGAGGATATCAAGCAGT
ACCGGGACTATAAGTTTAGCCACAGAGTGGACAAGAAACCTAACAGGCAGCTGATCAAC
GACACTCTGTACAGCACAAGAATGAAAGATGAGCACGATTACATCGTGCAGACCATTAC
CGACATCTACGGCAAGGACAACACCAATCTGAAGAAGCAGTTCAACAAAAATCCCGAGA
AGTTCCTGATGTACCAAAATGACCCTAAGACATTCGAGAAGCTGAGCATCATCATGAAA
CAGTACTCTGATGAGAAGAACCCACTGGCCAAGTACTACGAGGAAACAGGCGAATACCT
GACCAAGTACTCTAAGAAGAACAACGGCCCTATCGTGAAGAAGATCAAACTGCTGGGCA
ACAAAGTGGGAAATCATCTGGATGTGACCAATAAATACGAGAACTCTACAAAGAAACTG
GTGAAGCTGAGCATTAAGAACTACAGATTCGACGTCTACCTGACAGAAAAGGGATACAA
GTTCGTGACCATCGCCTACCTGAACGTGTTCAAGAAAGACAACTACTACTACATCCCAAA
AGACAAGTACCAAGAGTTAAAAGAGAAGAAGAAGATAAAGGATACCGACCAGTTTATC
GCTTCTTTCTACAAGAACGACCTGATCAAGCTGAACGGTGATCTGTACAAAATCATCGGA
GTGAATAGCGATGACAGAAATATCATCGAACTGGATTACTATGACATCAAGTACAAAGA
TTATTGTGAAATCAACAACATCAAGGGAGAGCCCAGAATTAAGAAAACCATCGGCAAGA
AAACAGAGAGCATCGAGAAATTCACCACAGATGTGCTGGGCAACCTGTACCTGCACAGC
ACAGAGAAAGCCCCTCAGCTCATCTTCAAGAGAGGCCTG.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7027 (designated herein as sRGN4):

MNQKFILGLDIGITSVGYGLIDYETKNIIDAGVRLFPEANVENNEGRRSKRGSRRLKRRRIHRL
ERVKKLLEDYNLLDQSQIPQSTNPYAIRVKGLSEALSKDELVIALLHIAKRRGIHNINVSSEDE
DASNELSTKEQINRNNKLLKDKYVCEVQLQRLKEGQIRGEKNRFKTTDILKEIDQLLKVQKD
YHNLDIDFINQYKEIVETRREYFEGPGKGSPYGWEGDPKAWYETLMGHCTYFPDELRSVKY
AYSADLFNALNDLNNLVIQRDGLSKLEYHEKYHIIENVFKQKKKPTLKQIANEINVNPEDIKG
YRITKSGKPEFTSFKLFHDLKKVVKDHAILDDIDLLNQIAEILTIYQDKDSIVAELGQLEYLMS
EADKQSISELTGYTGTHSLSLKCMNMIIDELWHSSMNQMEVFTYLNMRPKKYELKGYQRIPT
DMIDDAILSPVVKRTFIQSINVINKVIEKYGIPEDIIIELARENNSDDRKKFINNLQKKNEATRKR
INEIIGQTGNQNAKRIVEKIRLHDQQEGKCLYSLESIPLEDLLNNPNHYEVDHIIPRSVSFDNSY
HNKVLVKQSENSKKSNLTPYQYFNSGKSKLSYNQFKQHILNLSKSQDRISKKKKEYLLEERDI
NKFEVQKEFINRNLVDTRYATRELTNYLKAYFSANNMNVKVKTINGSFTDYLRKVWKFKKE
RNHGYKHHAEDALIIANADFLFKENKKLKAVNSVLEKPEIETKQLDIQVDSEDNYSEMFIIPK
QVQDIKDFRNFKYSHRVDKKPNRQLINDTLYSTRKKDNSTYIVQTIKDIYAKDNTTLKKQFD
KSPEKFLMYQHDPRTFEKLEVIMKQYANEKNPLAKYHEETGEYLTKYSKKNNGPIVKSLKYI
GNKLGSHLDVTHQFKSSTKKLVKLSIKPYRFDVYLTDKGYKFITISYLDVLKKDNYYYIPEQK
YDKLKLGKAIDKNAKFIASFYKNDLIKLDGEIYKIIGVNSDTRNMIELDLPDIRYKEYCELNNI
KGEPRIKKTIGKKVNSIEKLTTDVLGNVFTNTQYTKPQLLFKRGN.

In some embodiments, the Cas9 comprises an amino acid sequence that is encoded by a nucleic acid molecule at least 80%, 85%, 90%, 91%, 92%, 93%, 94%, 95%, 96%, 97%, 98%, 99% or 100% identical to the sequence of SEQ ID NO: 3 (an exemplary nucleic acid molecule encoding sRGN4):

ATGAATCAAAAGTTTATCCTGGGTCTGGACATCGGCATCACATCTGTGGGCTA
CGGCCTTATCGATTACGAGACCAAGAATATCATTGATGCTGGCGTACGGCTGTTCCCTGA
GGCTAACGTGGAAAACAACGAGGGTAGACGGAGCAAGAGAGGCAGCAGACGGCTGAAA
CGGCGTAGAATCCACCGGCTGGAGAGAGTGAAGAAGTTGCTGGAAGATTACAACTTGCT
GGATCAATCCCAGATCCCCCAGAGCACTAATCCTTATGCTATCCGGGTGAAGGGCCTGTC
TGAAGCCCTGAGCAAAGACGAGCTGGTGATTGCCCTGCTGCACATCGCGAAGAGAAGAG
GCATCCACAATATCAATGTGTCCTCTGAGGATGAGGATGCCAGCAACGAGCTGAGCACT
AAGGAACAGATCAATCGGAACAACAAGCTGCTGAAGGACAAGTACGTGTGTGAAGTGC
AGCTGCAGAGACTGAAGGAAGGCCAGATAAGAGGCGAAAAGAACAGATTCAAGACAAC
AGACATCCTGAAAGAAATCGACCAGCTGCTGAAGGTCCAGAAGGACTACCACAACCTCG
ACATCGATTTCATTAACCAGTACAAGGAAATCGTGGAAACCAGGAGAGAGTACTTCGAG
GGCCCTGGCAAGGGCTCCCCATACGGCTGGGAGGGCGACCCCAAGGCCTGGTACGAAAC
CCTGATGGGCCACTGCACCTACTTCCCCGACGAACTGAGAAGCGTCAAGTACGCCTACA
GCGCCGATCTGTTCAACGCCCTGAACGACCTGAACAACCTGGTGATCCAGCGGGACGGC
CTGAGCAAACTGGAGTACCATGAAAAGTATCATATCATCGAGAACGTGTTCAAGCAGAA
GAAAAAACCTACCCTGAAGCAGATCGCCAACGAGATCAACGTGAACCCTGAGGACATTA
AAGGCTACAGAATCACCAAAAGCGGCAAGCCAGAGTTCACCAGCTTCAAGCTGTTTCAC
GACCTGAAGAAGGTCGTGAAAGACCACGCCATCCTGGACGACATCGATCTGCTTAACCA
GATCGCTGAAATCCTCACAATCTACCAGGACAAGGACTCTATCGTGGCCGAGCTGGGAC
AGCTGGAATACCTGATGAGCGAGGCCGATAAGCAGAGCATCAGCGAGCTGACCGGCTAC
ACCGGAACCCACAGCCTGAGCCTGAAGTGTATGAACATGATCATCGACGAGCTGTGGCA
CAGCTCTATGAACCAGATGGAAGTATTCACCTACCTGAACATGAGACCTAAGAAGTACG
AACTGAAGGGCTATCAGAGAATCCCTACAGACATGATCGACGATGCCATCCTGTCTCCTG
TGGTGAAGAGAACCTTCATCCAGTCTATCAACGTGATCAACAAGGTGATCGAAAAGTAC
GGAATCCCTGAAGATATCATCATCGAACTGGCCAGAGAGAACAACTCCGACGACAGAAA
GAAATTCATCAACAACCTGCAGAAGAAGAATGAGGCCACACGGAAGCGGATTAATGAG
ATCATCGGCCAAACCGGCAACCAGAACGCCAAAAGAATCGTGGAAAAGATCCGGCTGCA
CGATCAGCAGGAGGGCAAATGCCTGTACAGCCTGGAGAGCATCCCCCTGGAGGACCTGC
TCAACAACCCCAACCACTACGAGGTGGATCACATCATCCCAAGATCTGTTAGCTTCGACA
ACAGCTACCACAACAAGGTGCTGGTGAAGCAAAGCGAAAACTCTAAGAAATCTAACCTG
ACACCTTACCAGTACTTTAACAGCGGCAAGTCCAAGCTGTCTTATAACCAGTTTAAGCAG
CACATCCTGAACCTGAGCAAGTCCCAGGATAGAATCAGCAAAAAAAAGAAGGAATACCT
GCTGGAGGAACGCGACATCAATAAATTTGAGGTGCAAAAGGAGTTCATCAACCGGAACC
TGGTGGACACCCGGTACGCCACCAGAGAACTGACCAACTACCTGAAAGCCTACTTCAGC
GCCAACAACATGAACGTGAAGGTGAAAACCATCAACGGAAGCTTCACCGACTACCTGCG
GAAGGTGTGGAAGTTCAAGAAAGAGCGGAATCACGGCTATAAGCACCACGCTGAGGAC
GCTCTGATCATCGCCAATGCCGATTTCCTGTTCAAGGAAAACAAGAAGTTAAAGGCCGTG
AACTCTGTCCTGGAGAAGCCCGAGATCGAGACAAAGCAGCTGGACATCCAAGTGGACTC
AGAGGACAATTACTCTGAGATGTTCATCATCCCCAAGCAGGTGCAGGACATCAAGGATT
TTAGAAATTTCAAGTACAGCCATAGAGTGGACAAGAAGCCCAATAGACAGCTGATCAAC
GATACACTGTACAGCACCAGAAAGAAGGACAACAGCACATATATCGTCCAGACCATCAA
GGACATTTACGCAAAGGATAACACCACACTGAAGAAGCAGTTCGACAAAAGCCCTGAGA
AGTTCCTGATGTACCAACACGACCCTCGGACCTTCGAGAAGTTAGAGGTTATCATGAAAC
AGTACGCCAACGAGAAAAACCCTCTGGCCAAGTACCACGAGGAAACCGGAGAATACCTG
ACAAAATATAGCAAGAAGAACAACGGCCCCATCGTGAAAAGCCTGAAGTACATCGGCA
ACAAGCTGGGCAGCCACCTGGACGTGACCCACCAGTTCAAGAGCAGCACCAAGAAGCTG
GTCAAGCTGAGCATCAAGCCTTATAGGTTCGACGTGTACCTGACAGATAAGGGATACAA
GTTCATCACCATCAGCTACCTGGATGTTCTGAAAAAGGACAATTACTACTACATCCCTGA
GCAGAAGTACGACAAACTCAAGCTGGGCAAGGCCATCGATAAGAATGCCAAGTTCATCG
CATCTTTTTACAAGAACGACCTGATCAAACTGGACGGCGAGATCTACAAGATCATAGGA
GTGAACAGCGACACCAGGAATATGATCGAGCTCGATCTGCCTGACATCAGATACAAGGA
ATACTGCGAGCTGAACAACATCAAGGGAGAGCCTAGAATCAAGAAAACCATCGGCAAG
AAGGTGAACAGCATCGAGAAACTTACAACAGATGTGCTCGGCAACGTGTTCACCAACAC
CCAGTACACCAAGCCACAGCTGCTGTTTAAGCGGGGGAAC.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7028 (designated herein as Staphylococcus hyicus Cas9 or ShyCas9):

MNNYILGLDIGITSVGYGIVDSDTREIKDAGVRLFPEANVDNNEGRRSKRGARRLKR
RRIHRLDRVKHLLAEYDLLDLTNIPKSTNPYQTRVKGLNEKLSKDELVIALLHIAKRRGIHNV
NVMMDDNDSGNELSTKDQLKKNAKALSDKYVCELQLERFEQDYKVRGEKNRFKTEDFVRE
ARKLLETQSKFFEIDQTFIMRYIELIETRREYFEGPGKGSPFGWEGNIKKWFEQMMGHCTYFP
EELRSVKYSYSAELFNALNDLNNLVITRDEDAKLNYGEKFQIIENVFKQKKTPNLKQIAIEIGV
HETEIKGYRVNKSGKPEFTQFKLYHDLKNIFKDPKYLNDIQLMDNIAEIITIYQDAESIIKELNQ
LPELLSEREKEKISALSGYSGTHRLSLKCINLLLDDLWESSLNQMELFTKLNLKPKKIDLSQQH
KIPSKLVDDFILSPVVKRAFIQSIQVVNAIIDKYGLPEDIIIELARENNSDDRRKFLNQLQKQNE
ETRKQVEKVLREYGNDNAKRIVQKIKLHNMQEGKCLYSLKDIPLEDLLRNPHHYEVDHIIPRS
VAFDNSMHNKVLVRADENSKKGNRTPYQYLNSSESSLSYNEFKQHILNLSKTKDRITKKKRE
YLLEERDINKFDVQKEFINRNLVDTRYATRELTSLLKAYFSANNLDVKVKTINGSFTNYLRKV
WKFDKDRNKGYKHHAEDALIIANADFLFKHNKKLRNINKVLDAPSKEVDKKRVTVQSEDEY
NQIFEDTQKAQAIKKFEIRKFSHRVDKKPNRQLINDTLYSTRNIDGIEYVVESIKDIYSVNNDK
VKTKFKKDPHRLLMYRNDPQTFEKFEKVFKQYESEKNPFAKYYEETGEKIRKFSKTGQGPYI
NKIKYLRERLGRHCDVTNKYINSRNKIVQLKIYSYRFDIYQYGNNYKMITISYIDLEQKSNYY
YISREKYEQKKKDKQIDDSYKFIGSFYKNDIINYNGEMYRVIGVNDSEKNKIQLDMIDISIKDY
MELNNIKKTGVIYKTIGKSTTHIEKYTTDILGNLYKAAPPKKPQLIFK.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7029 (designated herein as Staphylococcus microti Cas9 or Smi Cas9):

MEKDYILGLDIGIGSVGYGLIDYDTKSIIDAGVRLFPEANADNNLGRRAKRGARRLKRRRIHR
LERVKSLLSEYKIISGLAPTNNQPYNIRVKGLTEQLTKDELAVALLHIAKRRGIHNVDVAADK
EETASDSLSTKDQINKNAKFLESRYVCELQKERLENEGHVRGVENRFLTKDIVREAKKIIDTQ
MQYYPEIDETFKEKYISLVETRREYYEGPGKGSPYGWDADVKKWYQLMMGHCTYFPVEFRS
VKYAYTADLYNALNDLNNLTIARDDNPKLEYHEKYHIIENVFKQKRNPTLKQIAKEIGVNDI
NISGYRVTKSGKPQFTSFKLFHDLKKVVKDHAILDDIDLLNQIAEILTIYQDKDSIVAELGQLE
YLMSEADKQSISELTGYTGTHSLSLKCMNMIIDELWHSSMNQMEVFTYLNMRPKKYELKGY
QRIPTDMIDDAILSPVVKRSFKQAIGVVNAIIKKYGLPKDIIIELARESNSAEKSRYLRAIQKKN
EKTRERIEAIIKEYGNENAKGLVQKIKLHDAQEGKCLYSLKDIPLEDLLRNPNNYDIDHIIPRS
VSFDDSMHNKVLVRREQNAKKNNQTPYQYLTSGYADIKYSVFKQHVLNLAENKDRMTKKK
REYLLEERNINKYDVQKEFINRNLVDTRYTTRELTTLLKTYFTINNLDVKVKTINGSFTDFLR
KRWGFKKNRDEGYKHHAEDALIIANADYLFKEHKLLKEIKDVSDLAGDERNSNVKDEDQYE
EVFGGYFKIEDIKKYKIKKFSHRVDKKPNRQLINDTIYSTRVKDDKRYLINTLKNLYDKSNGD
LKERMQKDPESLLMYHHDPQTFEKLKIVMSQYENEKNPLAKYFEETGQYLTKYAKHDNGPA
IHKIKYYGNKLVEHLDITKNYHNPQNKVVQLSQKSFRFDVYQTDKGYKFISIAYLTLKNEKN
YYAISQEKYDQLKSEKKISNNAVFIGSFYTSDIIEINNEKFRVIGVNSDKNNLIEVDRIDIRQKEF
IELEEEKKNNRIKVTIGRKTTNIEKFHTDILGNMYKSKRPKAPQLVFKKG.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7030 (designated herein as Staphylococcus pasteuri Cas9 or Spa Cas9):

MKEKYILGLDLGITSVGYGIINFETKKIIDAGVRLFPEANVDNNEGRRSKRGSRRLKRRRIHRL
ERVKLLLTEYDLINKEQIPTSNNPYQIRVKGLSEILSKDELAIALLHLAKRRGIHNINVSSEDED
ASNELSTKEQINRNNKLLKDKYVCEVQLQRLKEGQIRGEKNRFKTTDILKEIDQLLKVQKDY
HNLDIDFINQYKEIVETRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPQFTEFKLYHDLKSIVFDKSILENEAILDQIAEILTIYQDEQSIKEELNKLPEILNEQDK
AEIAKLIGYNGTHRLSLKCIHLINEELWQTSRNQMEIFNYLNIKPNKVDLSEQNKIPKDMVND
FILSPVVKRTFIQSINVINKVIEKYGIPEDIIIELARENNSDDRKKFINNLQKKNEATRKRINEIIG
QTGNQNAKRIVEKIRLHDQQEGKCLYSLESIALMDLLNNPQNYEVDHIIPRSVAFDNSIHNKV
LVKQIENSKKGNRTPYQYLNSSDAKLSYNQFKQHILNLSKSKDRISKKKKDYLLEERDINKFE
VQKEFINRNLVDTRYATRELTSYLKAYFSANNMDVKVKTINGSFTNHLRKVWRFDKYRNHG
YKHHAEDALIIANADFLFKENKKLQNTNKILEKPTIENNTKKVTVEKEEDYNNVFETPKLVED
IKQYRDYKFSHRVDKKPNRQLINDTLYSTRMKDEHDYIVQTITDIYGKDNTNLKKQFNKNPE
KFLMYQNDPKTFEKLSIIMKQYSDEKNPLAKYYEETGEYLTKYSKKNNGPIVKKIKLLGNKV
GNHLDVTNKYENSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYDIKYKDYCEINNIKG
EPRIKKTIGKKTESIEKFTTDVLGNLYLHSTEKAPQLIFKRGL.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7031 (designated herein as Cas12i1):

MSNKEKNASETRKAYTTKMIPRSHDRMKLLGNFMDYLMDGTPIFFELWNQFGGGIDRDIISG
TANKDKISDDLLLAVNWFKVMPINSKPQGVSPSNLANLFQQYSGSEPDIQAQEYFASNFDTE
KHQWKDMRVEYERLLAELQLSRSDMHHDLKLMYKEKCIGLSLSTAHYITSVMFGTGAKNN
RQTKHQFYSKVIQLLEESTQINSVEQLASIILKAGDCDSYRKLRIRCSRKGATPSILKIVQDYEL
GTNHDDEVNVPSLIANLKEKLGRFEYECEWKCMEKIKAFLASKVGPYYLGSYSAMLENALS
PIKGMTTKNCKFVLKQIDAKNDIKYENEPFGKIVEGFFDSPYFESDTNVKWVLHPHHIGESNI
KTLWEDLNAIHSKYEEDIASLSEDKKEKRIKVYQGDVCQTINTYCEEVGKEAKTPLVQLLRY
LYSRKDDIAVDKIIDGITFLSKKHKVEKQKINPVIQKYPSFNFGNNSKLLGKIISPKDKLKHNL
KCNRNQVDNYIWIEIKVLNTKTMRWEKHHYALSSTRFLEEVYYPATSENPPDALAARFRTKT
NGYEGKPALSAEQIEQIRSAPVGLRKVKKRQMRLEAARQQNLLPRYTWGKDFNINICKRGN
NFEVTLATKVKKKKEKNYKVVLGYDANIVRKNTYAAIEAHANGDGVIDYNDLPVKPIESGF
VTVESQVRDKSYDQLSYNGVKLLYCKPHVESRRSFLEKYRNGTMKDNRGNNIQIDFMKDFE
AIADDETSLYYFNMKYCKLLQSSIRNHSSQAKEYREEIFELLRDGKLSVLKLSSLSNLSFVMF
KVAKSLIGTYFGHLLKKPKNSKSDVKAPPITDEDKQKADPEMFALRLALEEKRLNKVKSKKE
VIANKIVAKALELRDKYGPVLIKGENISDTTKKGKKSSTNSFLMDWLARGVANKVKEMVM
MHQGLEFVEVNPNFTSHQDPFVHKNPENTFRARYSRCTPSELTEKNRKEILSFLSDKPSKRPT
NAYYNEGAMAFLATYGLKKNDVLGVSLEKFKQIMANILHQRSEDQLLFPSRGGMFYLATYK
LDADATSVNWNGKQFWVCNADLVAAYNVGLVDIQKDFKKK.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7032 (designated herein as Cas12i2):

MSSAIKSYKSVLRPNERKNQLLKSTIQCLEDGSAFFFKMLQGLFGGITPEIVRFSTEQEKQQQD
IALWCAVNWFRPVSQDSLTHTIASDNLVEKFEEYYGGTASDAIKQYFSASIGESYYWNDCRQ
QYYDLCRELGVEVSDLTHDLEILCREKCLAVATESNQNNSIISVLFGTGEKEDRSVKLRITKKI
LEAISNLKEIPKNVAPIQEIILNVAKATKETFRQVYAGNLGAPSTLEKFIAKDGQKEFDLKKLQ
TDLKKVIRGKSKERDWCCQEELRSYVEQNTIQYDLWAWGEMFNKAHTALKIKSTRNYNFA
KQRLEQFKEIQSLNNLLVVKKLNDFFDSEFFSGEETYTICVHHLGGKDLSKLYKAWEDDPAD
PENAIVVLCDDLKNNFKKEPIRNILRYIFTIRQECSAQDILAAAKYNQQLDRYKSQKANPSVL
GNQGFTWTNAVILPEKAQRNDRPNSLDLRIWLYLKLRHPDGRWKKHHIPFYDTRFFQEIYAA
GNSPVDTCQFRTPRFGYHLPKLTDQTAIRVNKKHVKAAKTEARIRLAIQQGTLPVSNLKITEIS
ATINSKGQVRIPVKFDVGRQKGTLQIGDRFCGYDQNQTASHAYSLWEVVKEGQYHKELGCF
VRFISSGDIVSITENRGNQFDQLSYEGLAYPQYADWRKKASKFVSLWQITKKNKKKEIVTVE
AKEKFDAICKYQPRLYKFNKEYAYLLRDIVRGKSLVELQQIRQEIFRFIEQDCGVTRLGSLSLS
TLETVKAVKGIIYSYFSTALNASKNNPISDEQRKEFDPELFALLEKLELIRTRKKKQKVERIAN
SLIQTCLENNIKFIRGEGDLSTTNNATKKKANSRSMDWLARGVFNKIRQLAPMHNITLFGCGS
LYTSHQDPLVHRNPDKAMKCRWAAIPVKDIGDWVLRKLSQNLRAKNIGTGEYYHQGVKEF
LSHYELQDLEEELLKWRSDRKSNIPCWVLQNRLAEKLGNKEAVVYIPVRGGRIYFATHKVAT
GAVSIVFDQKQVWVCNADHVAAANIALTVKGIGEQSSDEENPDGSRIKLQLTS.

### Modified Guide RNAs

In some embodiments, the guide RNA is chemically modified. A guide RNA comprising one or more modified nucleosides or nucleotides is called a “modified” guide RNA or “chemically modified” guide RNA, to describe the presence of one or more non-naturally and/or naturally occurring components or configurations that are used instead of or in addition to the canonical A, G, C, and U residues. In some embodiments, a modified guide RNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified guide RNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a guide RNA is modified, e.g., all bases have a modified phosphate group, such as a phosphorothioate group. In certain embodiments, all, or substantially all, of the phosphate groups of a guide RNA molecule are replaced with phosphorothioate groups. In some embodiments, modified guide RNAs comprise at least one modified residue at or near the 5′ end of the RNA. In some embodiments, modified guide RNAs comprise at least one modified residue at or near the 3′ end of the RNA.

In some embodiments, the guide RNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified guide RNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the guide RNAs described herein can contain one or more modified nucleosides or nucleotides, e.g., to introduce stability toward intracellular or serum-based nucleases. In some embodiments, the modified guide RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁_₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

Modifications of 2′-O-methyl are encompassed.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

### Ribonucleoprotein Complex

In some embodiments, a composition is encompassed comprising: a) one or more guide RNAs comprising one or more guide sequences from Table 1A (for SaCas9) or Table 1B (for SluCas9) and b) SaCas9 (when combined with a gRNA comprising any one of or combination of SEQ ID Nos: 1000-3081) or SluCas9 (when combined with a gRNA comprising any one of or combination of SEQ ID Nos: 4000-5226), or any of the mutant Cas9 proteins disclosed herein. In some embodiments, the guide RNA together with a Cas9 is called a ribonucleoprotein complex (RNP).

In some embodiments, the disclosure provides for an RNP complex, wherein the guide RNA (e.g., any of the guide RNAs disclosed herein) binds to or is capable of binding to a target sequence in the dystrophin gene.

In some embodiments, chimeric Cas9 (SaCas9 or SluCas9) nucleases are used, where one domain or region of the protein is replaced by a portion of a different protein. In some embodiments, a Cas9 nuclease domain may be replaced with a domain from a different nuclease such as Fok1. In some embodiments, a Cas9 nuclease may be a modified nuclease.

In some embodiments, the Cas9 is modified to contain only one functional nuclease domain. For example, the agent protein may be modified such that one of the nuclease domains is mutated or fully or partially deleted to reduce its nucleic acid cleavage activity.

In some embodiments, a conserved amino acid within a Cas9 protein nuclease domain is substituted to reduce or alter nuclease activity. In some embodiments, a Cas9 nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas9 nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPF1_FRATN)). Further exemplary amino acid substitutions include D10A and N580A (based on the S. aureus Cas9 protein). See, e.g., Friedland et al., 2015, Genome Biol., 16:257.

In some embodiments, the Cas9 lacks cleavase activity. In some embodiments, the Cas9 comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the Cas9 lacking cleavase activity or the dCas DNA-binding polypeptide is a version of a Cas nuclease (e.g., a Cas9 nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the Cas9 comprises one or more heterologous functional domains (e.g., is or comprises a fusion polypeptide).

In some embodiments, the heterologous functional domain may facilitate transport of the Cas9 into the nucleus of a cell. For example, the heterologous functional domain may be a nuclear localization signal (NLS). In some embodiments, the Cas9 may be fused with 1-10 NLS(s). In some embodiments, the Cas9 may be fused with 1-5 NLS(s). In some embodiments, the Cas9 may be fused with 1-3 NLS(s). In some embodiments, the Cas9 may be fused with one NLS. Where one NLS is used, the NLS may be attached at the N-terminus or the C-terminus of the Cas9 sequence, and may be directly fused/attached. In some embodiments, where more than one NLS is used, one or more NLS may be attached at the N-terminus and/or one or more NLS may be attached at the C-terminus. In some embodiments, one or more NLSs are directly attached to the Cas9. In some embodiments, one or more NLSs are attached to the Cas9 by means of a linker. In some embodiments, the linker is between 3-25 amino acids in length. In some embodiments, the linker is between 3-6 amino acids in length. In some embodiments, the linker comprises glycine and serine. In some embodiments, the linker comprises the sequence of GSVD (SEQ ID NO: 550) or GSGS (SEQ ID NO: 551). It may also be inserted within the Cas9 sequence. In other embodiments, the Cas9 may be fused with more than one NLS. In some embodiments, the Cas9 may be fused with 2, 3, 4, or 5 NLSs. In some embodiments, the Cas9 may be fused with two NLSs. In certain circumstances, the two NLSs may be the same (e.g., two SV40 NLSs) or different. In some embodiments, the Cas9 protein is fused with one or more SV40 NLSs. In some embodiments, the SV40 NLS comprises the amino acid sequence of SEQ ID NO: 713 (PKKKRKV). In some embodiments, the Cas9 protein (e.g., the SaCas9 or SluCas9 protein) is fused to one or more nucleoplasmin NLSs. In some embodiments, the Cas protein is fused to one or more c-myc NLSs. In some embodiments, the Cas protein is fused to one or more E1A NLSs. In some embodiments, the Cas protein is fused to one or more BP (bipartite) NLSs. In some embodiments, the nucleoplasmin NLS comprises the amino acid sequence of SEQ ID NO: 714 (KRPAATKKAGQAKKKK). In some embodiments, the Cas9 protein is fused with a c-Myc NLS. In some embodiments, the c-Myc NLS is comprises the amino acid sequence of SEQ ID NO: 942 (PAAKKKKLD) and/or encoded by the nucleic acid sequence of SEQ ID NO: 722 (CCGGCAGCTAAGAAAAAGAAACTGGAT). In some embodiments, the Cas9 is fused to two SV40 NLS sequences linked at the carboxy terminus. In some embodiments, the Cas9 may be fused with two NLSs, one linked at the N-terminus and one at the C-terminus. In some embodiments, the Cas9 may be fused with 3 NLSs. In some embodiments, the Cas9 may be fused with 3 NLSs, two linked at the N-terminus and one linked at the C-terminus. In some embodiments, the Cas9 may be fused with 3 NLSs, one linked at the N-terminus and two linked at the C-terminus. In some embodiments, the Cas9 may be fused with no NLS. In some embodiments, the Cas9 may be fused with one NLS. In some embodiments, the Cas9 may be fused with an NLS on the C-terminus and does not comprise an NLS fused on the N-terminus. In some embodiments, the Cas9 may be fused with an NLS on the N-terminus and does not comprise an NLS fused on the C-terminus. In some embodiments, the Cas9 protein is fused to an SV40 NLS and to a nucleoplasmin NLS. In some embodiments, the Cas9 protein is fused to an SV40 NLS and to a c-Myc NLS. In some embodiments, the SV40 NLS is fused to the C-terminus of the Cas9, while the nucleoplasmin NLS is fused to the N-terminus of the Cas9 protein. In some embodiments, the SV40 NLS is fused to the C-terminus of the Cas9, while the c-Myc NLS is fused to the N-terminus of the Cas9 protein. In some embodiments, the SV40 NLS is fused to the N-terminus of the Cas9, while the nucleoplasmin NLS is fused to the C-terminus of the Cas9 protein. In some embodiments, the SV40 NLS is fused to the N-terminus of the Cas9, while the c-Myc NLS is fused to the C-terminus of the Cas9 protein. In some embodiments, the SV40 NLS is fused to the Cas9 protein by means of a linker. In some embodiments, the SV40 NLS and linker is encoded by the nucleic acid sequence of SEQ ID NO: 723 (ATGATGGCCCCAAAGAAGAAGCGGAAGGTCGGTATCCACGGAGTCCCAGCAGCC). In some embodiments, the nucleoplasmin NLS is fused to the Cas9 protein by means of a linker. In some embodiments, the c-Myc NLS is fused to the Cas9 protein by means of a linker. In some embodiments, an additional domain may be: a) fused to the N- or C-terminus of the Cas protein (e.g., a Cas9 protein), b) fused to the N-terminus of an NLS fused to the N-terminus of a Cas protein, or c) fused to the C-terminus of an NLS fused to the C-terminus of a Cas protein. In some embodiments, an NLS is fused to the N- and/or C-terminus of the Cas protein by means of a linker. In some embodiments, an NLS is fused to the N-terminus of an N-terminally-fused NLS on a Cas protein by means of a linker, and/or an NLS is fused to the C-terminus of a C-terminally fused NLS on a Cas protein by means of a linker. In some embodiments, the linker is GSVD (SEQ ID NO: 550) or GSGS (SEQ ID NO: 551). In some embodiments, the Cas protein comprises a c-Myc NLS fused to the N-terminus of the Cas protein (or to an N-terminally-fused NLS on the Cas protein), optionally by means of a linker. In some embodiments, the Cas protein comprises an SV40 NLS fused to the C-terminus of the Cas protein (or to a C-terminally-fused NLS on the Cas protein), optionally by means of a linker. In some embodiments, the Cas protein comprises a nucleoplasmin NLS fused to the C-terminus of the Cas protein (or to a C-terminally-fused NLS on the Cas protein), optionally by means of a linker. In some embodiments, the Cas protein comprises: a) a c-Myc NLS fused to the N-terminus of the Cas protein, optionally by means of a linker, b) an SV40 NLS fused to the C-terminus of the Cas protein, optionally by means of a linker, and c) a nucleoplasmin NLS fused to the C-terminus of the SV40 NLS, optionally by means of a linker. In some embodiments, the Cas protein comprises: a) a c-Myc NLS fused to the N-terminus of the Cas protein, optionally by means of a linker, b) a nucleoplasmin NLS fused to the C-terminus of the Cas protein, optionally by means of a linker, and c) an SV40 NLS fused to the C-terminus of the nucleoplasmin NLS, optionally by means of a linker. In some embodiments, a c-myc NLS is fused to the N-terminus of the Cas9 and an SV40 NLS and/or nucleoplasmin NLS is fused to the C-terminus of the Cas9. In some embodiments, a c-myc NLS is fused to the N-terminus of the Cas9 (e.g., by means of a linker such as GSVD), an SV40 NLS is fused to the C-terminus of the Cas9 (e.g., by means of a linker such as GSGS), and a nucleoplasmin NLS is fused to the C-terminus of the SV-40 NLS (e.g., by means of a linker such as GSGS).

In some embodiments, the heterologous functional domain may be capable of modifying the intracellular half-life of the Cas9. In some embodiments, the half-life of the Cas9 may be increased. In some embodiments, the half-life of the Cas9 may be reduced. In some embodiments, the heterologous functional domain may be capable of increasing the stability of the Cas9. In some embodiments, the heterologous functional domain may be capable of reducing the stability of the Cas9. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the Cas9 may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the Cas9 to a specific organelle, cell type, tissue, or organ. In some embodiments, the heterologous functional domain may target the Cas9 to muscle.

In further embodiments, the heterologous functional domain may be an effector domain. When the Cas9 is directed to its target sequence, e.g., when a Cas9 is directed to a target sequence by a guide RNA, the effector domain may modify or affect the target sequence. In some embodiments, the effector domain may be chosen from a nucleic acid binding domain or a nuclease domain (e.g., a non-Cas nuclease domain). In some embodiments, the heterologous functional domain is a nuclease, such as a FokI nuclease. See, e.g., U.S. Pat. No. 9,023,649.

In some embodiments, any of the compositions disclosed herein comprising any of the guides and/or endonucleases disclosed herein is sterile and/or substantially pyrogen-free. In particular embodiments, any of the compositions disclosed herein comprise a pharmaceutically acceptable carrier. The phrase “pharmaceutically or pharmacologically acceptable” refers to molecular entities and compositions that do not produce an adverse, allergic, or other untoward reaction when administered to an animal or human. As used herein “pharmaceutically acceptable carrier” includes any and all solvents (e.g., water), dispersion media, coatings, antibacterial and antifungal agents, isotonic and absorption delaying agents, and the like that are physiologically compatible, including pharmaceutically acceptable cell culture media. Pharmaceutically acceptable carriers include sterile aqueous solutions or dispersions and sterile powders for the extemporaneous preparation of sterile injectable solutions or dispersion. In some embodiments, the composition comprises a preservative to prevent the growth of microorganisms.

### Determination of Efficacy of Guide RNAs

In some embodiments, the efficacy of a guide RNA is determined when delivered or expressed together with other components forming an RNP. In some embodiments, the guide RNA is expressed together with a SaCas9 or SluCas9. In some embodiments, the guide RNA is delivered to or expressed in a cell line that already stably expresses an SaCas9 or SluCas9. In some embodiments the guide RNA is delivered to a cell as part of an RNP. In some embodiments, the guide RNA is delivered to a cell along with a nucleic acid (e.g., mRNA) encoding SaCas9 or SluCas9.

In some embodiments, the efficacy of particular guide RNAs is determined based on in vitro models. In some embodiments, the in vitro model is a cell line.

In some embodiments, the efficacy of particular guide RNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses a mutated dystrophin gene. In some embodiments, the in vivo model is a non-human primate, for example cynomolgus monkey.

### III. Methods of Gene Editing and Treating DMD

This disclosure provides methods for gene editing and treating Duchenne Muscular Dystrophy (DMD). In some embodiments, any of the compositions described herein may be administered to a subject in need thereof for use in making a double or single strand break, or excising a portion (e.g., less than about 250 nucleotides) in any one or more of exons 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 46, 47, 48, 49, 52, 54, 55, 56, 57, 58, 59, 60, 61, 62, 63, 64, 65, 66, 67, 68, 69, 70, 71, 72, 73, 74, 75, 76, 77, 78, or 79 of the dystrophin (DMD) gene and to treat DMD. In some embodiments, pairs of guide RNAs described herein, in any of the vector configurations described herein, may be administered to a subject in need thereof to make a single or double-strand break, excise a portion of a DMD exon, and treat DMD. In some embodiments, any of the compositions described herein may be administered to a subject in need thereof for use in treating DMD. In some embodiments, a nucleic acid molecule comprising a first nucleic acid encoding one or more guide RNAs of Table 1A (for SaCas9) or Table 1B (for SluCas9) and a second nucleic acid encoding either SaCas9 or SluCas9 (depending on the guide) is administered to a subject to treat DMD. In some embodiments, a single nucleic acid molecule (which may be a vector, including an AAV vector) comprising a first nucleic acid encoding one or more guide RNAs of Table 1A (for SaCas9) or Table 1B (for SluCas9) and a second nucleic acid encoding either SaCas9 or SluCas9 (depending on the guide) is administered to a subject to treat DMD. In some embodiments, more than one nucleic acid molecules (which may be a vectors, including an AAV vectors) are administered to a subject to treat DMD, wherein a first nucleic acid encoding one or more guide RNAs of Table 1A (for SaCas9) or Table 1B (for SluCas9) and a second nucleic acid encoding either SaCas9 or SluCas9 (depending on the guide) are together on one nucleic acid molecule or separated on different nucleic acid molecules.

In some embodiments, any of the compositions described herein is administered to a subject in need thereof to treat Duchenne Muscular Dystrophy (DMD).

In some embodiments, any of the compositions described herein is administered to a subject in need thereof to induce a double or single strand break in any one or more of exons 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 46, 47, 48, 49, 52, 54, 55, 56, 57, 58, 59, 60, 61, 62, 63, 64, 65, 66, 67, 68, 69, 70, 71, 72, 73, 74, 75, 76, 77, 78, or 79 of the dystrophin gene.

In some embodiments, any of the compositions described herein is administered to a subject in need thereof to delete a portion (e.g., excise a portion) any one of exons 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 46, 47, 48, 49, 52, 54, 55, 56, 57, 58, 59, 60, 61, 62, 63, 64, 65, 66, 67, 68, 69, 70, 71, 72, 73, 74, 75, 76, 77, 78, or 79 of the dystrophin gene. In some embodiments, any of the compositions described herein is administered to a subject to induce multiple double-strand breaks in a single exon (e.g., by administering two different guides that target two different regions in the same exon).

In some embodiments, a method of treating Duchenne Muscular Dystrophy (DMD) is provided, the method comprising delivering to a cell any one of the compositions described herein, wherein the cell comprises a mutation in the dystrophin gene that is known to be associated with DMD.

In some embodiments, methods are provided for treating Duchenne Muscular Dystrophy (DMD), the method comprising delivering to a cell at least one nucleic acid molecule comprising a pair of guide RNAs comprising a first and second spacer sequence, wherein the first and spacer sequences are selected from any two spacer sequences of Table 1A (SEQ ID NOs: 1000-3081); and a nucleic acid encoding a Staphylococcus aureus Cas9 (SaCas9), wherein the nucleic acid encoding the SaCas9 may be on the same or different nucleic acid molecule as the pair of guide RNAs.

In some embodiments, methods are provided for treating Duchenne Muscular Dystrophy (DMD), the method comprising delivering to a cell at least one nucleic acid molecule comprising a pair of guide RNAs comprising a first and second spacer sequence, wherein the first and spacer sequences are selected from any two spacer sequences of Table 1B (SEQ ID NOs: 4000-5226); and a nucleic acid encoding a Staphylococcus lugdunensis (SluCas9), wherein the nucleic acid encoding the SluCas9 may be on the same or different nucleic acid molecule as the pair of guide RNAs.

In some embodiments, a method for treating DMD is provided comprising administering a composition comprising one or more guide RNAs wherein each guide RNA comprises a guide sequence of Table 1A or Table 1B, or comprises a guide sequence comprising at least 16, 17, 18, 19, or 20 contiguous nucleotides of a guide sequence of Table 1A or Table 1B, or is at least 90% identical to guide sequence selected from Table 1A or Table 1B. In each instance, the composition may comprise a nucleic acid encoding the guide RNA(s). In some embodiments, a method for treating DMD is provided comprising administering a composition comprising one or more nucleic acid molecules encoding one or more guide RNAs, wherein each guide RNA comprises a guide sequence of Table 1A or Table 1B, or at least 16, 17, 18, 19, or 20 contiguous nucleotides of a guide sequence of Table 1A or Table 1B, or is at least 90% identical to guide sequence selected from Table 1A or Table 1B. In some embodiments, the method comprises treating DMD by administering at least two guide RNAs and an endonuclease (or a nucleic acid encoding an endonuclease), wherein the at least two guide RNAs target the same exon. In some embodiments, the method comprises treating DMD by administering at least two guide RNAs and an endonuclease (or a nucleic acid encoding an endonuclease), wherein the at least two guide RNAs target different sites in the same exon.

In some embodiments, a method for treating DMD is provided comprising administering a composition comprising a guide RNA wherein the guide RNA comprises a guide sequence of Table 1A or Table 1B, or at least 16, 17, 18, 19, or 20 contiguous nucleotides of a guide sequence of Table 1A or Table 1B, or is at least 90% identical to guide sequence of Table 1A or Table 1B. In each instance, the composition may comprise a nucleic acid encoding the guide RNA.

In some embodiments, a method for treating DMD is provided comprising administering a composition comprising a guide RNA comprising no more than 16, no more than 17, no more than 18, no more than 19, no more than 20, no more than 21, or no more than 22 contiguous nucleotides of a guide sequence of Tables 1A-B, or is at least 90% identical to no more than 16, no more than 17, no more than 18, no more than 19, no more than 20, no more than 21, or no more than 22 contiguous nucleotides of a guide sequence of Tables 1A-B. In each instance, the composition may comprise a nucleic acid encoding the guide RNA.

In some embodiments, a method for treating DMD is provided comprising administering a composition comprising two or more guide RNAs wherein each guide RNA comprises a guide sequence of Table 1A or Table 1B, or at least 16, 17, 18, 19, or 20 contiguous nucleotides of a guide sequence of Table 1A or Table 1B, or is at least 90% identical to guide sequence selected from Table 1A or Table 1B. In each instance, the composition may comprise a nucleic acid encoding the guide RNA.

In some embodiments, a method for treating DMD is provided comprising administering a composition comprising two or more guide RNAs wherein at least one of the two or more guide RNAs, optionally each guide RNA, comprises no more than 16, no more than 17, no more than 18, no more than 19, no more than 20, no more than 21, or no more than 22 contiguous nucleotides of a guide sequence of Tables 1A-B, is at least 90% identical to no more than 16, no more than 17, no more than 18, no more than 19, no more than 20, no more than 21, or no more than 22 contiguous nucleotides of a guide sequence selected from Tables 1A-B. In each instance, the composition may comprise a nucleic acid encoding the guide RNA.

In some embodiments, a method for treating DMD is provided comprising administering a composition comprising a SaCas9 or SluCas9 or one or more nucleic acid molecules encoding a SaCas9 or SluCas9 and a pair of guide RNAs comprising a first guide sequence and a second guide sequence, wherein the first and second guide sequence are selected from any two guide sequences selected from i) SEQ ID NOs: 1000-1353 (for SaCas9); ii) SEQ ID NOs: 1354-3081 (for SaCas9-KKH); and iii) SEQ ID NOs: 4000-5226 (for SluCas9). In some embodiments, the first and second guide sequences target the same exon. In some embodiments, the first and second guide sequences target different sites in the same exon.

In some embodiments, a method for treating DMD is provided comprising administering a composition comprising a Cas protein or a nucleic acid encoding a Cas protein and a pair of guide RNAs, wherein the pair of guide RNAs comprise any two of the SaCas9 guide sequences provided in Table 1A or any two of the SluCas9 guide sequences provided in Table 1B. In some embodiments, the two or more guide sequences target the same exon. In some embodiments, the two or more guide sequences target different sites in the same exon.

In some embodiments, a method for treating DMD is provided comprising administering a Cas protein or a nucleic acid encoding a Cas protein and a composition comprising one or more nucleic acid molecules encoding a pair of guide RNAs, wherein the pair of guide RNAs comprise any two guide sequences selected from i) SEQ ID NOs: 1000-1353 (for SaCas9); ii) SEQ ID NOs: 1354-3081 (for SaCas9-KKH); and iii) SEQ ID NOs: 4000-5226 (for SluCas9). In some embodiments, the two or more guide sequences target the same exon. In some embodiments, the two or more guide sequences target different sites in the same exon.

In some embodiments, a method for treating DMD further comprises administering a nucleic acid encoding an endonuclease. The appropriate endonuclease for use with each of the guide RNAs is provided herein, for example, in Table 1A and Table 1B, column “enzyme.”

In some embodiments, the subject is a mammal. In some embodiments, the subject is human.

For treatment of a subject (e.g., a human), any of the compositions disclosed herein may be administered in a manner compatible with the dosage formulation and in such amount as is therapeutically effective. The compositions may be readily administered in a variety of dosage forms, such as injectable solutions. For parenteral administration in an aqueous solution, for example, the solution will generally be suitably buffered and the liquid diluent first rendered isotonic with, for example, sufficient saline or glucose. Such aqueous solutions may be used, for example, for intravenous, intramuscular, subcutaneous, and/or intraperitoneal administration.

### Combination Therapy

In some embodiments, the invention comprises combination therapies comprising any of the methods or uses described herein together with an additional therapy suitable for ameliorating DMD.

### Delivery of Guide RNA Compositions

The methods and uses disclosed herein may use any suitable approach for delivering the guide RNAs and compositions described herein. Exemplary delivery approaches include vectors, such as viral vectors; lipid nanoparticles; transfection; and electroporation. In some embodiments, vectors or LNPs associated with the single-vector guide RNAs/Cas9's disclosed herein are for use in preparing a medicament for treating DMD.

Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

Electroporation is a well-known means for delivery of cargo, and any electroporation methodology may be used for delivering the single vectors disclosed herein.

In some embodiments, the invention comprises a method for delivering any one of the single vectors disclosed herein to an ex vivo cell, wherein the guide RNA is encoded by a vector, associated with an LNP, or in aqueous solution. In some embodiments, the guide RNA/LNP or guide RNA is also associated with a Cas9 or sequence encoding Cas9 (e.g., in the same vector, LNP, or solution).

In some embodiments, the disclosure provides for methods of using any of the guides, endonucleases, cells, or compositions disclosed herein in research methods. For example, any of the guides or endonucleases disclosed herein may be used alone or in combination in experiments under various parameters (e.g., temperatures, pH, types of cells) or combined with other reagents to evaluate the activity of the guides and/or endonucleases.

### EXAMPLES

The following examples are provided to illustrate certain disclosed embodiments and are not to be construed as limiting the scope of this disclosure in any way.

### Example 1: Exemplary DMD sgRNAs

Guide RNA comprising the guide sequences shown in Table 1A and Table 1B are prepared according to standard methods in a single guide (sgRNA) format. A single AAV vector, or two AAV vectors, is/are prepared that expresses one or more of the guide RNAs and a SaCas9 (for guide sequences having SEQ ID NOs: 1000-3081) or SluCas9 (for guide sequences having SEQ ID NOs: 4000-5226). The AAV vector is administered to cells in vitro to assess the ability of the AAV to express the guide RNA and Cas9, edit the targeted exon (see Tables 1A-B), and thereby treat DMD.

### Example 2: Evaluation of sgRNA Pairs

### A. Materials and Methods

### 1. sgRNA Selection

A subset of SaCas9-KKH or SluCas9 sgRNAs found within the DMD gene is selected for indel frequency and profile evaluation. The selected sgRNAs for pair evaluation are selected from Tables 1A-B and are prepared according to standard methods. The criteria used to select these sgRNAs included their potential to induce exon refraining and or skipping as a pair, in addition to the existence of a mouse, dog and NHP homologue counterpart. This selection includes sgRNAs located within exons 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 46, 47, 48, 49, 52, 54, 55, 56, 57, 58, 59, 60, 61, 62, 63, 64, 65, 66, 67, 68, 69, 70, 71, 72, 73, 74, 75, 76, 77, 78, or 79. The number of predicted off target sites is determined for each sgRNA. The pair of sgRNAs selected from Tables 1A and 1B are selected such that the pair of sgRNAs target different sites in the same exon.

### 2. Transfection of HEK293FT Cells

To evaluate indel frequency and profile, human HEK293FT cell line is used. HEK293FT cells are transfected in 12-well plates with 750 ng plasmid+2.5 μL of Lipofectamine 2000. Three days after transfection, cells are trypsinized and sorted for green fluorescent protein (GFP). GFP-positive cells are sorted directly into lysis buffer, and DNA extraction is performed using the GeneJet Genomic DNA Purification Kit. PCR is then performed on the genomic DNA using DMD exon-specific primers that targeted the relevant cut site.

### 3. Amplicon Deep Sequencing Library Preparation and Data Analysis

Exons are amplified by PCR and the products are used to prepare sequencing libraries using MiSeq reagent kit V3. Indel analysis was performed using CRISPResso2±10-nt quantification window. (See, e.g., Clement et al., Nat Biotechnol. 2019 March; 37(3):224-226). Indel profiling consists of six mutually exclusive indel categories, described below:
NE: non-edited;
Precise deletion (i.e. CleanCut);
RF.+1 (i.e. +1 bp): 1-nucleotide (nt) insertion leading to reframe;
RF.Other (i.e. Reframe): indels other than 1-nt insertion leading to reframe:
Deletion: not extending outside of the refraining window
Insertion: <17-nt (i.e., <6 amino acids);
Exon skipping: indels that disrupt the ±6-nt window of the exon/intron boundaries leading to potential exon skipping (outcome requiring validation):
The indel has >=9-nt overlap with the splicing window (to disrupt the GT/AG splicing sites)
OE: Other indels.

### 4. AAV Configurations for the Dual Cut Single Vector Candidates

A combination of promoter orientations, promoter configurations, NLSs and scaffolds are selected for generating AAV plasmids and evaluation on sgRNA transgene expression, AAV manufacturability, and editing efficiency in vitro and in vivo. AAV plasmid configurations are listed in Table 4.

TABLE 4

| Pol III | | | | | | | |
|---|---|---|---|---|---|---|---|
| Promoter | Orientation | Configuration | NLS1 | Endonuclease | NLS2 | NLS3 | Scaffold |
| hU6c:hU6c | ← ⇒ → | hU6c-Cas9- | c-Myc- | SluCas9 | SV-40-GSGS | Nucleoplasmin- | V5 |
| | | hU6c | GSVD | | | GSGS | |
| | ← ⇒ → | hU6c-Cas9- | c-Myc- | SaCas9-KKH | SV-40-GSGS | Nucleoplasmin- | V2 |
| | | hU6c | GSVD | | | GSGS | |
| | ← ⇒ → | hU6c-Cas9- | c-Myc- | SluCas9 | SV-40-MCS- | Nucleoplasmin- | V5 |
| | | hU6c | GSVD | | GSGS | GSGS | |
| | ← ⇒ → | hU6c-Cas9- | c-Myc- | SaCas9-KKH | SV-40-MCS- | Nucleoplasmin- | V2 |
| | | hU6c | GSVD | | GSGS | GSGS | |
| hU6:7SK2 | ← ⇒ → | hU6c-Cas9- | c-Myc- | SluCas9 | SV-40-GSGS | Nucleoplasmin- | V5 |
| | | 7SK2 | GSVD | | | GSGS | |
| | ← ⇒ → | hU6c-Cas9- | c-Myc- | SaCas9-KKH | SV-40-GSGS | Nucleoplasmin- | V2 |
| | | 7SK2 | GSVD | | | GSGS | |
| | ← ⇒ → | hU6c-Cas9- | c-Myc- | SluCas9 | SV-40-MCS- | Nucleoplasmin- | V5 |
| | | 7SK2 | GSVD | | GSGS | GSGS | |
| | ← ⇒ → | hU6c-Cas9- | c-Myc- | SaCas9-KKH | SV-40-MCS- | Nucleoplasmin- | V2 |
| | | 7SK2 | GSVD | | GSGS | GSGS | |
| hU6c:H1m | ← ⇒ → | hU6c-Cas9- | c-Myc- | SluCas9 | SV-40-GSGS | Nucleoplasmin- | V5 |
| | | H1m | GSVD | | | GSGS | |
| | ← ⇒ → | hU6c-Cas9- | c-Myc- | SaCas9-KKH | SV-40-GSGS | Nucleoplasmin- | V2 |
| | | H1m | GSVD | | | GSGS | |
| 7SK2:H1m | ← ⇒ → | 7SK2-Cas9- | SV-40-GS | SluCas9 | Nucleoplasmin | N/A | V5 |
| | | H1m | | | | | |
| | ← ⇒ → | 7SK2-Cas9- | SV-40-GS | SaCas9-KKH | Nucleoplasmin | N/A | V2 |
| | | H1m | | | | | |
| | ← ⇒ → | 7SK2-Cas9- | SV-40 (+) | SluCas9 | Nucleoplasmin | N/A | V5 |
| | | H1m | | | | | |
| | ← ⇒ → | 7SK2-Cas9- | SV-40 (+) | SaCas9-KKH | Nucleoplasmin | N/A | V2 |
| | | H1m | | | | | |
| | ← ⇒ → | 7SK2-Cas9- | c-Myc- | SluCas9 | SV-40-MCS- | Nucleoplasmin- | V5 |
| | | H1m | GSVD | | GSGS | GSGS | |
| | ← ⇒ → | 7SK2-Cas9- | c-Myc- | SaCas9-KKH | SV-40-MCS | Nucleoplasmin- | V2 |
| | | H1m | GSVD | | GSGS | GSGS | |
| | ← ⇒ → | 7SK2-Cas9- | c-Myc- | SluCas9 | SV-40-MCS | Nucleoplasmin- | V2 |
| | | H1m | GSVD | | GSGS | GSGS | |
| H1m:M11 | ← ⇒ → | H1m-Cas9- | c-Myc- | SluCas9 | SV-40-MCS- | Nucleoplasmin- | V5 |
| | | M11 | GSVD | | GSGS | GSGS | |
| | ← ⇒ → | Hlm-Cas9- | c-Myc- | SaCas9-KKH | SV-40-MCS- | Nucleoplasmin- | V5 |
| | | M11 | GSVD | | GSGS | GSGS | |

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

What is claimed is:

1. A composition comprising one or more guide RNAs or a nucleic acid encoding one or more guide RNAs, wherein the one or more guide RNAs comprise i) a guide sequence of Table 1A or Table 1B; ii) at least 16, 17, 18, 19, or 20 contiguous nucleotides of a guide sequence of Table 1A or Table 1B; iii) a guide sequence that is at least 90% identical to a guide sequence of Table 1A or Table 1B; optionally further comprising a SaCas9 or a nucleic acid encoding a SaCas9 (for SEQ ID NOs: 1000-3081 in Table 1A) or a SluCas9 or a nucleic acid encoding a SluCas9 (for SEQ ID NOs 4000-5226 in Table 1B).

2. A composition comprising a pair of guide RNAs or a nucleic acid encoding the pair of guide RNAs, wherein the pair of guide RNAs comprise a first and a second guide RNA, wherein the first and the second guide RNAs are selected from any two guide RNAs of Table 1A or any two guide RNAs of Table 1B.

3. A composition comprising: one or more nucleic acid molecules encoding
a. a SaCas9 or SluCas9; and
b. a first guide RNA and a second guide RNA, wherein the first and second guide RNAs are selected from any two guide RNAs of Table 1A or any two guide RNAs of Table 1B.

4. A composition comprising:
a. a single nucleic acid molecule comprising:
i. a nucleic acid encoding Staphylococcus aureus Cas9 (SaCas9) or Staphylococcus lugdunensis (SluCas9) and at least one, at least two, or at least three guide RNAs; or
ii. a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or
iii. a nucleic acid encoding Staphylococcus aureus Cas9 (SaCas9) or Staphylococcus lugdunensis (SluCas9) and 1, 2, or 3 guide RNAs;
wherein each guide RNA comprises at least one guide sequence of Table 1A or Table 1B; or
b. two nucleic acid molecules comprising:
i. a first nucleic acid encoding Staphylococcus aureus Cas9 (SaCas9) or Staphylococcus lugdunensis (SluCas9); and
a second nucleic acid that does not encode a SaCas9 or SluCas9 and encodes any one of the following:
1. at least one, at least two, at least three, at least four, at least five, or at least six guide RNAs; or
2. from one to n guide RNAs, wherein n is no more than the maximum number of guide RNAs that can be expressed from said nucleic acid; or
3. from one to six guide RNAs; or
ii. a first nucleic acid encoding Staphylococcus aureus Cas9 (SaCas9) or Staphylococcus lugdunensis (SluCas9) and
1. at least one, at least two, or at least three guide RNAs; or
2. from one to n guide RNAs, wherein n is no more than the maximum number of guide RNAs that can be expressed from said nucleic acid; or
3. 1, 2, or 3 guide RNAs; and
a second nucleic acid that does not encode a SaCas9 or SluCas9, optionally wherein the second nucleic acid comprises any one of the following:
1. at least one, at least two, at least three, at least four, at least five, or at least six guide RNAs; or
2. from one to n guide RNAs, wherein n is no more than the maximum number of guide RNAs that can be expressed from said nucleic acid; or
3. from one to six guide RNAs; or
iii. a first nucleic acid encoding Staphylococcus aureus Cas9 (SaCas9) or Staphylococcus lugdunensis (SluCas9) and at least one, at least two, or at least three guide RNAs; and
a second nucleic acid that does not encode a SaCas9 or SluCas9 and encodes from one to six guide RNAs; or
iv. a first nucleic acid encoding Staphylococcus aureus Cas9 (SaCas9) or Staphylococcus lugdunensis (SluCas9) and at least two guide RNAs, wherein a first guide RNA of the at least two guide RNAs binds upstream of a target sequence and a second guide RNA of the at least two guide RNAs binds downstream of the target sequence; and
a second nucleic acid that does not encode a SaCas9 or SluCas9 and encodes at least one additional copy of each of the guide RNAs encoded in the first nucleic acid;
wherein each guide RNA comprises at least one guide sequence of Table 1A (for SaCas9) or Table 1B (for SluCas9).

5. The composition of any one of claims 1-4, comprising a pair of guide RNAs, wherein the pair of guide RNAs is capable of excising a DNA fragment from the DMD gene; wherein the DNA fragment is between 5-250 nucleotides in length.

6. The composition of claim 5, wherein the excised DNA fragment does not comprise an entire exon of the DMD gene.

7. A composition comprising a single nucleic acid molecule encoding a pair of guide RNAs and a Cas9, wherein the single nucleic acid molecule comprises:
a. a first nucleic acid encoding the pair of guide RNAs, wherein the pair of guide RNAs comprises any two guide RNAs of SEQ ID NOs: 1000-3081; and a second nucleic acid encoding a Staphylococcus aureus Cas9 (SaCas9); or
b. a first nucleic acid encoding a pair of guide RNAs, wherein the pair of guide RNAs comprises any two guide RNAs of SEQ ID NOs: 4000-5226; and a second nucleic acid encoding a Staphylococcus lugdunensis (SluCas9).

8. A composition comprising one or more nucleic acid molecules encoding a Staphylococcus aureus Cas9 (SaCas9) and a first and a second guide RNA, wherein the first and the second guide RNA target different sequences in a DMD gene, wherein the first guide RNA comprises a sequence that is at least 90% identical to a first sequence selected from a guide RNA sequence of Table 1A, and the second guide RNA comprises a sequence that is at least 90% identical to a second sequence selected from a guide RNA sequence of Table 1A.

9. A composition comprising one or more nucleic acid molecules encoding a Staphylococcus lugdunensis (SluCas9) and a first and a second guide RNA, wherein the first and second guide RNA target different sequences in a DMD gene, wherein the first guide RNA comprises a sequence that is at least 90% identical to a first sequence selected from a guide RNA sequence of Table 1B, and the second guide RNA comprises a sequence that is at least 90% identical to a second sequence selected from a guide RNA sequence of Table 1B.

10. The composition of any one of claims 5-9, comprising at least two guide RNAs, wherein once expressed in vitro or in vivo in combination with an RNA-guided endonuclease, the guide RNAs excise a portion of the exon, wherein the size of the excised portion of the exon is between 5 and 250 nucleotides in length.

11. The composition of any one of the preceding claims, comprising at least two guide RNAs, wherein once expressed in vitro or in vivo in combination with an RNA-guided endonuclease, the at least two guide RNAs excise a portion of the DNA fragment from the DMD gene, wherein the size of the excised portion of the DNA fragment from the DMD gene is between 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, and 50 and 75 nucleotides.

12. The composition of any one of claims 5-11, comprising at least two guide RNAs, wherein once expressed in vitro or in vivo in combination with an RNA-guided endonuclease, the at least two guide RNAs excise a portion of the DNA fragment from the DMD gene, wherein the size of the excised portion of the exon is between 8 and 167 nucleotides.

13. The composition of any one of the preceding claims, wherein the guide RNA is an sgRNA.

14. The composition of any one of the preceding claims, wherein the guide RNA is modified.

15. The composition of any one of the preceding claims, wherein the one or more guide RNAs or nucleic acids are in a vector.

16. The composition of claim 15, wherein the vector is a viral vector.

17. The composition of claim 16, wherein the viral vector is an AAV vector.

18. The composition of claim 17, wherein the AAV vector is an AAV9 vector.

19. The composition of any one of the preceding claims, wherein the promoter for the one or more guide RNAs is hU6c.

20. The composition of any one of the preceding claims, wherein if the one or more guide RNAs is a guide RNA for SaCas9, the one or more guide RNAs comprise a scaffold comprising the sequence of SEQ ID NO: 504.

21. The composition of any one of the preceding claims, wherein if the one or more guide RNAs is a guide RNA for SluCas9, the one or more guide RNAs comprise a scaffold comprising the sequence of SEQ ID NO: 901.

22. The composition of any one of the preceding claims, wherein the one or more guide RNAs are in an AAV vector, wherein the vector comprises from 5′ to 3′ with respect to the plus strand: the reverse complement of a first guide RNA scaffold sequence; the reverse complement of a nucleic acid encoding a first sgRNA guide sequence; the reverse complement of a promoter for expression of the nucleic acid encoding the first sgRNA guide sequence; a promoter (e.g., CK8e) for expression of a nucleic acid encoding SaCas9, SluCas9, or a sRGN; a nucleic acid encoding a SaCas9, SluCas9 or a sRGN; a polyadenylation sequence; a promoter for expression of a second sgRNA guide sequence in the same direction as the promoter for SaCas9, SluCas9, or the sRGN; a second sgRNA guide sequence; and a second sgRNA scaffold sequence.

23. The composition of any one of the preceding claims, wherein the composition comprises a pair of guide RNAs, wherein the first and the second guide RNAs of the pair of guide RNAs target the same exon in the dystrophin gene.

24. The composition of claim 23, wherein the first guide RNA and the second guide RNA are not the same.

25. The composition of claim 23, wherein the first guide RNA targets a different site in the same exon targeted by the second guide RNA.

26. The composition of claim 23, wherein the exon is selected from the group consisting of exons: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 46, 47, 48, 49, 52, 54, 55, 56, 57, 58, 59, 60, 61, 62, 63, 64, 65, 66, 67, 68, 69, 70, 71, 72, 73, 74, 75, 76, 77, 78, or 79 of the dystrophin gene.

27. The composition of any one of claims 1-26, wherein the one or more guide RNA sequences, or the first and/or second guide RNA sequence of the pair of guide RNAs, is no more than 16, no more than 17, no more than 18, no more than 19, no more than 20, no more than 21, or no more than 22 nucleotides in length.

28. A method of treating Duchenne Muscular Dystrophy (DMD), the method comprising delivering to a cell a composition of any one of claims 1-27.

29. A method of excising a portion of the DMD gene, the method comprising delivering to a cell a composition of any one of claims 1-27, wherein the size of the excised portion is less than about 250 nucleotides.
